(19) United States
(12) Patent Application Publication
(10) Pub. No.: US 2024/0058271 A1
(43) Pub. Date: Feb. 22, 2024

(54) EXTRACELLULAR VESICLE PREPARATIONS

(71) Applicants: Kevin HUYNH, Cambridge, MA (US); Laura JACKSON, Cambridge, MA (US); Maria SIZOVA, Cambridge, MA (US); Evelo Biosciences, Inc., Cambridge, MA (US)

(72) Inventors: Derek Dorman, Billerica, MA (US); Kevin Huynh, Cambridge, MA (US); Laura Jackson, Boston, MA (US); Collin McKenna, North Reading, MA (US); Maria Sizova, Roslindale, MA (US); Bill Wang, Billerica, MA (US)

(21) Appl. No.: 18/267,017

(22) PCT Filed: Dec. 14, 2021
(86) PCT No.: PCT/US2021/063266

§ 371 Date: Jun. 13, 2023

Related U.S. Application Data
(60) Provisional application No. 63/196,992, filed on Jun. 4, 2021.
(60) Provisional application No. 63/125,177, filed on Dec. 14, 2020.

(51) Int. Cl. A61K 9/16; C12N 1/20; A61K 35/74

(52) U.S. Cl. CPC A61K 9/16; C12N 1/205; A61K 35/74

(57) ABSTRACT

Provided herein are solutions and dried forms of extracellular vesicles (EVs) that are useful as therapeutic agents, and therapeutic compositions thereof.

### CROSS-REFERENCE TO RELATED APPLICATIONS

This application claims the benefit of the following U.S. Provisional Application Nos. 63/125,177, filed Dec. 14, 2020, and 63/196,992, filed Jun. 4, 2021, the entire contents of each are incorporated herein by reference.

### SUMMARY

Therapeutic compositions comprising extracellular vesicles (EVs), such as EVs obtained from bacteria, have therapeutic effects and are useful for the treatment and/or prevention of disease and/or health disorders. As described herein, EVs from bacteria are prepared as solutions and as dried forms. In some embodiments, the solutions and dried forms are for use in preparing therapeutic compositions comprising EVs. In some embodiments, the dried forms comprising EVs described herein (for example, prepared using the excipients and/or methods described herein) have a moisture content of below about 6% upon completion of drying. In some embodiments, dried forms having a moisture content below about 6% are better suited for downstream processing, In some embodiments, dried forms having a moisture content below about 6% have improved stability. In some embodiments, the solutions comprising the EVs also comprise an excipient that contains a bulking agent, and optionally comprises one or more additional ingredients, such as a lyoprotectant. In some embodiments, the solutions comprising the EVs also comprise an excipient that contains a lyoprotectant, and optionally comprises one or more additional ingredients, such as a bulking agent. In some embodiments, the dried forms comprising the EVs also comprise an excipient that contains a bulking agent, and that optionally comprises one or more additional ingredients, such as a lyoprotectant. In some embodiments, the dried forms comprising the EVs also comprise an excipient that contains a lyoprotectant, and optionally comprise one or more additional ingredients, such as a bulking agent.

Bulking agents and/or lyoprotectants are used when preparing extracellular vesicles (EVs) for drying, such as freeze drying and spray drying. In some embodiments, bulking agents, including but not limited to sucrose, mannitol, polyethylene glycol (PEG, such as PEG 6000), cyclodextrin, maltodextrin, and dextran (such as dextran 40k), make dried forms (such as powders and/or lyophilates) easier to handle after drying. In some embodiments, bulking agents improve the properties of a dried form. In some embodiments, lyoprotectants, including but not limited to trehalose, sucrose, and lactose protect the EVs during drying, such as freeze-drying or spray drying. In some embodiments, the excipient functions to decrease drying cycle time. In some embodiments, the excipient functions to maintain therapeutic efficacy of the EVs.

In some embodiments, extracellular vesicles (EVs), such as EVs obtained from bacteria, have therapeutic effects and are useful for the treatment and/or prevention of disease and/or health disorders. In some embodiments, therapeutic compositions of the solutions and dried forms containing EVs are prepared.

In some aspects, the disclosure provides a lyophilate comprising extracellular vesicles (EVs) from bacteria, wherein the lyophilate has a moisture content (e.g., as determined by the Karl Fischer method (also referred to herein as “Karl Fischer”)) of below about 6%.

In some embodiments, the lyophilate has a moisture content (e.g., as determined by the Karl Fischer method) of below about 5%.

In some embodiments, the lyophilate has a moisture content (e.g., as determined by the Karl Fischer method) of below about 4%.

In some embodiments, the lyophilate has a moisture content (e.g., as determined by the Karl Fischer method) of between about 1% to about 4%.

In some embodiments, the lyophilate has a moisture content (e.g., as determined by the Karl Fischer method) of between about 2% to about 3%.

In some aspects, the disclosure provides a lyophilate comprising extracellular vesicles (EVs) from bacteria, wherein the lyophilate has a particle numeration of about 6.7e8 to about 2.55e10 particles/mg lyophilate.

In some aspects, the disclosure provides a lyophilate comprising extracellular vesicles (EVs) from bacteria, wherein the lyophilate has a particle numeration of about 6.7e8 to about 2.89e10 particles/mg lyophilate.

In some aspects, the disclosure provides a lyophilate comprising extracellular vesicles (EVs) from bacteria, wherein the particles have a charge of about −29.2 to about +2.67 mV, as measured by DLS of the charge of the most dominant DLS integrated peak of particles.

In some aspects, the disclosure provides a lyophilate comprising extracellular vesicles (EVs) from bacteria, wherein the particles have a charge of about −0.929 to about −24.80 mV, as measured by DLS of the charge of total particles.

In some aspects, the disclosure provides a lyophilate comprising extracellular vesicles (EVs) from bacteria, wherein the particles have a hydrodynamic diameter (Z average, Zₐᵥₑ) of about 101 nm to about 752 nm.

In some aspects, the disclosure provides a lyophilate comprising extracellular vesicles (EVs) from bacteria, wherein the particles have a mean size of the most dominant DLS integrated peak of between about 25.55 nm to about 458.9 nm or between about 25.55 nm to about 157.40 nm.

In some aspects, the disclosure provides a lyophilate comprising extracellular vesicles (EVs) from bacteria and an excipient, wherein the excipient comprises about 95% to about 99% of the total mass of the lyophilate.

In some aspects, the disclosure provides a lyophilate comprising extracellular vesicles (EVs) from bacteria and an excipient, wherein the EVs make up about 2% to about 6% of the total mass of the lyophilate.

In some embodiments of the lyophilate provided herein, the lyophilate comprises a lyophilized powder.

In some embodiments of the lyophilate provided herein, the lyophilate comprises a lyophilized cake.

In some embodiments of the lyophilate provided herein, the lyophilate comprises EVs from a bacterial strain that is associated with small intestinal mucus.

In some embodiments of the lyophilate provided herein, the lyophilate comprises EVs from anaerobic bacteria.

In some embodiments of the lyophilate provided herein, the anaerobic bacteria are obligate anaerobes.

In some embodiments of the lyophilate provided herein, the anaerobic bacteria are facultative anaerobes.

In some embodiments of the lyophilate provided herein, the anaerobic bacteria are aerotolerant anaerobes.

In some embodiments of the lyophilate provided herein, the lyophilate comprises EVs from monoderm bacteria.

In some embodiments of the lyophilate provided herein, the lyophilate comprises EVs from diderm bacteria.

In some embodiments of the lyophilate provided herein, the lyophilate comprises EVs from Gram negative bacteria.

In some embodiments of the lyophilate provided herein, the lyophilate comprises EVs from bacteria of the family: Prevotellaceae; Veillonellaceae; Tannerellaceae; Rikenellaceae; Selenomonadaceae; Sporomusaceae; Synergistaceae; Christensenellaceae; or Akkermaniaceae.

In some embodiments of the lyophilate provided herein, the lyophilate comprises EVs from Gram positive bacteria.

In some embodiments of the lyophilate provided herein, the lyophilate comprises EVs from bacteria of the family Oscillospiraceae; Clostridiaceae; or Lachnospiraceae.

In some embodiments of the lyophilate provided herein, the lyophilate comprises EVs from bacteria of the genus Prevotella.

In some embodiments of the lyophilate provided herein, the lyophilate comprises EVs from bacteria of the genus Veillonella.

In some embodiments of the lyophilate provided herein, the lyophilate comprises EVs from bacteria of the genus Parabacteroides.

In some embodiments of the lyophilate provided herein, the lyophilate comprises EVs from bacteria of the Oscillospiraceae family.

In some embodiments of the lyophilate provided herein, the lyophilate comprises EVs from bacteria of the Tannerellaceae family.

In some embodiments of the lyophilate provided herein, the lyophilate comprises EVs from bacteria of the Prevotellaceae family.

In some embodiments of the lyophilate provided herein, the lyophilate comprises EVs from bacteria of the Veillonellaceae family.

In some embodiments of the lyophilate provided herein, the lyophilate comprises EVs from bacteria of the species Veillonella parvula.

In some embodiments of the lyophilate provided herein, the lyophilate comprises EVs from bacteria of the species Fournierella massiliensis.

In some aspects, the disclosure provides a powder comprising extracellular vesicles (EVs) from bacteria, wherein the powder has a moisture content (e.g., as determined by the Karl Fischer method) of below about 6%.

In some embodiments, the powder has a moisture content (e.g., as determined by the Karl Fischer method) of below about 5%.

In some embodiments, the powder has a moisture content (e.g., as determined by the Karl Fischer method) of below about 4%.

In some embodiments, the powder has a moisture content (e.g., as determined by the Karl Fischer method) of between about 1% to about 4%.

In some embodiments, the powder has a moisture content (e.g., as determined by the Karl Fischer method) of between about 2% to about 3%.

In some aspects, the disclosure provides a powder comprising extracellular vesicles (EVs) from bacteria, wherein the powder has a particle numeration of about 6.7e8 to about 2.55e10 particles/mg powder.

In some aspects, the disclosure provides a powder comprising extracellular vesicles (EVs) from bacteria, wherein the powder has a particle numeration of about 6.7e8 to about 2.89e10 particles/mg powder.

In some aspects, the disclosure provides a powder comprising extracellular vesicles (EVs) from bacteria, wherein the particles have a charge of about −29.2 to about +2.67 mV, as measured by DLS of the charge of the most dominant DLS integrated peak of particles.

In some aspects, the disclosure provides a powder comprising extracellular vesicles (EVs) from bacteria, wherein the particles have a charge of about −0.929 to about −24.80 mV, as measured by DLS of the charge of total particles.

In some aspects, the disclosure provides a powder comprising extracellular vesicles (EVs) from bacteria, wherein the particles have a hydrodynamic diameter (Z average, Zₐᵥₑ) of about 101 nm to about 752 nm.

In some aspects, the disclosure provides a powder comprising extracellular vesicles (EVs) from bacteria, wherein the particles have a mean size of the most dominant DLS integrated peak of between about 25.55 nm to about 458.9 nm or between about 25.55 nm to about 157.40 nm.

In some aspects, the disclosure provides a powder comprising extracellular vesicles (EVs) from bacteria and an excipient, wherein the excipient comprises about 95% to about 99% of the total mass of the powder.

In some aspects, the disclosure provides a powder comprising extracellular vesicles (EVs) from bacteria and an excipient, wherein the EVs make up about 2% to about 6% of the total mass of the powder.

In some embodiments of the powder provided herein, the powder comprises a lyophilized powder.

In some embodiments of the powder provided herein, the powder comprises a spray-dried powder.

In some embodiments of the powder provided herein, the powder comprises EVs from a bacterial strain that is associated with small intestinal mucus.

In some embodiments of the powder provided herein, the powder comprises EVs from anaerobic bacteria.

In some embodiments of the powder provided herein, the anaerobic bacteria are obligate anaerobes.

In some embodiments of the powder provided herein, the anaerobic bacteria are facultative anaerobes.

In some embodiments of the powder provided herein, the anaerobic bacteria are aerotolerant anaerobes.

In some embodiments of the powder provided herein, the powder comprises EVs from monoderm bacteria.

In some embodiments of the powder provided herein, the powder comprises EVs from diderm bacteria.

In some embodiments of the powder provided herein, the powder comprises EVs from Gram negative bacteria.

In some embodiments of the powder provided herein, the powder comprises EVs from bacteria of the family: Prevotellaceae; Veillonellaceae; Tannerellaceae; Rikenellaceae; Selenomonadaceae; Sporomusaceae; Synergistaceae; Christensenellaceae; or Akkermaniaceae.

In some embodiments of the powder provided herein, the powder comprises EVs from Gram positive bacteria.

In some embodiments of the powder provided herein, the powder comprises EVs from bacteria of the family Oscillospiraceae; Clostridiaceae; or Lachnospiraceae.

In some embodiments of the powder provided herein, the powder comprises EVs from bacteria of the genus Prevotella.

In some embodiments of the powder provided herein, the powder comprises EVs from bacteria of the genus Veillonella.

In some embodiments of the powder provided herein, the powder comprises EVs from bacteria of the genus Parabacteroides.

In some embodiments of the powder provided herein, the powder comprises EVs from bacteria of the Oscillospiraceae family.

In some embodiments of the powder provided herein, the powder comprises EVs from bacteria of the Tannerellaceae family.

In some embodiments of the powder provided herein, the powder comprises EVs from bacteria of the Prevotellaceae family.

In some embodiments of the powder provided herein, the powder comprises EVs from bacteria of the Veillonellaceae family.

In some embodiments of the powder provided herein, the powder comprises EVs from bacteria of the species Veillonella parvula.

In some embodiments of the powder provided herein, the powder comprises EVs from bacteria of the species Fournierella massiliensis.

In some aspects, the disclosure provides a dried form comprising extracellular vesicles (EVs) from bacteria, wherein the dried form has a moisture content (e.g., as determined by the Karl Fischer method) of below about 6%.

In some embodiments, the dried form provided herein has a moisture content (e.g., as determined by the Karl Fischer method) of below about 5%.

In some embodiments, the dried form provided herein has a moisture content (e.g., as determined by the Karl Fischer method) of below about 4%.

In some embodiments, the dried form provided herein has a moisture content (e.g., as determined by the Karl Fischer method) of between about 1% to about 4%.

In some embodiments, the dried form provided herein has a moisture content (e.g., as determined by the Karl Fischer method) of between about 2% to about 3%.

In some aspects, the disclosure provides a dried form comprising extracellular vesicles (EVs) from bacteria, wherein the dried form has a particle numeration of about 6.7e8 to about 2.55e10 particles/mg dried form.

In some aspects, the disclosure provides a dried form comprising extracellular vesicles (EVs) from bacteria, wherein the dried form has a particle numeration of about 6.7e8 to about 2.89e10 particles/mg dried form.

In some aspects, the disclosure provides a dried form comprising extracellular vesicles (EVs) from bacteria, wherein the particles have a charge of about −29.2 to about +2.67 mV, as measured by DLS of the charge of the most dominant DLS integrated peak of particles.

In some aspects, the disclosure provides a dried form comprising extracellular vesicles (EVs) from bacteria, wherein the particles have a charge of about −0.929 to about −24.80 mV, as measured by DLS of the charge of total particles.

In some aspects, the disclosure provides a dried form comprising extracellular vesicles (EVs) from bacteria, wherein the particles have a hydrodynamic diameter (Z average, Zₐᵥₑ) of about 101 nm to about 752 nm.

In some aspects, the disclosure provides a dried form comprising extracellular vesicles (EVs) from bacteria, wherein the particles have a mean size of the most dominant DLS integrated peak of between about 25.55 nm to about 458.9 nm or between about 25.55 nm to about 157.40 nm.

In some aspects, the disclosure provides a dried form comprising extracellular vesicles (EVs) from bacteria and an excipient, wherein the excipient comprises about 95% to about 99% of the total mass of the dried form.

In some aspects, the disclosure provides a dried form comprising extracellular vesicles (EVs) from bacteria and an excipient, wherein the EVs make up about 2% to about 6% of the total mass of the dried form.

In some embodiments of the dried form provided herein, the dried form comprises a powder. In some embodiments, the powder comprises a lyophilized powder. In some embodiments the powder comprises a spray-dried powder.

In some embodiments of the dried form provided herein, the dried form comprises a lyophilate. In some embodiments, the lyophilate comprises a lyophilized powder. In some embodiments, the lyophilate comprises a lyophilized cake.

In some embodiments of the dried form provided herein, the dried form comprises EVs from a bacterial strain that is associated with small intestinal mucus.

In some embodiments of the dried form provided herein, the dried form comprises EVs from anaerobic bacteria.

In some embodiments of the dried form provided herein, the anaerobic bacteria are obligate anaerobes.

In some embodiments of the dried form provided herein, the anaerobic bacteria are facultative anaerobes.

In some embodiments of the dried form provided herein, the anaerobic bacteria are aerotolerant anaerobes.

In some embodiments of the dried form provided herein, the dried form comprises EVs from monoderm bacteria.

In some embodiments of the dried form provided herein, the dried form comprises EVs from diderm bacteria.

In some embodiments of the dried form e provided herein, the dried form comprises EVs from Gram negative bacteria.

In some embodiments of the dried form provided herein, the dried form comprises EVs from bacteria of the family: Prevotellaceae; Veillonellaceae; Tannerellaceae; Rikenellaceae; Selenomonadaceae; Sporomusaceae; Synergistaceae; Christensenellaceae; or Akkermaniaceae.

In some embodiments of the dried form provided herein, the dried form comprises EVs from Gram positive bacteria.

In some embodiments of the dried form provided herein, the dried form comprises EVs from bacteria of the family Oscillospiraceae; Clostridiaceae; or Lachnospiraceae.

In some embodiments of the dried form provided herein, the dried form comprises EVs from bacteria of the genus Prevotella.

In some embodiments of the dried form/provided herein, the dried form comprises EVs from bacteria of the genus Veillonella.

In some embodiments of the dried form provided herein, the dried form comprises EVs from bacteria of the genus Parabacteroides.

In some embodiments of the dried form provided herein, the dried form comprises EVs from bacteria of the Oscillospiraceae family.

In some embodiments of the dried form provided herein, the dried form comprises EVs from bacteria of the Tannerellaceae family.

In some embodiments of the dried form provided herein, the dried form comprises EVs from bacteria of the Prevotellaceae family.

In some embodiments of the dried form provided herein, the dried form comprises EVs from bacteria of the Veillonellaceae family.

In some embodiments of the dried form provided herein, the dried form comprises EVs from bacteria of the species Veillonella parvula.

In some embodiments of the dried form provided herein, the dried form comprises EVs from bacteria of the species Fournierella massiliensis.

In some aspects, the disclosure provides a solution comprising extracellular vesicles (EVs) from bacteria and an excipient that comprises a bulking agent.

In some aspects, the disclosure provides a solution consisting essentially of extracellular vesicles (EVs) from bacteria and an excipient that comprises a bulking agent.

In some aspects, the disclosure provides a solution comprising extracellular vesicles (EVs) from bacteria and an excipient that comprises a bulking agent and a lyoprotectant.

In some aspects, the disclosure provides a solution consisting essentially of extracellular vesicles (EVs) from bacteria and an excipient that comprises a bulking agent and a lyoprotectant.

In some aspects, the disclosure provides a solution comprising extracellular vesicles (EVs) from bacteria and an excipient that comprises a lyoprotectant.

In some aspects, the disclosure provides a solution consisting essentially of extracellular vesicles (EVs) from bacteria and an excipient that comprises a lyoprotectant.

In some aspects, the disclosure provides a therapeutic composition comprising the solution, wherein the composition further comprises a pharmaceutically acceptable excipient. In some embodiments, the pharmaceutically acceptable excipient comprises a glidant, lubricant, and/or diluent.

In some aspects, the disclosure provides a dried form comprising extracellular vesicles (EVs) from bacteria and an excipient that comprises a bulking agent.

In some aspects, the disclosure provides a dried form consisting essentially of extracellular vesicles (EVs) from bacteria and an excipient that comprises a bulking agent.

In some aspects, the disclosure provides a dried form comprising extracellular vesicles (EVs) from bacteria and an excipient that comprises a bulking agent and a lyoprotectant.

In some aspects, the disclosure provides a dried form consisting essentially of extracellular vesicles (EVs) from bacteria and an excipient that comprises a bulking agent and a lyoprotectant.

In some aspects, the disclosure provides a dried form comprising extracellular vesicles (EVs) from bacteria and an excipient that comprises a lyoprotectant.

In some aspects, the disclosure provides a dried form consisting essentially of extracellular vesicles (EVs) from bacteria and an excipient that comprises a lyoprotectant.

In some aspects, the disclosure provides a therapeutic composition comprising the dried form, wherein the composition further comprises a pharmaceutically acceptable excipient. In some embodiments, the pharmaceutically acceptable excipient comprises a glidant, lubricant, and/or diluent.

In some aspects, the disclosure provides a powder comprising extracellular vesicles (EVs) from bacteria and an excipient that comprises a bulking agent.

In some aspects, the disclosure provides a powder consisting essentially of extracellular vesicles (EVs) from bacteria and an excipient that comprises a bulking agent.

In some aspects, the disclosure provides a powder comprising extracellular vesicles (EVs) from bacteria and an excipient that comprises a bulking agent and a lyoprotectant.

In some aspects, the disclosure provides a powder consisting essentially of extracellular vesicles (EVs) from bacteria and an excipient that comprises a bulking agent and a lyoprotectant.

In some aspects, the disclosure provides a powder comprising extracellular vesicles (EVs) from bacteria and an excipient that comprises a lyoprotectant.

In some aspects, the disclosure provides a powder consisting essentially of extracellular vesicles (EVs) from bacteria and an excipient that comprises a lyoprotectant.

In some aspects, the disclosure provides a therapeutic composition comprising the powder, wherein the composition further comprises a pharmaceutically acceptable excipient. In some embodiments, the pharmaceutically acceptable excipient comprises a glidant, lubricant, and/or diluent.

In some aspects, the disclosure provides a spray-dried powder comprising extracellular vesicles (EVs) from bacteria and an excipient that comprises a bulking agent.

In some aspects, the disclosure provides a spray-dried powder consisting essentially of extracellular vesicles (EVs) from bacteria and an excipient that comprises a bulking agent.

In some aspects, the disclosure provides a spray-dried powder comprising extracellular vesicles (EVs) from bacteria and an excipient that comprises a bulking agent and a lyoprotectant.

In some aspects, the disclosure provides a spray-dried powder consisting essentially of extracellular vesicles (EVs) from bacteria and an excipient that comprises a bulking agent and a lyoprotectant.

In some aspects, the disclosure provides a spray-dried powder comprising extracellular vesicles (EVs) from bacteria and an excipient that comprises a lyoprotectant.

In some aspects, the disclosure provides a spray-dried powder consisting essentially of extracellular vesicles (EVs) from bacteria and an excipient that comprises a lyoprotectant.

In some aspects, the disclosure provides a therapeutic composition comprising the spray-dried powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilate comprising extracellular vesicles (EVs) from bacteria and an excipient that comprises a bulking agent.

In some aspects, the disclosure provides a lyophilate consisting essentially of extracellular vesicles (EVs) from bacteria and an excipient that comprises a bulking agent.

In some aspects, the disclosure provides a lyophilate comprising extracellular vesicles (EVs) from bacteria and an excipient that comprises a bulking agent and a lyoprotectant.

In some aspects, the disclosure provides a lyophilate consisting essentially of extracellular vesicles (EVs) from bacteria and an excipient that comprises a bulking agent and a lyoprotectant.

In some aspects, the disclosure provides a lyophilate comprising extracellular vesicles (EVs) from bacteria and an excipient that comprises a lyoprotectant.

In some aspects, the disclosure provides a lyophilate consisting essentially of extracellular vesicles (EVs) from bacteria and an excipient that comprises a lyoprotectant.

In some aspects, the disclosure provides a therapeutic composition comprising the lyophilate, wherein the composition further comprises a pharmaceutically acceptable excipient. In some embodiments, the pharmaceutically acceptable excipient comprises a glidant, lubricant, and/or diluent.

In some aspects, the disclosure provides a lyophilized powder comprising extracellular vesicles (EVs) from bacteria and an excipient that comprises a bulking agent.

In some aspects, the disclosure provides a lyophilized powder consisting essentially of extracellular vesicles (EVs) from bacteria and an excipient that comprises a bulking agent.

In some aspects, the disclosure provides a lyophilized powder comprising extracellular vesicles (EVs) from bacteria and an excipient that comprises a bulking agent and a lyoprotectant.

In some aspects, the disclosure provides a lyophilized powder consisting essentially of extracellular vesicles (EVs) from bacteria and from an excipient that comprises a bulking agent and a lyoprotectant.

In some aspects, the disclosure provides a lyophilized powder comprising extracellular vesicles (EVs) from bacteria and an excipient that comprises a lyoprotectant.

In some aspects, the disclosure provides a lyophilized powder consisting essentially of extracellular vesicles (EVs) from bacteria and from an excipient that comprises a lyoprotectant.

In some aspects, the disclosure provides a therapeutic composition comprising the lyophilized powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilized cake comprising extracellular vesicles (EVs) from bacteria and an excipient that comprises a bulking agent.

In some aspects, the disclosure provides a lyophilized cake consisting essentially of extracellular vesicles (EVs) from bacteria and an excipient that comprises a bulking agent.

In some aspects, the disclosure provides a lyophilized cake comprising extracellular vesicles (EVs) from bacteria and an excipient that comprises a bulking agent and a lyoprotectant.

In some aspects, the disclosure provides a lyophilized cake consisting essentially of extracellular vesicles (EVs) from bacteria and an excipient that comprises a bulking agent and a lyoprotectant.

In some aspects, the disclosure provides a lyophilized cake comprising extracellular vesicles (EVs) from bacteria and an excipient that comprises a lyoprotectant.

In some aspects, the disclosure provides a lyophilized cake consisting essentially of extracellular vesicles (EVs) from bacteria and an excipient that comprises a lyoprotectant.

In some aspects, the disclosure provides a therapeutic composition comprising extracellular vesicles (EVs) from bacteria and an excipient that comprises a bulking agent.

In some aspects, the disclosure provides a therapeutic composition consisting essentially of extracellular vesicles (EVs) from bacteria and an excipient that comprises a bulking agent.

In some aspects, the disclosure provides a therapeutic composition comprising extracellular vesicles (EVs) from bacteria and an excipient that comprises a bulking agent and a lyoprotectant.

In some aspects, the disclosure provides a therapeutic composition consisting essentially of extracellular vesicles (EVs) from bacteria and an excipient that comprises a bulking agent and a lyoprotectant.

In some aspects, the disclosure provides a therapeutic composition comprising extracellular vesicles (EVs) from bacteria and an excipient that comprises a lyoprotectant.

In some aspects, the disclosure provides a therapeutic composition consisting essentially of extracellular vesicles (EVs) from bacteria and an excipient that comprises a lyoprotectant.

In some aspects, the disclosure provides a solution comprising extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a solution consisting essentially of extracellular vesicles (EVs) and excipients of a stock comprising one or more excipients, wherein the stock comprises a formula provided in Table A, B, C, D, K, or P. In some embodiments, the EVs are EVs from bacteria.

In some aspects, the disclosure provides a therapeutic composition comprising such solution, wherein the composition further comprises a pharmaceutically acceptable excipient. In some embodiments, the pharmaceutically acceptable excipient comprises a glidant, lubricant, and/or diluent.

In some aspects, the disclosure provides a dried form comprising extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a dried form consisting essentially of extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a therapeutic composition comprising such dried form, wherein the composition further comprises a pharmaceutically acceptable excipient. In some embodiments, the pharmaceutically acceptable excipient comprises a glidant, lubricant, and/or diluent.

In some aspects, the disclosure provides a powder comprising extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a powder consisting essentially of extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a therapeutic composition comprising such powder, wherein the composition further comprises a pharmaceutically acceptable excipient. In some embodiments, the pharmaceutically acceptable excipient comprises a glidant, lubricant, and/or diluent.

In some aspects, the disclosure provides a spray-dried powder comprising extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a spray-dried powder consisting essentially of extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a therapeutic composition comprising such spray-dried powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilate comprising extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a lyophilate consisting essentially of extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a therapeutic composition comprising such lyophilate, wherein the composition further comprises a pharmaceutically acceptable excipient. In some embodiments, the pharmaceutically acceptable excipient comprises a glidant, lubricant, and/or diluent.

In some aspects, the disclosure provides a lyophilized powder comprising extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a lyophilized powder consisting essentially of extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a therapeutic composition comprising such lyophilized powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilized cake comprising extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a lyophilized cake consisting essentially of extracellular vesicles (EVs) and excipients of a stock comprising one or more excipients, wherein the stock comprises a formula provided in Table A, B, C, D, K or P. In some embodiments, the EVs are EVs from bacteria.

In some aspects, the disclosure provides a therapeutic composition comprising such lyophilized cake, wherein the composition further comprises a pharmaceutically acceptable excipient. In some embodiments, the pharmaceutically acceptable excipient comprises a glidant, lubricant, and/or diluent.

In some aspects, the disclosure provides a method of treating a subject (for example, human) (for example, a subject in need of treatment), the method comprising:
administering to the subject a solution, dried form, or therapeutic composition described herein.

In some embodiments, a solution, dried form, or therapeutic composition provided herein is for use in treating a subject (for example, human) (for example, a subject in need of treatment).

In some aspects, the disclosure provides use of a solution, dried form, or therapeutic composition provided herein for the preparation of a medicament for treating a subject (for example, human) (for example, a subject in need of treatment).

In some embodiments of the method, solution, dried form, therapeutic composition or use provided herein, the solution, dried form, or therapeutic composition is orally administered (for example, is for oral administration).

In some embodiments of the method, solution, dried form, therapeutic composition or use provided herein, the subject is in need of treatment (and/or prevention) of a cancer.

In some embodiments of the method, solution, dried form, therapeutic composition or use provided herein, the subject is in need of treatment (and/or prevention) of an autoimmune disease.

In some embodiments of the method, solution, dried form, therapeutic composition or use provided herein, the subject is in need of treatment (and/or prevention) of an inflammatory disease.

In some embodiments of the method, solution, dried form, therapeutic composition or use provided herein, the subject is in need of treatment (and/or prevention) of a metabolic disease.

In some embodiments of the method, solution, dried form, therapeutic composition or use provided herein, the subject is in need of treatment (and/or prevention) of dysbiosis.

In some embodiments of the method, solution, dried form, therapeutic composition or use provided herein, the solution, dried form, or therapeutic composition is administered in combination with an additional therapeutic agent.

In some embodiments of the method, solution, dried form, therapeutic composition or use provided herein, the dried form is a powder. In some embodiments, the powder is a lyophilized powder. In some embodiments, the powder is a spray-dried powder.

In some embodiments of the method, solution, dried form, therapeutic composition or use provided herein, the dried form is a lyophilate. In some embodiments, the lyophilate is a lyophilized powder. In some embodiments, the lyophilate is a lyophilized cake.

In some aspects, the disclosure provides a method of preparing a solution that comprises extracellular vesicles (EVs) from bacteria, the method comprising: combining a liquid preparation that comprises EVs from bacteria with an excipient that comprises (or consists essentially of) a bulking agent, thereby preparing the solution.

In some aspects, the disclosure provides a method of preparing a solution that comprises extracellular vesicles (EVs) from bacteria, the method comprising:
combining a liquid preparation that comprises EVs from bacteria with an excipient that comprises (or consists essentially of) a bulking agent and a lyoprotectant, thereby preparing the solution.

In some aspects, the disclosure provides a method of preparing a solution that comprises extracellular vesicles (EVs) from bacteria, the method comprising:
combining a liquid preparation that comprises EVs from bacteria with an excipient that comprises (or consists essentially of) a lyoprotectant, thereby preparing the solution.

In some embodiments, the disclosure provides a solution prepared by a method described herein.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a bulking agent to prepare a solution; and
drying the solution, thereby preparing the dried form.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a bulking agent to prepare a solution;
drying the solution to prepare a cake, and
milling (for example, grinding) the cake, thereby preparing the dried form.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
drying the solution, thereby preparing the dried form.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution;
drying the solution to prepare a cake, and
milling (for example, grinding) the cake, thereby preparing the dried form.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a lyoprotectant to prepare a solution; and
drying the solution, thereby preparing the dried form.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) lyoprotectant to prepare a solution;
drying the solution to prepare a cake, and
milling (for example, grinding) the cake, thereby preparing the dried form.

In some embodiments of the methods of preparing a dried form provided herein, the drying comprises lyophilization.

In some embodiments of the methods of preparing a dried form provided herein, the drying comprises spray drying.

In some embodiments of the methods of preparing a dried form provided herein, the method further comprises combining the dried form with an additional ingredient. In some embodiments, the additional ingredient comprises an excipient, for example, a glidant, lubricant, and/or diluent.

In some embodiments of the methods of preparing a dried form provided herein, the disclosure provides a dried form prepared by a method described herein.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a bulking agent to prepare a solution; and
drying the solution, thereby preparing the powder.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a bulking agent to prepare a solution;
drying the solution to prepare a cake, and
milling (for example, grinding) the cake, thereby preparing the powder.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
drying the solution, thereby preparing the powder.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution;
drying the solution to prepare a cake, and
milling (for example, grinding) the cake, thereby preparing the powder.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a lyoprotectant to prepare a solution; and
drying the solution, thereby preparing the powder.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a lyoprotectant to prepare a solution;
drying the solution to prepare a cake, and
milling (for example, grinding) the cake, thereby preparing the powder.

In some embodiments of the methods of preparing a powder provided herein, the drying comprises lyophilization.

In some embodiments of the methods of preparing a powder provided herein, the drying comprises spray drying.

In some embodiments of the methods of preparing a powder provided herein, the method further comprises combining the powder with an additional ingredient. In some embodiments, the additional ingredient comprises an excipient, for example, a glidant, lubricant, and/or diluent.

In some embodiments of the methods of preparing a powder provided herein, the disclosure provides a powder prepared by a method described herein.

In some aspects, the disclosure provides a method of preparing a spray-dried powder that comprises extracellular vesicles (EVs) from bacteria, the method comprising:
combining a liquid preparation that comprises EVs from bacteria with an excipient that comprises (or consists essentially of) a bulking agent to prepare a solution; and
spray drying the solution, thereby preparing the spray-dried powder.

In some aspects, the disclosure provides a method of preparing a spray-dried powder that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
spray drying the solution, thereby preparing the spray-dried powder.

In some aspects, the disclosure provides a method of preparing a spray-dried powder that comprises extracellular vesicles (EVs) from bacteria, the method comprising:
combining a liquid preparation that comprises EVs from bacteria with an excipient that comprises (or consists essentially of) a lyoprotectant to prepare a solution; and
spray drying the solution, thereby preparing the spray-dried powder.

In some embodiments of the methods of preparing a spray-dried powder provided herein, the method further comprises combining the spray-dried powder with an additional ingredient. In some embodiments, the additional ingredient comprises an excipient, for example, a glidant, lubricant, and/or diluent.

In some embodiments of the methods of preparing a spray-dried powder provided herein, the disclosure provides a spray-dried powder prepared by a method described herein.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a bulking agent to prepare a solution; and
freeze drying (lyophilizing) the solution, thereby preparing the lyophilate.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a bulking agent to prepare a solution;
freeze drying (lyophilizing) the solution to prepare a cake, and
milling (for example, grinding) the cake, thereby preparing the lyophilate.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution;
freeze drying (lyophilizing) the solution to prepare a cake, and
milling (for example, grinding) the cake, thereby preparing the lyophilate.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a lyoprotectant to prepare a solution;
freeze drying (lyophilizing) the solution to prepare a cake, and
milling (for example, grinding) the cake, thereby preparing the lyophilate.

In some embodiments of the methods of preparing a lyophilate provided herein, the method further comprises combining the lyophilate with an additional ingredient. In some embodiments, the additional ingredient comprises an excipient, for example, a glidant, lubricant, and/or diluent.

In some embodiments of the methods of preparing a lyophilate provided herein, the disclosure provides a lyophilate prepared by a method described herein.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a bulking agent to prepare a solution; and
freeze drying (lyophilizing) the solution, thereby preparing the lyophilized powder.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a bulking agent to prepare a solution;
freeze drying (lyophilizing) the solution to prepare a cake, and
milling (for example, grinding) the cake, thereby preparing the lyophilized powder.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution;
freeze drying (lyophilizing) the solution to prepare a cake, and
milling (for example, grinding) the cake, thereby preparing the lyophilized powder.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a lyoprotectant to prepare a solution;
freeze drying (lyophilizing) the solution to prepare a cake, and
milling (for example, grinding) the cake, thereby preparing the lyophilized powder.

In some embodiments of the methods of preparing a lyophilized powder provided herein, the method further comprises combining the lyophilized powder with an additional ingredient. In some embodiments, the additional ingredient comprises an excipient, for example, a glidant, lubricant, and/or diluent.

In some embodiments of the methods of preparing a lyophilized powder provided herein, the disclosure provides a lyophilized powder prepared by a method described herein.

In some aspects, the disclosure provides a method of preparing a lyophilized cake that comprises extracellular vesicles (EVs) from bacteria, the method comprising:
combining a liquid preparation that comprises EVs from bacteria with an excipient that comprises (or consists essentially of) a bulking agent to prepare a solution; and
freeze drying (lyophilizing) the solution, thereby preparing the lyophilized cake.

In some aspects, the disclosure provides a method of preparing a lyophilized cake that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
freeze drying (lyophilizing) the solution, thereby preparing the lyophilized cake.

In some aspects, the disclosure provides a method of preparing a lyophilized cake that comprises extracellular vesicles (EVs) from bacteria, the method comprising:
combining a liquid preparation that comprises EVs from bacteria with an excipient that comprises (or consists essentially of) a lyoprotectant to prepare a solution; and
freeze drying (lyophilizing) the solution, thereby preparing the lyophilized cake.

In some embodiments of the methods of preparing a lyophilized cake provided herein, the disclosure provides a lyophilized cake prepared by a method described herein.

In some aspects, the disclosure provides a method of preparing a solution that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution.

In some embodiments, the EVs are from bacteria.

In some embodiments, the disclosure provides a solution prepared by a method described herein.

In some aspects, the disclosure provides a method of preparing a dried form that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
drying the solution, thereby preparing the dried form.

In some aspects, the disclosure provides a method of preparing a dried form that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution;
drying the solution to prepare a cake, and
milling (for example, grinding) the cake, thereby preparing the dried form.

In some embodiments of the methods of preparing a dried form provided herein, the EVs are from bacteria.

In some embodiments of the methods of preparing a dried form provided herein, the drying comprises lyophilization.

In some embodiments of the methods of preparing a dried form provided herein, the drying comprises spray drying.

In some embodiments of the methods of preparing a dried form provided herein, the method further comprises combining the dried form with an additional ingredient. In some embodiments, the additional ingredient comprises an excipient, for example, a glidant, lubricant, and/or diluent.

In some embodiments of the methods of preparing a dried form provided herein, the disclosure provides a dried form prepared by a method described herein.

In some aspects, the disclosure provides a method of preparing a powder that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
drying the solution, thereby preparing the powder.

In some aspects, the disclosure provides a method of preparing a powder that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution;
drying the solution to prepare a cake, and
milling (for example, grinding) the cake, thereby preparing the powder.

In some embodiments of the methods of preparing a powder provided herein, the EVs are from bacteria.

In some embodiments of the methods of preparing a powder provided herein, the drying comprises lyophilization.

In some embodiments of the methods of preparing a powder provided herein, the drying comprises spray drying.

In some embodiments of the methods of preparing a powder provided herein, the method further comprises combining the powder with an additional ingredient. In some embodiments, the additional ingredient comprises an excipient, for example, a glidant, lubricant, and/or diluent.

In some embodiments of the methods of preparing a powder provided herein, the disclosure provides a powder prepared by a method described herein.

In some aspects, the disclosure provides a method of preparing a spray-dried powder that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
spray drying the solution, thereby preparing the spray-dried powder.

In some embodiments of the methods of preparing a spray-dried powder provided herein, the EVs are from bacteria.

In some embodiments of the methods of preparing a spray-dried powder provided herein, the method further comprises combining the spray-dried powder with an additional ingredient. In some embodiments, the additional ingredient comprises an excipient, for example, a glidant, lubricant, and/or diluent.

In some embodiments of the methods of preparing a spray-dried powder provided herein, the disclosure provides a spray-dried powder prepared by a method described herein.

In some aspects, the disclosure provides a method of preparing a lyophilate that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
freeze drying (lyophilizing) the solution, thereby preparing the lyophilate.

In some aspects, the disclosure provides a method of preparing a lyophilate that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution;
freeze drying (lyophilizing) the solution to prepare a cake, and
milling (for example, grinding) the cake, thereby preparing the lyophilate.

In some embodiments of the methods of preparing a lyophilate provided herein, the EVs are from bacteria.

In some embodiments of the methods of preparing a lyophilate provided herein, the method further comprises combining the lyophilate with an additional ingredient. In some embodiments, the additional ingredient comprises an excipient, for example, a glidant, lubricant, and/or diluent.

In some embodiments of the methods of preparing a lyophilate provided herein, the disclosure provides a lyophilate prepared by a method described herein.

In some aspects, the disclosure provides a method of preparing a lyophilized powder that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
freeze drying (lyophilizing) the solution, thereby preparing the lyophilized powder.

In some aspects, the disclosure provides a method of preparing a lyophilized powder that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution;
freeze drying (lyophilizing) the solution to prepare a cake, and
milling (for example, grinding) the cake, thereby preparing the lyophilized powder.

In some embodiments of the methods of preparing a lyophilized powder provided herein, the EVs are from bacteria.

In some embodiments of the methods of preparing a lyophilized powder provided herein, the method further comprises combining the lyophilized powder with an additional ingredient. In some embodiments, the additional ingredient comprises an excipient, for example, a glidant, lubricant, and/or diluent.

In some embodiments of the methods of preparing a lyophilized powder provided herein, the disclosure provides a lyophilized powder prepared by a method described herein.

In some aspects, the disclosure provides a method of preparing a lyophilized cake that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
freeze drying (lyophilizing) the solution, thereby preparing a lyophilized cake.

In some embodiments of the methods of preparing a lyophilized cake provided herein, the disclosure provides a lyophilized cake prepared by a method described herein.

In some embodiments of the methods that comprise a freeze drying step, the freeze drying comprises primary drying and secondary drying. In some embodiments, primary drying is performed at a temperature between about −35° C. to about −20° C. For example, primary drying is performed at a temperature of about −20° C., about −25° C., about −30° C., or about −35° C. In some embodiments, secondary drying is performed at a temperature between about +20° C. to about +30° C. For example, secondary drying is performed at a temperature of about +25° C.

In some embodiments of the solution, dried form, or therapeutic composition provided herein, the bulking agent comprises mannitol, sucrose, maltodextrin, dextran, Ficoll, polyethylene glycol (PEG, such as PEG 6000), cyclodextrin, or PVP-K30.

In some embodiments of the solution, dried form, or therapeutic composition provided herein, the bulking agent comprises mannitol.

In some embodiments of the solution, dried form, or therapeutic composition provided herein, the excipient comprises an additional ingredient.

In some embodiments of the solution, dried form, or therapeutic composition provided herein, the additional ingredient comprises trehalose, mannitol, sucrose, sorbitol, dextran, poloxamer 188, maltodextrin, PVP-K30, Ficoll, citrate, arginine, and/or hydroxypropyl-B-cyclodextrin.

In some embodiments of the solution, dried form, or therapeutic composition provided herein, the excipient comprises mannitol and trehalose.

In some embodiments of the solution, dried form, or therapeutic composition provided herein, the excipient consists essentially of mannitol and trehalose.

In some embodiments of the solution, dried form, or therapeutic composition provided herein, the excipient comprises mannitol, trehalose, and sorbitol.

In some embodiments of the solution, dried form, or therapeutic composition provided herein, the excipient consists essentially of mannitol, trehalose, and sorbitol.

In some embodiments of the solution, dried form, or therapeutic composition provided herein, the excipient comprises trehalose.

In some embodiments of the solution, dried form, or therapeutic composition provided herein, the excipient consists essentially of trehalose.

In some embodiments of the solution, dried form, or therapeutic composition provided herein, the excipient is from a stock comprising one or more excipients, wherein the stock comprises a formula provided in provided in Table A, B, C, D, K or P.

In some embodiments of the solution, dried form, or therapeutic composition provided herein, the dried form is a powder. In some embodiments, the powder is a lyophilized powder. In some embodiments, the powder is a spray-dried powder.

In some embodiments of the solution, dried form, or therapeutic composition provided herein, the dried form is a lyophilate. In some embodiments, the lyophilate is a lyophilized powder. In some embodiments, the lyophilate is a lyophilized cake.

In some embodiments of the solution, dried form, or therapeutic composition provided herein, the excipient solution comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient solution comprises more mannitol than trehalose, for example, on a weight basis or weight percent basis. In some embodiments, the excipient solution comprises at least two-fold more mannitol than trehalose, for example, on a weight basis or weight percent basis. In some embodiments, the excipient solution comprises at least three-fold more mannitol than trehalose, for example, on a weight basis or weight percent basis. In some embodiments, the excipient of the solution or dried form comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient of the solution or dried form comprises more mannitol than trehalose, for example, on a weight basis or weight percent basis. In some embodiments, the excipient of the solution or dried form comprises at least two-fold more mannitol than trehalose, for example, on a weight basis or weight percent basis. In some embodiments, the excipient of the solution or dried form comprises at least three-fold more mannitol than trehalose, for example, on a weight basis or weight percent basis.

In some embodiments of the solution, dried form, or therapeutic composition provided herein, the excipient solution consists essentially of mannitol and trehalose. In some embodiments, the excipient solution consists essentially of mannitol and trehalose, wherein the mannitol and the trehalose are not present in equal amounts (for example, the mannitol and the trehalose are present in unequal amounts; for example, on a weight basis or a weight percent basis). In some embodiments, the excipient solution consists essentially of mannitol and trehalose, wherein the excipient contains more mannitol than trehalose, for example, on a weight basis or weight percent basis. In some embodiments, the excipient solution consists essentially of mannitol and trehalose, wherein the excipient solution contains at least two-fold more mannitol than trehalose, for example, on a weight basis or weight percent basis. In some embodiments, the excipient solution consists essentially of mannitol and trehalose, wherein the excipient solution contains at least three-fold more mannitol than trehalose, for example, on a weight basis or weight percent basis. In some embodiments, the excipient of the solution or dried form consists essentially of mannitol and trehalose, wherein the excipient of the solution or dried form contains more mannitol than trehalose, for example, on a weight basis or weight percent basis. In some embodiments, the excipient of the solution or dried form consists essentially of mannitol and trehalose, wherein the excipient of the solution or dried form contains at least two-fold more mannitol than trehalose, for example, on a weight basis or weight percent basis. In some embodiments, the excipient of the solution or dried form consists essentially of mannitol and trehalose, wherein the excipient of the solution or dried form contains at least three-fold more mannitol than trehalose, for example, on a weight basis or weight percent basis.

In some embodiments of the solution, dried form, or therapeutic composition provided herein, the excipient solution comprises, or consists essentially of, mannitol and trehalose, wherein neither the mannitol nor the trehalose is present in an amount of 5 mg/ml to 15 mg/ml. In some embodiments, the excipient solution comprises, or consists essentially of, mannitol and trehalose, wherein the mannitol is not present in an amount of 5 mg/ml to 15 mg/ml. In some embodiments, the excipient solution comprises, or consists essentially of, mannitol and trehalose, wherein the trehalose is not present in an amount of 5 mg/ml to 15 mg/ml.

In some embodiments of the solution, dried form, or therapeutic composition provided herein, the excipient solution comprises, or consists essentially of, mannitol and trehalose, wherein neither the mannitol nor the trehalose is present in an amount of 9 mg/ml. In some embodiments, the excipient solution comprises, or consists essentially of, mannitol and trehalose, wherein the mannitol is not present in an amount of 9 mg/ml. In some embodiments, the excipient solution comprises, or consists essentially of, mannitol and trehalose, wherein the trehalose is not present in an amount of 9 mg/ml.

In some embodiments of the solution, dried form, or therapeutic composition provided herein, the excipient comprises, or consists essentially of, mannitol and trehalose, and does not comprise methionine.

In some embodiments of the dried form or therapeutic composition provided herein, the dried form or therapeutic composition comprises, or consists essentially of, mannitol and trehalose, and the mannitol and the trehalose are not present in equal amounts (for example, the mannitol and the trehalose are present in unequal amounts, for example, on a weight basis or a weight percent basis) in the dried form or therapeutic composition.

In some embodiments of the solution, dried form, or therapeutic composition provided herein, at least about 10% (by weight) of the solution or dried form is excipient stock.

In some embodiments of the solution, dried form, or therapeutic composition provided herein, about 10% to about 80% (by weight) of the solution or dried form is excipient stock.

In some embodiments of the solution, dried form, or therapeutic composition provided herein, wherein about 20% to about 70% (by weight) of the solution or dried form is excipient stock.

In some embodiments of the solution, dried form, or therapeutic composition provided herein, about 30% to about 60% (by weight) of the solution or dried form is excipient stock.

In some embodiments of the dried form or therapeutic composition provided herein, the EVs comprise at least about 1% of the total solids by weight of the dried form.

In some embodiments of the dried form or therapeutic composition provided herein, the EVs comprise about 1% to about 99% of the total solids by weight of the dried form.

In some embodiments of the dried form or therapeutic composition provided herein, the EVs comprise about 5% to about 90% of the total solids by weight of the dried form. In some embodiments of the dried form or therapeutic composition provided herein, the EVs comprise about 1% to about 60% of the total solids by weight of the dried form. In some embodiments of the dried form or therapeutic composition provided herein, the EVs comprise about 1% to about 20% of the total solids by weight of the powder or cake. In some embodiments of the dried form or therapeutic composition provided herein, the EVs comprise about 2% to about 10% of the total solids by weight of the dried form. In some embodiments of the dried form or therapeutic composition provided herein, the EVs comprise about 2% to about 6% of the total solids by weight of the dried form. In some embodiments of the dried form or therapeutic composition provided herein, the dried form comprises a moisture content below about 6% (for example, as determined by Karl Fischer titration).

In some embodiments of the dried form or therapeutic composition provided herein, the dried form comprises a moisture content below about 5% (for example, as determined by Karl Fischer titration).

In some embodiments of the dried form or therapeutic composition provided herein, the dried form comprises a moisture content about 0.5% to about 5% (for example, as determined by Karl Fischer titration).

In some embodiments of the dried form or therapeutic composition provided herein, the dried form comprises a moisture content about 1% to about 5% (for example, as determined by Karl Fischer titration).

In some embodiments of the dried form or therapeutic composition provided herein, the dried form comprises a moisture content about 1% to about 4% (for example, as determined by Karl Fischer titration).

In some embodiments of the dried form or therapeutic composition provided herein, the dried form comprises a moisture content about 2% to about 5% (for example, as determined by Karl Fischer titration).

In some embodiments of the dried form or therapeutic composition provided herein, the dried form comprises a moisture content about 2% to about 4% (for example, as determined by Karl Fischer titration).

In some embodiments of the dried form or therapeutic composition provided herein, the dried form comprises at least 1e10 particles per mg of the dried form (for example, as determined by particles per mg, such as by NTA).

In some embodiments of the dried form or therapeutic composition provided herein, the dried form comprises about 3e10 to about 8e10 particles per mg of the dried form (for example, as determined by particles per mg, such as by NTA).

In some embodiments of the dried form or therapeutic composition provided herein, the dried form comprises about 6e10 to about 8e10 particles per mg of the dried form (for example, as determined by particles per mg, such as by NTA).

In some embodiments of the dried form or therapeutic composition provided herein, the dried form comprises about 6.7e8 to about 2.55e10 particles/mg dried form.

In some embodiments of the dried form or therapeutic composition provided herein, the dried form comprises about 6.7e8 to about 2.89e10 particles/mg dried form.

In some embodiments, particle numeration is determined on a dried form by NTA. In some embodiments, particle numeration is determined on a dried form by NTA with use of a Zetaview camera.

In some embodiments, particle numeration is determined on dried form resuspended in water, by NTA and with use of a Zetaview camera.

In some embodiments of the dried form or therapeutic composition provided herein, the particles have a hydrodynamic diameter (Z average, Zₐᵥₑ) of about 200 nm after resuspension from the dried form (for example, resuspension in deionized water) (for example, as determined by dynamic light scattering).

In some embodiments of the dried form or therapeutic composition provided herein, the particles have a hydrodynamic diameter (Z average, Zₐᵥₑ) of about 200 nm after resuspension from the dried form (for example, resuspension in deionized water) (for example, as determined by dynamic light scattering).

In some embodiments of the dried form or therapeutic composition provided herein, the particles have a hydrodynamic diameter (Z average, Zₐᵥₑ) of about 101 nm to about 752 nm. In some embodiments, dynamic light scattering (DLS) is used to obtain the hydrodynamic diameter (Z average, Zₐᵥₑ) of particles present after the lyophilate is resuspended in deionized water or in a buffer such as PBS (for example, 0.1×PBS).

In some embodiments of the dried form or therapeutic composition provided herein, the particles have a mean size of the most dominant DLS integrated peak of between about 25.55 nm to about 458.9 nm.

In some embodiments of the dried form or therapeutic composition provided herein, the particles have a mean size of the most dominant DLS integrated peak of between about 25.55 nm to about 157.40 nm.

In some embodiments of the dried form or therapeutic composition provided herein, the particles have a charge (as measured by zeta potential (mV), for example, as measured by DLS of the charge of the most dominant DLS integrated peak of particles) of about −29.2 to about +2.67 mV.

In some embodiments of the dried form or therapeutic composition provided herein, the particles have a charge (as measured by zeta potential (mV), for example, as measured by DLS of total particles) of about −0.929 to about −24.80 mV.

In some embodiments of the solution, dried form, or therapeutic composition provided herein, the EVs are from Gram positive bacteria.

In some embodiments of the solution, dried form, or therapeutic composition provided herein, the EVs are from Gram negative bacteria.

In some embodiments of the solution, dried form, or therapeutic composition provided herein, the EVs are from aerobic bacteria.

In some embodiments of the solution, dried form, or therapeutic composition provided herein, the EVs are from anaerobic bacteria. In some embodiments, the anaerobic bacteria comprise obligate anaerobes. In some embodiments, the anaerobic bacteria comprise facultative anaerobes.

In some embodiments of the solution, dried form, or therapeutic composition provided herein, the EVs are from aerotolerant bacteria.

In some embodiments of the solution, dried form, or therapeutic composition provided herein, the EVs are from monoderm bacteria.

In some embodiments of the solution, dried form, or therapeutic composition provided herein, the EVs are from diderm bacteria.

In some embodiments of the solution, dried form, or therapeutic composition provided herein, the EVs are from bacteria of the family: Prevotellaceae; Veillonellaceae; Tannerellaceae; Rikenellaceae; Selenomonadaceae; Sporomusaceae; Synergistaceae; or Christensenellaceae; orAkkermaniaceae.

In some embodiments of the solution, dried form, or therapeutic composition provided herein, the EVs are from bacteria of the family Oscillospiraceae; Clostridiaceae; or Lachnospiraceae.

In some embodiments of the solution, dried form, or therapeutic composition provided herein, the EVs are from bacteria of the genus Prevotella.

In some embodiments of the solution, dried form, or therapeutic composition provided herein, the EVs are from bacteria of the genus Veillonella.

In some embodiments of the solution, dried form, or therapeutic composition provided herein, the EVs are from bacteria of the genus Parabacteroides.

In some embodiments of the solution, dried form, or therapeutic composition provided herein, the EVs are from a bacterial strain of the Oscillospiraceae family.

In some embodiments of the solution, dried form, or therapeutic composition provided herein, the EVs are from a bacterial strain of the Tannerellaceae family.

In some embodiments of the solution, dried form, or therapeutic composition provided herein, the EVs are from a bacterial strain of the Prevotellaceae family.

In some embodiments of the solution, dried form, or therapeutic composition provided herein, the EVs are from a bacterial strain of the Veillonellaceae family.

In some embodiments of the solution, dried form, or therapeutic composition provided herein, the EVs are from acidophile bacteria.

In some embodiments of the solution, dried form, or therapeutic composition provided herein, the EVs are from alkaliphile bacteria.

In some embodiments of the solution, dried form, or therapeutic composition provided herein, the EVs are from neutralophile bacteria.

In some embodiments of the solution, dried form, or therapeutic composition provided herein, the EVs are from fastidious bacteria.

In some embodiments of the solution, dried form, or therapeutic composition provided herein, the EVs are from nonfastidious bacteria.

In some embodiments of the solution, dried form, or therapeutic composition provided herein, the EVs are from bacteria from a taxonomic group (for example, class, order, family, genus, species or strain) listed in Table 1, Table 2, Table 3, and/or Table 4.

In some embodiments of the solution, dried form, or therapeutic composition provided herein, the EVs are from a bacterial strain listed in Table 1, Table 2, Table 3, and/or Table 4.

In some embodiments of the solution, dried form, or therapeutic composition provided herein, the EVs are from bacteria from a taxonomic group (for example, class, order, family, genus, species or strain) listed in Table J.

In some embodiments of the solution, dried form, or therapeutic composition provided herein, the EVs are from a bacterial species listed in Table J.

In some embodiments of the solution, dried form, or therapeutic composition provided herein, the EVs are from a bacterial strain listed in Table J.

In some embodiments, a solution, dried form, or therapeutic composition provided herein contains EVs from one or more bacterial strain. In some embodiments, a solution, dried form, or therapeutic composition provided herein contains EVs from one bacterial strain. In some embodiments, the bacterial strain used as a source of EVs is selected based on the properties of the bacteria (for example, growth characteristics, yield, ability to modulate an immune response in an assay or a subject).

In some embodiments, a solution, dried form, or therapeutic composition provided herein comprising EVs from bacteria is used for the treatment or prevention of a disease and/or a health disorder, for example, in a subject (for example, human).

In some embodiments, a dried form (or a therapeutic composition thereof) provided herein comprising EVs from bacteria is prepared as a solid dose form, such as a tablet, a minitablet, a capsule, or a powder; or a combination of these forms (for example, minitablets comprised in a capsule). In some embodiments, the solid dose form comprises a coating (for example, enteric coating).

In some embodiments, a dried form (or a therapeutic composition thereof) provided herein comprising EVs from bacteria is reconstituted. In some embodiments, a solution (or a therapeutic composition thereof) provided herein comprising EVs from bacteria is used as suspension, for example, diluted to a suspension or used in undiluted form.

In some embodiments, a therapeutic composition comprising a solution and/or dried form comprising EVs from bacteria is prepared as provided herein. In some embodiments, the therapeutic composition comprising a dried form is formulated into a solid dose form, such as a tablet, a minitablet, a capsule, or a powder. In some embodiments, the therapeutic composition comprising a dried form is reconstituted in a suspension.

In some embodiments, the therapeutic composition comprising a powder is formulated into a solid dose form, such as a tablet, a minitablet, a capsule, or a powder. In some embodiments, the therapeutic composition comprising a powder is reconstituted in a suspension.

In some embodiments, a solution, dried form, or therapeutic composition provided herein comprises gamma irradiated EVs from bacteria. In some embodiments, the gamma irradiated EVs from bacteria are formulated into therapeutic composition. In some embodiments, the gamma irradiated EVs from bacteria are formulated into a solid dose form, such as a tablet, a minitablet, a capsule, or a powder. In some embodiments, the gamma irradiated EVs from bacteria are formulated reconstituted in a suspension.

In some embodiments, a solution, dried form, or therapeutic composition provided herein comprising EVs from bacteria are orally administered.

In some embodiments, a solution, dried form, or therapeutic composition provided herein comprising EVs from bacteria are administered intranasally.

In some embodiments, a solution, dried form, or therapeutic composition provided herein comprising EVs from bacteria are administered by inhalation.

In some embodiments, a solution, dried form, or therapeutic composition provided herein comprising EVs from bacteria are administered intravenously.

In some embodiments, a solution, dried form, or therapeutic composition provided herein comprising EVs from bacteria are administered by injection, for example, intratumorally or subtumorally, for example, to a subject who has a tumor.

In some embodiments, a solution, dried form, or therapeutic composition provided herein comprising EVs from bacteria are administered topically.

In certain aspects, provided herein are therapeutic compositions comprising solutions and/or dried form comprising EVs from bacteria useful for the treatment and/or prevention of a disease or a health disorder (for example, adverse health disorders) (for example, a cancer, an autoimmune disease, an inflammatory disease, a dysbiosis, or a metabolic disease), as well as methods of making and/or identifying such solutions and/or dried form and/or therapeutic compositions, and methods of using such solutions and/or dried form, and/or therapeutic compositions thereof (for example, for the treatment of a cancer, an autoimmune disease, an inflammatory disease, a dysbiosis, or a metabolic disease, either alone or in combination with other therapeutics).

In some embodiments, the therapeutic compositions comprise both EVs from bacteria and whole bacteria, for example, bacteria from which the EVs were obtained, such as live bacteria, killed bacteria, attenuated bacteria. In some embodiments, the therapeutic compositions comprise EVs from bacteria in the absence of the bacteria from which they were obtained, such that over about 85%, over about 90%, or over about 95% (or over about 99%) of the bacteria-sourced content of the solutions and/or powders comprises EVs. In some embodiments, the gamma irradiated EVs from bacteria are formulated the EVs are isolated EVs, for example, isolated by a method described herein.

In some embodiments, the solution, dried form, or therapeutic composition comprises EVs from one or more bacteria of a taxonomic group (for example, class, order, family, genus, species or strain)) provided herein (for example, listed in Table 1, Table 2, Table 3, and/or Table 4 and/or elsewhere in the specification (for example, Table J or Example 10)). In some embodiments, the solution, dried form, or therapeutic composition comprises EVs from one or more of the bacteria strains or species provided herein (for example, listed in Table 1, Table 2, Table 3, and/or Table 4 and/or elsewhere in the specification (for example, Table J or Example 10)).

In some embodiments, the solution, dried form, or therapeutic composition comprises isolated EVs (for example, from one or more strains of bacteria. For example, wherein at least 50%, at least 75%, at least 80%, at least 85%, at least 90%, at least 95%, or at least 99% of the content (for example, of the content that does not exclude excipient) of the solution and/or dried form, is isolated EVs from bacteria (for example, bacteria of interest).

In some embodiments, the solution, dried form, or therapeutic composition comprises isolated EVs (for example, from one strain of bacteria (for example, bacteria of interest). For example, wherein at least 50%, at least 75%, at least 80%, at least 85%, at least 90%, at least 95%, or at least 99% of the content (for example, of the content that does not exclude excipient) of the solution and/or dried form is isolated EV of bacteria (for example, bacteria of interest, for example, bacteria disclosed herein).

In some embodiments, the solution, dried form, or therapeutic composition comprises EVs from one strain of bacteria.

In some embodiments, the solution, dried form, or therapeutic composition comprises EVs from more than one strain of bacteria.

In some embodiments, the EVs are lyophilized.

In some embodiments, the EVs are gamma irradiated.

In some embodiments, the EVs are UV irradiated.

In some embodiments, the EVs are heat inactivated (for example, at 50° C. for two hours or at 90° C. for two hours).

In some embodiments, the EVs are acid treated.

In some embodiments, the EVs are oxygen sparged (for example, at 0.1 vvm for two hours).

In some embodiments, the EVs are from Gram positive bacteria.

In some embodiments, the EVs are from Gram negative bacteria.

In some embodiments, the EVs are from bacterial species evaluated in Example 10.

In some embodiments, the EVs are from aerobic bacteria.

In some embodiments, the EVs are from anaerobic bacteria. In some embodiments, the anaerobic bacteria comprise obligate anaerobes. In some embodiments, the anaerobic bacteria comprise facultative anaerobes.

In some embodiments, the EVs are from acidophile bacteria.

In some embodiments, the EVs are from alkaliphile bacteria.

In some embodiments, the EVs are from neutralophile bacteria.

In some embodiments, the EVs are from fastidious bacteria.

In some embodiments, the EVs are from nonfastidious bacteria.

In some embodiments, the EVs are from bacteria of a taxonomic group (for example, class, order, family, genus, species or strain)) provided herein (for example, listed in Table 1, Table 2, Table 3, and/or Table 4 and/or elsewhere in the specification (for example, Table J or Example 10).

In some embodiments, the EVs are from a bacterial strain provided herein (for example, listed in Table 1, Table 2, Table 3, and/or Table 4 and/or elsewhere in the specification (for example, Table J or Example 10)).

In some embodiments, the EVs are from aerotolerant bacteria.

In some embodiments, EVs are selected from a bacterial strain that is associated with mucus. In some embodiments, the mucus is associated with the gut lumen. In some embodiments, the mucus is associated with the small intestine. In some embodiments, the mucus is associated with the respiratory tract.

In some embodiments, EVs are selected from a bacterial strain that is associated with an epithelial tissue, such as oral cavity, lung, nose, or vagina.

In some embodiments, the EVs are from bacteria that are human commensals.

In some embodiments, the EVs are from human commensal bacteria that originate from the human small intestine.

In some embodiments, the EVs are from human commensal bacteria that originate from the human small intestine and are associated there with the outer mucus layer.

In some embodiments, the EVs are from monoderm bacteria.

In some embodiments, the EVs are from diderm bacteria.

In some, the EVs are from bacteria of the family: Prevotellaceae; Veillonellaceae; Tannerellaceae; Rikenellaceae; Selenomonadaceae; Sporomusaceae; Synergistaceae; or Akkermaniaceae.

In some embodiments, the EVs are from bacteria of the family Oscillospiraceae; Clostridiaceae; Lachnospiraceae; or Christensenellaceae.

In some embodiments, the EVs are from bacteria of the genus Prevotella.

In some embodiments, the EVs are from bacteria of the genus Veillonella.

In some embodiments, the EVs are from bacteria of the genus Parabacteroides.

In some embodiments, the EVs are from bacteria of the Oscillospiraceae family.

In some embodiments, the EVs are from bacteria of the Tannerellaceae family.

In some embodiments, the EVs are from bacteria of the Prevotellaceae family.

In some embodiments, the EVs are from bacteria of the Veillonellaceae family.

In some embodiments, the Gram negative bacteria belong to class Negativicutes.

In some embodiments, the Gram negative bacteria belong to family Veillonellaceae, Selenomonadaceae, Acidaminococcaceae, or Sporomusaceae.

In some embodiments, the EVs are from bacteria of the genus Megasphaera, Selenomonas, Propionospora, or Acidaminococcus.

In some embodiments, the EVs are from Megasphaera sp., Selenomonas felix, Acidaminococcus intestine, or Propionospora sp. bacteria.

In some embodiments, the EVs are from bacteria of the genus Lactococcus, Prevotella, Bifidobacterium, or Veillonella.

In some embodiments, the EVs are from Lactococcus lactis cremoris bacteria.

In some embodiments, the EVs are from Prevotella histicola bacteria.

In some embodiments, the EVs are from Bifidobacterium animalis bacteria.

In some embodiments, the EVs are from Veillonella parvula bacteria.

In some embodiments, the EVs are from Lactococcus lactis cremoris bacteria. In some embodiments, the Lactococcus lactis cremoris bacteria are from a strain comprising at least 90% (or at least 97%) genomic, 16S and/or CRISPR sequence identity to the nucleotide sequence of the Lactococcus lactis cremoris Strain A (ATCC designation number PTA-125368). In some embodiments, the Lactococcus bacteria are from a strain comprising at least 99% genomic, 16S and/or CRISPR sequence identity to the nucleotide sequence of the Lactococcus lactis cremoris Strain A (ATCC designation number PTA-125368). In some embodiments, the Lactococcus bacteria are from Lactococcus lactis cremoris Strain A (ATCC designation number PTA-125368).

In some embodiments, the EVs are from Prevotella bacteria. In some embodiments, the Prevotella bacteria are from a strain comprising at least 90% (or at least 97%) genomic, 16S and/or CRISPR sequence identity to the nucleotide sequence of the Prevotella Strain B 50329 (NRRL accession number B 50329). In some embodiments, the Prevotella bacteria are from a strain comprising at least 99% genomic, 16S and/or CRISPR sequence identity to the nucleotide sequence of the Prevotella Strain B 50329 (NRRL accession number B 50329). In some embodiments, the Prevotella bacteria are from Prevotella Strain B 50329 (NRRL accession number B 50329).

In some embodiments, the EVs are from Bifidobacterium bacteria. In some embodiments, the Bifidobacterium bacteria are from a strain comprising at least 90% (or at least 97%) genomic, 16S and/or CRISPR sequence identity to the nucleotide sequence of the Bifidobacterium bacteria deposited as ATCC designation number PTA-125097. In some embodiments, the Bifidobacterium bacteria are from a strain comprising at least 99% genomic, 16S and/or CRISPR sequence identity to the nucleotide sequence of the Bifidobacterium bacteria deposited as ATCC designation number PTA-125097. In some embodiments, the Bifidobacterium bacteria are from Bifidobacterium bacteria deposited as ATCC designation number PTA-125097.

In some embodiments, the EVs are from Veillonella bacteria. In some embodiments, the Veillonella bacteria are from a strain comprising at least 90% (or at least 97%) genomic, 16S and/or CRISPR sequence identity to the nucleotide sequence of the Veillonella bacteria deposited as ATCC designation number PTA-125691. In some embodiments, the Veillonella bacteria are from a strain comprising at least 99% genomic, 16S and/or CRISPR sequence identity to the nucleotide sequence of the Veillonella bacteria deposited as ATCC designation number PTA-125691. In some embodiments, the Veillonella bacteria are from Veillonella bacteria deposited as ATCC designation number PTA-125691.

In some embodiments, the EVs are from Ruminococcus gnavus bacteria. In some embodiments, the Ruminococcus gnavus bacteria are from a strain comprising at least 90% (or at least 97%) genomic, 16S and/or CRISPR sequence identity to the nucleotide sequence of the Ruminococcus gnavus bacteria deposited as ATCC designation number PTA-126695. In some embodiments, the Ruminococcus gnavus bacteria are from a strain comprising at least 99% genomic, 16S and/or CRISPR sequence identity to the nucleotide sequence of the Ruminococcus gnavus bacteria deposited as ATCC designation number PTA-126695. In some embodiments, the Ruminococcus gnavus bacteria are from Ruminococcus gnavus bacteria deposited as ATCC designation number PTA-126695.

In some embodiments, the EVs are from Megasphaera sp. bacteria. In some embodiments, the Megasphaera sp. bacteria are from a strain comprising at least 90% (or at least 97%) genomic, 16S and/or CRISPR sequence identity to the nucleotide sequence of the Megasphaera sp. bacteria deposited as ATCC designation number PTA-126770. In some embodiments, the Megasphaera sp. bacteria are from a strain comprising at least 99% genomic, 16S and/or CRISPR sequence identity to the nucleotide sequence of the Megasphaera sp. bacteria deposited as ATCC designation number PTA-126770. In some embodiments, the Megasphaera sp. bacteria are from Megasphaera sp. bacteria deposited as ATCC designation number PTA-126770.

In some embodiments, the EVs are from Fournierella massiliensis bacteria. In some embodiments, the Fournierella massiliensis bacteria are from a strain comprising at least 90% (or at least 97%) genomic, 16S and/or CRISPR sequence identity to the nucleotide sequence of the Fournierella massiliensis bacteria deposited as ATCC designation number PTA-126696. In some embodiments, the Fournierella massiliensis bacteria are from a strain comprising at least 99% genomic, 16S and/or CRISPR sequence identity to the nucleotide sequence of the Fournierella massiliensis bacteria deposited as ATCC designation number PTA-126696. In some embodiments, the Fournierella massiliensis bacteria are from Fournierella massiliensis bacteria deposited as ATCC designation number PTA-126696.

In some embodiments, the EVs are from Harryflintia acetispora bacteria. In some embodiments, the Harryflintia acetispora bacteria are from a strain comprising at least 90% (or at least 97%) genomic, 16S and/or CRISPR sequence identity to the nucleotide sequence of the Harryflintia acetispora bacteria deposited as ATCC designation number PTA-126694. In some embodiments, the Harryflintia acetispora bacteria are from a strain comprising at least 99% genomic, 16S and/or CRISPR sequence identity to the nucleotide sequence of the Harryflintia acetispora bacteria deposited as ATCC designation number PTA-126694. In some embodiments, the Harryflintia acetispora bacteria are from Harryflintia acetispora bacteria deposited as ATCC designation number PTA-126694.

In some embodiments, the EVs are from bacteria of the family Acidaminococcaceae, Alcaligenaceae, Akkermansiaceae, Bacteriodaceae, Bidobacteriaceae, Burkholderiaceae, Catabacteriaceae, Clostridiaceae, Coriobacteriaceae, Enterobacteriaceae, Entemcoccaceae, Fusobacteriaceae, Lachnospiraceae, Listeraceae, Mycobacteriaceae, Neisseriaceae, Odoribacteraceae, Oscillospiraceae, Peptococcaceae, Peptostreptococcaceae, Porphyromonadaceae, Prevotellaceae, Propionibacteraceae, Rikenellaceae, Ruminococcaceae, Selenomonadaceae, Sporomusaceae, Streptococcaceae, Streptomycetaceae, Sutterellaceae, Synergistaceae, or Veillonellaceae.

In some embodiments, the EVs are from bacteria of the genus Akkermansia, Christensenella, Blautia, Enterococcus, Eubacterium, Roseburia, Bacteroides, Parabacteroides, or Erysipelatoclostridium.

In some embodiments, the EVs are from Blautia hydrogenotrophica, Blautia stercoris, Blautia wexlerae, Eubacterium faecium, Eubacterium contortum, Eubacterium rectale, Enterococcus faecalis, Enterococcus durans, Enterococcus villorum, Enterococcus gallinarum; Bifidobacterium lactis, Bifidobacterium bifidium, Bifidobacterium longum, Bifidobacterium animalis, or Bifidobacterium breve bacteria.

In some embodiments, the EVs are from BCG (bacillus Calmette-Guerin), Parabacteroides, Blautia, Veillonella, Lactobacillus salivarius, Agathobaculum, Ruminococcus gnavus, Paraclostridium benzoelyticum, Turicibacter sanguinus, Burkholderia, Klebsiella quasipneumoniae ssp similpneumoniae, Klebsiella oxytoca. Tyzzerela nexilis, or Neisseria bacteria.

In some embodiments, the EVs are from Blautia hydrogenotrophica bacteria.

In some embodiments, the EVs are from Blautia stercoris bacteria.

In some embodiments, the EVs are from Blautia wexlerae bacteria.

In some embodiments, the EVs are from Enterococcus gallinarum bacteria.

In some embodiments, the EVs are from Enterococcus faecium bacteria.

In some embodiments, the EVs are from Bifidobacterium bifidium bacteria.

In some embodiments, the EVs are from Bifidobacterium breve bacteria.

In some embodiments, the EVs are from Bifidobacterium longum bacteria.

In some embodiments, the EVs are from Roseburia hominis bacteria.

In some embodiments, the EVs are from Bacteroides thetaiotaomicron bacteria.

In some embodiments, the EVs are from Bacteroides coprocola bacteria.

In some embodiments, the EVs are from Erysipelatoclostridium ramosum bacteria.

In some embodiments, the EVs are from Megasphera massiliensis bacteria.

In some embodiments, the EVs are from Eubacterium bacteria.

In some embodiments, the EVs are from Parabacteroides distasonis bacteria.

In some embodiments, the EVs are from Lactobacillus plantarum bacteria.

In some embodiments, the EVs are from bacteria of the Negativicutes class.

In some embodiments, the EVs are from bacteria of the Veillonellaceae family.

In some embodiments, the EVs are from bacteria of the Selenomonadaceae family.

In some embodiments, the EVs are from bacteria of the Acidaminococcaceae family.

In some embodiments, the EVs are from bacteria of the Sporomusaceae family.

In some embodiments, the EVs are from bacteria of the Megasphaera genus.

In some embodiments, the EVs are from bacteria of the Selenomonas genus.

In some embodiments, the EVs are from bacteria of the Propionospora genus.

In some embodiments, the EVs are from bacteria of the Acidaminococcus genus.

In some embodiments, the EVs are from Megasphaera sp. bacteria.

In some embodiments, the EVs are from Selenomonas felix bacteria.

In some embodiments, the EVs are from Acidaminococcus intestini bacteria.

In some embodiments, the EVs are from Propionospora sp. bacteria.

In some embodiments, the EVs are from bacteria of the Clostridia class.

In some embodiments, the EVs are from bacteria of the Oscillospriraceae family.

In some embodiments, the EVs are from bacteria of the Faecalibacterium genus.

In some embodiments, the EVs are from bacteria of the Fournierella genus.

In some embodiments, the EVs are from bacteria of the Harryflintia genus.

In some embodiments, the EVs are from bacteria of the Agathobaculum genus.

In some embodiments, the EVs are from Faecalibacterium prausnitzii (for example, Faecalibacterium prausnitzii Strain A) bacteria.

In some embodiments, the EVs are from Fournierella massiliensis (for example, Fournierella massiliensis Strain A) bacteria.

In some embodiments, the EVs are from Harryflintia acetispora (for example, Harryflintia acetispora Strain A) bacteria.

In some embodiments, the EVs are from Agathobaculum sp. (for example, Agathobaculum sp. Strain A) bacteria.

In some embodiments, the EVs are from a strain of Agathobaculum sp. In some embodiments, the Agathobaculum sp. strain is a strain comprising at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, CRISPR sequence) of the Agathobaculum sp. Strain A (ATCC Deposit Number PTA-125892). In some embodiments, the Agathobaculum sp. strain is the Agathobaculum sp. Strain A (ATCC Deposit Number PTA-125892).

In some embodiments, the EVs are from bacteria of the class Bacteroidia [phylum Bacteroidota]. In some embodiments, the EVs are from bacteria of order Bacteroidales. In some embodiments, the EVs are from bacteria of the family Porphyromonoadaceae. In some embodiments, the EVs are from bacteria of the family Prevotellaceae. In some embodiments, the EVs are from bacteria of the class Bacteroidia wherein the cell envelope structure of the bacteria is diderm. In some embodiments, the EVs are from bacteria of the class Bacteroidia that stain Gram negative. In some embodiments, the EVs are from bacteria of the class Bacteroidia wherein the bacteria is diderm and the bacteria stain Gram negative.

In some embodiments, the EVs are from bacteria of the class Clostridia [phylum Firmicutes]. In some embodiments, the EVs are from bacteria of the order Eubacteriales. In some embodiments, the EVs are from bacteria of the family Oscillispiraceae. In some embodiments, the EVs are from bacteria of the family Lachnospiraceae. In some embodiments, the EVs are from bacteria of the family Peptostreptococcaceae. In some embodiments, the EVs are from bacteria of the family Clostridiales family XIII/Incertae sedis 41. In some embodiments, the EVs are from bacteria of the class Clostridia wherein the cell envelope structure of the bacteria is monoderm. In some embodiments, the EVs are from bacteria of the class Clostridia that stain Gram negative. In some embodiments, the EVs are from bacteria of the class Clostridia that stain Gram positive. In some embodiments, the EVs are from bacteria of the class Clostridia wherein the cell envelope structure of the bacteria is monoderm and the bacteria stain Gram negative. In some embodiments, the EVs are from bacteria of the class Clostridia wherein the cell envelope structure of the bacteria is monoderm and the bacteria stain Gram positive.

In some embodiments, the EVs are from bacteria of the class Negativicutes [phylum Firmicutes]. In some embodiments, the EVs are from bacteria of the order Veillonellales. In some embodiments, the EVs are from bacteria of the family Veillonelloceae. In some embodiments, the EVs are from bacteria of the order Selenomonadales. In some embodiments, the EVs are from bacteria of the family Selenomonadaceae. In some embodiments, the EVs are from bacteria of the family Sporomusaceae. In some embodiments, the EVs are from bacteria of the class Negativicutes wherein the cell envelope structure of the bacteria is diderm. In some embodiments, the EVs are from bacteria of the class Negativicutes that stain Gram negative. In some embodiments, the EVs are from bacteria of the class Negativicutes wherein the cell envelope structure of the bacteria is diderm and the bacteria stain Gram negative.

In some embodiments, the EVs are from bacteria of the class Synergistia [phylum Synergistota]. In some embodiments, the EVs are from bacteria of the order Synergistales. In some embodiments, the EVs are from bacteria of the family Synergistaceae. In some embodiments, the EVs are from bacteria of the class Synergistia wherein the cell envelope structure of the bacteria is diderm. In some embodiments, the EVs are from bacteria of the class Synergistia that stain Gram negative. In some embodiments, the EVs are from bacteria of the class Synergistia wherein the cell envelope structure of the bacteria is diderm and the bacteria stain Gram negative.

In some embodiments, the EVs are from bacteria that produce metabolites, for example, the bacteria produce butyrate, iosine, proprionate, or tryptophan metabolites.

In some embodiments, the EVs are from bacteria that produce butyrate. In some embodiments, the bacteria are from the genus Blautia; Christensella; Copracoccus; Eubacterium; Lachnosperacea; Megasphaera; or Roseburia.

In some embodiments, the EVs are from bacteria that produce iosine. In some embodiments, the bacteria are from the genus Bifidobacterium; Lactobacillus; or Olsenella.

In some embodiments, the EVs are from bacteria that produce proprionate. In some embodiments, the bacteria are from the genus Akkermansia; Bacteroides; Dialister; Eubacterium; Megasphaera; Parabacteriodes; Prevotella; Ruminococcus; or Veillonella.

In some embodiments, the EVs are from bacteria that produce tryptophan metabolites. In some embodiments, the bacteria are from the genus Lactobacillus or Peptostreptococcus.

In some embodiments, the EVs are from bacteria that produce inhibitors of histone deacetylase 3 (HDAC3). In some embodiments, the bacteria are from the species Bariatricus massiliensis. Faecalibacterium prausnitzii. Megasphaera massiliensis or Roseburia intestinalis.

In some embodiments, the EVs are from bacteria of the genus Alloiococcus; Bacillus; Catenibacterium; Corynebacterium; Cupriavidus; Enhydrobacter; Exiguobacterium; Faecalibacterium; Geobacillus; Methylobacterium; Micrococcus; Morganella; Proteus; Pseudomonas; Rhizobium; or Sphingomonas.

In some embodiments, the EVs are from bacteria of the genus Cutibacterium.

In some embodiments, the EVs are from bacteria of the species Cutibacterium avidum.

In some embodiments, the EVs are from bacteria of the genus Lactobacillus.

In some embodiments, the EVs are from bacteria of the species Lactobacillus gasseri.

In some embodiments, the EVs are from bacteria of the genus Dysosmobacter.

In some embodiments, the EVs are from bacteria of the species Dysosmobacter welbionis.

In some embodiments, the EVs are from bacteria of the genus Leuconostoc.

In some embodiments, the EVs are from bacteria of the genus Lactobacillus.

In some embodiments, the EVs are from bacteria of the genus Akkermansia muciniphila; Bacillus; Blautia; Cupriavidus; Enhydrobacter; Faecalibacterium; Lactobacillus; Lactococcus; Micrococcus; Morganella; Propionibacterium; Proteus; Rhizobium; or Streptococcus.

In some embodiments, the EVs are from Leuconostoc holzapfelii bacteria.

In some embodiments, the EVs are from Akkermansia muciniphila; Cupriavidus metallidurans; Faecalibacterium prausnitzii; Lactobacillus casei; Lactobacillus plantarum; Lactobacillus paracasei; Lactobacillus plantarum; Lactobacillus rhamnosus; Lactobacillus sakei; or Streptococcus pyogenes bacteria.

In some embodiments, the EVs are from Lactobacillus casei; Lactobacillus plantarum; Lactobacillus paracasei; Lactobacillus plantarum; Lactobacillus rhamnosus; or Lactobacillus sakei bacteria.

In some embodiments, the EVs described herein are obtained from a genus selected from the group consisting of Acinetobacter; Deinococcus; Helicobacter; Rhodococcus; Weissella cibaria; Alloiococcus; Atopobium; Catenibacterium; Corynebacterium; Exiguobacterium; Geobacillus; Methylobacterium; Micrococcus; Morganella; Proteus; Rhizobium; Rothia; Sphingomonas; Sphingomonas; and Leuconostoc.

In some embodiments, the EVs described herein are obtained from a species selected from the group consisting of Acinetobacter baumanii; Deinococcus radiodurans; Helicobacter pylori; Rhodococcus equi; Weissella cibaria; Alloiococcus otitis; Atopobium vaginae; Catenibacterium mituokai; Corynebacterium glutamicum; Exiguobacterium aurantiacum; Geobacillus stearothermophilus; Methylobacterium jeotgali; Micrococcus luteus; Morganella morganii; Proteus mirabilis; Rhizobium leguminosarum; Rothia amarae; Sphingomonas paucimobilis; and Sphingomonas koreens.

In some embodiments, the EVs are from Leuconostoc holzapfelii bacteria. In some embodiments, the EVs are from Leuconostoc holzapfelii Ceb-kc-003 (KCCM11830P) bacteria.

In some embodiments, the EVs are from Megasphaera sp. bacteria (for example, from the strain with accession number NCIMB 43385, NCIMB 43386 or NCIMB 43387).

In some embodiments, the EVs are from Megasphaera massiliensis bacteria (for example, from the strain with accession number NCIMB 42787, NCIMB 43388 or NCIMB 43389).

In some embodiments, the EVs are from Megasphaera massiliensis bacteria (for example, from the strain with accession number DSM 26228).

In some embodiments, the EVs are from Parabacteroides distasonis bacteria (for example, from the strain with accession number NCIMB 42382).

In some embodiments, the EVs are from Megasphaera massiliensis bacteria (for example, from the strain with accession number NCIMB 43388 or NCIMB 43389), or a derivative thereof. See, for example, WO 2020/120714. In some embodiments, the Megasphaera massiliensis bacteria is a strain comprising at least 80%, at least 85%, at least 90%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, and/or CRISPR sequence) of Megasphaera massiliensis bacteria from the strain with accession number NCIMB 43388 or NCIMB 43389. In some embodiments, the Megasphaera massiliensis bacteria is the strain with accession number NCIMB 43388 or NCIMB 43389.

In some embodiments, the EVs are from Megasphaera massiliensis bacteria strain deposited under accession number NCIMB 42787, or a derivative thereof. See, for example, WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, and/or CRISPR sequence) of the Megasphaera massiliensis bacteria strain deposited under accession number NCIMB 42787. In some embodiments, the Megasphaera massiliensis bacteria is the strain deposited under accession number NCIMB 42787.

In some embodiments, the EVs are from Megasphaera spp. bacteria from the strain with accession number NCIMB 43385, NCIMB 43386 or NCIMB 43387, or a derivative thereof. See, for example, WO 2020/120714. In some embodiments, the Megasphaera sp. bacteria is a strain comprising at least 80%, at least 85%, at least 90%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, and/or CRISPR sequence) of the Megasphaera sp. from a strain with accession number NCIMB 43385, NCIMB 43386 or NCIMB 43387. In some embodiments, the Megasphaera sp. bacteria is the strain with accession number NCIMB 43385, NCIMB 43386 or NCIMB 43387.

In some embodiments, the EVs are from Parabacteroides distasonis bacteria deposited under accession number NCIMB 42382, or a derivative thereof. See, for example, WO 2018/229216. In some embodiments, the Parabacteroides distasonis bacteria is a strain comprising at least 80%, at least 85%, at least 90%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, and/or CRISPR sequence) of the Parabacteroides distasonis bacteria deposited under accession number NCIMB 42382. In some embodiments, the Parabacteroides distasonis bacteria is the strain deposited under accession number NCIMB 42382.

In some embodiments, the EVs are from Megasphaera massiliensis bacteria deposited under accession number DSM 26228, or a derivative thereof. See, for example, WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, and/or CRISPR sequence) of Megasphaera massiliensis bacteria deposited under accession number DSM 26228. In some embodiments, the Megasphaera massiliensis bacteria is the strain deposited under accession number DSM 26228.

In certain aspects, the EVs obtained from bacteria that have been selected based on certain desirable properties, such as reduced toxicity and adverse effects (for example, by removing or deleting lipopolysaccharide (LPS)), enhanced oral delivery (for example, by improving acid resistance, muco-adherence and/or penetration and/or resistance to bile acids, resistance to anti-bacterial peptides and/or antibody neutralization), target desired cell types (for example, M-cells, goblet cells, enterocytes, dendritic cells, macrophages), improved bioavailability systemically or in an appropriate niche (for example, mesenteric lymph nodes, Peyer's patches, lamina propria, tumor draining lymph nodes, and/or blood), enhanced immunomodulatory and/or therapeutic effect (for example, either alone or in combination with another therapeutic agent), enhanced immune activation, and/or manufacturing attributes (for example, growth characteristics, yield, greater stability, improved freeze-thaw tolerance, shorter generation times).

In certain aspects, the EVs are from engineered bacteria that are modified to enhance certain desirable properties. In some embodiments, the engineered bacteria are modified so that EVs produced therefrom will have reduced toxicity and adverse effects (for example, by removing or deleting lipopolysaccharide (LPS)), enhanced oral delivery (for example, by improving acid resistance, muco-adherence and/or penetration and/or resistance to bile acids, resistance to anti-microbial peptides and/or antibody neutralization), target desired cell types (for example, M-cells, goblet cells, enterocytes, dendritic cells, macrophages), improved bioavailability systemically or in an appropriate niche (for example, mesenteric lymph nodes, Peyer's patches, lamina propria, tumor draining lymph nodes, and/or blood), enhanced immunomodulatory and/or therapeutic effect (for example, either alone or in combination with another therapeutic agent), enhanced immune activation, and/or improved manufacturing attributes (for example, growth characteristics, yield, greater stability, improved freeze-thaw tolerance, shorter generation times). In some embodiments, provided herein are methods of making such EVs.

In certain aspects, provided herein are solutions and/or dried form (or therapeutic compositions thereof) comprising EVs from bacteria useful for the treatment and/or prevention of a disease or a health disorder (for example, a cancer, an autoimmune disease, an inflammatory disease, dysbiosis, or a metabolic disease), as well as methods of making and/or identifying such solutions and/or dried form (or therapeutic compositions thereof), and methods of using such solutions and/or dried form (for example, for the treatment of a cancer, an autoimmune disease, an inflammatory disease, dysbiosis, or a metabolic disease), either alone or in combination with one or more other therapeutics.

In some embodiments, the gamma irradiated EVs from bacteria are formulated therapeutic compositions containing a solution and/or dried form (for example, lyophilate) and provide potency comparable to or greater than therapeutic compositions that contain the whole bacteria from which the EVs were obtained. For example, at the same dose of EVs (for example, based on particle count or protein content), a therapeutic composition containing solutions and/or powders provide potency comparable to or greater than a comparable therapeutic composition that contains whole bacteria of the same bacterial strain from which the EVs were obtained. In some embodiments, the gamma irradiated EVs from bacteria are formulated such solution—and/or dried form—(for example, lyophilate)—containing therapeutic compositions allow the administration of higher doses and elicit a comparable or greater (for example, more effective) response than observed with a comparable therapeutic composition that contains whole bacteria of the same bacterial strain from which the EVs were obtained.

As a further example, in some embodiments, the gamma irradiated EVs from bacteria are formulated at the same dose (for example, based on particle count or protein content), a therapeutic composition containing a solution and/or dried form (for example, lyophilate) contain less microbially-derived material (based on particle count or protein content), as compared to a therapeutic composition that contains the whole bacteria of the same bacterial strain from which the EVs were obtained, while providing an equivalent or greater therapeutic benefit to the subject receiving such therapeutic composition.

As a further example, in some embodiments, EVs from bacteria are administered at doses for example, of about 1×10⁷ to about 1×10¹⁵ particles, for example, as measured by NTA. In some embodiments, the dose of EVs is about 1×10⁵ to about 7×10¹³ particles (for example, wherein particle count is determined by NTA (nanoparticle tracking analysis)). In some embodiments, the dose of EVs from bacteria is about 1×10¹⁰ to about 7×10¹³ particles (for example, wherein particle count is determined by NTA (nanoparticle tracking analysis)).

As another example, in some embodiments, EVs from bacteria are administered at doses for example, of about 5 mg to about 900 mg total protein, for example, as measured by Bradford assay. As another example, in some embodiments, EVs from bacteria are administered at doses for example, of about 5 mg to about 900 mg total protein, for example, as measured by BCA assay.

In certain embodiments, provided herein are methods of treating a subject who has cancer comprising administering to the subject a therapeutic composition or a solution and/or dried form described herein. In certain embodiments, provided herein are methods of treating a subject who has an immune disorder (for example, an autoimmune disease, an inflammatory disease, an allergy) comprising administering to the subject a therapeutic composition or a solution and/or dried form described herein. In certain embodiments, provided herein are methods of treating a subject who has a metabolic disease comprising administering to the subject a therapeutic composition or a solution and/or dried form described herein. In certain embodiments, provided herein are methods of treating a subject who has a dysbiosis comprising administering to the subject a therapeutic composition or a solution and/or dried form described herein. In certain embodiments, provided herein are methods of treating a subject who has a neurologic disease comprising administering to the subject a therapeutic composition or a solution and/or dried form described herein.

In some embodiments, the method further comprises administering to the subject an antibiotic. In some embodiments, the method further comprises administering to the subject one or more other cancer therapies (for example, surgical removal of a tumor, the administration of a chemotherapeutic agent, the administration of radiation therapy, and/or the administration of a cancer immunotherapy, such as an immune checkpoint inhibitor, a cancer-specific antibody, a cancer vaccine, a primed antigen presenting cell, a cancer-specific T cell, a cancer-specific chimeric antigen receptor (CAR) T cell, an immune activating protein, and/or an adjuvant). In some embodiments, the method further comprises the administration of another therapeutic bacterium and/or EVs from bacteria from one or more other bacterial strains (for example, therapeutic bacterium). In some embodiments, the method further comprises the administration of an immune suppressant and/or an anti-inflammatory agent. In some embodiments, the therapeutic composition or a solution, and/or dried form are for use in combination with one or more other immune effect modulators. In some embodiments, the method further comprises the administration of a metabolic disease therapeutic agent.

In certain aspects, provided herein is a therapeutic composition or a solution and/or dried form for use in the treatment and/or prevention of a disease (for example, a cancer, an autoimmune disease, an inflammatory disease, a dysbiosis, or a metabolic disease) or a health disorder, either alone or in combination with one or more other (e.g., additional) therapeutic agent.

In certain embodiments, provided herein is a therapeutic composition or a solution and/or dried form for use in treating and/or preventing a cancer in a subject (for example, human). In some embodiments, the therapeutic composition or a solution and/or dried form is used either alone or in combination with one or more other therapeutic agent for the treatment of the cancer. In certain embodiments, provided herein is a therapeutic composition or a solution and/or dried form for use in treating and/or preventing an immune disorder (for example, an autoimmune disease, an inflammatory disease, an allergy) in a subject (for example, human). In some embodiments, the therapeutic composition or a solution and/or dried form is used either alone or in combination with one or more other therapeutic agent for the treatment of the immune disorder. In certain embodiments, provided herein is a therapeutic composition or a solution and/or dried form for use in treating and/or preventing a dysbiosis in a subject (for example, human). In some embodiments, the therapeutic composition or a solution and/or dried form is used either alone or in combination with therapeutic agent for the treatment of the dysbiosis. In certain embodiments, provided herein is a therapeutic composition or a solution and/or dried form for use in treating and/or preventing a metabolic disease in a subject (for example, human). In some embodiments, the therapeutic composition or a solution and/or dried form is used either alone or in combination with therapeutic agent for the treatment of the metabolic disease. In certain embodiments, provided herein is a therapeutic composition or a solution and/or dried form for use in treating and/or preventing a dysbiosis in a subject (for example, human). In some embodiments, the therapeutic composition or a solution and/or dried form is used either alone or in combination with therapeutic agent for the treatment of the dysbiosis. In certain embodiments, provided herein is a therapeutic composition or a solution and/or dried form for use in treating and/or preventing a neurologic disease in a subject (for example, human). In some embodiments, the therapeutic composition or a solution and/or dried form is used either alone or in combination with one or more other therapeutic agent for treatment of the neurologic disorder.

In some embodiments, the therapeutic composition or a solution and/or dried form is for use in combination with an antibiotic. In some embodiments, the therapeutic composition or a solution and/or dried form is for use in combination with one or more other cancer therapies (for example, surgical removal of a tumor, the use of a chemotherapeutic agent, the use of radiation therapy, and/or the use of a cancer immunotherapy, such as an immune checkpoint inhibitor, a cancer-specific antibody, a cancer vaccine, a primed antigen presenting cell, a cancer-specific T cell, a cancer-specific chimeric antigen receptor (CAR) T cell, an immune activating protein, and/or an adjuvant). In some embodiments, the therapeutic composition or a solution and/or dried form is for use in combination with another therapeutic bacterium and/or EVs obtained from one or more other bacterial strains (for example, therapeutic bacterium). In some embodiments, the therapeutic composition or a solution and/or dried form is for use in combination with one or more immune suppressant(s) and/or an anti-inflammatory agent(s). In some embodiments, the therapeutic composition or a solution and/or dried form is for use in combination with one or more other metabolic disease therapeutic agents.

In certain aspects, provided herein is use of a therapeutic composition or a solution and/or dried form for the preparation of a medicament for the treatment and/or prevention of a disease (for example, a cancer, an autoimmune disease, an inflammatory disease, a dysbiosis, or a metabolic disease), either alone or in combination with another therapeutic agent. In some embodiments, the use is in combination with another therapeutic bacterium and/or EVs obtained from one or more other bacterial strains (for example, therapeutic bacterium).

In certain embodiments, provided herein is use of a therapeutic composition or a solution and/or dried form for the preparation of a medicament for treating and/or preventing a cancer in a subject (for example, human). In some embodiments, the therapeutic composition or a solution and/or dried form is for use either alone or in combination with another therapeutic agent for the cancer. In certain embodiments, provided herein is use of a therapeutic composition or a solution and/or dried form (for the preparation of a medicament for treating and/or preventing an immune disorder (for example, an autoimmune disease, an inflammatory disease, an allergy) in a subject (for example, human). In some embodiments, the therapeutic composition or a solution and/or dried form is for use either alone or in combination with another therapeutic agent for the immune disorder. In certain embodiments, provided herein is use of a therapeutic composition or a solution and/or dried form for the preparation of a medicament for treating and/or preventing a dysbiosis in a subject (for example, human). In some embodiments, the therapeutic composition or a solution and/or dried form is for use either alone or in combination with another therapeutic agent for the dysbiosis. In certain embodiments, provided herein is use of a therapeutic composition or a solution and/or dried form for the preparation of a medicament for treating and/or preventing a metabolic disease in a subject (for example, human). In some embodiments, the therapeutic composition or a solution and/or dried form is for use either alone or in combination with another therapeutic agent for the metabolic disease. In certain embodiments, provided herein is use of a therapeutic composition or a solution and/or dried form for the preparation of a medicament for treating and/or preventing a dysbiosis in a subject (for example, human). In some embodiments, the therapeutic composition or a solution and/or dried form is for use either alone or in combination with another therapeutic agent for the dysbiosis. In certain embodiments, provided herein is use of a therapeutic composition or a solution and/or dried form for the preparation of a medicament for treating and or preventing a neurologic disease in a subject (for example, human). In some embodiments, the therapeutic composition or a solution and/or dried form is for use either alone or in combination with another therapeutic agent for the neurologic disorder.

In some embodiments, the therapeutic composition or a solution and/or dried form is for use in combination with an antibiotic. In some embodiments, the therapeutic composition or a solution and/or dried form is use in combination with one or more other cancer therapies (for example, surgical removal of a tumor, the use of a chemotherapeutic agent, the use of radiation therapy, and/or the use of a cancer immunotherapy, such as an immune checkpoint inhibitor, a cancer-specific antibody, a cancer vaccine, a primed antigen presenting cell, a cancer-specific T cell, a cancer-specific chimeric antigen receptor (CAR) T cell, an immune activating protein, and/or an adjuvant). In some embodiments, the therapeutic composition or a solution and/or dried form is for use in combination with another therapeutic bacterium and/or EVs obtained from one or more other bacterial strains (for example, therapeutic bacterium). In some embodiments, the therapeutic composition or a solution and/or dried form is for use in combination with one or more other immune suppressant(s) and/or an anti-inflammatory agent(s). In some embodiments, the therapeutic composition or a solution and/or dried form is for use in combination with one or more other metabolic disease therapeutic agent(s).

In some embodiments, a therapeutic composition or a solution and/or dried form, for example, as described herein, comprising EVs from bacteria provides a therapeutically effective amount of EVs to a subject, for example, a human.

In some embodiments, a therapeutic composition or a solution and/or dried form, for example, as described herein, comprising EVs from bacteria provides a non-natural amount of the therapeutically effective components (for example, present in the EVs) to a subject, for example, a human.

In some embodiments, a therapeutic composition or a solution and/or dried form, for example, as described herein, comprising EVs from bacteria provides unnatural quantity of the therapeutically effective components (for example, present in the EVs) to a subject, for example, a human.

In some embodiments, a therapeutic composition or a solution and/or dried form, for example, as described herein, comprising EVs from bacteria brings about one or more changes to a subject, for example, human, for example, to treat or prevent a disease or a health disorder.

In some embodiments, a therapeutic composition or a solution and/or dried form, for example, as described herein, comprising EVs from bacteria has potential for significant utility, for example, to affect a subject, for example, a human, for example, to treat or prevent a disease or a health disorder.

In certain aspects, provided herein is a stock comprising one or more excipients, wherein the stock comprises a bulking agent, wherein the stock is for use in combination with extracellular vesicles (EVs) from bacteria (for example, a liquid preparation thereof), for example, EVs from a source provided herein.

In certain aspects, provided herein is a stock comprising one or more excipients, wherein the stock comprises a bulking agent and a lyoprotectant, wherein the stock is for use in combination with extracellular vesicles (EVs) from bacteria (for example, a liquid preparation thereof), for example, EVs from a source provided herein.

In certain aspects, provided herein is a stock comprising one or more excipients, wherein the stock comprises a lyoprotectant, wherein the stock is for use in combination with extracellular vesicles (EVs) from bacteria (for example, a liquid preparation thereof), for example, EVs from a source provided herein.

In some embodiments, the bulking agent comprises mannitol, sucrose, maltodextrin, dextran, Ficoll, or PVP-K30.

In some embodiments, the bulking agent comprises mannitol.

In some embodiments, the excipient solution comprises an additional ingredient.

In some embodiments, the additional ingredient comprises trehalose, mannitol, sucrose, sorbitol, dextran, poloxamer 188, maltodextrin, PVP-K30, Ficoll, citrate, arginine, and/or hydroxypropyl-B-cyclodextrin.

In some embodiments, the excipient solution comprises mannitol and trehalose.

In some embodiments, the excipient solution consists essentially of mannitol and trehalose.

In some embodiments, the excipient solution comprises mannitol, trehalose, and sorbitol.

In some embodiments, the excipient solution consists essentially of mannitol, trehalose, and sorbitol.

In some embodiments, the excipient solution comprises trehalose.

In some embodiments, the excipient solution consists essentially of trehalose.

In some embodiments, the excipient solution comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient solution comprises more mannitol than trehalose, for example, on a weight basis or weight percent basis. In some embodiments, the excipient solution comprises at least two-fold more mannitol than trehalose, for example, on a weight basis or weight percent basis. In some embodiments, the excipient solution comprises at least three-fold more mannitol than trehalose, for example, on a weight basis or weight percent basis. In some embodiments, the excipient of the solution or dried form comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient of the solution or dried form comprises more mannitol than trehalose, for example, on a weight basis or weight percent basis. In some embodiments, the excipient of the solution or dried form comprises at least two-fold more mannitol than trehalose, for example, on a weight basis or weight percent basis. In some embodiments, the excipient of the solution or dried form comprises at least three-fold more mannitol than trehalose, for example, on a weight basis or weight percent basis.

In some embodiments, the excipient solution consists essentially of mannitol and trehalose. In some embodiments, the excipient solution consists essentially of mannitol and trehalose, wherein the mannitol and the trehalose are not present in equal amounts (for example, the mannitol and the trehalose are present in unequal amounts; for example, on a weight basis or a weight percent basis). In some embodiments, the excipient solution consists essentially of mannitol and trehalose, wherein the excipient solution contains more mannitol than trehalose, for example, on a weight basis or weight percent basis. In some embodiments, the excipient solution consists essentially of mannitol and trehalose, wherein the excipient solution contains at least two-fold more mannitol than trehalose, for example, on a weight basis or weight percent basis. In some embodiments, the excipient solution consists essentially of mannitol and trehalose, wherein the excipient solution contains at least three-fold more mannitol than trehalose, for example, on a weight basis or weight percent basis. In some embodiments, the excipient of the solution or dried form consists essentially of mannitol and trehalose, wherein the excipient of the solution or dried form contains more mannitol than trehalose, for example, on a weight basis or weight percent basis. In some embodiments, the excipient of the solution or dried form consists essentially of mannitol and trehalose, wherein the excipient of the solution or dried form contains at least two-fold more mannitol than trehalose, for example, on a weight basis or weight percent basis. In some embodiments, the excipient of the solution or dried form consists essentially of mannitol and trehalose, wherein the excipient of the solution or dried form contains at least three-fold more mannitol than trehalose, for example, on a weight basis or weight percent basis.

In some embodiments, the excipient solution comprises, or consists essentially of, mannitol and trehalose, wherein neither the mannitol nor the trehalose is present in an amount of 5 mg/ml to 15 mg/ml. In some embodiments, the excipient solution comprises, or consists essentially of, mannitol and trehalose, wherein the mannitol is not present in an amount of 5 mg/ml to 15 mg/ml. In some embodiments, the excipient solution comprises, or consists essentially of, mannitol and trehalose, wherein the trehalose is not present in an amount of 5 mg/ml to 15 mg/ml.

In some embodiments, the excipient solution comprises, or consists essentially of, mannitol and trehalose, wherein neither the mannitol nor the trehalose is present in an amount of 9 mg/ml. In some embodiments, the excipient solution comprises, or consists essentially of, mannitol and trehalose, wherein the mannitol is not present in an amount of 9 mg/ml. In some embodiments, the excipient solution comprises, or consists essentially of, mannitol and trehalose, wherein the trehalose is not present in an amount of 9 mg/ml.

In some embodiments, the excipient solution comprises, or consists essentially of, mannitol and trehalose, and does not comprise methionine.

In certain aspects, provided herein is a stock comprising one or more excipients, wherein the stock comprises a formula provided in Table A, B, C, D, K or P.

In certain aspects, provided herein is a stock comprising one or more excipients, wherein the stock comprises a formula provided in Table A, B, C, D, K or P wherein the stock is for use in combination with extracellular vesicles (EVs) (for example, a liquid preparation thereof), such as bacterial EVs, such as EVs from a source provided herein.

In some embodiments of the solutions and dried forms and methods described herein, a liquid preparation comprises a cell culture supernatant, such as a bacterial cell culture supernatant, for example, as described herein. In some embodiments of the solution and dried forms and methods described herein, the liquid preparation comprises a retentate, such as a concentrated retentate, for example, as described herein.

In some embodiments of the methods provided herein, excipients are present in (for example, provided in) an excipient solution. Examples of an excipient solution include the stocks comprising one or more excipients provided in Tables A, B, C, D, K and P. For example, the dried forms provided herein contain excipients from the excipient solution (such as a stock) once the moisture has been removed, such as by drying. For example, a liquid preparation that comprises EVs is combined with the stock of formula 7a (which comprises the excipients mannitol and trehalose) from Table A to prepare a solution. The solution is dried to prepare a dried form. The dried form comprises EVs, mannitol, and trehalose.

### BRIEF DESCRIPTION OF THE FIGURES

FIG. 1 is a graph showing the effects of orally-administered Prevotella EVs powder prepared in formula 7a in a delayed type hypersensitivity (DTH) model of inflammation. Inflammation is assessed as change in ear thickness (mm).

FIG. 2 is a graph showing powder concentration (particles/mg) for Oscillospiraceae Family.

FIG. 3 is a graph showing powder concentration (particles/mg) for Veillonellaceae Family.

FIG. 4 is a graph showing powder concentration (particles/mg) for Prevotellaceae Family.

FIG. 5 is a graph showing powder concentration (particles/mg) for Tannerellaceae Family.

FIG. 6 is a graph showing powder concentration (particles/mg) for Clostridiaceae, Lachnospiraceae, Rikenellaceae, Sporomusaceae, Christensenellaceae, Selenomonadaceae, Synergistaceae, and Akkermansiaceae Families.

FIG. 7 is a graph showing size by DLS for Oscillospiraceae Family.

FIG. 8 is a graph showing size by DLS for Tannerellaceae Family.

FIG. 9 is a graph showing size by DLS for Veillonellaceae Family.

FIG. 10 is a graph showing size by DLS for Prevotellaceae Family.

FIG. 11 is a graph showing size by DLS for Clostridiaceae, Lachnospiraceae, Rikenellaceae, Sporomusaceae, Christensenellaceae, Selenomonadaceae, Synergistaceae, and Akkermansiaceae Families.

FIG. 12 is a graph showing charge (Zeta Potential) by DLS for Oscillospiraceae Family.

FIG. 13 is a graph showing charge (Zeta Potential) by DLS for Tannerellaceae Family.

FIG. 14 is a graph showing charge (Zeta Potential) by DLS for Veillonellaceae Family.

FIG. 15 is a graph showing charge (Zeta Potential) by DLS for Prevotellaceae Family

FIG. 16 is a graph showing charge (Zeta Potential) by DLS for Clostridiaceae, Lachnospiraceae, Rikenellaceae, Sporomusaceae, Christensenellaceae, Selenomonadaceae, Synergistaceae, and Akkermansiaceae Families.

FIG. 17 is a graph showing Zₐᵥₑ Size for Oscillospiraceae Family.

FIG. 18 is a graph showing Zₐᵥₑ Size for Prevotellaceae Family.

FIG. 19 is a graph showing Zₐᵥₑ Size for Tannerellaceae Family.

FIG. 20 is a graph showing Zₐᵥₑ Size for Veillonellaceae Family.

FIG. 21 is a graph showing Zₐᵥₑ Size for Clostridiaceae, Lachnospiraceae, Rikenellaceae, Sporomusaceae, Christensenellaceae, Selenomonadaceae, Synergistaceae, and Akkermansiaceae Families.

FIG. 22 is a graph showing charge (Zeta Potential) by DLS for Oscillospiraceae Family.

FIG. 23 is a graph showing charge (Zeta Potential) by DLS for Veillonellaceae Family.

FIG. 24 is a graph showing charge (Zeta Potential) by DLS for Prevotellaceae Family.

FIG. 25 is a graph showing charge (Zeta Potential) by DLS for Tannerellaceae Family.

FIG. 26 is a graph showing charge (Zeta Potential) by DLS for Clostridiaceae, Lachnospiraceae, Rikenellaceae, Sporomusaceae, Christensenellaceae, Selenomonadaceae, Synergistaceae, and Akkermansiaceae Families.

FIG. 27 is a graph showing Karl Fischer Water Content of Prevotellaceae powders.

FIG. 28 is a graph showing Karl Fischer Water Content of Tannerellaceae powders.

FIG. 29 is a graph showing Karl Fischer Water Content of Oscillospiraceae powders.

FIG. 30 is a graph showing Karl Fischer Water Content of Veillonellaceae powders.

FIG. 31 is a graph showing Karl Fischer Water Content of Clostridiaceae, Lachnospiraceae, Rikenellaceae, Sporomusaceae, Christensenellaceae, Selenomonadaceae, Synergistaceae, and Akkermansiaceae powders.

FIG. 32 is a graph showing levels of IL-10 normalized to LPS for Prevotellaceae family. For FIGS. 32-61, the y-axis represents the fold change relative to 10 ng/mL LPS plate control. Particle concentration is reported in particles per well on the x-axis (10⁶, 10⁷, 10⁸, and 10⁹). Bars represent the mean and standard deviation of triplicate wells from a single experiment.

FIG. 33 is a graph showing levels of IL-10 normalized to LPS or Tannerellaceae Family.

FIG. 34 is a graph showing levels of IL-10 normalized to LPS for Oscillospiraceae Family.

FIG. 35: IL-10 normalized to LPS for Veillonellaceae Family.

FIG. 36 is a graph showing levels of IL-10 normalized to LPS for Clostridiaceae. Lachnospiraceae. and Sporomuscae Families.

FIG. 37 is a graph showing levels of IL-10 normalized to LPS for Rikenellaceae, Selenomonadaceae, Christensenellaceae, Synergistaceae, and Akkermansiaceae Families.

FIG. 38 is a graph showing levels of IP-10 normalized to LPS for Tannerellaceae Family.

FIG. 39 is a graph showing levels of IP-10 normalized to LPS for Prevotellaceae Family.

FIG. 40 is a graph showing levels of IP-10 normalized to LPS for Oscillospiraceae Family.

FIG. 41 is a graph showing levels of IP-10 normalized to LPS for Veillonellaceae Family.

FIG. 42 is a graph showing levels of IP-10 normalized to LPS for Clostndiaceae, Lachnospiraceae, and Sporomuscae Families.

FIG. 43 is a graph showing levels of IP-10 normalized to LPS for Rikenellaceae, Selenomonadaceae, Christensenellaceae, Synergistaceae, and Akkermansiaceae Families.

FIG. 44 is a graph showing levels of IL-1β normalized to LPS for Tannerellaceae.

FIG. 45 is a graph showing levels of IL-1β normalized to LPS for Prevotellaceae.

FIG. 46 is a graph showing levels of IL-1β normalized to LPS for Veillonellaceae.

FIG. 47 is a graph showing levels of IL-1β normalized to LPS for Oscillospiraceae.

FIG. 48 is a graph showing levels of IL-1β normalized to LPS for Rikenellaceae, Selenomonadaceae, Christensenellaceae, Synergistaceae, and Akkermansiaceae Families.

FIG. 49 is a graph showing levels of IL-1β normalized to LPS for Clostridiaceae, Lachnospiraceae, and Sporomuscae Families.

FIG. 50 is a graph showing levels of TNFα normalized to LPS for Tannerellaceae.

FIG. 51 is a graph showing levels of TNFα normalized to LPS for Prevotellaceae.

FIG. 52: TNFα normalized to LPS for Oscillospiraceae.

FIG. 53 is a graph showing levels of TNFα normalized to LPS for Veillonellaceae.

FIG. 54 is a graph showing levels of TNFα normalized to LPS for Clostridiaceae, Lachnospiraceae, and Sporomuscae Families.

FIG. 55: TNFα normalized to LPSfor Rikenellaceae, Selenomonadaceae, Christensenellaceae, Synergistaceae, and Akkermansiaceae Families.

FIG. 56 is a graph showing levels of IL-6 normalized to LPS for the Oscillospiraceae Family.

FIG. 57 is a graph showing levels of IL-6 normalized to LPS for Veillonellaceae Family.

FIG. 58 is a graph showing levels of IL-6 normalized to LPS for Tannerellaceae Family.

FIG. 59 is a graph showing levels of IL-6 normalized to LPS for Prevotellaceae Family.

FIG. 60 is a graph showing levels of IL-6 normalized to LPS for Rikenellaceae, Selenomonadaceae, Christensenellaceae, Synergistaceae, and Akkermansiaceae Families.

FIG. 61 is a graph showing levels of IL-6 normalized to LPS for Clostridiaceae, Lachnospiraceae, and Sporomuscae Families.

FIG. 62 is a graph showing moisture content of lyophilized EV powders.

FIG. 63 is a graph showing particle count of lyophilized EV powders.

FIG. 64 is a graph showing average particle size by DLS of lyophilized EV powders.

FIG. 65 is a graph showing electrokinetic potential of the dominant subpopulation of lyophilized EV powder by DLS.

FIG. 66 is a graph showing particle size of the dominant subpopulation of lyophilized EV powders.

### DETAILED DESCRIPTION

The disclosure provides solutions and dried forms that contain extracellular vesicles (EVs) from bacteria, and methods for preparing and using the same. The disclosure also provides therapeutic compositions that contain the solutions and/or dried forms. In some embodiments, EVs are secreted (for example, produced) by bacterial cells in culture. Such secreted extracellular vesicles may be referred to as secreted microbial extracellular vesicles (smEVs). In some embodiments, EVs are prepared (for example, artificially prepared) by processing bacterial cells, for example, by methods that disrupt the bacterial membrane, such as sonication. Such artificially prepared may be referred to as processed microbial extracellular vesicles (pmEVs).

As used herein, a “dried form” that contains extracellular vesicles (EVs) (for example, from bacteria) refers to the product resulting from drying a solution that contains EVs. In some embodiments, the drying is performed, for example, by freeze drying (lyophilization) or spray drying. In some embodiments, the dried form is a powder. As used herein, a powder refers to a type of dried form and includes a lyophilized powder, and a spray-dried powder, obtained by a method such as spray drying.

When freeze drying (lyophilization) is performed, the resulting dried form is a lyophilate. In some embodiments, the dried form is a lyophilate. For example, in some embodiments, a lyophilate is a lyophilized powder or a lyophilized cake. In some embodiments, the lyophilized cake is milled to produce a lyophilized powder.

In some embodiments, the solutions and dried forms that contain EVs from bacteria also comprise one or more excipients, such as a bulking agent, and/or a lyoprotectant.

In some embodiments, bulking agents and lyoprotectants are used when preparing extracellular vesicles (EVs) for freeze drying. In some embodiments, bulking agents, including but not limited to sucrose, mannitol, polyethylene glycol (PEG, such as PEG 6000), cyclodextrin, maltodextrin, and dextran (such as dextran 40k), are added (for example, as a stock containing the same) to a liquid preparation of EVs (for example, obtained by isolating EVs from a bacterial culture) to prepare a dried form such as a lyophilate, making it easier to handle (and optionally, further formulate, for example, into a therapeutic composition) after drying. In some embodiments, lyoprotectants, including but not limited to trehalose, sucrose, and lactose, are added (for example, as a stock containing the same) to a liquid preparation of EVs (for example, obtained by isolating EVs from a bacterial culture) to protect the EVs while lyophilizing or spray drying. In some embodiments, a bulking agent and/or lyoprotectant is included from an excipient stock that is added to EVs (for example, purified and/or concentrated EVs) to produce a solution, and/or to produce a dried form upon subsequent drying, for example, of the solution. In some embodiments, a dried form such as a lyophilate contains between about 5% and about 100% EV solids by weight. In some embodiments, prior to drying (such as by lyophilization), the total solids, including EVs and excipients, are between about 2% and about 20% by weight.

As described herein, in some embodiments, in a lyophilate containing EVs, the excipients make up about 95% to about 99% of the total mass of the powder or cake.

As described herein, in some embodiments, in a lyophilate containing EVs, the EVs make up about 2% to about 6% (for example, about 2% to about 5%, about 2% to about 3%, or about 3% to about 5%) of the total mass of the lyophilate.

In some embodiments, the excipient functions to maintain EV efficacy and/or decrease drying (for example, lyophilization) cycle time. In some embodiments, lyoprotectants protect EVs (for example, protein components thereof) during the freeze-drying process. In some embodiments, bulking agents improve the lyophilate properties, for example, for further downstream processing (such as milling, blending, and/or preparing therapeutic compositions).

The length of the lyophilization cycle is important for cost considerations. Critical temperature modifiers such as bulking agents and/or lyoprotectants can significantly reduce drying time. In some embodiments, an excipient stock containing one or more excipients (for example, that contain a bulking agent and/or lyoprotectant) is added to concentrated EVs (for example, a liquid preparation thereof) to bring the total solids to between about 2% to about 20%. In some embodiments, the EVs are concentrated to 5 to 100 times or volume concentration factors (VCF). Examples provided herein targeted about 10% total solids with actual dissolved solids ranging from about 6% to about 8%. In some embodiments, an excipient stock containing one or more excipients (for example, that contain a bulking agent and/or lyoprotectant) (for example, a stock comprising excipients of a formula provided in one of Tables A, B, C, D, K, and P) is prepared as a stock solution in deionized water and sterile filtered with a 0.2 mm filter prior to use. In some embodiments, the stock solution is added to the concentrated EVs, for example, based on weight up to 80%. In some embodiments, the percentage to add is based on the estimated solids contribution of EVs plus the dissolved solids of the excipient stock to achieve the desired total solids content prior to lyophilization.

After freeze drying EVs (for example, with an excipient that comprises a bulking agent, for example, as described herein), in some embodiments, the resulting lyophilate (for example, lyophilized cake) has a uniform appearance, and is a white to off-white. In some embodiments, the resulting lyophilate (for example, lyophilized cake) obtained after freeze-drying is a white to off-white, fine and smooth granular powder (for example, after milling (for example, grinding) the lyophilized cake). In some embodiments, dynamic light scattering (DLS) is used to obtain the hydrodynamic diameter (Z average, Zₐᵥₑ) of particles present after the lyophilate (for example, lyophilized powder) is resuspended in deionized water or in a buffer such as PBS (for example, 0.1×PBS). In some embodiments, the Zₐᵥₑ is used to quantify the effectiveness of the stabilizer. For example, if the idealized Zₐᵥₑ particle size is 200 nm; therefore, the resuspended EVs with the lowest Zₐᵥₑ closest to this particle size is considered to be sufficiently stabilized. In some embodiments, the particle size ranges, for example, from 130 nm to 300 nm. In some embodiments, dynamic light scattering (DLS) is used to obtain the mean size of the most dominant DLS integrated peak of particles present after the lyophilate (for example, lyophilized powder) is resuspended in deionized water or in a buffer such as PBS (for example, 0.1×PBS). Notably, the mean size of the particles, whether measured by Z average or by the mean size of the most dominant DLS integrated peak, is not necessarily identical to the mean size of the EVs prior to lyophilization. For example, in some embodiments, the mean size of the particles after lyophilization (for example, after the lyophilate is resuspended in deionized water or in a buffer such as PBS (for example, 0.1×PBS)) is larger or smaller than the mean EV size prior to lyophilization, or the mean size after EV isolation or preparation from a bacterial culture (for example, the mean size after gradient purification of EVs from a bacterial culture). Particles in a lyophilate (after a solution containing EVs is lyophilized) contain EVs, and may also include other components from the culture media, such as cell debris, LPS, and/or proteins.

A lyophilate obtained after freeze-drying with the excipients and/or conditions provided herein does not have a porous sponge shape. In some embodiments, after milling, the lyophilate obtained after freeze-drying with the excipients and/or conditions provided herein is a white to off-white, fine and smooth granular lyophilate powder.

Also as described herein, use of the excipients provided herein allows a solution comprising EVs to be freeze dried at higher temperatures and shorter drying times. For example, the excipients and methods provided herein allow for EVs to be freeze dried in less than 4000 minutes, for example, freeze dried in about 2800 to about 3200 minutes. As another example, in some embodiments, the freezing step is performed in less than 225 minutes, as opposed to 10 to 15 hours (600 to 900 minutes). As another example, in some embodiments, using the excipients and methods provided herein, primary drying is performed at a temperature between about −35° C. to about −20° C., for example, about −20° C., about −25° C., about −30° C., or about −35° C., as opposed to, for example, −50° C. As another example, in some embodiments, using the excipients and methods provided herein, primary drying is performed for about 42 hours or less (for example, 2500 minutes or less), as opposed to, for example, 50-60 hours (3000 to 3600 minutes). In some embodiments, using the excipients and methods provided herein, total dry times are, for example, about 72 hours or less, for example, about 48 to about 72 hours, for example, less than about 48 hours. In some embodiments, using the excipients and methods provided herein, primary drying is performed for about 65 hours or less (for example, about 60 hours or less). In some embodiments, using the excipients and methods provided herein, secondary drying is performed for about 12 hours or less (for example, about 10 to about 12 hours, about 5 to about 10 hours, about 10 hours or less, or about 5 hours or less). As another example, in some embodiments, using the excipients and methods provided herein, secondary drying is performed at a temperature between about +20° C. to about +30° C., for example, room temperature, for example, about +25° C., as opposed to, for example, −20° C. In some embodiments, use of shorter drying times and/or higher drying temperatures makes the lyophilization process for EVs more commercially feasible.

As demonstrated in the examples herein, in some embodiments, lyophilates of EVs are prepared from Gram negative and from Gram positive bacteria. For example, EV lyophilates were prepared from the following Gram negative bacteria families: Prevotellaceae; Veillonellaceae; Tannerellaceae; Rikenellaceae; Selenomonadaceae; Sporomusaceae; Synergistaceae; and Akkermaniaceae. For example, EV lyophilates were prepared from the following Gram positive bacteria families: Oscillospiraceae; Clostndiaceae; Lachnospiraceae; and Christensenellaceae.

In some embodiments, the lyophilates containing EVs described herein (for example, prepared using the excipients and/or methods described herein) are prepared to have a moisture content (for example, as determined by the Karl Fischer method) of below about 10% (for example, below about 9%, below about 8%, below about 7%, below about 6%, below about 5% or below about 4%, for example, about 1% to about 4%, about 1.5% to about 4%, about 2% to about 3%) upon completion of freeze drying. In some embodiments, the lyophilates containing EVs described herein (for example, prepared using the excipients and/or methods described herein) are prepared to have a moisture content (for example, as determined by the Karl Fischer method) of below about 6% (for example, below about 5% or below about 4%, for example, about 1% to about 4%, about 1.5% to about 4%, about 2% to about 3%) upon completion of freeze drying. In some embodiments, by preparing lyophilates to have a moisture content below about 6%, the lyophilate are better suited for downstream processing, for example, for use in a therapeutic composition. In some embodiments, by preparing lyophilates to have a moisture content below about 6%, the lyophilate has improved stability, e.g., upon storage.

As described in the examples provided herein, the moisture content (determined by Karl Fis Fischer her) of lyophilates containing EVs of various bacterial families had moisture contents of between about 2.32% to about 5.18%. As described in the examples provided herein, the moisture content (determined by Karl Fischer) of lyophilates containing EVs of the Oscillospiraceae family had moisture contents of between about 4.22% to about 4.98%. As described in the examples provided herein, the moisture content (determined by Karl Fischer) of lyophilates containing EVs of the Tannerellaceae family had moisture contents of between about 3.61% to about 5.09%. As described in the examples provided herein, the moisture content (determined by Karl Fischer) of lyophilates containing EVs of the Prevotellaceae family had moisture contents of between about 3.72% to about 5.23%. As described in the examples provided herein, the moisture content (determined by Karl Fischer) of lyophilates containing EVs of the Veillonellaceae family had moisture contents of between about 2.9% to about 4.35%. Additional examples are provided of lyophilates containing EVs of other bacterial families that had moisture contents of between about 2.32% to about 5.18%. Lyophilates containing EVs of the Veillonella parvula strain exemplified herein had a moisture content (determined by Karl Fischer) of between about 1.24% to about 6.35%. Lyophilates containing EVs of the Fournierella massiliensis strain exemplified herein had a moisture content (determined by Karl Fischer) of between about 1.51% to about 7.01%. Components of the excipient can be selected to obtain the desired moisture content. The drying conditions can be selected to obtain the desired moisture content.

In some embodiments, the lyophilates containing EVs described herein (for example, prepared using the excipients and/or methods described herein) are prepared to have a particle numeration of about 6.7e8 to about 2.55e10 particles/mg lyophilate. In some embodiments, the lyophilates containing EVs described herein (for example, prepared using the excipients and/or methods described herein) are prepared to have a particle numeration of about 6.7e8 to about 2.89e10 particles/mg lyophilate. In some embodiments, particle numeration is determined, for example, on lyophilate resuspended in water by NTA and with use of a Zetaview camera.

As described in the examples provided herein, lyophilates containing EVs of various bacterial families had particle numerations of about 6.7e8 to about 2.55e10 particles/mg lyophilate. As described in the examples provided herein, lyophilates containing EVs of the Oscillospiraceae family had particle numerations of between about 7e8 to about 2.55e10. As described in the examples provided herein, lyophilates containing EVs of the Tannerellaceae family had particle numerations of between about 6.7e8 to about 3.05e8. As described in the examples provided herein, lyophilates containing EVs of the Prevotellaceae family had particle numerations of between about 1.65e9 to about 1.6e10. As described in the examples provided herein, lyophilates containing EVs of the Veillonellaceae family had particle numerations of between about 7.15e8 to about 8.5e9. Lyophilates containing EVs of the Veillonella parvula strain exemplified herein had particle numerations of between about 5e9 to about 1.55e10. Lyophilates containing EVs of the Fournierella massiliensis strain exemplified had particle numerations of between about 6.24e9 to about 2.89e10. Components of the excipient can be selected to obtain the desired particle numeration. The drying conditions can be selected to obtain the desired particle numeration.

In some embodiments, DLS is used to measure the charge of the most dominant DLS integrated peak of particles. In some embodiments, DLS is used to measure the charge of the total particles present in a lyophilate. Notably, the charge of the particles, whether measured for total particles or for the most dominant DLS integrated peak, is not necessarily identical to the charge of the EVs prior to lyophilization. For example, in some embodiments, the charge of the particles after lyophilization (for example, after the lyophilate (for example, lyophilized powder) is resuspended in deionized water or in a buffer such as PBS (for example, 0.1×PBS)) is more or less negative than the charge of EVs prior to lyophilization, or the charge after EV isolation or preparation from a bacterial culture (for example, the charge after gradient purification of EVs from a bacterial culture).

As described in the examples provided herein, the charge of particles of lyophilates of various bacterial families had charges (as measured by zeta potential (mV) for example, by use of dynamic light scattering (DLS) to measure the charge of the total particles present in a lyophilate) of about −29.2 to about +2.67 mV.

In some embodiments, the particles in the lyophilates described herein (for example, prepared using the excipients and/or methods described herein) are prepared to have a charge (as measured by zeta potential (mV), for example, as measured by DLS of the charge of the most dominant DLS integrated peak of particles) of about −29.2 to about +2.67 mV.

As described in the examples provided herein, the charge of particles of lyophilates of the Oscillospiraceae family was between about −15.5 to about −24.2 mV, as measured by DLS of the charge of the most dominant DLS integrated peak of particles. As described in the examples provided herein, the charge of particles of lyophilates of the Tannerellaceae family was between about −4.5 to about −20.7 mV, as measured by DLS of the charge of the most dominant DLS integrated peak of particles. As described in the examples provided herein the charge of particles of lyophilates of the Prevotellaceae family was between about −17.4 to about +2.67 mV, as measured by DLS of the charge of the most dominant DLS integrated peak of particles. As described in the examples provided herein, the charge of particles of lyophilates of the Veillonellaceae family was between about −7.45 to about −29.2 mV, as measured by DLS of the charge of the most dominant DLS integrated peak of particles. The charge of particles of lyophilates of the Veillonella parvula strain exemplified herein was between about −7.54 to about −13.5 mV. The charge of particles of lyophilates of the Fournierella massiliensis strain exemplified was between about −25.3 to about −32 mV. Components of the excipient can be selected to obtain the desired charge. The drying conditions can be selected to obtain the desired charge.

In some embodiments, the particles in the lyophilates described herein (for example, prepared using the excipients and/or methods described herein) are prepared to have a charge (as measured by zeta potential (mV), for example, as measured by DLS of total particles) of about −0.929 to about −24.80 mV.

As described in the examples provided herein, the charge of particles of lyophilates of the Oscillospiraceae family was between about −13.3 to about −24.80 mV, as measured by DLS of the charge of total particles. As described in the examples provided herein, the charge of particles of lyophilates of the Tannerellaceae family was between about −0.929 to about −20.60 mV, as measured by DLS of the charge of total particles. As described in the examples provided herein the charge of particles of lyophilates of the Prevotellaceae family was between about −1.49 to about −11.70 mV, as measured by DLS of the charge of total particles. As described in the examples provided herein, the charge of particles of lyophilates of the Veillonellaceae family was between about −1.88 to about −19.30 mV, as measured by DLS of total particles. The charge of particles of lyophilates of the Veillonella parvula strain exemplified herein was similar to the values calculated for the most dominant DLS integrated peak of particles. The charge of particles of lyophilates of the Fournierella massiliensis strain exemplified was similar to the values calculated for the most dominant DLS integrated peak of particles. Components of the excipient can be selected to obtain the desired charge. The drying conditions can be selected to obtain the desired charge.

In some embodiments, the particles in the lyophilates (for example, lyophilized powders) described herein (for example, prepared using the excipients and/or methods described herein) are prepared to have a hydrodynamic diameter (Z average, Zₐᵥₑ) of about 101 nm to about 752 nm. In some embodiments, dynamic light scattering (DLS) is used to obtain the hydrodynamic diameter (Z average, Zₐᵥₑ) of particles present after the lyophilate is resuspended in deionized water or in a buffer such as PBS (for example, 0.1×PBS).

As described in the examples provided herein, the Zₐᵥₑ of particles of lyophilates of various bacterial families was about 101 nm to about 752 nm (as measured by DLS as measured by DLS after the lyophilate was resuspended in 0.1×PBS). As described in the examples provided herein, the Zₐᵥₑ of particles of lyophilates of the Oscillospiraceae family was between about 101 nm to about 752 nm. As described in the examples provided herein, the Zₐᵥₑ of particles of lyophilates of the Tannerellaceae family was between about 133 nm to about 291 nm. As described in the examples provided herein the Zₐᵥₑ of particles of lyophilates of the Prevotellaceae family was between about 192 nm to about 530 nm. As described in the examples provided herein, the Zₐᵥₑ of particles of lyophilates of the Veillonellaceae family was between about 106 nm to about 178 nm. The Zₐᵥₑ of particles of lyophilates of the Veillonella parvula strain exemplified herein was between about 130.4 nm to about 323.5 nm. The Zₐᵥₑ of particles of lyophilates of the Fournierella massiliensis strain exemplified was between about 132 nm to about 315.2 nm. Components of the excipient can be selected to obtain the desired Zₐᵥₑ. The drying conditions can be selected to obtain the desired Zₐᵥₑ.

In some embodiments, the particles in the lyophilates described herein (for example, prepared using the excipients and/or methods described herein) are prepared to a mean size of the most dominant DLS integrated peak of between about 25.55 nm to about 458.9 nm or between about 25.55 nm to about 157.40 nm. In some embodiments, dynamic light scattering (DLS) is used to obtain the mean size of the most dominant DLS integrated peak of particles present after the lyophilate is resuspended in deionized water or in a buffer such as PBS (for example, 0.1×PBS).

As described in the examples provided herein, the mean size of the most dominant DLS integrated peak of particles of lyophilates of various bacterial families was between about 25.55 nm to about 458.9 nm or between about 25.55 nm to about 157.40 nm (as measured by DLS after the lyophilate was resuspended in 0.1×PBS). As described in the examples provided herein, the mean size of particles of lyophilates of the Oscillospiraceae family was between about 25.55 nm to about 134.8 nm. As described in the examples provided herein, the mean size of particles of lyophilates of the Tannerellaceae family was between about 34.81 nm to about 80.44 nm. As described in the examples provided herein the mean size of particles of lyophilates of the Prevotellaceae family was between about 47.38 nm to about 458.9 nm. As described in the examples provided herein the mean size of particles of lyophilates of the Prevotellaceae family was between about 47.58 nm to about 157.40 nm, for example, if aggregates are excluded. As described in the examples provided herein, the mean size of particles of lyophilates of the Veillonellaceae family was between about 39.86 to about 71.30 nm. The mean size of particles of lyophilates of the Veillonella parvula strain exemplified herein was between about 40 nm to about 78.8 nm. The mean size of particles of lyophilates of the Fournierella massiliensis strain exemplified was between about 43.72 nm to about 79.18 nm. Components of the excipient can be selected to obtain the desired mean size. The drying conditions can be selected to obtain the desired mean size.

As described in the examples provided herein, in some embodiments, lyophilates containing EVs have biological activity, for example, in a U937 cytokine secretion assay. For example, in some embodiments, lyophilates of EVs prepared as described herein affect levels of secreted IL-10, IP-10, IL-1β, TNF-α, and IL-6 levels from U937 cells, for example, as compared to control levels.

In some embodiments, the spray-dried powders containing EVs described herein (for example, prepared using the excipients and/or methods described herein) are prepared to have a moisture content (for example, as determined by the Karl Fischer method) of below about 10% (for example, below about 9%, below about 8%, below about 7%, below about 6%, below about 5% or below about 4%, for example, about 1% to about 4%, about 1.5% to about 4%, about 2% to about 3%) upon completion of spray drying. In some embodiments, the spray-dried powders containing EVs described herein (for example, prepared using the excipients and/or methods described herein) are prepared to have a moisture content (for example, as determined by the Karl Fischer method) of below about 6% (for example, below about 5% or below about 4%, for example, about 1% to about 4%, about 1.5% to about 4%, about 2% to about 3%) upon completion of spray drying. In some embodiments, by preparing spray-dried powders to have a moisture content below about 6%, the spray-dried powders are better suited for downstream processing, for example, for use in a therapeutic composition. In some embodiments, by preparing spray-dried powders to have a moisture content below about 6%, the spray-dried powder has improved stability, e.g., upon storage.

As described in the examples provided herein, the moisture content (determined by Karl Fischer) of spray-dried powders containing Prevotella histicola EVs had moisture contents of between about 2.54% to about 8.38%. Components of the excipient can be selected to obtain the desired moisture content. The drying conditions can be selected to obtain the desired moisture content.

In some embodiments, the spray-dried powders containing EVs described herein (for example, prepared using the excipients and/or methods described herein) are prepared to have a particle numeration of about 6.7e8 to about 2.55e10 particles/mg spray-dried powder. In some embodiments, the spray-dried powders containing EVs described herein (for example, prepared using the excipients and/or methods described herein) are prepared to have a particle numeration of about 6.7e8 to about 2.89e10 particles/mg spray-dried powder. In some embodiments, particle numeration is determined, for example, by NTA, such as with Zetaview.

As described in the examples provided herein, spray-dried powders containing Prevotella histicola EVs had particle numerations of about 8.05e9 to about 2.e10 particles/mg spray-dried powder. Components of the excipient can be selected to obtain the desired particle numeration. The drying conditions can be selected to obtain the desired particle numeration.

### Definitions

Unless specifically stated or obvious from context, as used herein, the term “or” is understood to be inclusive. Unless specifically stated or obvious from context, as used herein, the terms “a,” “an,” and “the” are understood to be singular or plural.

“Adjuvant” or “adjuvant therapy” broadly refers to an agent that affects an immunological or physiological response in a patient or subject (for example, human). For example, an adjuvant might increase the presence of an antigen over time or to an area of interest like a tumor, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition (for example, a pharmaceutical composition) to a subject. Examples of routes of administration include oral administration, rectal administration, topical administration, inhalation (nasal) or injection. Administration by injection includes intravenous (IV), intramuscular (IM), intratumoral (IT) and subcutaneous (SC) administration. In some embodiments, a therapeutic composition described herein is administered in any form by any effective route, including but not limited to intratumoral, oral, parenteral, enteral, intravenous, intraperitoneal, topical, transdermal (for example, using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (for example, sublingual, lingual, (trans)buccal, (trans)urethral, vaginal (for example, trans- and perivaginally), implanted, intravesical, intrapulmonary, intraduodenal, intragastrical, and intrabronchial. In preferred embodiments, a therapeutic composition described herein is administered orally, rectally, intratumorally, topically, intravesically, by injection into or adjacent to a draining lymph node, intravenously, by inhalation or aerosol, or subcutaneously. In another preferred embodiment, a therapeutic composition described herein is administered orally, intratumorally, or intravenously. In another preferred embodiment, a therapeutic composition described herein is administered orally.

“Cancer” broadly refers to an uncontrolled, abnormal growth of a host's own cells leading to invasion of surrounding tissue and potentially tissue distal to the initial site of abnormal cell growth in the host. Major classes include carcinomas which are cancers of the epithelial tissue (for example, skin, squamous cells); sarcomas which are cancers of the connective tissue (for example, bone, cartilage, fat, muscle, blood vessels, etc.); leukemias which are cancers of blood forming tissue (for example, bone marrow tissue); lymphomas and myelomas which are cancers of immune cells; and central nervous system cancers which include cancers from brain and spinal tissue. “Cancer(s)” and “neoplasm(s)” are used herein interchangeably. As used herein, “cancer” refers to all types of cancer or neoplasm or malignant tumors including leukemias, carcinomas and sarcomas, whether new or recurring. Specific examples of cancers are: carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma. In some embodiments, the cancer comprises a solid tumor. In some embodiments, the cancer comprises a metastasis.

A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (for example, raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (for example, 2′-fluororibose, deoxyribose, and hexose). Carbohydrates may exist in many different forms, for example, conformers, cyclic forms, acyclic forms, stereoisomers, tautomers, anomers, and isomers.

The term “carcinoma” refers to a malignant growth made up of epithelial cells tending to infiltrate the surrounding tissues, and/or resist physiological and non-physiological cell death signals and gives rise to metastases.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells. Environments of particular interest are the microenvironments where cancer cells reside or locate. In some instances, the microenvironment is a tumor microenvironment or a tumor draining lymph node. In other instances, the microenvironment is a pre-cancerous tissue site or the site of local administration of a composition or a site where the composition will accumulate after remote administration.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

A “combination” can refer to EVs from one source strain with another agent, for example, another EV (for example, from another strain), with bacteria (for example, of the same or different strain that the EV was obtained from), or with another therapeutic agent. The combination can be in physical co-existence, either in the same material or product or in physically connected products, as well as the temporal co-administration or co-localization of the EVs and other agent.

As used herein, the term “consists essentially of” (or “consisting essentially of”) means limited to the recited elements and/or steps and those that do not materially affect the basic and novel characteristics of the claimed invention.

“Dysbiosis” refers to a state of the microbiota or microbiome of the gut or other body area, including, for example, mucosal or skin surfaces (or any other microbiome niche) in which the normal diversity and/or function of the host gut microbiome ecological networks (“microbiome”) are disrupted. A state of dysbiosis may result in a diseased state, or it may be unhealthy under only certain conditions or only if present for a prolonged period. Dysbiosis may be due to a variety of factors, including, environmental factors, infectious agents, host genotype, host diet and/or stress. A dysbiosis may result in: a change (for example, increase or decrease) in the prevalence of one or more bacteria types (for example, anaerobic), species and/or strains, change (for example, increase or decrease) in diversity of the host microbiome population composition; a change (for example, increase or reduction) of one or more populations of symbiont organisms resulting in a reduction or loss of one or more beneficial effects; overgrowth of one or more populations of pathogens (for example, pathogenic bacteria); and/or the presence of, and/or overgrowth of, symbiotic organisms that cause disease only when certain conditions are present.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include the number of immune cells, bacterial cells, stromal cells, myeloid derived suppressor cells, fibroblasts, metabolites; the level of a cytokine; or another physical parameter (such as ear thickness (for example, in a DTH animal model) or tumor size (for example, in an animal tumor model)).

The term “effective dose” is the amount of the therapeutic composition that is effective to achieve the desired therapeutic response for a particular subject, composition, and mode of administration, with the least toxicity to the subject.

As used herein, “engineered bacteria” are any bacteria that have been genetically altered from their natural state by human activities, and the progeny of any such bacteria. Engineered bacteria include, for example, the products of targeted genetic modification, the products of random mutagenesis screens and the products of directed evolution.

The term “epitope” means a protein determinant capable of specific binding to an antibody or T cell receptor. Epitopes usually consist of chemically active surface groupings of molecules such as amino acids or sugar side chains. Certain epitopes can be defined by a particular sequence of amino acids to which an antibody is capable of binding.

“Extracellular vesicles” (EVs) may be naturally-produced vesicles derived from bacteria, such as smEVs. EVs are comprised of bacterial lipids and/or bacterial proteins and/or bacterial nucleic acids and/or bacterial carbohydrate moieties, and are isolated from culture supernatant. The natural production of these vesicles can be artificially enhanced (for example, increased) or decreased through manipulation of the environment in which the bacterial cells are being cultured (for example, by media or temperature alterations). Further, EV compositions may be modified to reduce, increase, add, or remove bacterial components or foreign substances to alter efficacy, immune stimulation, stability, immune stimulatory capacity, stability, organ targeting (for example, lymph node), absorption (for example, gastrointestinal), and/or yield (for example, thereby altering the efficacy). As used herein, the term “purified EV composition” or “EV composition” refers to a preparation of EVs that have been separated from at least one associated substance found in a source material (for example, separated from at least one other bacterial component) or any material associated with the EVs in any process used to produce the preparation. It can also refer to a composition that has been significantly enriched for specific components. Extracellular vesicles may also be obtained from mammalian cells and from can be obtained from microbes such as archaea, fungi, microscopic algae, protozoans, and parasites. Extracellular vesicles from any of these sources can be prepared into a solution and/or dried form as described herein. Extracellular vesicles may be artificially-produced vesicles prepared from bacteria, such as pmEVs, for example, obtained by chemically disrupting (for example, by lysozyme and/or lysostaphin) and/or physically disrupting (for example, by mechanical force) bacterial cells and separating the bacterial membrane components from the intracellular components through centrifugation and/or ultracentrifugation, or other methods, can also be prepared into a solution and/or dried form as described herein.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (for example, psoriasis, atopic dermatitis, lupus, scleroderma, hemolytic anemia, vasculitis, type one diabetes, Grave's disease, rheumatoid arthritis, multiple sclerosis, Goodpasture's syndrome, pernicious anemia and/or myopathy), inflammatory diseases (for example, acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (for example, food allergies, drug allergies and/or environmental allergies).

“Immunotherapy” is treatment that uses a subject's immune system to treat disease (for example, immune disease, inflammatory disease, metabolic disease, cancer) and includes, for example, checkpoint inhibitors, cancer vaccines, cytokines, cell therapy, CAR-T cells, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold greater after treatment when compared to a pre-treatment state. Properties that may be increased include the number of immune cells, bacterial cells, stromal cells, myeloid derived suppressor cells, fibroblasts, metabolites; the level of a cytokine; or another physical parameter (such as ear thickness (for example, in a DTH animal model) or tumor size (for example, in an animal tumor model).

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. immuno-adjuvants are a specific class of broader adjuvant or adjuvant therapy. Examples of STING agonists include, but are not limited to, 2′3′-cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLR10 and TLR11. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The “internal transcribed spacer” or “ITS” is a piece ofnon-functional RNA located between structural ribosomal RNAs (rRNA) on a common precursor transcript often used for identification of eukaryotic species in particular fungi. The rRNA of fungi that forms the core of the ribosome is transcribed as a signal gene and consists of the 8S, 5.8S and 28S regions with ITS4 and 5 between the 8S and 5.8S and 5.8S and 28S regions, respectively. These two intercistronic segments between the 18S and 5.8S and 5.8S and 28S regions are removed by splicing and contain significant variation between species for barcoding purposes as previously described (Schoch et al Nuclear ribosomal internal transcribed spacer (ITS) region as a universal DNA barcode marker for Fungi. PNAS 109:6241-6246. 2012). 18S rDNA is traditionally used for phylogenetic reconstruction however the ITS can serve this function as it is generally highly conserved but contains hypervariable regions that harbor sufficient nucleotide diversity to differentiate genera and species of most fungus.

The term “isolated” or “enriched” encompasses a microbe, an EV (such as a bacterial EV) or other entity or substance that has been (1) separated from at least some of the components with which it was associated when initially produced (whether in nature or in an experimental setting), and/or (2) produced, prepared, purified, and/or manufactured by the hand of man. Isolated bacteria or EVs may be separated from at least about 10%, about 20%, about 30%, about 40%, about 50%, about 60%, about 70%, about 80%, about 90%, or more of the other components with which they were initially associated. In some embodiments, isolated bacteria or EVs are more than about 80%, about 85%, about 90%, about 91%, about 92%, about 93%, about 94%, about 95%, about 96%, about 97%, about 98%, about 99%, or more than about 99% pure, for example, substantially free of other components.

The term “leukemia” includes broadly progressive, malignant diseases of the hematopoietic organs/systems and is generally characterized by a distorted proliferation and development of leukocytes and their precursors in the blood and bone marrow.

As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans).

The term “melanoma” is taken to mean a tumor arising from the melanocytic system of the skin and other organs.

“Metabolite” as used herein refers to any and all molecular compounds, compositions, molecules, ions, co-factors, catalysts or nutrients used as substrates in any cellular or bacterial metabolic reaction or resulting as product compounds, compositions, molecules, ions, co-factors, catalysts or nutrients from any cellular or bacterial metabolic reaction.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state or dysbiotic microbiome. The microbiome may be native to the subject or patient, or components of the microbiome may be modulated, introduced, or depleted due to changes in health state (for example, precancerous or cancerous state) or treatment conditions (for example, antibiotic treatment, exposure to different microbes). In some aspects, the microbiome occurs at a mucosal surface. In some aspects, the microbiome is a gut microbiome. In some aspects, the microbiome is a tumor microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more cancer-associated bacterial strains are present in a sample. In some embodiments, the microbiome profile indicates the relative or absolute amount of each bacterial strain detected in the sample. In some embodiments, the microbiome profile is a cancer-associated microbiome profile. A cancer-associated microbiome profile is a microbiome profile that occurs with greater frequency in a subject who has cancer than in the general population. In some embodiments, the cancer-associated microbiome profile comprises a greater number of or amount of cancer-associated bacteria than is normally present in a microbiome of an otherwise equivalent tissue or sample taken from an individual who does not have cancer.

“Modified” in reference to a bacteria broadly refers to a bacteria that has undergone a change from its wild-type form. Bacterial modification can result from engineering bacteria. Examples of bacterial modifications include genetic modification, gene expression modification, phenotype modification, formulation modification, chemical modification, and dose or concentration. Examples of improved properties are described throughout this specification and include, for example, attenuation, auxotrophy, homing, or antigenicity. Phenotype modification might include, by way of example, bacteria growth in media that modify the phenotype of a bacterium such that it increases or decreases virulence.

An “oncobiome” as used herein comprises tumorigenic and/or cancer-associated microbiota, wherein the microbiota comprises one or more of a virus, a bacterium, a fungus, a protist, a parasite, or another microbe.

“Oncotrophic” or “oncophilic” microbes and bacteria are microbes that are highly associated or present in a cancer microenvironment. They may be preferentially selected for within the environment, preferentially grow in a cancer microenvironment or hone to a said environment.

“Operational taxonomic units” and “OTU(s)” refer to a terminal leaf in a phylogenetic tree and is defined by a nucleic acid sequence, for example, the entire genome, or a specific genetic sequence, and all sequences that share sequence identity to this nucleic acid sequence at the level of species. In some embodiments the specific genetic sequence may be the 16S sequence or a portion of the 16S sequence. In some embodiments, the entire genomes of two entities are sequenced and compared. In some embodiments, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See for example, Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940. For complete genomes, MLSTs, specific genes, other than 16S, or sets of genes OTUs that share ≥95% average nucleotide identity are considered the same OTU. See for example, Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (for example, “house-keeping” genes), or a combination thereof. Operational Taxonomic Units (OTUs) with taxonomic assignments made to, for example, genus, species, and phylogenetic clade are provided herein.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a substance is “pure” if it is substantially free of other components. The terms “purify,” “purifying,” and “purified” refer to an EV (such as an EV from bacteria) preparation or other material that has been separated from at least some of the components with which it was associated either when initially produced or generated (for example, whether in nature or in an experimental setting), or during any time after its initial production. An EV preparation or compositions may be considered purified if it is isolated at or after production, such as from one or more other bacterial components, and a purified microbe or bacterial population may contain other materials up to about 10%, about 20%, about 30%, about 40%, about 50%, about 60%, about 70%, about 80%, about 90%, or above about 90% and still be considered “purified.” In some embodiments, purified EVs are more than about 80%, about 85%, about 90%, about 91%, about 92%, about 93%, about 94%, about 95%, about 96%, about 97%, about 98%, about 99%, or more than about 99% pure. EV compositions (or preparations) are, for example, purified from residual habitat products.

As used herein, the term “purified EV composition” or “EV composition” refers to a preparation that includes EVs from bacteria that have been separated from at least one associated substance found in a source material (for example, separated from at least one other bacterial component) or any material associated with the EVs in any process used to produce the preparation. It also refers to a composition that has been significantly enriched or concentrated. In some embodiments, the EVs are concentrated by 2-fold, 3-fold, 4-fold, 5-fold, 10-fold, 100-fold, 1000-fold, 10,000-fold or more than 10,000-fold.

“Residual habitat products” refers to material derived from the habitat for microbiota within or on a subject. For example, fermentation cultures of microbes can contain contaminants, for example, other microbe strains or forms (for example, bacteria, virus, mycoplasm, and/or fungus). For example, microbes live in feces in the gastrointestinal tract, on the skin itself, in saliva, mucus of the respiratory tract, or secretions of the genitourinary tract (i.e., biological matter associated with the microbial community). Substantially free of residual habitat products means that the microbial composition no longer contains the biological matter associated with the microbial environment on or in the culture or human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the microbial composition contains no detectable cells from a culture contaminant or a human or animal and that only microbial cells are detectable. In one embodiment, substantially free of residual habitat products may also mean that the microbial composition contains no detectable viral (including bacteria, viruses (for example, phage)), fungal, mycoplasmal contaminants. In some embodiments, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the microbial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (for example, a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (for example, PCR, DNA sequencing), serology and antigen analysis, enzymatic and metabolic analysis, and methods using instrumentation such as flow cytometry with reagents that distinguish desired constituents from contaminants.

The term “sarcoma” generally refers to a tumor which is made up of a substance like the embryonic connective tissue and is generally composed of closely packed cells embedded in a fibrillar, heterogeneous, or homogeneous substance.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (for example, BSA, casein). Alternatively, specific binding applies more broadly to a two component system where one component is a protein, lipid, or carbohydrate or combination thereof and engages with the second component which is a protein, lipid, carbohydrate or combination thereof in a specific way.

As used herein, a “stock” refers to a solution comprising one or more excipients but no active ingredient (such as an extracellular vesicle). In some embodiments, a stock is used to introduce one or more excipients into a preparation (such as a liquid preparation) comprising EVs. In some embodiments, the stock is a concentrated solution comprising a known amount of one or more excipients. In some embodiments, the stock is combined with a preparation (such as a liquid preparation) that comprises EVs to prepare a solution or dried form provided herein.

“Strain” refers to a member of a bacterial species with a genetic signature such that it may be differentiated from closely-related members of the same bacterial species. The genetic signature may be the absence of all or part of at least one gene, the absence of all or part of at least on regulatory region (for example, a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (for example, a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The terms “subject” or “patient” refers to any mammal. A subject or a patient described as “in need thereof” refers to one in need of a treatment (or prevention) for a disease. Mammals (i.e., mammalian animals) include humans, laboratory animals (for example, primates, rats, mice), livestock (for example, cows, sheep, goats, pigs), and household pets (for example, dogs, cats, rodents). The subject may be a human. The subject may be a non-human mammal including but not limited to a dog, a cat, a cow, a horse, a pig, a donkey, a goat, a camel, a mouse, a rat, a guinea pig, a sheep, a llama, a monkey, a gorilla, or a chimpanzee. The subject may be healthy, or may be suffering from a cancer at any developmental stage, wherein any of the stages are either caused by or opportunistically supported of a cancer associated or causative pathogen, or may be at risk of developing a cancer, or transmitting to others a cancer associated or cancer causative pathogen. In some embodiments, a subject has lung cancer, bladder cancer, prostate cancer, plasmacytoma, colorectal cancer, rectal cancer, Merkel Cell carcinoma, salivary gland carcinoma, ovarian cancer, and/or melanoma. The subject may have a tumor. The subject may have a tumor that shows enhanced macropinocytosis with the underlying genomics of this process including Ras activation. In some embodiments, the subject has another cancer. In some embodiments, the subject has undergone a cancer therapy.

As used herein, the term “therapeutic agent” refers to an agent for therapeutic use. In some embodiments, a therapeutic agent is a composition comprising EVs (“an EV composition”) that can be used to treat and/or prevent a disease and/or condition. In some embodiments, the therapeutic agent is a pharmaceutical agent. In some embodiments, a medicinal product, medical food, a food product, or a dietary supplement comprises a therapeutic agent. In some embodiments, the therapeutic agent is in a solution, and in some embodiments, a dried form. The dried form embodiments may be produced, for example, by lyophilization or spray drying. In some embodiments, the dried form of the therapeutic agent is a lypholized cake or powder. In some embodiments, the dried form of the therapeutic agent is a spray-dried powder.

As used herein, the term “therapeutic composition” or “pharmaceutical composition” refers to a composition that comprises a therapeutically effective amount of a therapeutic agent (for example an EV composition described herein). In some embodiments, the therapeutic composition is (or is present in) a medicinal product, medical food, a food product, or a dietary supplement.

As used herein, the term “treating” a disease in a subject or“treating” a subject having or suspected of having a disease refers to administering to the subject to a pharmaceutical treatment, for example, the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof. As used herein, the term “preventing” a disease in a subject refers to administering to the subject to a pharmaceutical treatment, for example, the administration of one or more agents, such that onset of at least one symptom of the disease is delayed or prevented.

### Bacterial Extracellular Vesicles

In certain aspects, provided herein are solutions and/or dried form, and therapeutic compositions, that comprise extracellular vesicles (EVs). In certain aspects, provided herein are solutions and/or dried form, and therapeutic compositions, that comprise EVs obtained from bacteria.

Bacteria propagated as sources of EVs can be selected based on assays in the art that identify bacteria with properties of interest. For example, in some embodiments, bacteria are selected for the ability to modulate host immune response and/or affect cytokine levels. For example, a strain of bacteria is selected for affecting cytokine levels (such as TNFα, IL10, IL-6, IL-1β, and/or IP-10 levels) in a U937 assay, as described herein.

In some embodiments, EVs are selected from a bacterial strain that is associated with mucus. In some embodiments, the mucus is associated with the gut lumen. In some embodiments, the mucus is associated with the small intestine. In some embodiments, the mucus is associated with the respiratory tract.

In some embodiments, EVs are selected from a bacterial strain that is associated with an epithelial tissue, such as oral cavity, lung, nose, or vagina.

In some embodiments, the EVs are from bacteria that are human commensals.

In some embodiments, the EVs are from human commensal bacteria that originate from the human small intestine.

In some embodiments, the EVs are from human commensal bacteria that originate from the human small intestine and are associated there with the outer mucus layer.

Examples of taxonomic groups (such as class, order, family, genus, species and/or strain) of bacteria that can be used as a source of EVs described herein are provided in Table 1, Table 2, Table 3, and/or Table 4 and/or elsewhere throughout the specification (for example, Table J or Example 10). In some embodiments, the bacterial strain is a bacterial strain having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain provided herein (for example, listed in Table 1, Table 2, Table 3, and/or Table 4 and/or elsewhere in the specification (for example, Table J or Example 10)). In some embodiments, the EVs are from an oncotrophic bacteria. In some embodiments, the EVs are from an immunostimulatory bacteria. In some embodiments, the EVs are from an immunosuppressive bacteria. In some embodiments, the EVs are from an immunomodulatory bacteria. In certain embodiments, EVs are generated from a combination of bacterial strains provided herein. In some embodiments, the combination is a combination of at least 2, 3, 4, 5, 6, 7, 8, 9, 10, 11, 12, 13, 14, 15, 20, 25, 30, 35, 40, 45 or 50 bacterial strains. In some embodiments, the combination includes EVs from bacterial strains provided herein (for example, listed in Table 1, Table 2, Table 3, and/or Table 4 and/or elsewhere in the specification (for example, Table J or Example 10)) and/or bacterial strains having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain provided herein (for example, listed in Table 1, Table 2, Table 3, and/or Table 4 and/or elsewhere in the specification (for example, Table J or Example 10)). In some embodiments, bacteria from a taxonomic group (for example, class, order, family, genus, species or strain)) listed in Table 1, Table 2, Table 3, and/or Table 4 and/or elsewhere in the specification (for example, Table J or Example 10) can be used as a source of EVs.

In some embodiments, the EVs are obtained from Gram negative bacteria.

In some embodiments, the Gram negative bacteria belong to the class Negativicutes. The Negativicutes represent a unique class of microorganisms as they are the only diderm members of the Firmicutes phylum. These anaerobic organisms can be found in the environment and are normal commensals of the oral cavity and GI tract of humans. Because these organisms have an outer membrane, the yields of EVs from this class were investigated. It was found that on a per cell basis these bacteria produce a high number of vesicles (10-150 EVs/cell). The EVs from these organisms are broadly stimulatory and highly potent in in vitro assays. Investigations into their therapeutic applications in several oncology and inflammation in vivo models have shown their therapeutic potential. The Negativicutes class includes the families Veillonellaceae, Selenomonadaceae, Acidaminococcaceae, and Sporomusaceae. The Negativicutes class includes the genera Megasphaera, Selenomonas, Propionospora, and Acidaminococcus. Exemplary Negativicutes species include, but are not limited to, Megasphaera sp., Selenomonas felix, Acidaminococcus intestine, and Propionospora sp.

In some embodiments, the EVs are obtained from Gram positive bacteria.

In some embodiments, the EVs are from aerotolerant bacteria.

In some embodiments, the EVs are from monoderm bacteria.

In some embodiments, the EVs are from diderm bacteria.

In some, the EVs are from bacteria of the family: Prevotellaceae; Veillonellaceae; Tannerellaceae; Rikenellaceae; Selenomonadaceae; Sporomusaceae; Synergistaceae; or Akkermaniaceae.

In some embodiments, the EVs are from bacteria of the family Oscillospiraceae; Clostridiaceae; Lachnospiraceae; or Christensenellaceae.

In some embodiments, the EVs are from bacteria of the genus Prevotella.

In some embodiments, the EVs are from bacteria of the genus Veillonella.

In some embodiments, the EVs are from bacteria of the genus Parabacteroides.

In some embodiments, the EVs are from a bacterial strain of the Oscillospiraceae family.

In some embodiments, the EVs are from a bacterial strain of the Tannerellaceae family.

In some embodiments, the EVs are from a bacterial strain of the Prevotellaceae family.

In some embodiments, the EVs are from a bacterial strain of the Veillonellaceae family.

In some embodiments, the EVs are from a bacterial family evaluated in Example 10. In some embodiments, the EVs are from a bacterial genus evaluated in Example 10. In some embodiments, the EVs are from a bacterial species evaluated in Example 10.

In some embodiments, the EVs are obtained from aerobic bacteria.

In some embodiments, the EVs are obtained from anaerobic bacteria. In some embodiments, the anaerobic bacteria comprise obligate anaerobes. In some embodiments, the anaerobic bacteria comprise facultative anaerobes.

In some embodiments, the EVs are obtained from acidophile bacteria.

In some embodiments, the EVs are obtained from alkaliphile bacteria.

In some embodiments, the EVs are obtained from neutralophile bacteria.

In some embodiments, the EVs are obtained from fastidious bacteria.

In some embodiments, the EVs are obtained from nonfastidious bacteria.

In some embodiments, bacteria from which EVs are obtained are lyophilized.

In some embodiments, bacteria from which EVs are obtained are gamma irradiated (for example, at 17.5 or 25 kGy).

In some embodiments, bacteria from which EVs are obtained are UV irradiated.

In some embodiments, bacteria from which EVs are obtained are heat inactivated (for example, at 50° C. for two hours or at 90° C. for two hours).

In some embodiments, bacteria from which EVs are obtained are acid treated.

In some embodiments, bacteria from which EVs are obtained are oxygen sparged (for example, at 0.1 vvm for two hours).

In some embodiments, the EVs are lyophilized.

In some embodiments, the EVs are gamma irradiated (for example, at 17.5 or 25 kGy).

In some embodiments, the EVs are UV irradiated.

In some embodiments, the EVs are heat inactivated (for example, at 50° C. for two hours or at 90° C. for two hours).

In some embodiments, the EVs are acid treated.

In some embodiments, the EVs are oxygen sparged (for example, at 0.1 vvm for two hours).

The phase of growth can affect the amount or properties of bacteria and/or EVs produced by bacteria. For example, in the methods of EVs preparation provided herein, EVs can be isolated, for example, from a culture, at the start of the log phase of growth, midway through the log phase, and/or once stationary phase growth has been reached.

In certain embodiments, the EVs described herein are obtained from obligate anaerobic bacteria. Examples of obligate anaerobic bacteria include gram-negative rods (including the genera of Bacteroides, Prevotella, Porphyromonas, Fusobacterium, Bilophila and Sutterella spp.), gram-positive cocci (primarily Peptostreptococcus spp.), gram-positive spore-forming (Clostridium spp.), non-spore-forming bacilli (Actinomyces, Propionibacterium, Eubacterium, Lactobacillus and Bifidobacterium spp.), and gram-negative cocci (mainly Veillonella spp.). In some embodiments, the obligate anaerobic bacteria are of a genus selected from the group consisting of Agathobaculum, Atopobium, Blautia, Burkholderia, Dielma, Longicatena, Paraclostridium, Turicibacter, and Tyzzerella.

The Negativicutes class includes the families Veillonellaceae, Selenomonadaceae, Acidaminococcaceae, and Sporomusaceae. The Negativicutes class includes the genera Megasphaera, Selenomonas, Propionospora, and Acidaminococcus. Exemplary Negativicutes species include, but are not limited to, Megasphaera sp., Selenomonas felix, Acidaminococcus intestini, and Propionospora sp.

In some embodiments, the EVs are from bacteria of the Negativicutes class.

In some embodiments, the EVs are from bacteria of the Veillonellaceae family.

In some embodiments, the EVs are from bacteria of the Selenomonadaceae family.

In some embodiments, the EVs are from bacteria of the Acidaminococcaceae family.

In some embodiments, the EVs are from bacteria of the Sporomusaceae family.

In some embodiments, the EVs are from bacteria of the Megasphaera genus.

In some embodiments, the EVs are from bacteria of the Selenomonas genus.

In some embodiments, the EVs are from bacteria of the Propionospora genus.

In some embodiments, the EVs are from bacteria of the Acidaminococcus genus.

In some embodiments, the EVs are from Megasphaera sp. bacteria.

In some embodiments, the EVs are from Selenomonas felix bacteria.

In some embodiments, the EVs are from Acidaminococcus intestini bacteria.

In some embodiments, the EVs are from Propionospora sp. bacteria.

The Oscillospriraceae family within the Clostridia class of microorganisms are common commensal organisms of vertebrates.

In some embodiments, the EVs are from bacteria of the Clostridia class.

In some embodiments, the EVs are from bacteria of the Oscillospriraceae family.

In some embodiments, the EVs are from bacteria of the Faecalibacterium genus.

In some embodiments, the EVs are from bacteria of the Fournierella genus.

In some embodiments, the EVs are from bacteria of the Harryflintia genus.

In some embodiments, the EVs are from bacteria of the Agathobaculum genus.

In some embodiments, the EVs are from Faecalibacterium prausnitzii (for example, Faecalibacterium prausnitzii Strain A) bacteria.

In some embodiments, the EVs are from Fournierella massiliensis (for example, Fournierella massiliensis Strain A) bacteria.

In some embodiments, the EVs are from Harryflintia acetispora (for example, Harryflintia acetispora Strain A) bacteria.

In some embodiments, the EVs are from Agathobaculum sp. (for example, Agathobaculum sp. Strain A) bacteria.

In some embodiments, the EVs described herein are obtained from bacterium of a genus selected from the group consisting of Escherichia, Klebsiella, Lactobacillus, Shigella, and Staphylococcus.

In some embodiments, the EVs described herein are obtained from a species selected from the group consisting of Blautia massiliensis, Paraclostridium benzoelyticum, Dielma fastidiosa, Longicatena caecimuris, Lactococcus lactis cremoris, Tyzzerella nexilis, Hungatella effluvia, Klebsiella quasipneumoniae subsp. Similipneumoniae. Klebsiella oxytoca, and Veillonella tobetsuensis.

In some embodiments, the EVs described herein are obtained from a Prevotella bacteria selected from the group consisting of Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, and Prevotella veroralis.

In some embodiments, the EVs described herein are obtained from a strain of bacteria comprising a genomic sequence that is at least 90%, at least 91%, at least 92%, at least 93%, at least 94%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the genomic sequence of the strain of bacteria deposited with the ATCC Deposit number as provided in Table 3. In some embodiments, the EVs described herein are obtained from a strain of bacteria comprising a 16S sequence that is at least 90%, at least 91%, at least 92%, at least 93%, at least 94%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the 16S sequence of the strain of bacteria deposited with the ATCC Deposit number as provided in Table 3.

The Negativicutes class includes the families Veillonellaceae, Selenomonadaceae, Acidaminococcaceae, and Sporomusaceae. The Negativicutes class includes the genera Megasphaera, Selenomonas, Propionospora, and Acidaminococcus. Exemplary Negativicutes species include, but are not limited to, Megasphaera sp., Selenomonas felix, Acidaminococcus intestini, and Propionospora sp.

The Negativicutes class includes the families Veillonellaceae, Selenomonadaceae, Acidaminococcaceae, and Sporomusaceae. The Negativicutes class includes the genera Megasphaera. Selenomonas, Propionospora, and Acidaminococcus. Exemplary Negativicutes species include, but are not limited to, Megasphaera sp., Selenomonas felix, Acidaminococcus intestini, and Propionospora sp.

In some embodiments, the bacteria from which the EVs are obtained are of the Negativicutes class.

In some embodiments, the bacteria from which the EVs are obtained are of the Veillonellaceae family.

In some embodiments, the bacteria from which the EVs are obtained are of the Selenomonadaceae family.

In some embodiments, the bacteria from which the EVs are obtained are of the Acidaminococcaceae family.

In some embodiments, the bacteria from which the EVs are obtained are of the Sporomusaceae family.

In some embodiments, the bacteria from which the EVs are obtained are of the Megasphaera genus.

In some embodiments, the bacteria from which the EVs are obtained are of the Selenomonas genus.

In some embodiments, the bacteria from which the EVs are obtained are of the Propionospora genus.

In some embodiments, the bacteria from which the EVs are obtained are of the Acidaminococcus genus.

In some embodiments, the bacteria from which the EVs are obtained are Megasphaera sp. bacteria.

In some embodiments, the bacteria from which the EVs are obtained are Selenomonas felix bacteria.

In some embodiments, the bacteria from which the EVs are obtained are Acidaminococcus intestini bacteria.

In some embodiments, the bacteria from which the EVs are obtained are Propionospora sp. bacteria.

The Oscillospriraceae family within the Clostridia class of microorganisms are common commensal organisms of vertebrates.

In some embodiments, the bacteria from which the EVs are obtained are of the Clostridia class.

In some embodiments, the bacteria from which the EVs are obtained are of the Oscillospriraceae family.

In some embodiments, the bacteria from which the EVs are obtained are of the Faecalibacterium genus.

In some embodiments, the bacteria from which the EVs are obtained are of the Fournierella genus.

In some embodiments, the bacteria from which the EVs are obtained are of the Harryflintia genus.

In some embodiments, the bacteria from which the EVs are obtained are of the Agathobaculum genus.

In some embodiments, the bacteria from which the EVs are obtained are Faecalibacterium prausnitzii (for example, Faecalibacterium prausnitzii Strain A) bacteria.

In some embodiments, the bacteria from which the EVs are obtained are Fournierella massiliensis (for example, Fournierella massiliensis Strain A) bacteria.

In some embodiments, the bacteria from which the EVs are obtained are Harryflintia acetispora (for example, Harryflintia acetispora Strain A) bacteria.

In some embodiments, the bacteria from which the EVs are obtained are Agathobaculum sp. (for example, Agathobaculum sp. Strain A) bacteria.

In some embodiments, the bacteria from which the EVs are obtained are bacteria of a genus selected from the group consisting of Escherichia, Klebsiella, Lactobacillus, Shigella, and Staphylococcus.

In some embodiments, the bacteria from which the EVs are obtained are a species selected from the group consisting of Blautia massiliensis, Paraclostridium benzoelyticum, Dielma fastidiosa, Longicatena caecimuris, Lactococcus lactis cremoris, Tyzzerella nexilis, Hungatella effluvia, Klebsiella quasipneumoniae subsp. Similipneumoniae, Klebsiella oxytoca, and Veillonella tobetsuensis.

In some embodiments, the bacteria from which the EVs are obtained are a Prevotella bacteria selected from the group consisting of Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, and Prevotella veroralis.

In some embodiments, the bacteria from which the EVs are obtained are a strain of bacteria comprising a genomic sequence that is at least 90%, at least 91%, at least 92%, at least 93%, at least 94%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the genomic sequence of the strain of bacteria deposited with the ATCC Deposit number as provided in Table 3. In some embodiments, the bacteria from which the EVs are obtained are a strain of bacteria comprising a 16S sequence that is at least 90%, at least 91%, at least 92%, at least 93%, at least 94%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the 16S sequence of the strain of bacteria deposited with the ATCC Deposit number as provided in Table 3.

The Negativicutes class includes the families Veillonellaceae, Selenomonadaceae, Acidaminococcaceae, and Sporomusaceae. The Negativicutes class includes the genera Megasphaera, Selenomonas, Propionospora, and Acidaminococcus. Exemplary Negativicutes species include, but are not limited to, Megasphaera sp., Selenomonas felix, Acidaminococcus intestini, and Propionospora sp.

In some embodiments, the bacteria from which the EVs are obtained are of the Negativicutes class.

In some embodiments, the bacteria from which the EVs are obtained are of the Veillonellaceae family.

In some embodiments, the bacteria from which the EVs are obtained are of the Selenomonadaceae family.

In some embodiments, the bacteria from which the EVs are obtained are of the Acidaminococcaceae family.

In some embodiments, the bacteria from which the EVs are obtained are of the Sporomusaceae family.

In some embodiments, the bacteria from which the EVs are obtained are of the Prevotellaceae; Veillonellaceae; Tannerellaceae; Rikenellaceae; Selenomonadaceae; Sporomusaceae; Synergistaceae; Christensenellaceae; or Akkermaniaceae family.

In some embodiments, the bacteria from which the EVs are obtained are of the Oscillospiraceae; Clostridiaceae; or Lachnospiraceae family.

In some embodiments, the bacteria from which the EVs are obtained are of the Megasphaera genus.

In some embodiments, the bacteria from which the EVs are obtained are of the Selenomonas genus.

In some embodiments, the bacteria from which the EVs are obtained are of the Propionospora genus.

In some embodiments, the bacteria from which the EVs are obtained are of the Acidaminococcus genus.

In some embodiments, the bacteria from which the EVs are obtained are Megasphaera sp. bacteria.

In some embodiments, the bacteria from which the EVs are obtained are Selenomonas felix bacteria.

In some embodiments, the bacteria from which the EVs are obtained are Acidaminococcus intestini bacteria.

In some embodiments, the bacteria from which the EVs are obtained are Propionospora sp. bacteria.

The Oscillospriraceae family within the Clostridia class of microorganisms are common commensal organisms of vertebrates.

In some embodiments, the bacteria from which the EVs are obtained are of the Clostridia class.

In some embodiments, the bacteria from which the EVs are obtained are of the Oscillospriraceae family.

In some embodiments, the bacteria from which the EVs are obtained are of the Faecalibacterium genus.

In some embodiments, the bacteria from which the EVs are obtained are of the Fournierella genus.

In some embodiments, the bacteria from which the EVs are obtained are of the Harryflintia genus.

In some embodiments, the bacteria from which the EVs are obtained are of the Agathobaculum genus.

In some embodiments, the bacteria from which the EVs are obtained are Faecalibacterium prausnitzii (for example, Faecalibacterium prausnitzii Strain A) bacteria.

In some embodiments, the bacteria from which the EVs are obtained are Fournierella massiliensis (for example, Fournierella massiliensis Strain A) bacteria.

In some embodiments, the bacteria from which the EVs are obtained are Harryflintia acetispora (for example, Harryflintia acetispora Strain A) bacteria.

In some embodiments, the bacteria from which the EVs are obtained are Agathobaculum sp. (for example, Agathobaculum sp. Strain A) bacteria.

In some embodiments, the bacteria from which the EVs are obtained are a strain of Agathobaculum sp. In some embodiments, the Agathobaculum sp. strain is a strain comprising at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, CRISPR sequence) of the Agathobaculum sp. Strain A (ATCC Deposit Number PTA-125892). In some embodiments, the Agathobaculum sp. strain is the Agathobaculum sp. Strain A (ATCC Deposit Number PTA-125892).

In some embodiments, the bacteria from which the EVs are obtained are of the class Bacteroidia [phylum Bacteroidota]. In some embodiments, the bacteria from which the EVs are obtained are bacteria of order Bacteroidales. In some embodiments, the bacteria from which the EVs are obtained are of the family Porphyromonoadaceae. In some embodiments, the bacteria from which the EVs are obtained are of the family Prevotellaceae. In some embodiments, the bacteria from which the EVs are obtained are bacteria of the class Bacteroidia wherein the cell envelope structure of the bacteria is diderm. In some embodiments, the bacteria from which the EVs are obtained are bacteria of the class Bacteroidia that stain Gram negative. In some embodiments, the bacteria from which the EVs are obtained are bacteria of the class Bacteroidia wherein the bacteria is diderm and the bacteria stain Gram negative.

In some embodiments, the bacteria from which the EVs are obtained are bacteria of the class Clostridia [phylum Firmicutes]. In some embodiments, the bacteria from which the EVs are obtained are of the order Eubacteriales. In some embodiments, the bacteria from which the EVs are obtained are of the family Oscillispiraceae. In some embodiments, the bacteria from which the EVs are obtained are of the family Lachnospiraceae. In some embodiments, the bacteria from which the EVs are obtained are of the family Peptostreptococcaceae. In some embodiments, the bacteria from which the EVs are obtained are of the family Clostridiales family XIII/Incertae sedis 41. In some embodiments, the bacteria from which the EVs are obtained are of the class Clostridia wherein the cell envelope structure of the bacteria is monoderm. In some embodiments, the bacteria from which the EVs are obtained are of the class Clostridia that stain Gram negative. In some embodiments, the bacteria from which the EVs are obtained are of the class Clostridia that stain Gram positive. In some embodiments, the bacteria from which the EVs are obtained are of the class Clostridia wherein the cell envelope structure of the bacteria is monoderm and the bacteria stain Gram negative. In some embodiments, the bacteria from which the EVs are obtained are of the class Clostridia wherein the cell envelope structure of the bacteria is monoderm and the bacteria stain Gram positive.

In some embodiments, the bacteria from which the EVs are obtained are of the class Negativicutes [phylum Firmicutes]. In some embodiments, the bacteria from which the EVs are obtained are of the order Veillonellales. In some embodiments, the bacteria from which the EVs are obtained are of the family Veillonelloceae. In some embodiments, the bacteria from which the EVs are obtained are of the order Selenomonadales. In some embodiments, the bacteria from which the EVs are obtained are bacteria of the family Selenomonadaceae. In some embodiments, the bacteria from which the EVs are obtained are of the family Sporomusaceae. In some embodiments, t the bacteria from which the EVs are obtained are of the class Negativicutes wherein the cell envelope structure of the bacteria is diderm. In some embodiments, the bacteria from which the EVs are obtained are of the bacteria from which the EVs are obtained are the EVs are from bacteria of the class Negativicutes wherein the cell envelope structure of the bacteria is diderm and the bacteria stain Gram negative.

In some embodiments, the bacteria from which the EVs are obtained are of the class Synergistia [phylum Synergistota]. In some embodiments, the bacteria from which the EVs are obtained are of the order Synergistales. In some embodiments, the bacteria from which the EVs are obtained are of the family Synergistaceae. In some embodiments, the bacteria from which the EVs are obtained are of the class Synergistia wherein the cell envelope structure of the bacteria is diderm. In some embodiments, the bacteria from which the EVs are obtained are of the class Synergistia that stain Gram negative. In some embodiments, the bacteria from which the EVs are obtained are of the class Synergistia wherein the cell envelope structure of the bacteria is diderm and the bacteria stain Gram negative.

In some embodiments, the bacteria from which the EVs are obtained are from one strain of bacteria, for example, a strain provided herein.

In some embodiments, the bacteria from which the EVs are obtained are from one strain of bacteria (for example, a strain provided herein) or from more than one strain provided herein.

In some embodiments, the bacteria from which the EVs are obtained are Lactococcus lactis cremoris bacteria, for example, a strain comprising at least 90% or at least 99% genomic, 16S and/or CRISPR sequence identity to the nucleotide sequence of the Lactococcus lactis cremoris Strain A (ATCC designation number PTA-125368). In some embodiments, the bacteria from which the EVs are obtained are Lactococcus bacteria, for example, Lactococcus lactis cremoris Strain A (ATCC designation number PTA-125368).

In some embodiments, the bacteria from which the EVs are obtained are Prevotella bacteria, for example, a strain comprising at least 90% or at least 99% genomic, 16S and/or CRISPR sequence identity to the nucleotide sequence of the Prevotella Strain B 50329 (NRRL accession number B 50329). In some embodiments, the bacteria from which the EVs are obtained are Prevotella bacteria, for example, Prevotella Strain B 50329 (NRRL accession number B 50329).

In some embodiments, the bacteria from which the EVs are obtained are Bifidobacterium bacteria, for example, a strain comprising at least 90% or at least 99% genomic, 16S and/or CRISPR sequence identity to the nucleotide sequence of the Bifidobacterium bacteria deposited as ATCC designation number PTA-125097. In some embodiments, the bacteria from which the EVs are obtained are Bifidobacterium bacteria, for example, Bifidobacterium bacteria deposited as ATCC designation number PTA-125097.

In some embodiments, the bacteria from which the EVs are obtained are Veillonella bacteria, for example, a strain comprising at least 90% or at least 99% genomic, 16S and/or CRISPR sequence identity to the nucleotide sequence of the Veillonella bacteria deposited as ATCC designation number PTA-125691. In some embodiments, the bacteria from which the EVs are obtained are Veillonella bacteria, for example, Veillonella bacteria deposited as ATCC designation number PTA-125691.

In some embodiments, the bacteria from which the EVs are obtained are Ruminococcus gnavus bacteria. In some embodiments, the Ruminococcus gnavus bacteria are a strain comprising at least 90% (or at least 97%) genomic, 16S and/or CRISPR sequence identity to the nucleotide sequence of the Ruminococcus gnavus bacteria deposited as ATCC designation number PTA-126695. In some embodiments, the Ruminococcus gnavus bacteria are a strain comprising at least 99% genomic, 16S and/or CRISPR sequence identity to the nucleotide sequence of the Ruminococcus gnavus bacteria deposited as ATCC designation number PTA-126695. In some embodiments, the Ruminococcus gnavus bacteria are Ruminococcus gnavus bacteria deposited as ATCC designation number PTA-126695.

In some embodiments, the bacteria from which the EVs are obtained are Megasphaera sp. bacteria. In some embodiments, the Megasphaera sp. bacteria are a strain comprising at least 90% (or at least 97%) genomic, 16S and/or CRISPR sequence identity to the nucleotide sequence of the Megasphaera sp. bacteria deposited as ATCC designation number PTA-126770. In some embodiments, the Megasphaera sp. bacteria are a strain comprising at least 99/genomic, 16S and/or CRISPR sequence identity to the nucleotide sequence of the Megasphaera sp. bacteria deposited as ATCC designation number PTA-126770. In some embodiments, the Megasphaera sp. bacteria are Megasphaera sp. bacteria deposited as ATCC designation number PTA-126770.

In some embodiments, the bacteria from which the EVs are obtained are Fournierella massiliensis bacteria. In some embodiments, the Fournierella massiliensis bacteria are a strain comprising at least 90% (or at least 97%) genomic, 16S and/or CRISPR sequence identity to the nucleotide sequence of the Fournierella massiliensis bacteria deposited as ATCC designation number PTA-126696. In some embodiments, the Fournierella massiliensis bacteria are a strain comprising at least 99% genomic, 16S and/or CRISPR sequence identity to the nucleotide sequence of the Fournierella massiliensis bacteria deposited as ATCC designation number PTA-126696. In some embodiments, the Fournierella massiliensis bacteria are Fournierella massiliensis bacteria deposited as ATCC designation number PTA-126696.

In some embodiments, the bacteria from which the EVs are obtained are Harryflintia acetispora bacteria. In some embodiments, the Harryflintia acetispora bacteria are a strain comprising at least 90% (or at least 97%) genomic, 16S and/or CRISPR sequence identity to the nucleotide sequence of the Harryflintia acetispora bacteria deposited as ATCC designation number PTA-126694. In some embodiments, the Harryflintia acetispora bacteria are a strain comprising at least 99% genomic, 16S and/or CRISPR sequence identity to the nucleotide sequence of the Harryflintia acetispora bacteria deposited as ATCC designation number PTA-126694. In some embodiments, the Harryflintia acetispora bacteria are Harryflintia acetispora bacteria deposited as ATCC designation number PTA-126694.

In some embodiments, the bacteria from which the EVs are obtained are bacteria that produce metabolites, for example, the bacteria produce butyrate, iosine, proprionate, or tryptophan metabolites.

In some embodiments, the bacteria from which the EVs are obtained are bacteria that produce butyrate. In some embodiments, the bacteria are from the genus Blautia; Christensella; Copracoccus; Eubacterium; Lachnosperacea; Megasphaera; or Roseburia.

In some embodiments, the bacteria from which the EVs are obtained are bacteria that produce iosine. In some embodiments, the bacteria are from the genus Bifidobacterium; Lactobacillus; or Olsenella.

In some embodiments, the bacteria from which the EVs are obtained are bacteria that produce proprionate. In some embodiments, the bacteria are from the genus Akkermansia; Bacteroides; Dialister; Eubacterium; Megasphaera; Parabacteriodes; Prevotella; Ruminococcus; or Veillonella.

In some embodiments, the bacteria from which the EVs are obtained are bacteria that produce tryptophan metabolites. In some embodiments, the bacteria are from the genus Lactobacillus or Peptostreptococcus.

In some embodiments, the bacteria from which the EVs are obtained are bacteria that produce inhibitors of histone deacetylase 3 (HDAC3). In some embodiments, the bacteria are from the species Bariatricus massiliensis, Faecalibacterium prausnitzii, Megasphaera massiliensis or Roseburia intestinalis.

In some embodiments, the bacteria are from the genus Alloiococcus; Bacillus; Catenibacterium; Corynebacterium; Cupriavidus; Enhydrobacter; Exiguobacterium; Faecalibacterium; Geobacillus; Methylobacterium; Micrococcus; Morganella; Proteus; Pseudomonas; Rhizobium; or Sphingomonas. In some embodiments, the bacteria are from the genus Cutibacterium. In some embodiments, the bacteria are from the species Cutibacterium avidum. In some embodiments, the bacteria are from the genus Lactobacillus. In some embodiments, the bacteria are from the species Lactobacillus gasseri. In some embodiments, the bacteria are from the genus Dysosmobacter. In some embodiments, the bacteria are from the species Dysosmobacter welbionis.

In some embodiments, the bacteria from which the EVs are obtained are of the genus Alloiococcus; Bacillus; Catenibacterium; Corynebacterium; Cupriavidus; Enhydrobacter; Exiguobacterium; Faecalibacterium; Geobacillus; Methylobacterium; Micrococcus; Morganella; Proteus; Pseudomonas; Rhizobium; or Sphingomonas.

In some embodiments, the bacteria from which the EVs are obtained are of the Cutibacterium genus. In some embodiments, the bacteria from which the EVs are obtained are Cutibacterium avidum bacteria.

In some embodiments, the bacteria from which the EVs are obtained are of the genus Leuconostoc.

In some embodiments, the bacteria from which the EVs are obtained are of the genus Lactobacillus.

In some embodiments, the bacteria from which the EVs are obtained are of the genus Akkermansia; Bacillus; Blautia; Cupriavidus; Enhydrobacter; Faecalibacterium; Lactobacillus; Lactococcus; Micrococcus; Morganella; Propionibacterium; Proteus; Rhizobium; or Streptococcus.

In some embodiments, the bacteria from which the EVs are obtained are Leuconostoc holzapfelii bacteria.

In some embodiments, the bacteria from which the EVs are obtained are Akkermansia muciniphila; Cupriavidus metallidurans; Faecalibacterium prausnitzii; Lactobacillus casei; Lactobacillus plantarum; Lactobacillus paracasei; Lactobacillus plantarum; Lactobacillus rhamnosus; Lactobacillus sakei; or Streptococcus pyogenes bacteria.

In some embodiments, the bacteria from which the EVs are obtained are Lactobacillus casei; Lactobacillus plantarum; Lactobacillus paracasei; Lactobacillus plantarum; Lactobacillus rhamnosus; or Lactobacillus sakei bacteria.

In some embodiments, the EVs described herein are obtained from a genus selected from the group consisting of Acinetobacter; Deinococcus; Helicobacter; Rhodococcus; Weissella cibaria; Alloiococcus; Atopobium; Catenibacterium; Corynebacterium; Exiguobacterium; Geobacillus; Methylobacterium; Micrococcus; Morganella; Proteus; Rhizobium; Rothia; Sphingomonas; Sphingomonas; and Leuconostoc.

In some embodiments, the EVs described herein are obtained from a species selected from the group consisting of Acinetobacter baumanii; Deinococcus radiodurans; Helicobacter pylori; Rhodococcus equi; Weissella cibaria; Alloiooccus otitis; Atopobium vaginae; Catenibacterium mituokai; Corynebacterium glutamicum; Exiguobacterium aurantiacum; Geobacillus stearothermophilus; Methylobacterium jeotgali; Micrococcus luteus; Morganella morganii; Proteus mirabilis; Rhizobium leguminosarum; Rothia amarae; Sphingomonas paucimobilis; and Sphingomonas koreens.

In some embodiments, the EVs are from Leuconostoc holzapfelii bacteria. In some embodiments, the EVs are from Leuconostoc holzapfelii Ceb-kc-003 (KCCM11830P) bacteria.

In some embodiments, the bacteria from which the EVs are obtained are Megasphaera sp. bacteria (for example, from the strain with accession number NCIMB 43385, NCIMB 43386 or NCIMB 43387).

In some embodiments, the bacteria from which the EVs are obtained are Megasphaera massiliensis bacteria (for example, from the strain with accession number NCIMB 42787, NCIMB 43388 or NCIMB 43389).

In some embodiments, the bacteria from which the EVs are obtained are Megasphaera massiliensis bacteria (for example, from the strain with accession number DSM 26228).

In some embodiments, the bacteria from which the EVs are obtained are Parabacteroides distasonis bacteria (for example, from the strain with accession number NCIMB 42382).

In some embodiments, the bacteria from which the EVs are obtained are Megasphaera massiliensis bacteria (for example, from the strain with accession number NCIMB 43388 or NCIMB 43389), or a derivative thereof. See, for example, WO 2020/120714. In some embodiments, the Megasphaera massiliensis bacteria is a strain comprising at least 80%, at least 85%, at least 90%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, and/or CRISPR sequence) of Megasphaera massiliensis bacteria from the strain with accession number NCIMB 43388 or NCIMB 43389. In some embodiments, the Megasphaera massiliensis bacteria is the strain with accession number NCIMB 43388 or NCIMB 43389.

In some embodiments, the bacteria from which the EVs are obtained are Megasphaera massiliensis bacteria strain deposited under accession number NCIMB 42787, or a derivative thereof. See, for example, WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, and/or CRISPR sequence) of the Megasphaera massiliensis bacteria strain deposited under accession number NCIMB 42787. In some embodiments, the Megasphaera massiliensis bacteria is the strain deposited under accession number NCIMB 42787.

In some embodiments, the bacteria from which the EVs are obtained are Megasphaera spp. bacteria from the strain with accession number NCIMB 43385, NCIMB 43386 or NCIMB 43387, or a derivative thereof. See, for example, WO 2020/120714. In some embodiments, the Megasphaera sp. bacteria is a strain comprising at least 80%, at least 85%, at least 90%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, and/or CRISPR sequence) of the Megasphaera sp. from a strain with accession number NCIMB 43385, NCIMB 43386 or NCIMB 43387. In some embodiments, the Megasphaera sp. bacteria is the strain with accession number NCIMB 43385, NCIMB 43386 or NCIMB 43387.

In some embodiments, the bacteria from which the EVs are obtained are Parabacteroides distasonis bacteria deposited under accession number NCIMB 42382, or a derivative thereof. See, for example, WO 2018/229216. In some embodiments, the Parabacteroides distasonis bacteria is a strain comprising at least 80%, at least 85%, at least 90%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, and/or CRISPR sequence) of the Parabacteroides distasonis bacteria deposited under accession number NCIMB 42382. In some embodiments, the Parabacteroides distasonis bacteria is the strain deposited under accession number NCIMB 42382.

In some embodiments, the bacteria from which the EVs are obtained are Megasphaera massiliensis bacteria deposited under accession number DSM 26228, or a derivative thereof. See, for example, WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (for example, at least 99.5% sequence identity, at least 99.6% sequence identity, at least 99.7% sequence identity, at least 99.8% sequence identity, at least 99.9% sequence identity) to the nucleotide sequence (for example, genomic sequence, 16S sequence, and/or CRISPR sequence) of Megasphaera massiliensis bacteria deposited under accession number DSM 26228. In some embodiments, the Megasphaera massiliensis bacteria is the strain deposited under accession number DSM 26228.

In some embodiments, the bacteria from which the EVs are obtained are modified (for example, engineered) to reduce toxicity or other adverse effects, to enhance delivery) (for example, oral delivery) of the EVs (for example, by improving acid resistance, muco-adherence and/or penetration and/or resistance to bile acids, digestive enzymes, resistance to anti-microbial peptides and/or antibody neutralization), to target desired cell types (for example, M-cells, goblet cells, enterocytes, dendritic cells, macrophages), to enhance their immunomodulatory and/or therapeutic effect of the EVs (for example, either alone or in combination with another therapeutic agent), and/or to enhance immune activation or suppression by the EVs (for example, through modified production of polysaccharides, pili, fimbriae, adhesins). In some embodiments, the engineered bacteria described herein are modified to improve EV manufacturing (for example, higher oxygen tolerance, stability, improved freeze-thaw tolerance, shorter generation times). For example, in some embodiments, the engineered bacteria described include bacteria harboring one or more genetic changes, such change being an insertion, deletion, translocation, or substitution, or any combination thereof, of one or more nucleotides contained on the bacterial chromosome or endogenous plasmid and/or one or more foreign plasmids, wherein the genetic change may results in the overexpression and/or underexpression of one or more genes. The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

TABLE 1
Bacteria by Class

| Class | Order | Family | Genus* | Species |
|---|---|---|---|---|
| Actinobacter | Actinomycetales | Mycobacteriaceae | Mycobacterium | |
| | | Streptomycetaceae | Streptomyces (S.) | S. lividans, S coelicolor, |
| | | | | S sudanesis, S |
| | | | | somaliensis |
| | Bifidobacteriales | Bifidobacteriaceae | Bifidobacterium (B.) | B. adolescentis, |
| | | | | B. animalis, B. bifidum, |
| | | | | B. breve, B. lactis, B. |
| | | | | longum, B. |
| | | | | pseudocatenulatum |
| | Coriobacteriales | Coriobacteriaceae | Collinsella | Collinsella aerofaciens |
| | | | Olsenella | Olsenella faecalis |
| | Propionibacteriales | Propionibacteraceae | Propionibacterium | |
| Bacilli | Bacillales | Bacillales incertae | Gemella (G.) | G. haemolysans, G. |
| | | sedis family XI | | morbillorum |
| | | Listeraceae | Listeria (L.) | L. monocytogenes, L. |
| | | | | welshimeri |
| | Lactobacilloles | Enterococcaceae | Enterococcus (E.) | E. durans, E. faecium, E. |
| | | | | faecalis, E. gallinarum, |
| | | | | E. villorum, |
| | | | Lactobacillus (L.) | L. casei, L. fermentum, |
| | | | | L. mucosae, L. |
| | | | | plantarum, L. reuteri, L. |
| | | | | rhamnosus, L. salvarius |
| | | Streptococcaceae | Lactococcus | Lactococcus lactis |
| | | | | cremoris |
| | | | Staphylococcus | Staphylococcus aureus |
| | | | Streptococcus (S.) | S. agalactiae, S. |
| | | | | aureus, S. australi, S. |
| | | | | mutans, S. |
| | | | | parasanguinis, S. |
| | | | | pneumoniae, S. |
| | | | | pyogenes, S. salivarius |
| Bacteriodes | Bacteroidales | Bacteriodaceae | Bacteriodes (B.) | B. caccae, B. |
| | | | | cellulosilyticus, B. |
| | | | | coprocola, B. dorei, B. |
| | | | | fragilis, B. ovatus, B. |
| | | | | putredinis, B. |
| | | | | salanitronis, B. |
| | | | | thetaiotaomicron, B. |
| | | | | vulgatus |
| | | Odoribacteraceae | Odoribacter | Odoribacter |
| | | | | splanchnicus |
| | | Porphyromonadaceae | Parabacteriodes (P.) | P. distasonis, P. |
| | | | | goldsteinii, P. merdae |
| | | | Porphyromonas | Porphyromonas |
| | | | | gingivalis |
| | | Prevotellaceae | Prevotella (P.) | P. albensis, P. amnii, P. |
| | | | | aurantiaca, P. |
| | | | | baroniae, P. bergensis, |
| | | | | P. bivia, P. brevis, P. |
| | | | | bryantii, P. buccae, P. |
| | | | | buccalis, P. colorans, P. |
| | | | | corporis, P. copri, P. |
| | | | | dentalis, P. dentasini, P. |
| | | | | denticola, P. disiens,, P. |
| | | | | enoeca, P. falsenii, P. |
| | | | | fusca, P. heparinolytica, |
| | | | | P. histicola, P. |
| | | | | intermedia, P. jejuni,, |
| | | | | P. loescheii, P. |
| | | | | maculosa, P. marshii, P. |
| | | | | melaninogenica, P. |
| | | | | micans, P. multiformis, |
| | | | | P. multisaccharivorax, |
| | | | | P. nanceiensis, P. |
| | | | | nigrescens, P. oralis, P. |
| | | | | oris,, P. oryzae, P. |
| | | | | oulorum, P. pallens, P. |
| | | | | paludivivens, P. |
| | | | | pleuritidis P. |
| | | | | ruminicola, P. |
| | | | | saccharolytica, P. |
| | | | | salivae, P. scopos, P. |
| | | | | shahii, P. stercorea, P. |
| | | | | tannerae, P. |
| | | | | timonensis, P. veroralis, |
| | | | | P. zoogleoformans |
| | | Rikenellaceae | Alstipes (A.) | A. communis, A. dispar, |
| | | | | A. finegoldii, A. |
| | | | | indistinctus, A. ihumii, |
| | | | | A. inops, A. |
| | | | | massiliensis, A. |
| | | | | megaguti, A. obesi, A. |
| | | | | onderdonkii, A. |
| | | | | provencensis, A. |
| | | | | putredinis, A. |
| | | | | senegalensis, A. shahii, |
| | | | | A. timonensis |
| Betaproteobacteria | Burkholderiales | Alcaligenaceae | Paenalcaligenes | Paenalcaligenes |
| | | | | hominis |
| | | | Bordella | Bordella pertussis |
| | | Burkholderiaceae | Burkholderia (B.) | B. mallei, B. |
| | | | | pseudomallei |
| | | | Ralstonia | Ralstonia solanacearum |
| | | Neisseriaceae | Neisseria | Neisseria meningitidis |
| | | Sutterellaceae | Sutterella (S.) | S. parvirubra, S. |
| | | | | stercoricanis, S. |
| | | | | wadsworthensis |
| Clostridia | Clostridiales | Catabacteriaceae | Catabacter | Catabacter |
| | | | | hongkongensis |
| | | Clostridiaceae | Aminiphila | Anaerosphaera |
| | | | | aminiphila |
| | | | Christensenellaceae (C.) | C. massiliensis, C. |
| | | | | minuta, C. timonensis |
| | | | Hungatella | Hungatella effluvia |
| | | Eubacteriaceae | Eubacterium (E.) | E. contortum, E. |
| | | | | eligens, E. faecium, E. |
| | | | | hadrum, E. hallii, E. |
| | | | | limosum, E. ramulus, E. |
| | | | | rectale |
| | | Lachnospiraceae | Anaerostipes (A.) | A. caccae, A. hadrus |
| | | | Blautia (B.) | B. hydrogenotrophica, |
| | | | | B. massiliensis, B. |
| | | | | stercoris, B. wexlerae |
| | | | Catonella | Catonella morbi |
| | | | Coprococcus (C.) | C. catus, C. comes, C. |
| | | | | eutactus |
| | | | Dialister (D.) | D. invisus, D. |
| | | | | micraeophilus, D. |
| | | | | succinatiphilus |
| | | | Dorea (D.) | D. formicigenerans, D. |
| | | | | longicatena |
| | | | Johnsonella | Johnsonella ignava |
| | | | Oribacterium (O.) | O. parvum, O. sinus |
| | | | Lachnobacterium | |
| | | | Lachnoclostridium | |
| | | | Lacrimispora (L.) | L. sacchaarolytica |
| | | | Roseburia (R.) | R. hominis, R. |
| | | | | intestinalis |
| | | | Tyzzerella | Tyzzerella nexilis |
| | | Oscillospiraceae | Oscillibacter | Oscillibacter |
| | | | | valericigenes |
| | | | Harryflintia | Harryflinta acetispora |
| | | Peptococcaceae | | |
| | | Peptostreptococcaceae | Paraclostridium | Paraclostridium |
| | | | | benzoelyticum |
| | | | Peptostreptococcus | Peptostreptococcus |
| | | | | russellii |
| | | Ruminococcaceae | Agathobaculum | Agathobaculum sp. |
| | | | Fournierella | Fournierella |
| | | | | masssiliensis |
| | | | Ruminococcus (R.) | R. albus, R. bromii, R. |
| | | | | callidus, R. gnavus, R. |
| | | | | inulinivorans, R. |
| | | | | obeum, R. torques |
| | | | Faecalibacterium | Faecalibacterium |
| | | | | prasusnitzii |
| | | Clostridiales family | | Intestimonas |
| | | XIII/Incertae sedis | | butyriciproducens |
| Fusobacteria | Fusobacteriales | Fusobacteriaceae | Fusobacterium (F.) | F. nucleatum, F. |
| | | | | naviforme |
| | | Leptotrichiaceae | Leptotrichia | |
| | | | Sneathia | |
| Gammaproteobacteria | Enterobacterales | Enterobacteriaceae | Klebsiella (K.) | K. oxytoca, K. |
| | | | | pneumoniae, K. |
| | | | | quasipneumoniae |
| | | | | subsp. |
| | | | | Similipneumoniae, |
| | | | Escherichia (E.) | E. coli strain Nissle |
| | | | | 1917 (EcN), E. coli |
| | | | | strain ECOR12, E. coli |
| | | | | strain ECOR63 |
| | | | Shigella | |
| Negativicutes | | Acidaminococcaceae | Acidaminococcus (A.) | A. fermentans, A. |
| | | | | intestine |
| | | | Phascolarctobacterium (P.) | P. faecium, P. |
| | | | | succinatutens |
| | | Selenomonadaceae | Selenomonas (S.) | S. felix, S. incertae |
| | | | | sedis, S. sputigena |
| | | Sporomusaceae | Selenomonadales | |
| | | Veillonellaceae | Allisonella | |
| | | | Anaeroglobus | Anaeroglobus |
| | | | | germinatus |
| | | | Caecibacter | |
| | | | Colibacter | |
| | | | Megasphaera (M.) | M. elsedenii, M. |
| | | | | massiliensis, M. |
| | | | | micronuciformis, |
| | | | | Megasphaera sp |
| | | | Massilibacillus | Massilibacillus |
| | | | | massiliensis |
| | | | Propionispira | |
| | | | Negativicoccus | Negativicoccus |
| | | | | succinicivornas |
| | | | Veillonella (V.) | V. dispar, V. parvula, V. |
| | | | | ratti, V. tobetsuensis |
| | Synergistales | Synergistaceae | Aminobacterium | Aminobacterium |
| | | | | mobile |
| | | | Cloacibacillus | Cloacibacillus evryensis |
| | | | Rarimicrobium | Rarimicrobium hominis |
| Verrucomicrobia | Verrucomicrobiales | Akkermansiaceae | Akkermansia | Akkermansia |
| | | | | mucinophila |

*The abbreviation given in the parenthetical is for the species in the row in which it is listed.

TABLE 2
Exemplary Bacterial Strains

| OTU | Public DB Accession |
|---|---|
| Actinobacillus actinomycetemcomitans | AY362885 |
| Actinobacillus minor | ACFT01000025 |
| Actinobacillus pleuropneumoniae | NR_074857 |
| Actinobacillus succinogenes | CP000746 |
| Actinobacillus ureae | AEVG01000167 |
| Actinobaculum massiliae | AF487679 |
| Actinobaculum schaalii | AY957507 |
| Actinobaculum sp. BM#101342 | AY282578 |
| Actinobaculum sp. P2P_19 P1 | AY207066 |
| Akkermansia muciniphila | CP001071 |
| Alistipes finegoldii | NR_043064 |
| Alistipes indistinctus | AB490804 |
| Alistipes onderdonkii | NR_043318 |
| Alistipes putredinis | ABFK02000017 |
| Alistipes shahii | FP929032 |
| Alistipes sp. HGB5 | AENZ01000082 |
| Alistipes sp. JC50 | JF824804 |
| Alistipes sp. RMA 9912 | GQ140629 |
| Anaerostipes caccae | ABAX03000023 |
| Anaerostipes sp. 3_2_56FAA | ACWB01000002 |
| Bacillus aeolius | NR_025557 |
| Bacillus aerophilus | NR_042339 |
| Bacillus aestuarii | GQ980243 |
| Bacillus alcalophilus | X76436 |
| Bacillus amyloliquefaciens | NR_075005 |
| Bacillus anthracis | AAEN01000020 |
| Bacillus atrophaeus | NR_075016 |
| Bacillus badius | NR_036893 |
| Bacillus cereus | ABDJ01000015 |
| Bacillus circulans | AB271747 |
| Bacillus clausii | FN397477 |
| Bacillus coagulans | DQ297928 |
| Bacillus firmus | NR_025842 |
| Bacillus flexus | NR_024691 |
| Bacillus fordii | NR_025786 |
| Bacillus gelatini | NR_025595 |
| Bacillus halmapalus | NR_026144 |
| Bacillus halodurans | AY144582 |
| Bacillus herbersteinensis | NR_042286 |
| Bacillus horti | NR_036860 |
| Bacillus idriensis | NR_043268 |
| Bacillus lentus | NR_040792 |
| Bacillus licheniformis | NC_006270 |
| Bacillus megaterium | GU252124 |
| Bacillus nealsonii | NR_044546 |
| Bacillus niabensis | NR_043334 |
| Bacillus niacini | NR_024695 |
| Bacillus pocheonensis | NR_041377 |
| Bacillus pumilus | NR_074977 |
| Bacillus safensis | JQ624766 |
| Bacillus simplex | NR_042136 |
| Bacillus sonorensis | NR_025130 |
| Bacillus sp. 10403023 MM10403188 | CAET01000089 |
| Bacillus sp. 2_A_57_CT2 | ACWD01000095 |
| Bacillus sp. 2008724126 | GU252108 |
| Bacillus sp. 2008724139 | GU252111 |
| Bacillus sp. 7_16AIA | FN397518 |
| Bacillus sp. 9_3AIA | FN397519 |
| Bacillus sp. AP8 | JX101689 |
| Bacillus sp. B27(2008) | EU362173 |
| Bacillus sp. BT1B_CT2 | ACWC01000034 |
| Bacillus sp. GB1.1 | FJ897765 |
| Bacillus sp. GB9 | FJ897766 |
| Bacillus sp. HU19.1 | FJ897769 |
| Bacillus sp. HU29 | FJ897771 |
| Bacillus sp. HU33.1 | FJ897772 |
| Bacillus sp. JC6 | JF824800 |
| Bacillus sp. oral taxon F26 | HM099642 |
| Bacillus sp. oral taxon F28 | HM099650 |
| Bacillus sp. oral taxon F79 | HM099654 |
| Bacillus sp. SRC_DSF1 | GU797283 |
| Bacillus sp. SRC_DSF10 | GU797292 |
| Bacillus sp. SRC_DSF2 | GU797284 |
| Bacillus sp. SRC_DSF6 | GU797288 |
| Bacillus sp. tc09 | HQ844242 |
| Bacillus sp. zh168 | FJ851424 |
| Bacillus sphaericus | DQ286318 |
| Bacillus sporothermodurans | NR_026010 |
| Bacillus subtilis | EU627588 |
| Bacillus thermoamylovorans | NR_029151 |
| Bacillus weihenstephanensis | NR_074926 |
| Bacteroidales bacterium ph8 | JN837494 |
| Bacteroidales genomosp. P1 | AY341819 |
| Bacteroidales genomosp. P2 oral | DQ003613 |
| clone MB1_G13 | |
| Bacteroidales genomosp. P3 oral | DQ003615 |
| clone MB1_G34 | |
| Bacteroidales genomosp. P4 oral | DQ003617 |
| clone MB2_G17 | |
| Bacteroidales genomosp. P5 oral | DQ003619 |
| clone MB2_P04 | |
| Bacteroidales genomosp. P6 oral | DQ003634 |
| clone MB3_C19 | |
| Bacteroidales genomosp. P7 oral | DQ003623 |
| clone MB3_P19 | |
| Bacteroidales genomosp. P8 oral | DQ003626 |
| clone MB4_G15 | |
| Bacteroides acidifaciens | NR_028607 |
| Bacteroides barnesiae | NR_041446 |
| Bacteroides caccae | EU136686 |
| Bacteroides cellulosilyticus | ACCH01000108 |
| Bacteroides clarus | AFBM01000011 |
| Bacteroides coagulans | AB547639 |
| Bacteroides coprocola | ABIY02000050 |
| Bacteroides coprophilus | ACBW01000012 |
| Bacteroides dorei | ABWZ01000093 |
| Bacteroides eggerthii | ACWG01000065 |
| Bacteroides faecis | GQ496624 |
| Bacteroides finegoldii | AB222699 |
| Bacteroides fluxus | AFBN01000029 |
| Bacteroides fragilis | AP006841 |
| Bacteroides galacturonicus | DQ497994 |
| Bacteroides helcogenes | CP002352 |
| Bacteroides heparinolyticus | JN867284 |
| Bacteroides intestinalis | ABJL02000006 |
| Bacteroides massiliensis | AB200226 |
| Bacteroides nordii | NR_043017 |
| Bacteroides oleiciplenus | AB547644 |
| Bacteroides ovatus | ACWH01000036 |
| Bacteroides pectinophilus | ABVQ01000036 |
| Bacteroides plebeius | AB200218 |
| Bacteroides pyogenes | NR_041280 |
| Bacteroides salanitronis | CP002530 |
| Bacteroides salyersiae | EU136690 |
| Bacteroides sp. 1_1_14 | ACRP01000155 |
| Bacteroides sp. 1_1_30 | ADCL01000128 |
| Bacteroides sp. 1_1_6 | ACIC01000215 |
| Bacteroides sp. 2_1_22 | ACPQ01000117 |
| Bacteroides sp. 2_1_56FAA | ACWI01000065 |
| Bacteroides sp. 2_2_4 | ABZZ01000168 |
| Bacteroides sp. 20_3 | ACRQ01000064 |
| Bacteroides sp. 3_1_19 | ADCJ01000062 |
| Bacteroides sp. 3_1_23 | ACRS01000081 |
| Bacteroides sp. 3_1_33FAA | ACPS01000085 |
| Bacteroides sp. 3_1_40A | ACRT01000136 |
| Bacteroides sp. 3_2_5 | ACIB01000079 |
| Bacteroides sp. 315_5 | FJ848547 |
| Bacteroides sp. 31SF15 | AJ583248 |
| Bacteroides sp. 31SF18 | AJ583249 |
| Bacteroides sp. 35AE31 | AJ583244 |
| Bacteroides sp. 35AE37 | AJ583245 |
| Bacteroides sp. 35BE34 | AJ583246 |
| Bacteroides sp. 35BE35 | AJ583247 |
| Bacteroides sp. 4_1_36 | ACTC01000133 |
| Bacteroides sp. 4_3_47FAA | ACDR02000029 |
| Bacteroides sp. 9_1_42FAA | ACAA01000096 |
| Bacteroides sp. AR20 | AF139524 |
| Bacteroides sp. AR29 | AF139525 |
| Bacteroides sp. B2 | EU722733 |
| Bacteroides sp. D1 | ACAB02000030 |
| Bacteroides sp. D2 | ACGA01000077 |
| Bacteroides sp. D20 | ACPT01000052 |
| Bacteroides sp. D22 | ADCK01000151 |
| Bacteroides sp. F_4 | AB470322 |
| Bacteroides sp. NB_8 | AB117565 |
| Bacteroides sp. WH2 | AY895180 |
| Bacteroides sp. XB12B | AM230648 |
| Bacteroides sp. XB44A | AM230649 |
| Bacteroides stercoris | ABFZ02000022 |
| Bacteroides thetaiotaomicron | NR_074277 |
| Bacteroides uniformis | AB050110 |
| Bacteroides ureolyticus | GQ167666 |
| Bacteroides vulgatus | CP000139 |
| Bacteroides xylanisolvens | ADKP01000087 |
| Bacteroidetes bacterium oral | HM099638 |
| taxon D27 | |
| Bacteroidetes bacterium oral | HM099643 |
| taxon F31 | |
| Bacteroidetes bacterium oral | HM099649 |
| taxon F44 | |
| Barnesiella intestinihominis | AB370251 |
| Bifidobacteriaceae genomosp. C1 | AY278612 |
| Bifidobacterium adolescentis | AAXD02000018 |
| Bifidobacterium angulatum | ABYS02000004 |
| Bifidobacterium animalis | CP001606 |
| Bifidobacterium bifidum | ABQP01000027 |
| Bifidobacterium breve | CP002743 |
| Bifidobacterium catenulatum | ABXY01000019 |
| Bifidobacterium dentium | CP001750 |
| Bifidobacterium gallicum | ABXB03000004 |
| Bifidobacterium infantis | AY151398 |
| Bifidobacterium kashiwanohense | AB491757 |
| Bifidobacterium longum | ABQQ01000041 |
| Bifidobacterium pseudocatenulatum | ABXX02000002 |
| Bifidobacterium pseudolongum | NR_043442 |
| Bifidobacterium scardovii | AJ307005 |
| Bifidobacterium sp. HM2 | AB425276 |
| Bifidobacterium sp. HMLN12 | JF519685 |
| Bifidobacterium sp. M45 | HM626176 |
| Bifidobacterium sp. MSX5B | HQ616382 |
| Bifidobacterium sp. TM_7 | AB218972 |
| Bifidobacterium thermophilum | DQ340557 |
| Bifidobacterium urinalis | AJ278695 |
| Blautia coccoides | AB571656 |
| Blautia glucerasea | AB588023 |
| Blautia glucerasei | AB439724 |
| Blautia hansenii | ABYU02000037 |
| Blautia hydrogenotrophica | ACBZ01000217 |
| Blautia luti | AB691576 |
| Blautia producta | AB600998 |
| Blautia schinkii | NR_026312 |
| Blautia sp. M25 | HM626178 |
| Blautia stercoris | HM626177 |
| Blautia wexlerae | EF036467 |
| Bordetella bronchiseptica | NR_025949 |
| Bordetella holmesii | AB683187 |
| Bordetella parapertussis | NR_025950 |
| Bordetella pertussis | BX640418 |
| Borrelia afzelii | ABCU01000001 |
| Borrelia burgdorferi | ABGI01000001 |
| Borrelia crocidurae | DQ057990 |
| Borrelia duttonii | NC_011229 |
| Borrelia garinii | ABJV01000001 |
| Borrelia hermsii | AY597657 |
| Borrelia hispanica | DQ057988 |
| Borrelia persica | HM161645 |
| Borrelia recurrentis | AF107367 |
| Borrelia sp. NE49 | AJ224142 |
| Borrelia spielmanii | ABKB01000002 |
| Borrelia turicatae | NC_008710 |
| Borrelia valaisiana | ABCY01000002 |
| Brucella ovis | NC_009504 |
| Brucella sp. 83_13 | ACBQ01000040 |
| Brucella sp. BO1 | EU053207 |
| Brucella suis | ACBK01000034 |
| Burkholderia ambifaria | AAUZ01000009 |
| Burkholderia cenocepacia | AAHI01000060 |
| Burkholderia cepacia | NR_041719 |
| Burkholderia mallei | CP000547 |
| Burkholderia multivorans | NC_010086 |
| Burkholderia oklahomensis | DQ108388 |
| Burkholderia pseudomallei | CP001408 |
| Burkholderia rhizoxinica | HQ005410 |
| Burkholderia sp. 383 | CP000151 |
| Burkholderia xenovorans | U86373 |
| Burkholderiales bacterium 1_1_47 | ADCQ01000066 |
| Butyrivibrio crossotus | ABWN01000012 |
| Butyrivibrio fibrisolvens | U41172 |
| Chlamydia muridarum | AE002160 |
| Chlamydia psittaci | NR_036864 |
| Chlamydia trachomatis | U68443 |
| Chlamydiales bacterium NS11 | JN606074 |
| Citrobacter amalonaticus | FR870441 |
| Citrobacter braakii | NR_028687 |
| Citrobacter farmeri | AF025371 |
| Citrobacter freundii | NR_028894 |
| Citrobacter gillenii | AF025367 |
| Citrobacter koseri | NC_009792 |
| Citrobacter murliniae | AF025369 |
| Citrobacter rodentium | NR_074903 |
| Citrobacter sedlakii | AF025364 |
| Citrobacter sp. 30_2 | ACDJ01000053 |
| Citrobacter sp. KMSI_3 | GQ468398 |
| Citrobacter werkmanii | AF025373 |
| Citrobacter youngae | ABWL02000011 |
| Cloacibacillus evryensis | GQ258966 |
| Clostridiaceae bacterium END_2 | EF451053 |
| Clostridiaceae bacterium JC13 | JF824807 |
| Clostridiales bacterium 1_7_47FAA | ABQR01000074 |
| Clostridiales bacterium 9400853 | HM587320 |
| Clostridiales bacterium 9403326 | HM587324 |
| Clostridiales bacterium oral clone | AY207065 |
| P4PA_66 P1 | |
| Clostridiales bacterium oral taxon | GQ422712 |
| 093 | |
| Clostridiales bacterium oral taxon | HM099644 |
| F32 | |
| Clostridiales bacterium ph2 | JN837487 |
| Clostridiales bacterium SY8519 | AB477431 |
| Clostridiales genomosp. BVAB3 | CP001850 |
| Clostridiales sp. SM4_1 | FP929060 |
| Clostridiales sp. SS3_4 | AY305316 |
| Clostridiales sp. SSC_2 | FP929061 |
| Clostridium acetobutylicum | NR_074511 |
| Clostridium aerotolerans | X76163 |
| Clostridium aldenense | NR_043680 |
| Clostridium aldrichii | NR_026099 |
| Clostridium algidicarnis | NR_041746 |
| Clostridium algidixylanolyticum | NR_028726 |
| Clostridium aminovalericum | NR_029245 |
| Clostridium amygdalinum | AY353957 |
| Clostridium argentinense | NR_029232 |
| Clostridium asparagiforme | ACCJ01000522 |
| Clostridium baratii | NR_029229 |
| Clostridium bartlettii | ABEZ02000012 |
| Clostridium beijerinckii | NR_074434 |
| Clostridium bifermentans | X73437 |
| Clostridium bolteae | ABCC02000039 |
| Clostridium botulinum | NC_010723 |
| Clostridium butyricum | ABDT01000017 |
| Clostridium cadaveris | AB542932 |
| Clostridium carboxidivorans | FR733710 |
| Clostridium carnis | NR_044716 |
| Clostridium celatum | X77844 |
| Clostridium celerecrescens | JQ246092 |
| Clostridium cellulosi | NR_044624 |
| Clostridium chauvoei | EU106372 |
| Clostridium citroniae | ADLJ01000059 |
| Clostridium clariflavum | NR_041235 |
| Clostridium clostridiiformes | M59089 |
| Clostridium clostridioforme | NR_044715 |
| Clostridium coccoides | EF025906 |
| Clostridium cochlearium | NR_044717 |
| Clostridium cocleatum | NR_026495 |
| Clostridium colicanis | FJ957863 |
| Clostridium colinum | NR_026151 |
| Clostridium difficile | NC_013315 |
| Clostridium disporicum | NR_026491 |
| Clostridium estertheticum | NR_042153 |
| Clostridium fallax | NR_044714 |
| Clostridium favososporum | X76749 |
| Clostridium felsineum | AF270502 |
| Clostridium frigidicarnis | NR_024919 |
| Clostridium gasigenes | NR_024945 |
| Clostridium ghonii | AB542933 |
| Clostridium glycolicum | FJ384385 |
| Clostridium glycyrrhizinilyticum | AB233029 |
| Clostridium haemolyticum | NR_024749 |
| Clostridium hathewayi | AY552788 |
| Clostridium hiranonis | AB023970 |
| Clostridium histolyticum | HF558362 |
| Clostridium hylemonae | AB023973 |
| Clostridium indolis | AF028351 |
| Clostridium innocuum | M23732 |
| Clostridium irregulare | NR_029249 |
| Clostridium isatidis | NR_026347 |
| Clostridium kluyveri | NR_074165 |
| Clostridium lactatifermentans | NR_025651 |
| Clostridium lavalense | EF564277 |
| Clostridium leptum | AJ305238 |
| Clostridium limosum | FR870444 |
| Clostridium magnum | X77835 |
| Clostridium malenominatum | FR749893 |
| Clostridium mayombei | FR733682 |
| Clostridium methylpentosum | ACEC01000059 |
| Clostridium nexile | X73443 |
| Clostridium novyi | NR_074343 |
| Clostridium orbiscindens | Y18187 |
| Clostridium oroticum | FR749922 |
| Clostridium paraputrificum | AB536771 |
| Clostridium perfringens | ABDW01000023 |
| Clostridium phytofermentans | NR_074652 |
| Clostridium piliforme | D14639 |
| Clostridium putrefaciens | NR_024995 |
| Clostridium quinii | NR_026149 |
| Clostridium ramosum | M23731 |
| Clostridium rectum | NR_029271 |
| Clostridium saccharogumia | DQ100445 |
| Clostridium saccharolyticum | CP002109 |
| Clostridium sardiniense | NR_041006 |
| Clostridium sartagoforme | NR_026490 |
| Clostridium scindens | AF262238 |
| Clostridium septicum | NR_026020 |
| Clostridium sordellii | AB448946 |
| Clostridium sp. 7_2_43FAA | ACDK01000101 |
| Clostridium sp. D5 | ADBG01000142 |
| Clostridium sp. HGF2 | AENW01000022 |
| Clostridium sp. HPB_46 | AY862516 |
| Clostridium sp. JC122 | CAEV01000127 |
| Clostridium sp. L2_50 | AAYW02000018 |
| Clostridium sp. LMG 16094 | X95274 |
| Clostridium sp. M62_1 | ACFX02000046 |
| Clostridium sp. MLG055 | AF304435 |
| Clostridium sp. MT4 E | FJ159523 |
| Clostridium sp. NMBHI_1 | JN093130 |
| Clostridium sp. NML 04A032 | EU815224 |
| Clostridium sp. SS2_1 | ABGC03000041 |
| Clostridium sp. SY8519 | AP012212 |
| Clostridium sp. TM_40 | AB249652 |
| Clostridium sp. YIT 12069 | AB491207 |
| Clostridium sp. YIT 12070 | AB491208 |
| Clostridium sphenoides | X73449 |
| Clostridium spiroforme | X73441 |
| Clostridium sporogenes | ABKW02000003 |
| Clostridium sporosphaeroides | NR_044835 |
| Clostridium stercorarium | NR_025100 |
| Clostridium sticklandii | L04167 |
| Clostridium straminisolvens | NR_024829 |
| Clostridium subterminale | NR_041795 |
| Clostridium sulfidigenes | NR_044161 |
| Clostridium symbiosum | ADLQ01000114 |
| Clostridium tertium | Y18174 |
| Clostridium tetani | NC_004557 |
| Clostridium thermocellum | NR_074629 |
| Clostridium tyrobutyricum | NR_044718 |
| Clostridium viride | NR_026204 |
| Clostridium xylanolyticum | NR_037068 |
| Collinsella aerofaciens | AAVN02000007 |
| Collinsella intestinalis | ABXH02000037 |
| Collinsella stercoris | ABXJ01000150 |
| Collinsella tanakaei | AB490807 |
| Coprobacillus cateniformis | AB030218 |
| Coprobacillus sp. 29_1 | ADKX01000057 |
| Coprobacillus sp. D7 | ACDT01000199 |
| Coprococcus catus | EU266552 |
| Coprococcus comes | ABVR01000038 |
| Coprococcus eutactus | EF031543 |
| Coprococcus sp. ART55_1 | AY350746 |
| Dialister invisus | ACIM02000001 |
| Dialister micraerophilus | AFBB01000028 |
| Dialister microaerophilus | AENT01000008 |
| Dialister pneumosintes | HM596297 |
| Dialister propionicifaciens | NR_043231 |
| Dialister sp. oral taxon 502 | GQ422739 |
| Dialister succinatiphilus | AB370249 |
| Dorea formicigenerans | AAXA02000006 |
| Dorea longicatena | AJ132842 |
| Enhydrobacter aerosaccus | ACYI01000081 |
| Enterobacter aerogenes | AJ251468 |
| Enterobacter asburiae | NR_024640 |
| Enterobacter cancerogenus | Z96078 |
| Enterobacter cloacae | FP929040 |
| Enterobacter cowanii | NR_025566 |
| Enterobacter hormaechei | AFHR01000079 |
| Enterobacter sp. 247BMC | HQ122932 |
| Enterobacter sp. 638 | NR_074777 |
| Enterobacter sp. JC163 | JN657217 |
| Enterobacter sp. SCSS | HM007811 |
| Enterobacter sp. TSE38 | HM156134 |
| Enterobacteriaceae bacterium | ADCU01000033 |
| 9_2_54FAA | |
| Enterobacteriaceae bacterium | AJ489826 |
| CF01Ent_1 | |
| Enterobacteriaceae bacterium | AY538694 |
| Smarlab 3302238 | |
| Enterococcus avium | AF133535 |
| Enterococcus caccae | AY943820 |
| Enterococcus casseliflavus | AEWT01000047 |
| Enterococcus durans | AJ276354 |
| Enterococcus faecalis | AE016830 |
| Enterococcus faecium | AM157434 |
| Enterococcus gallinarum | AB269767 |
| Enterococcus gilvus | AY033814 |
| Enterococcus hawaiiensis | AY321377 |
| Enterococcus hirae | AF061011 |
| Enterococcus italicus | AEPV01000109 |
| Enterococcus mundtii | NR_024906 |
| Enterococcus raffinosus | FN600541 |
| Enterococcus sp. BV2CASA2 | JN809766 |
| Enterococcus sp. CCRI_16620 | GU457263 |
| Enterococcus sp. F95 | FJ463817 |
| Enterococcus sp. RfL6 | AJ133478 |
| Enterococcus thailandicus | AY321376 |
| Erysipelotrichaceae bacterium | ACTJ01000113 |
| 3_1_53 | |
| Erysipelotrichaceae bacterium | ACZW01000054 |
| 5_2_54FAA | |
| Escherichia albertii | ABKX01000012 |
| Escherichia coli | NC_008563 |
| Escherichia fergusonii | CU928158 |
| Escherichia hermannii | HQ407266 |
| Escherichia sp. 1_1_43 | ACID01000033 |
| Escherichia sp. 4_1_40B | ACDM02000056 |
| Escherichia sp. B4 | EU722735 |
| Escherichia vulneris | NR_041927 |
| Eubacteriaceae bacterium P4P_50 P4 | AY207060 |
| Eubacterium barkeri | NR_044661 |
| Eubacterium biforme | ABYT01000002 |
| Eubacterium brachy | U13038 |
| Eubacterium budayi | NR_024682 |
| Eubacterium callanderi | NR_026330 |
| Eubacterium cellulosolvens | AY178842 |
| Eubacterium contortum | FR749946 |
| Eubacterium coprostanoligenes | HM037995 |
| Eubacterium cylindroides | FP929041 |
| Eubacterium desmolans | NR_044644 |
| Eubacterium dolichum | L34682 |
| Eubacterium eligens | CP001104 |
| Eubacterium fissicatena | FR749935 |
| Eubacterium hadrum | FR749933 |
| Eubacterium hallii | L34621 |
| Eubacterium infirmum | U13039 |
| Eubacterium limosum | CP002273 |
| Eubacterium moniliforme | HF558373 |
| Eubacterium multiforme | NR_024683 |
| Eubacterium nitritogenes | NR_024684 |
| Eubacterium nodatum | U13041 |
| Eubacterium ramulus | AJ011522 |
| Eubacterium rectale | FP929042 |
| Eubacterium ruminantium | NR_024661 |
| Eubacterium saburreum | AB525414 |
| Eubacterium saphenum | NR_026031 |
| Eubacterium siraeum | ABCA03000054 |
| Eubacterium sp. 3_1_31 | ACTL01000045 |
| Eubacterium sp. AS15b | HQ616364 |
| Eubacterium sp. OBRC9 | HQ616354 |
| Eubacterium sp. oral clone GI038 | AY349374 |
| Eubacterium sp. oral clone IR009 | AY349376 |
| Eubacterium sp. oral clone JH012 | AY349373 |
| Eubacterium sp. oral clone JI012 | AY349379 |
| Eubacterium sp. oral clone JN088 | AY349377 |
| Eubacterium sp. oral clone JS001 | AY349378 |
| Eubacterium sp. oral clone OH3A | AY947497 |
| Eubacterium sp. WAL 14571 | FJ687606 |
| Eubacterium tenue | M59118 |
| Eubacterium tortuosum | NR_044648 |
| Eubacterium ventriosum | L34421 |
| Eubacterium xylanophilum | L34628 |
| Eubacterium yurii | AEES01000073 |
| Fusobacterium canifelinum | AY162222 |
| Fusobacterium genomosp. C1 | AY278616 |
| Fusobacterium genomosp. C2 | AY278617 |
| Fusobacterium gonidiaformans | ACET01000043 |
| Fusobacterium mortiferum | ACDB02000034 |
| Fusobacterium naviforme | HQ223106 |
| Fusobacterium necrogenes | X55408 |
| Fusobacterium necrophorum | AM905356 |
| Fusobacterium nucleatum | ADVK01000034 |
| Fusobacterium periodonticum | ACJY01000002 |
| Fusobacterium russii | NR_044687 |
| Fusobacterium sp. 1_1_41FAA | ADGG01000053 |
| Fusobacterium sp. 11_3_2 | ACUO01000052 |
| Fusobacterium sp. 12_1B | AGWJ01000070 |
| Fusobacterium sp. 2_1_31 | ACDC02000018 |
| Fusobacterium sp. 3_1_27 | ADGF01000045 |
| Fusobacterium sp. 3_1_33 | ACQE01000178 |
| Fusobacterium sp. 3_1_36A2 | ACPU01000044 |
| Fusobacterium sp. 3_1_5R | ACDD01000078 |
| Fusobacterium sp. AC18 | HQ616357 |
| Fusobacterium sp. ACB2 | HQ616358 |
| Fusobacterium sp. AS2 | HQ616361 |
| Fusobacterium sp. CM1 | HQ616371 |
| Fusobacterium sp. CM21 | HQ616375 |
| Fusobacterium sp. CM22 | HQ616376 |
| Fusobacterium sp. D12 | ACDG02000036 |
| Fusobacterium sp. oral clone | AY923141 |
| ASCF06 | |
| Fusobacterium sp. oral clone | AY953256 |
| ASCF11 | |
| Fusobacterium ulcerans | ACDH01000090 |
| Fusobacterium varium | ACIE01000009 |
| Gemella haemolysans | ACDZ02000012 |
| Gemella morbillorum | NR_025904 |
| Gemella morbillorum | ACRX01000010 |
| Gemella sanguinis | ACRY01000057 |
| Gemella sp. oral clone ASCE02 | AY923133 |
| Gemella sp. oral clone ASCF04 | AY923139 |
| Gemella sp. oral clone ASCF12 | AY923143 |
| Gemella sp. WAL 1945J | EU427463 |
| Klebsiella oxytoca | AY292871 |
| Klebsiella pneumoniae | CP000647 |
| Klebsiella sp. AS10 | HQ616362 |
| Klebsiella sp. Co9935 | DQ068764 |
| Klebsiella sp. enrichment culture | HM195210 |
| clone SRC_DSD25 | |
| Klebsiella sp. OBRC7 | HQ616353 |
| Klebsiella sp. SP_BA | FJ999767 |
| Klebsiella sp. SRC_DSD1 | GU797254 |
| Klebsiella sp. SRC_DSD11 | GU797263 |
| Klebsiella sp. SRC_DSD12 | GU797264 |
| Klebsiella sp. SRC_DSD15 | GU797267 |
| Klebsiella sp. SRC_DSD2 | GU797253 |
| Klebsiella sp. SRC_DSD6 | GU797258 |
| Klebsiella variicola | CP001891 |
| Lachnobacterium bovis | GU324407 |
| Lachnospira multipara | FR733699 |
| Lachnospira pectinoschiza | L14675 |
| Lachnospiraceae bacterium | ACTM01000065 |
| 1_1_57FAA | |
| Lachnospiraceae bacterium | ACTN01000028 |
| 1_4_56FAA | |
| Lachnospiraceae bacterium | ADLB01000035 |
| 2_1_46FAA | |
| Lachnospiraceae bacterium | ACTO01000052 |
| 2_1_58FAA | |
| Lachnospiraceae bacterium | ACTP01000124 |
| 3_1_57FAA_CT1 | |
| Lachnospiraceae bacterium | ADCR01000030 |
| 4_1_37FAA | |
| Lachnospiraceae bacterium | ACTR01000020 |
| 5_1_57FAA | |
| Lachnospiraceae bacterium | ACTS01000081 |
| 5_1_63FAA | |
| Lachnospiraceae bacterium | ACTV01000014 |
| 6_1_63FAA | |
| Lachnospiraceae bacterium | ACWQ01000079 |
| 8_1_57FAA | |
| Lachnospiraceae bacterium | ACTX01000023 |
| 9_1_43BFAA | |
| Lachnospiraceae bacterium A4 | DQ789118 |
| Lachnospiraceae bacterium DJF VP30 | EU728771 |
| Lachnospiraceae bacterium ICM62 | HQ616401 |
| Lachnospiraceae bacterium MSX33 | HQ616384 |
| Lachnospiraceae bacterium oral | ADDS01000069 |
| taxon 107 | |
| Lachnospiraceae bacterium oral | HM099641 |
| taxon F15 | |
| Lachnospiraceae genomosp. C1 | AY278618 |
| Lactobacillus acidipiscis | NR_024718 |
| Lactobacillus acidophilus | CP000033 |
| Lactobacillus alimentarius | NR_044701 |
| Lactobacillus amylolyticus | ADNY01000006 |
| Lactobacillus amylovorus | CP002338 |
| Lactobacillus antri | ACLL01000037 |
| Lactobacillus brevis | EU194349 |
| Lactobacillus buchneri | ACGH01000101 |
| Lactobacillus casei | CP000423 |
| Lactobacillus catenaformis | M23729 |
| Lactobacillus coleohominis | ACOH01000030 |
| Lactobacillus coryniformis | NR_044705 |
| Lactobacillus crispatus | ACOG01000151 |
| Lactobacillus curvatus | NR_042437 |
| Lactobacillus delbrueckii | CP002341 |
| Lactobacillus dextrinicus | NR_036861 |
| Lactobacillus farciminis | NR_044707 |
| Lactobacillus fermentum | CP002033 |
| Lactobacillus gasseri | ACOZ01000018 |
| Lactobacillus gastricus | AICN01000060 |
| Lactobacillus genomosp. C1 | AY278619 |
| Lactobacillus genomosp. C2 | AY278620 |
| Lactobacillus helveticus | ACLM01000202 |
| Lactobacillus hilgardii | ACGP01000200 |
| Lactobacillus hominis | FR681902 |
| Lactobacillus iners | AEKJ01000002 |
| Lactobacillus jensenii | ACQD01000066 |
| Lactobacillus johnsonii | AE017198 |
| Lactobacillus kalixensis | NR_029083 |
| Lactobacillus kefiranofaciens | NR_042440 |
| Lactobacillus kefiri | NR_042230 |
| Lactobacillus kimchii | NR_025045 |
| Lactobacillus leichmannii | JX986966 |
| Lactobacillus mucosae | FR693800 |
| Lactobacillus murinus | NR_042231 |
| Lactobacillus nodensis | NR_041629 |
| Lactobacillus oeni | NR_043095 |
| Lactobacillus oris | AEKL01000077 |
| Lactobacillus parabrevis | NR_042456 |
| Lactobacillus parabuchneri | NR_041294 |
| Lactobacillus paracasei | ABQV01000067 |
| Lactobacillus parakefiri | NR_029039 |
| Lactobacillus pentosus | JN813103 |
| Lactobacillus perolens | NR_029360 |
| Lactobacillus plantarum | ACGZ02000033 |
| Lactobacillus pontis | HM218420 |
| Lactobacillus reuteri | ACGW02000012 |
| Lactobacillus rhamnosus | ABWJ01000068 |
| Lactobacillus rogosae | GU269544 |
| Lactobacillus ruminis | ACGS02000043 |
| Lactobacillus sakei | DQ989236 |
| Lactobacillus salivarius | AEBA01000145 |
| Lactobacillus saniviri | AB602569 |
| Lactobacillus senioris | AB602570 |
| Lactobacillus sp. 66c | FR681900 |
| Lactobacillus sp. BT6 | HQ616370 |
| Lactobacillus sp. KLDS | EU600905 |
| 1.0701 | |
| Lactobacillus sp. KLDS | EU600906 |
| 1.0702 | |
| Lactobacillus sp. KLDS | EU600907 |
| 1.0703 | |
| Lactobacillus sp. KLDS | EU600908 |
| 1.0704 | |
| Lactobacillus sp. KLDS | EU600909 |
| 1.0705 | |
| Lactobacillus sp. KLDS | EU600911 |
| 1.0707 | |
| Lactobacillus sp. KLDS | EU600913 |
| 1.0709 | |
| Lactobacillus sp. KLDS | EU600915 |
| 1.0711 | |
| Lactobacillus sp. KLDS | EU600916 |
| 1.0712 | |
| Lactobacillus sp. KLDS | EU600917 |
| 1.0713 | |
| Lactobacillus sp. KLDS | EU600921 |
| 1.0716 | |
| Lactobacillus sp. KLDS | EU600922 |
| 1.0718 | |
| Lactobacillus sp. KLDS | EU600923 |
| 1.0719 | |
| Lactobacillus sp. oral clone | AY349382 |
| HT002 | |
| Lactobacillus sp. oral clone | AY349383 |
| HT070 | |
| Lactobacillus sp. oral taxon | GQ422710 |
| 052 | |
| Lactobacillus tucceti | NR_042194 |
| Lactobacillus ultunensis | ACGU01000081 |
| Lactobacillus vaginalis | ACGV01000168 |
| Lactobacillus vini | NR_042196 |
| Lactobacillus vitulinus | NR_041305 |
| Lactobacillus zeae | NR_037122 |
| Lactococcus garvieae | AF061005 |
| Lactococcus lactis | CP002365 |
| Lactococcus raffinolactis | NR_044359 |
| Listeria grayi | ACCR02000003 |
| Listeria innocua | JF967625 |
| Listeria ivanovii | X56151 |
| Listeria monocytogenes | CP002003 |
| Listeria welshimeri | AM263198 |
| Megasphaera elsdenii | AY038996 |
| Megasphaera genomosp. C1 | AY278622 |
| Megasphaera genomosp. type_1 | ADGP01000010 |
| Megasphaera micronuciformis | AECS01000020 |
| Megasphaera sp. BLPYG_07 | HM990964 |
| Megasphaera sp. UPII 199_6 | AFIJ01000040 |
| Microbacterium gubbeenense | NR_025098 |
| Microbacterium lacticum | EU714351 |
| Mitsuokella jalaludinii | NR_028840 |
| Mitsuokella multacida | ABWK02000005 |
| Mitsuokella sp. oral taxon 521 | GU413658 |
| Mitsuokella sp. oral taxon G68 | GU432166 |
| Mycobacterium abscessus | AGQU01000002 |
| Mycobacterium africanum | AF480605 |
| Mycobacterium alsiensis | AJ938169 |
| Mycobacterium avium | CP000479 |
| Mycobacterium chelonae | AB548610 |
| Mycobacterium colombiense | AM062764 |
| Mycobacterium elephantis | AF385898 |
| Mycobacterium gordonae | GU142930 |
| Mycobacterium intracellulare | GQ153276 |
| Mycobacterium kansasii | AF480601 |
| Mycobacterium lacus | NR_025175 |
| Mycobacterium leprae | FM211192 |
| Mycobacterium lepromatosis | EU203590 |
| Mycobacterium mageritense | FR798914 |
| Mycobacterium mantenii | FJ042897 |
| Mycobacterium marinum | NC_010612 |
| Mycobacterium microti | NR_025234 |
| Mycobacterium neoaurum | AF268445 |
| Mycobacterium parascrofulaceum | ADNV01000350 |
| Mycobacterium paraterrae | EU919229 |
| Mycobacterium phlei | GU142920 |
| Mycobacterium seoulense | DQ536403 |
| Mycobacterium smegmatis | CP000480 |
| Mycobacterium sp. 1761 | EU703150 |
| Mycobacterium sp. 1776 | EU703152 |
| Mycobacterium sp. 1781 | EU703147 |
| Mycobacterium sp. 1791 | EU703148 |
| Mycobacterium sp. 1797 | EU703149 |
| Mycobacterium sp. AQ1GA4 | HM210417 |
| Mycobacterium sp. | HQ174245 |
| B10_07.09.0206 | |
| Mycobacterium sp. | FJ497243 |
| GN_10546 | |
| Mycobacterium sp. | FJ497247 |
| GN_10827 | |
| Mycobacterium sp. | FJ652846 |
| GN_11124 | |
| Mycobacterium sp. | FJ497240 |
| GN_9188 | |
| Mycobacterium sp. | FJ555538 |
| GR_2007_210 | |
| Mycobacterium sp. HE5 | AJ012738 |
| Mycobacterium sp. | HM627011 |
| NLA001000736 | |
| Mycobacterium sp. W | DQ437715 |
| Mycobacterium tuberculosis | CP001658 |
| Mycobacterium ulcerans | AB548725 |
| Mycobacterium vulneris | EU834055 |
| Mycoplasma agalactiae | AF010477 |
| Mycoplasma amphoriforme | AY531656 |
| Mycoplasma arthritidis | NC_011025 |
| Mycoplasma bovoculi | NR_025987 |
| Mycoplasma faucium | NR_024983 |
| Mycoplasma fermentans | CP002458 |
| Mycoplasma flocculare | X62699 |
| Mycoplasma genitalium | L43967 |
| Mycoplasma hominis | AF443616 |
| Mycoplasma orale | AY796060 |
| Mycoplasma ovipneumoniae | NR_025989 |
| Mycoplasma penetrans | NC_004432 |
| Mycoplasma pneumoniae | NC_000912 |
| Mycoplasma putrefaciens | U26055 |
| Mycoplasma salivarium | M24661 |
| Mycoplasmataceae genomosp. | DQ003614 |
| P1 oral clone MB1_G23 | |
| Neisseria bacilliformis | AFAY01000058 |
| Neisseria cinerea | ACDY01000037 |
| Neisseria elongata | ADBF01000003 |
| Neisseria flavescens | ACQV01000025 |
| Neisseria genomosp. | DQ003630 |
| P2 oral clone MB5_P15 | |
| Neisseria gonorrhoeae | CP002440 |
| Neisseria lactamica | ACEQ01000095 |
| Neisseria macacae | AFQE01000146 |
| Neisseria meningitidis | NC_003112 |
| Neisseria mucosa | ACDX01000110 |
| Neisseria pharyngis | AJ239281 |
| Neisseria polysaccharea | ADBE01000137 |
| Neisseria sicca | ACKO02000016 |
| Neisseria sp. KEM232 | GQ203291 |
| Neisseria sp. oral clone | AY005027 |
| AP132 | |
| Neisseria sp. oral clone | AY349388 |
| JC012 | |
| Neisseria sp. oral strain | AY005028 |
| B33KA | |
| Neisseria sp. oral taxon 014 | ADEA01000039 |
| Neisseria sp. SMC_A9199 | FJ763637 |
| Neisseria sp. TM10_1 | DQ279352 |
| Neisseria subflava | ACEO01000067 |
| Odoribacter laneus | AB490805 |
| Odoribacter splanchnicus | CP002544 |
| Oscillibacter sp. G2 | HM626173 |
| Oscillibacter valericigenes | NR_074793 |
| Oscillospira guilliermondii | AB040495 |
| Paenibacillus barcinonensis | NR_042272 |
| Paenibacillus barengoltzii | NR_042756 |
| Paenibacillus chibensis | NR_040885 |
| Paenibacillus cookii | NR_025372 |
| Paenibacillus durus | NR_037017 |
| Paenibacillus glucanolyticus | D78470 |
| Paenibacillus lactis | NR_025739 |
| Paenibacillus lautus | NR_040882 |
| Paenibacillus pabuli | NR_040853 |
| Paenibacillus polymyxa | NR_037006 |
| Paenibacillus popilliae | NR_040888 |
| Paenibacillus sp. CIP 101062 | HM212646 |
| Parabacteroides distasonis | CP000140 |
| Parabacteroides goldsteinii | AY974070 |
| Parabacteroides gordonii | AB470344 |
| Parabacteroides johnsonii | ABYH01000014 |
| Parabacteroides merdae | EU136685 |
| Parabacteroides sp. D13 | ACPW01000017 |
| Parabacteroides sp. NS31_3 | JN029805 |
| Peptococcus niger | NR_029221 |
| Peptococcus sp. oral clone | AY349389 |
| JM048 | |
| Peptococcus sp. oral taxon | GQ422727 |
| 167 | |
| Peptoniphilus asaccharolyticus | D14145 |
| Peptoniphilus duerdenii | EU526290 |
| Peptoniphilus harei | NR_026358 |
| Peptoniphilus indolicus | AY153431 |
| Peptoniphilus ivorii | Y07840 |
| Peptoniphilus lacrimalis | ADDO01000050 |
| Peptoniphilus sp. gpac007 | AM176517 |
| Peptoniphilus sp. gpac018A | AM176519 |
| Peptoniphilus sp. gpac077 | AM176527 |
| Peptoniphilus sp. gpac148 | AM176535 |
| Peptoniphilus sp. JC140 | JF824803 |
| Peptoniphilus sp. oral taxon 386 | ADCS01000031 |
| Peptoniphilus sp. oral taxon 836 | AEAA01000090 |
| Peptostreptococcaceae bacterium | JN837495 |
| ph1 | |
| Peptostreptococcus anaerobius | AY326462 |
| Peptostreptococcus micros | AM176538 |
| Peptostreptococcus sp. 9succ1 | X90471 |
| Peptostreptococcus sp. oral | AB175072 |
| clone AP24 | |
| Peptostreptococcus sp. oral | AY349390 |
| clone FJ023 | |
| Peptostreptococcus sp. | AY207059 |
| P4P_31 P3 | |
| Peptostreptococcus stomatis | ADGQ01000048 |
| Porphyromonadaceae bacterium | EF184292 |
| NML 060648 | |
| Porphyromonas asaccharolytica | AENO01000048 |
| Porphyromonas endodontalis | ACNN01000021 |
| Porphyromonas gingivalis | AE015924 |
| Porphyromonas levii | NR_025907 |
| Porphyromonas macacae | NR_025908 |
| Porphyromonas somerae | AB547667 |
| Porphyromonas sp. oral | AY005068 |
| clone BB134 | |
| Porphyromonas sp. oral | AY005069 |
| clone F016 | |
| Porphyromonas sp. oral | AY207054 |
| clone P2PB_52 P1 | |
| Porphyromonas sp. oral | AY207057 |
| clone P4GB_100 P2 | |
| Porphyromonas sp. UQD 301 | EU012301 |
| Porphyromonas uenonis | ACLR01000152 |
| Prevotella albensis | NR_025300 |
| Prevotella amnii | AB547670 |
| Prevotella bergensis | ACKS01000100 |
| Prevotella bivia | ADFO01000096 |
| Prevotella brevis | NR_041954 |
| Prevotella buccae | ACRB01000001 |
| Prevotella buccalis | JN867261 |
| Prevotella copri | ACBX02000014 |
| Prevotella corporis | L16465 |
| Prevotella dentalis | AB547678 |
| Prevotella denticola | CP002589 |
| Prevotella disiens | AEDO01000026 |
| Prevotella genomosp. C1 | AY278624 |
| Prevotella genomosp. C2 | AY278625 |
| Prevotella genomosp. P7 | DQ003620 |
| oral clone MB2_P31 | |
| Prevotella genomosp. P8 | DQ003622 |
| oral clone MB3_P13 | |
| Prevotella genomosp. P9 | DQ003633 |
| oral clone MB7_G16 | |
| Prevotella heparinolytica | GQ422742 |
| Prevotella histicola | JN867315 |
| Prevotella intermedia | AF414829 |
| Prevotella loescheii | JN867231 |
| Prevotella maculosa | AGEK01000035 |
| Prevotella marshii | AEEI01000070 |
| Prevotella melaninogenica | CP002122 |
| Prevotella micans | AGWK01000061 |
| Prevotella multiformis | AEWX01000054 |
| Prevotella multisaccharivorax | AFJE01000016 |
| Prevotella nanceiensis | JN867228 |
| Prevotella nigrescens | AFPX01000069 |
| Prevotella oralis | AEPE01000021 |
| Prevotella oris | ADDV01000091 |
| Prevotella oulorum | L16472 |
| Prevotella pallens | AFPY01000135 |
| Prevotella ruminicola | CP002006 |
| Prevotella salivae | AB108826 |
| Prevotella sp. BI_42 | AJ581354 |
| Prevotella sp. CM38 | HQ610181 |
| Prevotella sp. ICM1 | HQ616385 |
| Prevotella sp. ICM55 | HQ616399 |
| Prevotella sp. JCM 6330 | AB547699 |
| Prevotella sp. oral clone | AY005057 |
| AA020 | |
| Prevotella sp. oral clone | AY923148 |
| ASCG10 | |
| Prevotella sp. oral clone | DQ272511 |
| ASCG12 | |
| Prevotella sp. oral clone | AY005062 |
| AU069 | |
| Prevotella sp. oral clone | AY005063 |
| CY006 | |
| Prevotella sp. oral clone | AY005065 |
| DA058 | |
| Prevotella sp. oral clone | AY349392 |
| FL019 | |
| Prevotella sp. oral clone | AY349393 |
| FU048 | |
| Prevotella sp. oral clone | AY349394 |
| FW035 | |
| Prevotella sp. oral clone | AY349395 |
| GI030 | |
| Prevotella sp. oral clone | AY349396 |
| GI032 | |
| Prevotella sp. oral clone | AY349397 |
| GI059 | |
| Prevotella sp. oral clone | AY349398 |
| GU027 | |
| Prevotella sp. oral clone | AY349399 |
| HF050 | |
| Prevotella sp. oral clone | AY349400 |
| ID019 | |
| Prevotella sp. oral clone | AY550997 |
| IDR_CEC_0055 | |
| Prevotella sp. oral clone | AY349401 |
| IK053 | |
| Prevotella sp. oral clone | AY349402 |
| IK062 | |
| Prevotella sp. oral clone | AY207050 |
| P4PB_83 P2 | |
| Prevotella sp. oral taxon | GQ422735 |
| 292 | |
| Prevotella sp. oral taxon | ACWZ01000026 |
| 299 | |
| Prevotella sp. oral taxon | GU409549 |
| 300 | |
| Prevotella sp. oral taxon | ACZK01000043 |
| 302 | |
| Prevotella sp. oral taxon | GQ422737 |
| 310 | |
| Prevotella sp. oral taxon | ACQH01000158 |
| 317 | |
| Prevotella sp. oral taxon | ACZS01000106 |
| 472 | |
| Prevotella sp. oral taxon | GQ422744 |
| 781 | |
| Prevotella sp. oral taxon | GQ422745 |
| 782 | |
| Prevotella sp. oral taxon | HM099652 |
| F68 | |
| Prevotella sp. oral taxon | GU432133 |
| G60 | |
| Prevotella sp. oral taxon | GU432179 |
| G70 | |
| Prevotella sp. oral taxon | GU432180 |
| G71 | |
| Prevotella sp. SEQ053 | JN867222 |
| Prevotella sp. SEQ065 | JN867234 |
| Prevotella sp. SEQ072 | JN867238 |
| Prevotella sp. SEQ116 | JN867246 |
| Prevotella sp. SG12 | GU561343 |
| Prevotella sp. sp24 | AB003384 |
| Prevotella sp. sp34 | AB003385 |
| Prevotella stercorea | AB244774 |
| Prevotella tannerae | ACIJ02000018 |
| Prevotella timonensis | ADEF01000012 |
| Prevotella veroralis | ACVA01000027 |
| Prevotellaceae bacterium | AY207061 |
| P4P_62 P1 | |
| Propionibacteriaceae bacterium | EF599122 |
| NML 02_0265 | |
| Propionibacterium acidipropionici | NC_019395 |
| Propionibacterium acnes | ADJM01000010 |
| Propionibacterium avidum | AJ003055 |
| Propionibacterium freudenreichii | NR_036972 |
| Propionibacterium granulosum | FJ785716 |
| Propionibacterium jensenii | NR_042269 |
| Propionibacterium propionicum | NR_025277 |
| Propionibacterium sp. 434_HC2 | AFIL01000035 |
| Propionibacterium sp. H456 | AB177643 |
| Propionibacterium sp. LG | AY354921 |
| Propionibacterium sp. oral | GQ422728 |
| taxon 192 | |
| Propionibacterium sp. S555a | AB264622 |
| Propionibacterium thoenii | NR_042270 |
| Pseudomonas aeruginosa | AABQ07000001 |
| Pseudomonas fluorescens | AY622220 |
| Pseudomonas gessardii | FJ943496 |
| Pseudomonas mendocina | AAUL01000021 |
| Pseudomonas monteilii | NR_024910 |
| Pseudomonas poae | GU188951 |
| Pseudomonas pseudoalcaligenes | NR_037000 |
| Pseudomonas putida | AF094741 |
| Pseudomonas sp. 2_1_26 | ACWU01000257 |
| Pseudomonas sp. G1229 | DQ910482 |
| Pseudomonas sp. NP522b | EU723211 |
| Pseudomonas stutzeri | AM905854 |
| Pseudomonas tolaasii | AF320988 |
| Pseudomonas viridiflava | NR_042764 |
| Ralstonia pickettii | NC_010682 |
| Ralstonia sp. 5_7_47FAA | ACUF01000076 |
| Roseburia cecicola | GU233441 |
| Roseburia faecalis | AY804149 |
| Roseburia faecis | AY305310 |
| Roseburia hominis | AJ270482 |
| Roseburia intestinalis | FP929050 |
| Roseburia inulinivorans | AJ270473 |
| Roseburia sp. 11SE37 | FM954975 |
| Roseburia sp. 11SE38 | FM954976 |
| Rothia aeria | DQ673320 |
| Rothia dentocariosa | ADDW01000024 |
| Rothia mucilaginosa | ACVO01000020 |
| Rothia nasimurium | NR_025310 |
| Rothia sp. oral taxon 188 | GU470892 |
| Ruminobacter amylophilus | NR_026450 |
| Ruminococcaceae bacterium D16 | ADDX01000083 |
| Ruminococcus albus | AY445600 |
| Ruminococcus bromii | EU266549 |
| Ruminococcus callidus | NR_029160 |
| Ruminococcus champanellensis | FP929052 |
| Ruminococcus flavefaciens | NR_025931 |
| Ruminococcus gnavus | X94967 |
| Ruminococcus hansenii | M59114 |
| Ruminococcus lactaris | ABOU02000049 |
| Ruminococcus obeum | AY169419 |
| Ruminococcus sp. 18P13 | AJ515913 |
| Ruminococcus sp. 5_1_39BFAA | ACII01000172 |
| Ruminococcus sp. 9SE51 | FM954974 |
| Ruminococcus sp. ID8 | AY960564 |
| Ruminococcus sp. K_1 | AB222208 |
| Ruminococcus torques | AAVP02000002 |
| Salmonella bongori | NR_041699 |
| Salmonella enterica | NC_011149 |
| Salmonella enterica | NC_011205 |
| Salmonella enterica | DQ344532 |
| Salmonella enterica | ABEH02000004 |
| Salmonella enterica | ABAK02000001 |
| Salmonella enterica | NC_011080 |
| Salmonella enterica | EU118094 |
| Salmonella enterica | NC_011094 |
| Salmonella enterica | AE014613 |
| Salmonella enterica | ABFH02000001 |
| Salmonella enterica | ABEM01000001 |
| Salmonella enterica | ABAM02000001 |
| Salmonella typhimurium | DQ344533 |
| Salmonella typhimurium | AF170176 |
| Selenomonas artemidis | HM596274 |
| Selenomonas dianae | GQ422719 |
| Selenomonas flueggei | AF287803 |
| Selenomonas genomosp. C1 | AY278627 |
| Selenomonas genomosp. C2 | AY278628 |
| Selenomonas genomosp. P5 | AY341820 |
| Selenomonas genomosp. P6 | DQ003636 |
| oral clone MB3_C41 | |
| Selenomonas genomosp. P7 | DQ003627 |
| oral clone MB5_C08 | |
| Selenomonas genomosp. P8 | DQ003628 |
| oral clone MB5_P06 | |
| Selenomonas infelix | AF287802 |
| Selenomonas noxia | GU470909 |
| Selenomonas ruminantium | NR_075026 |
| Selenomonas sp. FOBRC9 | HQ616378 |
| Selenomonas sp. oral clone | AY349403 |
| FT050 | |
| Selenomonas sp. oral clone | AY349404 |
| GI064 | |
| Selenomonas sp. oral clone | AY349405 |
| GT010 | |
| Selenomonas sp. oral clone | AY349406 |
| HU051 | |
| Selenomonas sp. oral clone | AY349407 |
| IK004 | |
| Selenomonas sp. oral clone | AY349408 |
| IQ048 | |
| Selenomonas sp. oral clone | AY349409 |
| JI021 | |
| Selenomonas sp. oral clone | AY349410 |
| JS031 | |
| Selenomonas sp. oral clone | AY947498 |
| OH4A | |
| Selenomonas sp. oral clone | AY207052 |
| P2PA_80 P4 | |
| Selenomonas sp. oral taxon | AENV01000007 |
| 137 | |
| Selenomonas sp. oral taxon | AEEJ01000007 |
| 149 | |
| Selenomonas sputigena | ACKP02000033 |
| Serratia fonticola | NR_025339 |
| Serratia liquefaciens | NR_042062 |
| Serratia marcescens | GU826157 |
| Serratia odorifera | ADBY01000001 |
| Serratia proteamaculans | AAUN01000015 |
| Shigella boydii | AAKA01000007 |
| Shigella dysenteriae | NC_007606 |
| Shigella flexneri | AE005674 |
| Shigella sonnei | NC_007384 |
| Sphingobacterium faecium | NR_025537 |
| Sphingobacterium mizutaii | JF708889 |
| Sphingobacterium multivorum | NR_040953 |
| Sphingobacterium spiritivorum | ACHA02000013 |
| Sphingomonas echinoides | NR_024700 |
| Sphingomonas sp. oral | AY349411 |
| clone FI012 | |
| Sphingomonas sp. oral | AY349412 |
| clone FZ016 | |
| Sphingomonas sp. oral | HM099639 |
| taxon A09 | |
| Sphingomonas sp. oral | HM099645 |
| taxon F71 | |
| Staphylococcaceae bacterium | AY841362 |
| NML 92_0017 | |
| Staphylococcus aureus | CP002643 |
| Staphylococcus auricularis | JQ624774 |
| Staphylococcus capitis | ACFR01000029 |
| Staphylococcus caprae | ACRH01000033 |
| Staphylococcus carnosus | NR_075003 |
| Staphylococcus cohnii | JN175375 |
| Staphylococcus condimenti | NR_029345 |
| Staphylococcus epidermidis | ACHE01000056 |
| Staphylococcus equorum | NR_027520 |
| Staphylococcus fleurettii | NR_041326 |
| Staphylococcus haemolyticus | NC_007168 |
| Staphylococcus hominis | AM157418 |
| Staphylococcus lugdunensis | AEQA01000024 |
| Staphylococcus pasteuri | FJ189773 |
| Staphylococcus pseudintermedius | CP002439 |
| Staphylococcus saccharolyticus | NR_029158 |
| Staphylococcus saprophyticus | NC_007350 |
| Staphylococcus sciuri | NR_025520 |
| Staphylococcus sp. clone | AF467424 |
| bottae7 | |
| Staphylococcus sp. H292 | AB177642 |
| Staphylococcus sp. H780 | AB177644 |
| Staphylococcus succinus | NR_028667 |
| Staphylococcus vitulinus | NR_024670 |
| Staphylococcus warneri | ACPZ01000009 |
| Staphylococcus xylosus | AY395016 |
| Streptobacillus moniliformis | NR_027615 |
| Streptococcus agalactiae | AAJO01000130 |
| Streptococcus alactolyticus | NR_041781 |
| Streptococcus anginosus | AECT01000011 |
| Streptococcus australis | AEQR01000024 |
| Streptococcus bovis | AEEL01000030 |
| Streptococcus canis | AJ413203 |
| Streptococcus constellatus | AY277942 |
| Streptococcus cristatus | AEVC01000028 |
| Streptococcus downei | AEKN01000002 |
| Streptococcus dysgalactiae | AP010935 |
| Streptococcus equi | CP001129 |
| Streptococcus equinus | AEVB01000043 |
| Streptococcus gallolyticus | FR824043 |
| Streptococcus genomosp. C1 | AY278629 |
| Streptococcus genomosp. C2 | AY278630 |
| Streptococcus genomosp. C3 | AY278631 |
| Streptococcus genomosp. C4 | AY278632 |
| Streptococcus genomosp. C5 | AY278633 |
| Streptococcus genomosp. C6 | AY278634 |
| Streptococcus genomosp. C7 | AY278635 |
| Streptococcus genomosp. C8 | AY278609 |
| Streptococcus gordonii | NC_009785 |
| Streptococcus infantarius | ABJK02000017 |
| Streptococcus infantis | AFNN01000024 |
| Streptococcus intermedius | NR_028736 |
| Streptococcus lutetiensis | NR_037096 |
| Streptococcus massiliensis | AY769997 |
| Streptococcus milleri | X81023 |
| Streptococcus mitis | AM157420 |
| Streptococcus mutans | AP010655 |
| Streptococcus oligofermentans | AY099095 |
| Streptococcus oralis | ADMV01000001 |
| Streptococcus parasanguinis | AEKM01000012 |
| Streptococcus pasteurianus | AP012054 |
| Streptococcus peroris | AEVF01000016 |
| Streptococcus pneumoniae | AE008537 |
| Streptococcus porcinus | EF121439 |
| Streptococcus pseudopneumoniae | FJ827123 |
| Streptococcus pseudoporcinus | AENS01000003 |
| Streptococcus pyogenes | AE006496 |
| Streptococcus ratti | X58304 |
| Streptococcus salivarius | AGBV01000001 |
| Streptococcus sanguinis | NR_074974 |
| Streptococcus sinensis | AF432857 |
| Streptococcus sp. 16362 | JN590019 |
| Streptococcus sp. 2_1_36FAA | ACOI01000028 |
| Streptococcus sp. 2285_97 | AJ131965 |
| Streptococcus sp. 69130 | X78825 |
| Streptococcus sp. AC15 | HQ616356 |
| Streptococcus sp. ACS2 | HQ616360 |
| Streptococcus sp. AS20 | HQ616366 |
| Streptococcus sp. BS35a | HQ616369 |
| Streptococcus sp. C150 | ACRI01000045 |
| Streptococcus sp. CM6 | HQ616372 |
| Streptococcus sp. CM7 | HQ616373 |
| Streptococcus sp. ICM10 | HQ616389 |
| Streptococcus sp. ICM12 | HQ616390 |
| Streptococcus sp. ICM2 | HQ616386 |
| Streptococcus sp. ICM4 | HQ616387 |
| Streptococcus sp. ICM45 | HQ616394 |
| Streptococcus sp. M143 | ACRK01000025 |
| Streptococcus sp. M334 | ACRL01000052 |
| Streptococcus sp. OBRC6 | HQ616352 |
| Streptococcus sp. oral clone | AY923121 |
| ASB02 | |
| Streptococcus sp. oral clone | DQ272504 |
| ASCA03 | |
| Streptococcus sp. oral clone | AY923116 |
| ASCA04 | |
| Streptococcus sp. oral clone | AY923119 |
| ASCA09 | |
| Streptococcus sp. oral clone | AY923123 |
| ASCB04 | |
| Streptococcus sp. oral clone | AY923124 |
| ASCB06 | |
| Streptococcus sp. oral clone | AY923127 |
| ASCC04 | |
| Streptococcus sp. oral clone | AY923128 |
| ASCC05 | |
| Streptococcus sp. oral clone | DQ272507 |
| ASCC12 | |
| Streptococcus sp. oral clone | AY923129 |
| ASCD01 | |
| Streptococcus sp. oral clone | AY923130 |
| ASCD09 | |
| Streptococcus sp. oral clone | DQ272509 |
| ASCD10 | |
| Streptococcus sp. oral clone | AY923134 |
| ASCE03 | |
| Streptococcus sp. oral clone | AY953253 |
| ASCE04 | |
| Streptococcus sp. oral clone | DQ272510 |
| ASCE05 | |
| Streptococcus sp. oral clone | AY923135 |
| ASCE06 | |
| Streptococcus sp. oral clone | AY923136 |
| ASCE09 | |
| Streptococcus sp. oral clone | AY923137 |
| ASCE10 | |
| Streptococcus sp. oral clone | AY923138 |
| ASCE12 | |
| Streptococcus sp. oral clone | AY923140 |
| ASCF05 | |
| Streptococcus sp. oral clone | AY953255 |
| ASCF07 | |
| Streptococcus sp. oral clone | AY923142 |
| ASCF09 | |
| Streptococcus sp. oral clone | AY923145 |
| ASCG04 | |
| Streptococcus sp. oral clone | AY005042 |
| BW009 | |
| Streptococcus sp. oral clone | AY005044 |
| CH016 | |
| Streptococcus sp. oral clone | AY349413 |
| GK051 | |
| Streptococcus sp. oral clone | AY349414 |
| GM006 | |
| Streptococcus sp. oral clone | AY207051 |
| P2PA_41 P2 | |
| Streptococcus sp. oral clone | AY207064 |
| P4PA_30 P4 | |
| Streptococcus sp. oral taxon | AEEP01000019 |
| 071 | |
| Streptococcus sp. oral taxon | GU432132 |
| G59 | |
| Streptococcus sp. oral taxon | GU432146 |
| G62 | |
| Streptococcus sp. oral taxon | GU432150 |
| G63 | |
| Streptococcus sp. SHV515 | Y07601 |
| Streptococcus suis | FM252032 |
| Streptococcus thermophilus | CP000419 |
| Streptococcus uberis | HQ391900 |
| Streptococcus urinalis | DQ303194 |
| Streptococcus vestibularis | AEKO01000008 |
| Streptococcus viridans | AF076036 |
| Sutterella morbirenis | AJ832129 |
| Sutterella parvirubra | AB300989 |
| Sutterella sanguinus | AJ748647 |
| Sutterella sp. YIT 12072 | AB491210 |
| Sutterella stercoricanis | NR_025600 |
| Sutterella wadsworthensis | ADMF01000048 |
| Synergistes genomosp. C1 | AY278615 |
| Synergistes sp. RMA 14551 | DQ412722 |
| Synergistetes bacterium | GQ258968 |
| ADV897 | |
| Synergistetes bacterium | GQ258969 |
| LBVCM1157 | |
| Synergistetes bacterium | GU410752 |
| oral taxon 362 | |
| Synergistetes bacterium | GU430992 |
| oral taxon D48 | |
| Turicibacter sanguinis | AF349724 |
| Veillonella atypica | AEDS01000059 |
| Veillonella dispar | ACIK02000021 |
| Veillonella genomosp. P1 | DQ003631 |
| oral clone MB5_P17 | |
| Veillonella montpellierensis | AF473836 |
| Veillonella parvula | ADFU01000009 |
| Veillonella sp. 3_1_44 | ADCV01000019 |
| Veillonella sp. 6_1_27 | ADCW01000016 |
| Veillonella sp. ACP1 | HQ616359 |
| Veillonella sp. AS16 | HQ616365 |
| Veillonella sp. BS32b | HQ616368 |
| Veillonella sp. ICM51a | HQ616396 |
| Veillonella sp. MSA12 | HQ616381 |
| Veillonella sp. NVG 100cf | EF108443 |
| Veillonella sp. OK11 | JN695650 |
| Veillonella sp. oral clone | AY923118 |
| ASCA08 | |
| Veillonella sp. oral clone | AY923122 |
| ASCB03 | |
| Veillonella sp. oral clone | AY923144 |
| ASCG01 | |
| Veillonella sp. oral clone | AY953257 |
| ASCG02 | |
| Veillonella sp. oral clone | AY947495 |
| OH1A | |
| Veillonella sp. oral taxon | AENU01000007 |
| 158 | |
| Veillonellaceae bacterium | GU402916 |
| oral taxon 131 | |
| Veillonellaceae bacterium | GU470897 |
| oral taxon 155 | |
| Vibrio cholerae | AAUR01000095 |
| Vibrio fluvialis | X76335 |
| Vibrio furnissii | CP002377 |
| Vibrio mimicus | ADAF01000001 |
| Vibrio parahaemolyticus | AAWQ01000116 |
| Vibrio sp. RC341 | ACZT01000024 |
| Vibrio vulnificus | AE016796 |
| Yersinia aldovae | AJ871363 |
| Yersinia aleksiciae | AJ627597 |
| Yersinia bercovieri | AF366377 |
| Yersinia enterocolitica | FR729477 |
| Yersinia frederiksenii | AF366379 |
| Yersinia intermedia | AF366380 |
| Yersinia kristensenii | ACCA01000078 |
| Yersinia mollaretii | NR_027546 |
| Yersinia pestis | AE013632 |
| Yersinia pseudotuberculosis | NC_009708 |
| Yersinia rohdei | ACCD01000071 |

TABLE 3
Exemplary Bacterial Strains

| Strain | Deposit Number |
|---|---|
| Parabacteroides goldsteinii | PTA-126574 |
| Bifidobacterium animalis ssp. | PTA-125097 |
| lactis Strain A | |
| Blautia Massiliensis Strain A | PTA-125134 |
| Prevotella Strain B | NRRL accession Number B 50329 |
| Prevotella Histicola | PTA-126140 |
| Blautia Strain A | PTA-125346 |
| Lactococcus lactis cremoris Strain A | PTA-125368 |
| Lactobacillus salivarius | PTA-125893 |
| Ruminococcus gnavus strain | PTA-125706 |
| Tyzzerella nexilis strain | PTA-125707 |
| Paraclostridium benzoelyticum | PTA-125894 |
| Ruminococcus gnavus (also referred | PTA-126695 |
| to as Mediterraneibacter gnavus) | |
| Veillonella parvula | PTA-125710 |
| Veillonella atypica Strain A | PTA-125709 |
| Veillonella atypica Strain B | PTA-125711 |
| Veillonella parvula Strain A | PTA-125691 |
| Veillonella tobetsuensis Strain A | PTA-125708 |
| Agathobaculum sp. | PTA-125892 |
| Turicibacter sanguinis | PTA-125889 |
| Klebsiella quasipneumoniae subsp. | PTA-125891 |
| similipneumoniae | |
| Klebsiella oxytoca | PTA-125890 |
| Megasphaera Sp. Strain A | PTA-126770 |
| Megasphaera Sp. | PTA-126837 |
| Harryflintia acetispora | PTA-126694 |
| Fournierella massiliensis | PTA-126696 |

TABLE 4
Exemplary Bacterial Strains

| | |
|---|---|
| Escherichia coli | NCIMB 12210 |
| Enterococcus faecalis | NCIMB 13280 |
| Bacteroides fragilis | DSM 2151 |
| Bacteroides vulgatus | DSM 1447 |
| Bacteroides ovatus | DSM 1896 |
| Megasphaera massiliensis | DSM 26228 |
| Megasphaera elsdenii | NCIMB 8927 |
| Megasphaera massiliensis | NCIMB 42787 |
| Bifidobacterium breve | DSM 20213 |
| Bifidobacterium longum subsp. longum | DSM 20219 |
| Faecalibacterium prausnitzii | DSM 17677 |
| Anaerostipes hadrus | DSM 3319 |
| Blautia coccoides | DSM 935 |
| Dorea longicatena | DSM 13814 |
| Parabacteroides distasonis | DSM 20701 |
| Faecalicatena contorta | DSM 3982 |
| Ruminococcus gnavus | ATCC 29149 |
| Megasphaera massiliensis | NCIMB 43388 |
| Megasphaera massiliensis | NCIMB 43389 |
| Megasphaera spp. | NCIMB 43385 |
| Megasphaera spp. | NCIMB 43386 |
| Megasphaera spp. | NCIMB 43387 |
| Parabacteroides distasonis (also referred | NCIMB 42382 |
| to as “Parabacteroides sp 755”) | |

### Modified EVs

In some aspects, the EVs described herein are modified such that they comprise, are linked to, and/or are bound by a therapeutic moiety.

In some embodiments, the therapeutic moiety is a cancer-specific moiety. In some embodiments, the cancer-specific moiety has binding specificity for a cancer cell (for example, has binding specificity for a cancer-specific antigen). In some embodiments, the cancer-specific moiety comprises an antibody or antigen binding fragment thereof. In some embodiments, the cancer-specific moiety comprises a T cell receptor or a chimeric antigen receptor (CAR). In some embodiments, the cancer-specific moiety comprises a ligand for a receptor expressed on the surface of a cancer cell or a receptor-binding fragment thereof. In some embodiments, the cancer-specific moiety is a bipartite fusion protein that has two parts: a first part that binds to and/or is linked to the bacterium and a second part that is capable of binding to a cancer cell (for example, by having binding specificity for a cancer-specific antigen). In some embodiments, the first part is a fragment of or a full-length peptidoglycan recognition protein, such as PGRP. In some embodiments the first part has binding specificity for the EV (for example, by having binding specificity for a bacterial antigen). In some embodiments, the first and/or second part comprises an antibody or antigen binding fragment thereof. In some embodiments, the first and/or second part comprises a T cell receptor or a chimeric antigen receptor (CAR). In some embodiments, the first and/or second part comprises a ligand for a receptor expressed on the surface of a cancer cell or a receptor-binding fragment thereof. In certain embodiments, co-administration of the cancer-specific moiety with the EVs (either in combination or in separate administrations) increases the targeting of the EVs to the cancer cells.

In some embodiments, the EVs described herein are engineered such that they comprise, are linked to, and/or are bound by a magnetic and/or paramagnetic moiety (for example, a magnetic bead). In some embodiments, the magnetic and/or paramagnetic moiety is comprised by and/or directly linked to the bacteria. In some embodiments, the magnetic and/or paramagnetic moiety is linked to and/or a part of an EV-binding moiety that that binds to the EV. In some embodiments, the EV-binding moiety is a fragment of or a full-length peptidoglycan recognition protein, such as PGRP. In some embodiments the EV-binding moiety has binding specificity for the EV (for example, by having binding specificity for a bacterial antigen). In some embodiments, the EV-binding moiety comprises an antibody or antigen binding fragment thereof. In some embodiments, the EV-binding moiety comprises a T cell receptor or a chimeric antigen receptor (CAR). In some embodiments, the EV-binding moiety comprises a ligand for a receptor expressed on the surface of a cancer cell or a receptor-binding fragment thereof. In certain embodiments, co-administration of the magnetic and/or paramagnetic moiety with the EVs (either together or in separate administrations) can be used to increase the targeting of the EVs (for example, to cancer cells and/or a part of a subject where cancer cells are present.

### Production of Bacterial Extracellular Vesicles (EVs)

Secreted EVs. In certain aspects, the EVs (such as secreted EVs (smEVs) from bacteria described herein are prepared using any method known in the art.

In some embodiments, the EVs (such as secreted EVs (smEVs) are prepared without an EV purification step. For example, in some embodiments, bacteria described herein are killed using a method that leaves the EVs intact and the resulting bacterial components, including the EVs, are used in the methods and compositions described herein. In some embodiments, the bacteria are killed using an antibiotic (for example, using an antibiotic described herein). In some embodiments, the bacteria are killed using UV irradiation. In some embodiments, the bacteria are heat-killed.

In some embodiments, the EVs described herein are purified from one or more other bacterial components. Methods for purifying EVs from bacteria are known in the art. In some embodiments, EVs are prepared from bacterial cultures using methods described in S. Bin Park, et al. PLoS ONE. 6(3):e17629 (2011) or G. Norheim, et al. PLoS ONE. 10(9): e0134353 (2015) or Jeppesen, et al. Cell 177:428 (2019), each of which is hereby incorporated by reference in its entirety. In some embodiments, the bacteria are cultured to high optical density and then centrifuged to pellet bacteria (for example, at 10,000×g for 30 min at 4° C., at 15,500×g for 15 min at 4° C.). In some embodiments, the culture supernatants are then passed through filters to exclude intact bacterial cells (for example, a 0.22 μm filter). In some embodiments, the supernatants are then subjected to tangential flow filtration, during which the supernatant is concentrated, species smaller than 100 kDa are removed, and the media is partially exchanged with PBS. In some embodiments, filtered supernatants are centrifuged to pellet bacterial EVs (for example, at 100,000-150,000×g for 1-3 hours at 4° C., at 200,000×g for 1-3 hours at 4° C.). In some embodiments, the EVs are further purified by resuspending the resulting EV pellets (for example, in PBS), and applying the resuspended EVs to an Optiprep (iodixanol) gradient or gradient (for example, a 30-60% discontinuous gradient, a 0-45% discontinuous gradient), followed by centrifugation (for example, at 200,000×g for 4-20 hours at 4° C.). EV bands can be collected, diluted with PBS, and centrifuged to pellet the EVs (for example, at 150,000×g for 3 hours at 4° C., at 200,000×g for 1 hour at 4° C.). The purified EVs can be stored, for example, at −80° C., or −20° C. until use. In some embodiments, the EVs are further purified by treatment with DNase and/or proteinase K.

For example, in some embodiments, cultures of bacteria can be centrifuged at 11,000×g for 20-40 min at 4° C. to pellet bacteria. Culture supernatants may be passed through a 0.22 μm filter to exclude intact bacterial cells. Filtered supernatants may then be concentrated using methods that may include, but are not limited to, ammonium sulfate precipitation, ultracentrifugation, or filtration. For example, for ammonium sulfate precipitation, 1.5-3 M ammonium sulfate can be added to filtered supernatant slowly, while stirring at 4° C. Precipitations can be incubated at 4° C. for 8-48 hours and then centrifuged at 11,000×g for 20-40 min at 4° C. The resulting pellets contain bacteria EVs and other debris. Using ultracentrifugation, filtered supernatants can be centrifuged at 100,000-200,000×g for 1-16 hours at 4° C. The pellet of this centrifugation contains bacterial EVs and other debris such as large protein complexes. In some embodiments, using a filtration technique, such as through the use of an Amicon Ultra spin filter or by tangential flow filtration, supernatants can be filtered so as to retain species of molecular weight >50 or 100 kDa.

Alternatively, EVs can be obtained from bacteria cultures continuously during growth, or at selected time points during growth, for example, by connecting a bioreactor to an alternating tangential flow (ATF) system (for example, XCell ATF from Repligen). The ATF system retains intact cells (>0.22 μm) in the bioreactor, and allows smaller components (for example, EVs, free proteins) to pass through a filter for collection. For example, the system may be configured so that the <0.22 μm filtrate is then passed through a second filter of 100 kDa, allowing species such as EVs between 0.22 μm and 100 kDa to be collected, and species smaller than 100 kDa to be pumped back into the bioreactor. Alternatively, the system may be configured to allow for medium in the bioreactor to be replenished and/or modified during growth of the culture. EVs collected by this method may be further purified and/or concentrated by ultracentrifugation or filtration as described above for filtered supernatants.

EVs obtained by methods provided herein may be further purified by size-based column chromatography, by affinity chromatography, by ion-exchange chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in PBS and 3 volumes of 60% Optiprep are added to the sample. In some embodiments, if filtration was used to concentrate the filtered supernatant, the concentrate is diluted using 60% Optiprep to a final concentration of 35% Optiprep. Samples are applied to a 0-45% discontinuous Optiprep gradient and centrifuged at 200,000×g for 3-24 hours at 4° C., for example, 4-24 hours at 4° C.

In some embodiments, to confirm sterility and isolation of the EV preparations, EVs are serially diluted onto agar medium used for routine culture of the bacteria being tested, and incubated using routine conditions. Non-sterile preparations are passed through a 0.22 um filter to exclude intact cells. To further increase purity, isolated EVs may be DNase or proteinase K treated.

In some embodiments, for preparation of EVs used for in vivo injections, purified EVs are processed as described previously (G. Norheim, et al. PLoS ONE. 10(9): e0134353 (2015)). Briefly, after sucrose gradient centrifugation, bands containing EVs are resuspended to a final concentration of 50 μg/mL in a solution containing 3% sucrose or other solution suitable for in vivo injection known to one skilled in the art. This solution may also contain adjuvant, for example aluminum hydroxide at a concentration of 0-0.5% (w/v). In some embodiments, for preparation of EVs used for in vivo injections, EVs in PBS are sterile-filtered to <0.22 μm.

In certain embodiments, to make samples compatible with further testing (for example, to remove sucrose prior to TEM imaging or in vitro assays), samples are buffer exchanged into PBS or 30 mM Tris, pH 8.0 using filtration (for example, Amicon Ultra columns), dialysis, or ultracentrifugation (200,000×g, ≥3 hours, 4° C.) and resuspension.

In some embodiments, the sterility of the EV preparations can be confirmed by plating a portion of the EVs onto agar medium used for standard culture of the bacteria used in the generation of the EVs and incubating using standard conditions.

In some embodiments, select EVs are isolated and enriched by chromatography and binding surface moieties on EVs. In some embodiments, select EVs are isolated and/or enriched by fluorescent cell sorting by methods using affinity reagents, chemical dyes, recombinant proteins or other methods known to one skilled in the art.

In some embodiments, EVs are analyzed, for example, as described in Jeppesen, et al. Cell 177:428 (2019).

In some embodiments, EVs are lyophilized.

In some embodiments, EVs are gamma irradiated (for example, at 17.5 or 25 kGy).

In some embodiments, EVs are UV irradiated.

In some embodiments, EVs are heat inactivated (for example, at 50° C. for two hours or at 90° C. for two hours).

In some embodiments, EVs are acid treated.

In some embodiments, EVs are oxygen sparged (for example, at 0.1 vvm for two hours).

The phase of growth can affect the amount or properties of bacteria and/or EVs produced by bacteria. For example, in the methods of EV preparation provided herein, EVs can be isolated, for example, from a culture, at the start of the log phase of growth, midway through the log phase, and/or once stationary phase growth has been reached.

The growth environment (for example, culture conditions) can affect the amount of EVs produced by bacteria. For example, the yield of EVs can be increased by an EV inducer, as provided in Table 5.

TABLE 5
Culture Techniques to Increase EV Production

| EV inducement | EV inducer | Acts on |
|---|---|---|
| Temperature | Heat | stress response |
| | RT to 37° C. temp change | simulates infection |
| | 37 to 40° C. temp change | febrile infection |
| ROS | Plumbagin | oxidative stress response |
| | Cumene hydroperoxide | oxidative stress response |
| | Hydrogen Peroxide | oxidative stress response |
| Antibiotics | Ciprofloxacin | bacterial SOS response |
| | Gentamycin | protein synthesis |
| | Polymyxin B | outer membrane |
| | D-cylcloserine | cell wall |
| Osmolyte | NaCl | osmotic stress |
| Metal Ion | Iron Chelation | iron levels |
| Stress | EDTA | removes divalent cations |
| | Low Hemin | iron levels |
| Media | Lactate | growth |
| additives | Amino acid deprivation | stress |
| or removal | Hexadecane | stress |
| | Glucose | growth |
| | Sodium bicarbonate | ToxT induction |
| | PQS | vesiculator (from bacteria) |
| | Diamines + DFMO | membrane anchoring |
| | High nutrients | (negativicutes only) |
| | Low nutrients | enhanced growth |
| Other | Oxygen | oxygen stress in anaerobe |
| mechanisms | No Cysteine | oxygen stress in anaerobe |
| | Inducing biofilm or | |
| | floculation | |
| | Diauxic Growth | |
| | Phage | |
| | Urea | |

In the methods of EVs preparation provided herein, the method can optionally include exposing a culture of bacteria to an EV inducer prior to isolating EVs from the bacterial culture. The culture of bacteria can be exposed to an EV inducer at the start of the log phase of growth, midway through the log phase, and/or once stationary phase growth has been reached.

Processed EVs. In certain aspects, the EVs (such as processed EVs (pmEVs) described herein are prepared (for example, artificially prepared) using any method known in the art.

In some embodiments, the pmEVs are prepared without a pmEV purification step. For example, in some embodiments, bacteria from which the pmEVs described herein are released are killed using a method that leaves the bacterial pmEVs intact, and the resulting bacterial components, including the pmEVs, are used in the methods and compositions described herein. In some embodiments, the bacteria are killed using an antibiotic (for example, using an antibiotic described herein). In some embodiments, the bacteria are killed using UV irradiation.

In some embodiments, the pmEVs described herein are purified from one or more other bacterial components. Methods for purifying pmEVs from bacteria (and optionally, other bacterial components) are known in the art. In some embodiments, pmEVs are prepared from bacterial cultures using methods described in Thein, et al. (J. Proteome Res. 9(12):6135-6147 (2010)) or Sandrini, et al. (Bio-protocol 4(21): e1287 (2014)), each of which is hereby incorporated by reference in its entirety. In some embodiments, the bacteria are cultured to high optical density and then centrifuged to pellet bacteria (for example, at 10,000-15,000×g for 10-15 min at room temperature or 4° C.). In some embodiments, the supernatants are discarded and cell pellets are frozen at −80° C. In some embodiments, cell pellets are thawed on ice and resuspended in 100 mM Tris-HCl, pH 7.5 supplemented with 1 mg/mL DNase I. In some embodiments, cells are lysed using an Emulsiflex C-3 (Avestin, Inc.) under conditions recommended by the manufacturer. In some embodiments, debris and unlysed cells are pelleted by centrifugation at 10,000×g for 15 min at 4° C. In some embodiments, supernatants are then centrifuged at 120,000×g for 1 hour at 4° C. In some embodiments, pellets are resuspended in ice-cold 100 mM sodium carbonate, pH 11, incubated with agitation for 1 hour at 4° C., and then centrifuged at 120,000×g for 1 hour at 4° C. In some embodiments, pellets are resuspended in 100 mM Tris-HCl, pH 7.5, re-centrifuged at 120,000×g for 20 min at 4° C., and then resuspended in 0.1 M Tris-HCl, pH 7.5 or in PBS. In some embodiments, samples are stored at −20° C.

In certain aspects, pmEVs are obtained by methods adapted from Sandrini et al, 2014. In some embodiments, bacterial cultures are centrifuged at 10,000-15,500×g for 10-15 min at room temp or at 4° C. In some embodiments, cell pellets are frozen at −80° C. and supernatants are discarded. In some embodiments, cell pellets are thawed on ice and resuspended in 10 mM Tris-HCl, pH 8.0, 1 mM EDTA supplemented with 0.1 mg/mL lysozyme. In some embodiments, samples are incubated with mixing at room temp or at 37° C. for 30 min. In some embodiments, samples are re-frozen at −80° C. and thawed again on ice. In some embodiments, DNase I is added to a final concentration of 1.6 mg/mL and MgCl₂ to a final concentration of 100 mM. In some embodiments, samples are sonicated using a QSonica Q500 sonicator with 7 cycles of 30 sec on and 30 sec off. In some embodiments, debris and unlysed cells are pelleted by centrifugation at 10,000×g for 15 min. at 4° C. In some embodiments, supernatants are then centrifuged at 110,000×g for 15 min at 4° C. In some embodiments, pellets are resuspended in 10 mM Tris-HCl, pH 8.0, 2% Triton X-100 and incubated 30-60 min with mixing at room temperature. In some embodiments, samples are centrifuged at 110,000×g for 15 min at 4° C. In some embodiments, pellets are resuspended in PBS and stored at −20° C.

In certain aspects, a method of forming (for example, preparing) isolated bacterial pmEVs, described herein, comprises the steps of: (a) centrifuging a bacterial culture, thereby forming a first pellet and a first supernatant, wherein the first pellet comprises cells; (b) discarding the first supernatant; (c) resuspending the first pellet in a solution; (d) lysing the cells; (e) centrifuging the lysed cells, thereby forming a second pellet and a second supernatant; (f) discarding the second pellet and centrifuging the second supernatant, thereby forming a third pellet and a third supernatant; (g) discarding the third supernatant and resuspending the third pellet in a second solution, thereby forming the isolated bacterial pmEVs.

In some embodiments, the method further comprises the steps of: (h) centrifuging the solution of step (g), thereby forming a fourth pellet and a fourth supernatant; (i) discarding the fourth supernatant and resuspending the fourth pellet in a third solution. In some embodiments, the method further comprises the steps of: (j) centrifuging the solution of step (i), thereby forming a fifth pellet and a fifth supernatant; and (k) discarding the fifth supernatant and resuspending the fifth pellet in a fourth solution.

In some embodiments, the centrifugation of step (a) is at 10,000×g. In some embodiments the centrifugation of step (a) is for 10-15 minutes. In some embodiments, the centrifugation of step (a) is at 4° C., or room temperature. In some embodiments, step (b) further comprises freezing the first pellet at −80° C. In some embodiments, the solution in step (c) is 100 mM Tris-HCl, pH 7.5 supplemented with 1 mg/ml DNaseI. In some embodiments, the solution in step (c) is 10 mM Tris-HCl, pH 8.0, 1 mM EDTA, supplemented with 0.1 mg/ml lysozyme. In some embodiments, step (c) further comprises incubating for 30 minutes at 37° C., or room temperature. In some embodiments, step (c) further comprises freezing the first pellet at −80° C. In some embodiments, step (c) further comprises adding DNase I to a final concentration of 1.6 mg/ml. In some embodiments, step (c) further comprises adding MgCl₂ to a final concentration of 100 mM. In some embodiments, the cells are lysed in step (d) via homogenization. In some embodiments, the cells are lysed in step (d) via emulsiflex C3. In some embodiments, the cells are lysed in step (d) via sonication. In some embodiments, the cells are sonicated in 7 cycles, wherein each cycle comprises 30 seconds of sonication and 30 seconds without sonication. In some embodiments, the centrifugation of step (e) is at 10,000×g. In some embodiments, the centrifugation of step (e) is for 15 minutes. In some embodiments, the centrifugation of step (e) is at 4° C., or room temperature.

In some embodiments, the centrifugation of step (f) is at 120,000×g. In some embodiments, the centrifugation of step (f) is at 110,000×g. In some embodiments, the centrifugation of step (f) is for 1 hour. In some embodiments, the centrifugation of step (f) is for 15 minutes. In some embodiments, the centrifugation of step (f) is at 4° C., or room temperature. In some embodiments, the second solution in step (g) is 100 mM sodium carbonate, pH 11. In some embodiments, the second solution in step (g) is 10 mM Tris-HCl pH 8.0, 2% triton X-100. In some embodiments, step (g) further comprises incubating the solution for 1 hour at 4° C. In some embodiments, step (g) further comprises incubating the solution for 30-60 minutes at room temperature. In some embodiments, the centrifugation of step (h) is at 120,000×g. In some embodiments, the centrifugation of step (h) is at 110,000×g. In some embodiments, the centrifugation of step (h) is for 1 hour. In some embodiments, the centrifugation of step (h) is for 15 minutes. In some embodiments, the centrifugation of step (h) is at 4° C., or room temperature. In some embodiments, the third solution in step (i) is 100 mM Tris-HCl, pH 7.5. In some embodiments, the third solution in step (i) is PBS. In some embodiments, the centrifugation of step (j) is at 120,000×g. In some embodiments, the centrifugation of step (j) is for 20 minutes. In some embodiments, the centrifugation of step (j) is at 4° C., or room temperature. In some embodiments, the fourth solution in step (k) is 100 mM Tris-HCl, pH 7.5 or PBS.

pmEVs obtained by methods provided herein may be further purified by size based column chromatography, by affinity chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in 35% Optiprep in PBS. In some embodiments, if filtration was used to concentrate the filtered supernatant, the concentrate is diluted using 60% Optiprep to a final concentration of 35% Optiprep. Samples are applied to a 35-60% discontinuous sucrose gradient and centrifuged at 200,000×g for 3-24 hours at 4° C.

In some embodiments, to confirm sterility and isolation of the pmEV preparations, pmEVs are serially diluted onto agar medium used for routine culture of the bacteria being tested, and incubated using routine conditions. Non-sterile preparations are passed through a 0.22 μm filter to exclude intact cells. To further increase purity, isolated pmEVs may be DNase or proteinase K treated.

In some embodiments, the sterility of the pmEV preparations can be confirmed by plating a portion of the pmEVs onto agar medium used for standard culture of the bacteria used in the generation of the pmEVs and incubating using standard conditions.

In some embodiments select pmEVs are isolated and enriched by chromatography and binding surface moieties on pmEVs. In some embodiments, select pmEVs are isolated and/or enriched by fluorescent cell sorting by methods using affinity reagents, chemical dyes, recombinant proteins or other methods known to one skilled in the art.

In some embodiments, pmEVs are analyzed, for example, as described in Jeppesen, et al. Cell 177:428 (2019).

In some embodiments, pmEVs are lyophilized.

In some embodiments, pmEVs are gamma irradiated (for example, at 17.5 or 25 kGy).

In some embodiments, pmEVs are UV irradiated.

In some embodiments, pmEVs are heat inactivated (for example, at 50° C. for two hours or at 90° C. for two hours).

In some embodiments, pmEVs are acid treated.

In some embodiments, pmEVs are oxygen sparged (for example, at 0.1 vvm for two hours).

The phase of growth can affect the amount or properties of bacteria. In the methods of pmEV preparation provided herein, pmEVs can be isolated, for example, from a culture, at the start of the log phase of growth, midway through the log phase, and/or once stationary phase growth has been reached.

### Solutions and Dried Forms

The disclosure provides solutions (for example, liquid mixtures) that comprise EVs (for example, EVs and/or a combination of EVs described herein). For example, in some embodiments, a solution includes EVs and an excipient that comprises a bulking agent. As another example, in some embodiments, a solution includes EVs and an excipient that comprises a bulking agent and a lyoprotectant. As another example, in some embodiments, a solution includes EVs and an excipient that comprises a lyoprotectant.

The disclosure provides solutions that comprise. For example, in some embodiments, the bulking agent comprises mannitol, sucrose, maltodextrin, dextran, Ficoll, or PVP-K30. In some embodiments, the excipient optionally includes an additional component such as trehalose, mannitol, sucrose, sorbitol, maltodextrin, dextran, poloxamer 188, maltodextrin, PVP-K30, Ficoll, citrate, arginine, and/or hydroxypropyl-B-cyclodextrin. For example, in some embodiments, a solution contains a liquid preparation of EVs and an excipient that comprises a bulking agent, for example, an excipient from a stock of a formula provided in one of Tables A, B, C, D, K, or P. For example, in some embodiments, a solution includes a liquid preparation containing EVs (for example, obtained by isolating EVs from a bacterial culture (such as the supernatant) or a retentate) and an excipient that comprises a bulking agent, for example, a liquid preparation containing EVs is combined with an excipient stock that comprises a bulking agent, for example, an excipient stock of a formula provided in one of Tables A, B, C, D, K, or P, to prepare the solution.

A “dried form” that contains extracellular vesicles (EVs) (for example, from bacteria) refers to the product resulting from drying a solution that contains EVs. In some embodiments, the drying is performed by freeze drying (lyophilization) or spray drying. In some embodiments, the dried form is a powder. As used herein, a powder refers to a type of dried form and includes a lyophilized powder, but includes powders, such as spray-dried powders, obtained by methods such as spray drying.

When freeze drying (lyophilization) is performed, the resulting product is a lyophilate. In some embodiments, the dried form is a lyophilate. As used herein, a lyophilate refers to a type of dried form and includes a lyophilized powder and lyophilized cake. In some embodiments, the lyophilized cake is milled (for example, ground) to produce a lyophilized powder. Milling refers to mechanical size reduction of solids. Grinding is a type of milling, for example, that can be performed on dried forms. See, for example, Seibert et al., “MILLING OPERATIONS IN THE PHARMACEUTICAL INDUSTRY” in Chemical Engineering in the Pharmaceutical Industry: R&D to Manufacturing, Edited by David J. am Ende (2011).

The disclosure also provides dried forms, in some embodiments, such as lyophilates, that comprise EVs (for example, EVs and/or a combination of EVs described herein), and an excipient. For example, a dried form can include EVs and an excipient that comprises a bulking agent. As another example, a dried form can include EVs and an excipient that comprises a bulking agent and a lyoprotectant. As another example, a dried form can include EVs and an excipient that comprises a lyoprotectant. For example, as described herein, in some embodiments, EVs are combined with an excipient that comprises a bulking agent and/or lyoprotectant, for example, to prepare a solution. In some embodiments, the solution is dried. The resulting dried form (for example, lyophilate) contains EVs and a component(s) of the excipient, for example, bulking agent and/or lyoprotectant (for example, in dried form).

The disclosure also provides dried forms of EVs and an excipient. In some embodiments, the dried form is a lyophilate, for example, such as a lyophilized cake or lyophilized powder. In some embodiments, the dried form is a powder, for example, such as a spray-dried powder or lyophilized powder. For example, in some embodiments, the bulking agent comprises mannitol, sucrose, maltodextrin, dextran, Ficoll, or PVP-K30. In some embodiments, the excipient includes an additional component such as trehalose, mannitol, sucrose, sorbitol, dextran, poloxamer 188, maltodextrin, PVP-K30, Ficoll, citrate, arginine, and/or hydroxypropyl-B-cyclodextrin. For example, in some embodiments, a dried form contains EVs and an excipient, for example, that comprises a bulking agent, for example, an excipient from a stock of a formula provided in one of Tables A, B, C, D, K, or P. In some embodiments, the dried form has a moisture content below about 6% (or below about 5%) (for example, as determined by Karl Fischer titration). In some embodiments, the dried form has about 10% to about 80% (by weight) of an excipient, for example, an excipient that comprises a bulking agent. In some embodiments, the dried form has about 10% to about 80% (by weight) of an excipient, for example, an excipient from a stock of a formula provided in one of Tables A, B, C, D, K, or P. In some embodiments, the EVs comprise about 1% to about 99% of the total solids by weight of the dried form. In some embodiments, the dried form has at least about 1e10 particles per mg of the dried form (for example, as determined by particles per mg, such as by NTA). In some embodiments, the particles of the dried form have a hydrodynamic diameter (Z average, Zₐᵥₑ) of about 130 nm to about 300 nm after resuspension from the dried form (for example, resuspension in deionized water) (for example, as determined by dynamic light scattering).

In some embodiments, the solutions and/or dried form comprise EVs substantially or entirely free of whole bacteria (for example, live bacteria, killed bacteria, and/or attenuated bacteria). In some embodiments, the solutions and/or dried form comprise both EVs and whole bacteria (for example, live bacteria, killed bacteria, and/or attenuated bacteria). In some embodiments, the solutions and/or dried form comprise EVs from one or more (for example, 1, 2, 3, 4, 5, 6, 7, 8, 9, 10 or more) of the bacteria from a taxonomic group (for example, class, order, family, genus, species or strain) listed in Table 1, Table 2, Table 3, and/or Table 4 and/or elsewhere in the specification (for example, Table J or Example 10). In some embodiments, the solutions and/or dried form comprise EVs from one or more (for example, 1, 2, 3, 4, 5, 6, 7, 8, 9, 10 or more) of the bacteria strains or species provided herein (for example, listed in Table 1, Table 2, Table 3, and/or Table 4 and/or elsewhere in the specification (for example, Table J or Example 10)). In some embodiments, the solutions and/or dried form comprise EVs from one of the bacteria from a taxonomic group (for example, class, order, family, genus, species or strain) listed in Table 1, Table 2, Table 3, and/or Table 4 and/or elsewhere in the specification (for example, Table J or Example 10). In some embodiments, the solutions dried form comprise EVs from one of the bacteria strains or species provided herein (for example, listed in Table 1, Table 2, Table 3, and/or Table 4 and/or elsewhere in the specification (for example, Table J or Example 10)). In some embodiments, the solutions and/or dried form comprise gamma irradiated EVs. In some embodiments, the EVs are gamma irradiated after the EVs are isolated (for example, prepared).

In some embodiments, to quantify the numbers of EVs from bacteria from bacteria) and/or bacteria present in a bacterial sample, electron microscopy (for example, EM of ultrathin frozen sections) is used to visualize the EVs and/or bacteria and count their relative numbers. Alternatively, nanoparticle tracking analysis (NTA), Coulter counting, or dynamic light scattering (DLS) or a combination of these techniques is used. NTA and the Coulter counter count particles and show their sizes. DLS gives the size distribution of particles, but not the concentration. Bacteria frequently have diameters of 1-2 um (microns). The full range is 0.2-20 um. Combined results from Coulter counting and NTA can reveal the numbers of bacteria and/or EVs from bacteria in a given sample. Coulter counting reveals the numbers of particles with diameters of 0.7-10 μm. For most bacterial and/or EV samples, the Coulter counter alone can reveal the number of bacteria and/or EVs in a sample. For NTA, a Nanosight instrument can be obtained from Malvern Pananlytical. For example, the NS300 can visualize and measure particles in suspension in the size range 10-2000 nm. NTA allows for counting of the numbers of particles that are, for example, 50-1000 nm in diameter. DLS reveals the distribution of particles of different diameters within an approximate range of 1 nm-3 um.

In some embodiments, EVs are characterized by analytical methods known in the art (for example, Jeppesen, et al. Cell 177:428 (2019)).

In some embodiments, the EVs are quantified based on particle count. For example, particle count of an EV preparation can be measured using NTA. For example, particle count of an EV preparation can be measured using NTA using Zetaview.

In some embodiments, the EVs are quantified based on the amount of protein, lipid, or carbohydrate. For example, in some embodiments, total protein content of an EV preparation is measured using the Bradford assay or BCA.

In some embodiments, the EVs are isolated away from one or more other bacterial components of the source bacteria. In some embodiments, the solution and/or dried form further comprises other bacterial components.

In certain embodiments, the EV liquid preparation obtained from the source bacteria may be fractionated into subpopulations based on the physical properties (for example, size, density, protein content, and/or binding affinity) of the subpopulations. One or more of the EV subpopulations (for example, as a liquid preparation) can then be incorporated into the solutions, powders and/or lyophilates of the invention.

In certain aspects, provided herein are solutions and/or dried forms (and therapeutic compositions thereof) comprising EVs from bacteria useful for the treatment and/or prevention of disease (for example, a cancer, an autoimmune disease, an inflammatory disease, dysbiosis, or a metabolic disease), as well as methods of making and/or identifying such EVs, and methods of using such solutions and/or dried form (and therapeutic compositions thereof) (for example, for the treatment of a cancer, an autoimmune disease, an inflammatory disease, dysbiosis, or a metabolic disease, either alone or in combination with other therapeutics). In some embodiments, the therapeutic compositions comprise both EVs, and whole bacteria (for example, live bacteria, killed bacteria, and/or attenuated bacteria). In some embodiments, the therapeutic compositions comprise EVs in the absence of bacteria (for example, at least about 85%, at least about 90%, at least about 95%, or at least about 99% free of bacteria). In some embodiments, the therapeutic compositions comprise EVs and/or bacteria from one or more (for example, 1, 2, 3, 4, 5, 6, 7, 8, 9, 10 or more) of the bacteria from a taxonomic group (for example, class, order, family, genus, species or strain) listed in Table 1, Table 2 Table 3, and/or Table 4 and/or elsewhere in the specification (for example, Table J or Example 10). In some embodiments, the therapeutic compositions comprise EVs and/or bacteria from one or more of the bacteria strains or species provided herein (for example, listed in Table 1, Table 2, Table 3, and/or Table 4 and/or elsewhere in the specification (for example, Table J or Example 10)). In some embodiments, the therapeutic compositions comprise EVs and/or bacteria from one of the bacteria from a taxonomic group (for example, class, order, family, genus, species or strain) listed in Table 1, Table 2, Table 3, and/or Table 4 and/or elsewhere in the specification (for example, Table J or Example 10). In some embodiments, the therapeutic compositions comprise EVs and/or bacteria from one of the bacteria strains or species provided herein (for example, listed in Table 1, Table 2, Table 3, and/or Table 4 and/or elsewhere in the specification (for example, Table J or Example 10)).

In some embodiments, the solution and/or dried form is added to or incorporated into a food product (for example, a food or beverage) such as a health food or beverage, a food or beverage for infants, a food or beverage for pregnant women, athletes, senior citizens or other specified group, a functional food, a beverage, a food or beverage for specified health use, a dietary supplement, a probiotic, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments, the solution and/or dried form is added to a food product or food supplement for animals, including humans. The animals, other than humans, are not particularly limited, and the composition can be used for various livestock, poultry, pets, experimental animals, and the like. Specific examples of the animals include pigs, cattle, horses, sheep, goats, chickens, ducks, ostriches, turkeys, dogs, cats, rabbits, hamsters, mice, rats, monkeys, and the like, but the animals are not limited thereto.

### Therapeutic Compositions

In some embodiments, a solution and/or dried form provided herein is formulated into a therapeutic composition.

In certain embodiments, provided herein are therapeutic compositions comprising a solution and/or dried form described herein. In some embodiments, the therapeutic composition comprises a solution and/or dried form provided herein and a pharmaceutically acceptable carrier. In some embodiments, the therapeutic composition comprises a pharmaceutically acceptable excipient, such as a glidant, lubricant, and/or diluent.

In certain aspects, provided herein are therapeutic compositions comprising EVs from bacteria useful for the treatment and/or prevention of disease (for example, a cancer, an autoimmune disease, an inflammatory disease, dysbiosis, or a metabolic disease), as well as methods of making and/or identifying such EVs, and methods of using such therapeutic compositions (for example, for the treatment of a cancer, an autoimmune disease, an inflammatory disease, dysbiosis, or a metabolic disease, either alone or in combination with other therapeutics). In some embodiments, the therapeutic compositions comprise both EVs and whole bacteria (for example, live bacteria, killed bacteria, attenuated bacteria). In some embodiments, the therapeutic compositions comprise EVs in the absence of bacteria (for example, at least about 85%, at least about 90%, at least about 95%, or at least about 99% free of bacteria). In some embodiments, the therapeutic compositions comprise EVs and/or bacteria from one or more (for example, 1, 2, 3, 4, 5, 6, 7, 8, 9, 10 or more) of the bacteria from a taxonomic group (for example, class, order, family, genus, species or strain) listed in Table 1, Table 2, Table 3, and/or Table 4 and/or elsewhere in the specification (for example, Table J or Example 10). In some embodiments, the therapeutic compositions comprise EVs and/or bacteria from one or more of the bacteria strains or species provided herein (for example, listed in Table 1, Table 2, Table 3, and/or Table 4 and/or elsewhere in the specification (for example, Table J or Example 10)). In some embodiments, the therapeutic compositions comprise EVs and/or bacteria from one of the bacteria from a taxonomic group (for example, class, order, family, genus, species or strain) listed in Table 1, Table 2, Table 3, and/or Table 4 and/or elsewhere in the specification (for example, Table J or Example 10). In some embodiments, the therapeutic compositions comprise EVs and/or bacteria from one of the bacteria strains or species provided herein (for example, listed in Table 1, Table 2, Table 3, and/or Table 4 and/or elsewhere in the specification (for example, Table J or Example 10)).

In certain aspects, provided are therapeutic compositions for administration to a subject (for example, human subject). In some embodiments, the therapeutic compositions are combined with additional active and/or inactive materials in order to produce a final product, which may be in single dosage unit or in a multi-dose format. In some embodiments, the therapeutic composition is combined with an adjuvant such as an immuno-adjuvant (for example, a STING agonist, a TLR agonist, or a NOD agonist).

In some embodiments, the therapeutic composition comprises at least one carbohydrate.

In some embodiments, the therapeutic composition comprises at least one lipid. In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments, the therapeutic composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the therapeutic composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the therapeutic composition comprises an excipient, such as a pharmaceutically acceptable excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the therapeutic composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the therapeutic composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the therapeutic composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the therapeutic composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the therapeutic composition is a food product (for example, a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments, the therapeutic composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Dose Forms

In some embodiments, a therapeutic composition comprising a dried form is formulated as a solid dosage form, (also referred to as “solid dose form”) for example, for oral administration. In some embodiments, the solid dosage form comprises one or more excipients, for example, pharmaceutically acceptable excipients, in addition to the dried form. The dried form in the solid dosage form contains isolated EVs. Optionally, the EVs in the solid dosage form are gamma irradiated. In some embodiments, the solid dosage form comprises a tablet, a minitablet, a capsule, or a powder; or a combination of these forms (for example, minitablets comprised in a capsule).

In some embodiments, the solid dosage form described herein is a capsule. In some embodiments, the solid dosage form described herein is a tablet or a minitablet. Further, in some embodiments, a plurality of minitablets are in (for example, loaded into) a capsule.

In certain embodiments, the solid dosage form comprises a capsule. In some embodiments, the capsule is a size 00, size 0, size 1, size 2, size 3, size 4, or size 5 capsule. In some embodiments, the capsule is a size 0 capsule. As used herein, the size of the capsule refers to the size of the tablet prior to application of an enteric coating. In some embodiments, the capsule is banded after loading (and prior to enterically coating the capsule). In some embodiments, the capsule is banded with an HPMC-based banding solution.

In some embodiments, the solid dosage form comprises a tablet (>4 mm) (for example, 5 mm-17 mm). For example, the tablet is a 5 mm, 6 mm, 7 mm, 8 mm, 9 mm, 10 mm, 11 mm, 12 mm, 13 mm, 14 mm, 15 mm, 16 mm, 17 mm, or 18 mm tablet. The size refers to the diameter of the tablet, as is known in the art. As used herein, the size of the tablet refers to the size of the tablet prior to application of an enteric coating.

In some embodiments, the solid dosage form comprises a minitablet. In some embodiments, the minitablet is in the size range of 1 mm-4 mm range. In some embodiments, the minitablet is a 1 mm minitablet, 1.5 mm minitablet, 2 mm minitablet, 3 mm minitablet, or 4 mm minitablet. The size refers to the diameter of the minitablet, as is known in the art. As used herein, the size of the minitablet refers to the size of the minitablet prior to application of an enteric coating.

In some embodiments, the minitablets are in a capsule. In some embodiments, the capsule is a size 00, size 0, size 1, size 2, size 3, size 4, or size 5 capsule. In some embodiments, the capsule that contains the minitablets comprises HPMC (hydroxyl propyl methyl cellulose) or gelatin. In some embodiments, the minitablets are inside a capsule: the number of minitablets inside a capsule will depend on the size of the capsule and the size of the minitablets. As an example, a size 0 capsule can contain 31-35 (an average of 33) minitablets that are 3 mm minitablets. In some embodiments, the capsule is banded after loading. In some embodiments, the capsule is banded with an HPMC-based banding solution.

In some embodiments, a therapeutic composition comprising a solution and/or dried is formulated as a suspension, for example a dried form is reconstituted or a solution is diluted), for example, for oral administration or for injection. Administration by injection includes intravenous (IV), intramuscular (IM), intratumoral (IT) and subcutaneous (SC) administration. For a suspension, in some embodiments, EVs are in a buffer, for example, a pharmaceutically acceptable buffer, for example, saline or PBS. In some embodiments, a therapeutic composition comprising a solution and/or dried form (for example, that comprises EVs and a bulking agent) is formulated as a suspension, for example, a dried form is reconstituted; a solution is diluted), for example, for topical administration. In some embodiments, the suspension comprises one or more excipients, for example, pharmaceutically acceptable excipients. In some embodiments, the suspension comprises sucrose or glucose. In some embodiments, the EVs in the solution or dried form are isolated EVs. Optionally, the EVs in the suspension are gamma irradiated.

### Coating

In some embodiments, a solid dosage form (for example, capsule, tablet or minitablet) described herein is enterically coated, for example, with one enteric coating layer or with two layers of enteric coating, for example, an inner enteric coating and an outer enteric coating. The inner enteric coating and outer enteric coating are not identical (for example, the inner enteric coating and outer enteric coating do not contain the same components in the same amounts). The enteric coating allows for release of the therapeutic agent (such as bacterial EVs, dried forms, and/or solid dosage forms thereof), for example, in the small intestine.

Release of the therapeutic agent in the small intestine allows the therapeutic agent to target and affect cells (for example, epithelial cells and/or immune cells) located at these specific locations, for example, which can cause a local effect in the gastrointestinal tract and/or cause a systemic effect (for example, an effect outside of the gastrointestinal tract).

EUDRAGIT is the brand name for a diverse range of polymethacrylate-based copolymers. It includes anionic, cationic, and neutral copolymers based on methacrylic acid and methacrylic/acrylic esters or their derivatives.

Examples of other materials that can be used in the enteric coating (for example, the one enteric coating or the inner enteric coating and/or the outer enteric coating) include cellulose acetate phthalate (CAP), cellulose acetate trimellitate (CAT), poly(vinyl acetate phthalate) (PVAP), hydroxypropyl methylcellulose phthalate (HPMCP), fatty acids, waxes, shellac (esters of aleurtic acid), plastics, plant fibers, zein, Aqua-Zein® (an aqueous zein formulation containing no alcohol), amylose starch, starch derivatives, dextrins, methyl acrylate-methacrylic acid copolymers, cellulose acetate succinate, hydroxypropyl methyl cellulose acetate succinate (hypromellose acetate succinate), methyl methacrylate-methacrylic acid copolymers, and/or sodium alginate.

In some embodiments, the enteric coating (for example, the one enteric coating or the inner enteric coating and/or the outer enteric coating) includes a methacrylic acid ethyl acrylate (MAE) copolymer (1:1).

In some embodiments, the one enteric coating includes methacrylic acid ethyl acrylate (MAE) copolymer (1:1) (such as Kollicoat MAE 100P).

In some embodiments, the one enteric coating includes a Eudragit copolymer, for example, a Eudragit L (for example, Eudragit L 100-55; Eudragit L 30 D-55), a Eudragit S, a Eudragit RL, a Eudragit RS, a Eudragit E, or a Eudragit FS (for example, Eudragit FS 30 D).

Other examples of materials that can be used in the enteric coating (for example, the one enteric coating or the inner enteric coating and/or the outer enteric coating) include those described in, for example, U.S. Pat. Nos. 6,312,728; 6,623,759; 4,775,536; 5,047,258; 5,292,522; 6,555,124; 6,638,534; U.S. 2006/0210631; U.S. 2008/200482; U.S. 2005/0271778; U.S. 2004/0028737; WO 2005/044240.

See also, for example, U.S. Pat. No. 9,233,074, which provides pH dependent, enteric polymers that can be used with the solid dosage forms provided herein, including methacrylic acid copolymers, polyvinylacetate phthalate, hydroxypropylmethyl cellulose acetate succinate, hydroxypropylmethyl cellulose phthalate and cellulose acetate phthalate; suitable methacrylic acid copolymers include: poly(methacrylic acid, methyl methacrylate) 1:1 sold, for example, under the Eudragit L100 trade name; poly(methacrylic acid, ethyl acrylate) 1:1 sold, for example, under the Eudragit L100-55 trade name; partially-neutralized poly(methacrylic acid, ethyl acrylate) 1:1 sold, for example, under the Kollicoat MAE-100P trade name; and poly(methacrylic acid, methyl methacrylate) 1:2 sold, for example, under the Eudragit S100 trade name.

In some embodiments, the solid dose form (for example, a capsule) comprises a single layer coating, for example, a non-enteric coating such as HPMC (hydroxyl propyl methyl cellulose) or gelatin.

### Method of Making Solutions and Dried Forms

The disclosure also provides methods of preparing solutions of EVs and an excipient that comprises a bulking agent. For example, in some embodiments, the bulking agent comprises mannitol, sucrose, polyethylene glycol (PEG, such as PEG 6000), cyclodextrin, maltodextrin, dextran, Ficoll, or PVP-K30. In some embodiments, the excipient comprises a lyoprotectant. In some embodiments, the excipient optionally includes an additional component such as trehalose, mannitol, sucrose, sorbitol, dextran, poloxamer 188, maltodextrin, PVP-K30, Ficoll, citrate, arginine, and/or hydroxypropyl-B-cyclodextrin. For example, in some embodiments, a liquid preparation of EVs and an excipient that comprises a bulking agent are combined to prepare a solution. For example, in some embodiments, a liquid preparation of EVs (for example, obtained by isolating EVs from a bacterial culture (such as a supernatant or a retentate) and an excipient that comprises a bulking agent, for example, an excipient stock of a formula provided in one of Tables A, B, C, D, K, or P, are combined to prepare a solution. For example, in some embodiments, a liquid preparation containing EVs (for example, obtained by isolating EVs from a bacterial culture (such as a supernatant or a retentate) and an excipient that comprises a bulking agent are combined, for example, a liquid preparation containing EVs (for example, obtained by isolating EVs from a bacterial culture (such as a supernatant or a retentate) or a retentate) are combined with an excipient that comprises a bulking agent, for example, such as mannitol or an excipient of an excipient stock of a formula provided in one of Tables A, B, C, D, K, or P, to prepare the solution.

The disclosure also provides methods of preparing dried forms of EVs. For example, in some embodiments, the method is used to prepare a lyophilate such as a lyophilized powder and/or a lyophilized cake. For example, in some embodiments, the method is used to prepare a powder such as a lyophilized powder and/or a spray-dried powder. In some embodiments, the excipient comprises a bulking agent. For example, in some embodiments, the bulking agent comprises mannitol, sucrose, polyethylene glycol (PEG, such as PEG 6000), cyclodextrin, maltodextrin, dextran, Ficoll, or PVP-K30. In some embodiments, the excipient comprises a lyoprotectant. In some embodiments, the excipient optionally includes an additional component such as trehalose, mannitol, sucrose, sorbitol, dextran, poloxamer 188, maltodextrin, PVP-K30, Ficoll, citrate, arginine, and/or hydroxypropyl-B-cyclodextrin. For example, in some embodiments, a liquid preparation containing EVs (for example, obtained by isolating EVs from a bacterial culture (such as a supernatant or a retentate) is be combined with an excipient that comprises a bulking agent, such as mannitol or an excipient of an excipient stock of a formula provided in one of Tables A, B, C, D, K, or P; and dried (for example, by lyophilization or spray drying) to thereby prepare a dried form. In some embodiments, the dried form has a moisture content below about 6%, below about 5%, below about 4%, between about 0.5% to about 5%, between about 1% to about 5%, between about 1% to about 4%, between about 1.5% to about 4%, or between about 2% to about 3%, (for example, as determined by Karl Fischer titration). In some embodiments, the dried form has about 10% to about 80% (by weight) of an excipient, for example, an excipient that comprises a bulking agent. In some embodiments, the dried form has about 10% to about 80% (by weight) of an excipient, for example, an excipient from a stock of a formula provided in one of Tables A, B, C, D, K, or P. In some embodiments, the EVs comprise about 1% to about 99% of the total solids by weight of the dried form. In some embodiments, the dried form has at least about 1e10 particles per mg of the dried form (for example, as determined by particles per mg, such as by NTA). In some embodiments, the particles in the dried form have a hydrodynamic diameter (Z average, Zₐᵥₑ) of about 130 nm to about 300 nm after resuspension from the dried form (for example, resuspension in deionized water) (for example, as determined by dynamic light scattering).

In some embodiments, the dried form is a lyophilate. In some embodiments, the lyophilate is a lyophilized powder or a lyophilized cake. In some embodiments, the dried form is a powder. In some embodiments, the powder is a lyophilized powder or a spray-dried powder.

In some embodiments, a method of preparing a solution that comprises extracellular vesicles (EVs) from bacteria includes:
combining a liquid preparation that comprises EVs from bacteria with an excipient that comprises a bulking agent, thereby preparing the solution.

In some embodiments, a method of preparing a solution that comprises extracellular vesicles (EVs) from bacteria includes:
combining a liquid preparation that comprises EVs from bacteria with an excipient that comprises a bulking agent and a lyoprotectant, thereby preparing the solution.

In some embodiments, a method of preparing a solution that comprises extracellular vesicles (EVs) from bacteria includes:
combining a liquid preparation that comprises EVs from bacteria with an excipient that comprises a lyoprotectant, thereby preparing the solution.

In some embodiments, a method of preparing a solution that comprises extracellular vesicles (EVs) from bacteria includes:
combining a liquid preparation that comprises EVs with a stock comprising one or more excipients, wherein the stock comprises a formula provided in Table A, B, C, D, K or P, thereby preparing a solution.

In some embodiments, the EVs are from bacteria.

In some embodiments, the disclosure provides a solution prepared by a method described herein.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a bulking agent to prepare a solution; and
drying the solution, thereby preparing the dried form.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a bulking agent to prepare a solution;
drying the solution to prepare a cake, and
milling (for example, grinding the cake, thereby preparing the dried form.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
drying the solution, thereby preparing the dried form.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution;
drying the solution to prepare a cake, and
milling (for example, grinding) the cake, thereby preparing the dried form.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a lyoprotectant to prepare a solution; and
drying the solution, thereby preparing the dried form.

In some aspects, the disclosure provides a method of preparing a dried form that comprises extracellular vesicles (EVs) from bacteria, the method comprising:
combining a liquid preparation that comprises EVs from bacteria with an excipient that comprises (or consists essentially of) a lyoprotectant to prepare a solution;
drying the solution to prepare a cake, and
milling (for example, grinding) the cake, thereby preparing the dried form.

In some embodiments, the drying comprises lyophilization.

In some embodiments, the drying comprises spray drying.

In some embodiments, the method further comprises combining the dried form with an additional ingredient. In some embodiments, the additional ingredient comprises an excipient, for example, a glidant, lubricant, and/or diluent.

In some embodiments, the disclosure provides a dried form prepared by a method described herein.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a bulking agent to prepare a solution; and
drying the solution, thereby preparing the powder.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a bulking agent to prepare a solution;
drying the solution to prepare a cake, and
milling (for example, grinding) the cake, thereby preparing the powder.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
drying the solution, thereby preparing the powder.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution;
drying the solution to prepare a cake, and
milling (for example, grinding) the cake, thereby preparing the powder.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a lyoprotectant to prepare a solution; and
the solution, thereby preparing the powder.

In some aspects, the disclosure provides a method of preparing a powder that comprises extracellular vesicles (EVs) from bacteria, the method comprising:
combining a liquid preparation that comprises EVs from bacteria with an excipient that comprises (or consists essentially of) a lyoprotectant to prepare a solution;
drying the solution to prepare a cake, and
milling (for example, grinding) the cake, thereby preparing the powder.

In some embodiments, the drying comprises lyophilization.

In some embodiments, the drying comprises spray drying.

In some embodiments, the method further comprises combining the powder with an additional ingredient. In some embodiments, the additional ingredient comprises an excipient, for example, a glidant, lubricant, and/or diluent.

In some embodiments, the disclosure provides a powder prepared by a method described herein.

In some aspects, the disclosure provides a method of preparing a spray-dried powder that comprises extracellular vesicles (EVs) from bacteria, the method comprising:
combining a liquid preparation that comprises EVs from bacteria with an excipient that comprises (or consists essentially of) a bulking agent to prepare a solution; and
spray drying the solution, thereby preparing the spray-dried powder.

In some aspects, the disclosure provides a method of preparing a spray-dried powder that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
spray drying the solution, thereby preparing the spray-dried powder.

In some aspects, the disclosure provides a method of preparing a spray-dried powder that comprises extracellular vesicles (EVs) from bacteria, the method comprising:
combining a liquid preparation that comprises EVs from bacteria with an excipient that comprises (or consists essentially of) a lyoprotectant to prepare a solution; and
spray drying the solution, thereby preparing the spray-dried powder.

In some embodiments, the method further comprises combining the spray-dried powder with an additional ingredient. In some embodiments, the additional ingredient comprises an excipient, for example, a glidant, lubricant, and/or diluent.

In some embodiments, the disclosure provides a spray-dried powder prepared by a method described herein.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a bulking agent to prepare a solution; and
freeze drying (lyophilizing) the solution, thereby preparing the lyophilate.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a bulking agent to prepare a solution;
freeze drying (lyophilizing) the solution to prepare a cake, and
milling (for example, grinding) the cake, thereby preparing the lyophilate.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution;
freeze drying (lyophilizing) the solution to prepare a cake, and
milling (for example, grinding) the cake, thereby preparing the lyophilate.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from bacteria, the method comprising:
combining a liquid preparation that comprises EVs from bacteria with an excipient that comprises (or consists essentially of) a lyoprotectant to prepare a solution;
freeze drying (lyophilizing) the solution to prepare a cake, and
milling (for example, grinding) the cake, thereby preparing the lyophilate.

In some embodiments, the method further comprises combining the lyophilate with an additional ingredient. In some embodiments, the additional ingredient comprises an excipient, for example, a glidant, lubricant, and/or diluent.

In some embodiments, the disclosure provides a lyophilate prepared by a method described herein.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a bulking agent to prepare a solution; and
freeze drying (lyophilizing) the solution, thereby preparing the lyophilized powder.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a bulking agent to prepare a solution;
freeze drying (lyophilizing) the solution to prepare a cake, and
milling (for example, grinding) the cake, thereby preparing the lyophilized powder.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution;
freeze drying (lyophilizing) the solution to prepare a cake, and
milling (for example, grinding) the cake, thereby preparing the lyophilized powder.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from bacteria, the method comprising:
combining a liquid preparation that comprises EVs from bacteria with an excipient that comprises (or consists essentially of) a lyoprotectant to prepare a solution;
freeze drying (lyophilizing) the solution to prepare a cake, and
milling (for example, grinding) the cake, thereby preparing the lyophilized powder.

In some embodiments, the method further comprises combining the lyophilized powder with an additional ingredient. In some embodiments, the additional ingredient comprises an excipient, for example, a glidant, lubricant, and/or diluent.

In some embodiments, the disclosure provides a lyophilized powder prepared by a method described herein.

In some aspects, the disclosure provides a method of preparing a lyophilized cake that comprises extracellular vesicles (EVs) from bacteria, the method comprising:
combining a liquid preparation that comprises EVs from bacteria with an excipient that comprises (or consists essentially of) a bulking agent to prepare a solution; and
freeze drying (lyophilizing) the solution, thereby preparing the lyophilized cake.

In some aspects, the disclosure provides a method of preparing a lyophilized cake that comprises extracellular vesicles (EVs) from bacteria, the method comprising:
combining a liquid preparation that comprises EVs from bacteria with an excipient that comprises (or consists essentially of) a bulking agent and a lyoprotectant to prepare a solution; and
freeze drying (lyophilizing) the solution, thereby preparing the lyophilized cake.

In some aspects, the disclosure provides a method of preparing a lyophilized cake that comprises extracellular vesicles (EVs) from bacteria, the method comprising:
combining a liquid preparation that comprises EVs from bacteria with an excipient that comprises (or consists essentially of) a lyoprotectant to prepare a solution; and
freeze drying (lyophilizing) the solution, thereby preparing the lyophilized cake.

In some embodiments, the disclosure provides a lyophilized cake prepared by a method described herein.

In some aspects, the disclosure provides a method of preparing a solution that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution

In some embodiments, the EVs are from bacteria.

In some embodiments, the disclosure provides a solution prepared by a method described herein.

In some aspects, the disclosure provides a method of preparing a dried form that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
drying the solution, thereby preparing the dried form.

In some aspects, the disclosure provides a method of preparing a dried form that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution;
drying the solution to prepare a cake, and
milling (for example, grinding) the cake, thereby preparing the dried form.

In some embodiments, the EVs are from bacteria.

In some embodiments, the drying comprises lyophilization.

In some embodiments, the drying comprises spray drying.

In some embodiments, the method further comprises combining the dried form with an additional ingredient. In some embodiments, the additional ingredient comprises an excipient, for example, a glidant, lubricant, and/or diluent.

In some embodiments, the disclosure provides a dried form prepared by a method described herein.

In some aspects, the disclosure provides a method of preparing a powder that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
drying the solution, thereby preparing the powder.

In some aspects, the disclosure provides a method of preparing a powder that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution;
drying the solution to prepare a cake, and
milling (for example, grinding) the cake, thereby preparing the powder.

In some embodiments, the EVs are from bacteria.

In some embodiments, the drying comprises lyophilization.

In some embodiments, the drying comprises spray drying.

In some embodiments, the method further comprises combining the powder with an additional ingredient. In some embodiments, the additional ingredient comprises an excipient, for example, a glidant, lubricant, and/or diluent.

In some embodiments, the disclosure provides a powder prepared by a method described herein.

In some aspects, the disclosure provides a method of preparing a spray-dried powder that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
spray drying the solution, thereby preparing the spray-dried powder.

In some embodiments, the EVs are from bacteria.

In some embodiments, the method further comprises combining the spray-dried powder with an additional ingredient. In some embodiments, the additional ingredient comprises an excipient, for example, a glidant, lubricant, and/or diluent.

In some embodiments, the disclosure provides a spray-dried powder prepared by a method described herein.

In some aspects, the disclosure provides a method of preparing a lyophilate that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
freeze drying (lyophilizing) the solution, thereby preparing the lyophilate.

In some aspects, the disclosure provides a method of preparing a lyophilate that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution;
freeze drying (lyophilizing) the solution to prepare a cake, and
milling (for example, grinding) the cake, thereby preparing the lyophilate.

In some embodiments, the EVs are from bacteria.

In some embodiments, the method further comprises combining the lyophilate with an additional ingredient. In some embodiments, the additional ingredient comprises an excipient, for example, a glidant, lubricant, and/or diluent.

In some embodiments, the disclosure provides a lyophilate prepared by a method described herein.

In some aspects, the disclosure provides a method of preparing a lyophilized powder that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
freeze drying (lyophilizing) the solution, thereby preparing the lyophilized powder.

In some aspects, the disclosure provides a method of preparing a lyophilized powder that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution;
freeze drying (lyophilizing) the solution to prepare a cake, and
milling (for example, grinding) the cake, thereby preparing the lyophilized powder.

In some embodiments, the EVs are from bacteria.

In some embodiments, the method further comprises combining the lyophilized powder with an additional ingredient. In some embodiments, the additional ingredient comprises an excipient, for example, a glidant, lubricant, and/or diluent.

In some embodiments, the disclosure provides a lyophilized powder prepared by a method described herein.

In some aspects, the disclosure provides a method of preparing a lyophilized cake that comprises extracellular vesicles (EVs), the method comprising:
combining a liquid preparation that comprises EVs with a stock comprising one or more excipients, wherein the stock comprises a formula provided in Table A, B, C, D, K or P, thereby preparing a solution; and
freeze drying (lyophilizing) the solution, thereby preparing a lyophilized cake.

In some embodiments, the disclosure provides a lyophilized cake prepared by a method described herein.

### Method of Preparing Therapeutic Compositions

The disclosure also provides methods of preparing therapeutic compositions. In some embodiments, the method includes combining a solution or dried form described herein with a pharmaceutically acceptable excipient, such as a glidant, lubricant, and/or diluent, thereby preparing a therapeutic composition.

The disclosure also provides methods of preparing therapeutic compositions, such as solid dosage forms, that contain a dried form described herein. In some embodiments, the solid dosage form is a capsule, tablet, or minitablet.

The disclosure also provides methods of making a solid dosage form (for example, for oral administration) (for example, for pharmaceutical use) that comprises a dried form. In some embodiments, the dried form comprises extracellular vesicles (EVs) and an excipient that comprises a bulking agent. In some embodiments, the dried form comprises extracellular vesicles (EVs) and an excipient that comprises a lyoprotectant. In some embodiments, the dried form comprises extracellular vesicles (EVs) and an excipient that comprises a bulking agent and a lyoprotectant. In some embodiments, the dried form also contains one or more additional components. In some embodiments, the dried form is combined with one or more pharmaceutically acceptable excipients. In some embodiments, the solid dosage form is enterically coated, for example, with a coating described herein.

In some aspects, a method of making the solid dosage form includes:
Loading a dried form into a capsule, thereby preparing a capsule, and thereby preparing the solid dosage form;
Optionally combining the dried form with a pharmaceutically acceptable excipient prior to loading into the capsule; and/or
Optionally banding the capsule after loading the capsule (for example, optionally banding the capsule after loading the capsule).

In some aspects, a method of making the solid dosage form includes:
Compressing a dried form described herein into a minitablet, thereby preparing a minitablet and thereby preparing the solid dosage form;
Optionally combining the dried form with a pharmaceutically acceptable excipient prior to compressing;
Optionally filling a capsule with a plurality of enterically coated minitablets.

In some aspects, a method of making the solid dosage form includes:
Compressing a powder described herein into a tablet, thereby preparing a tablet, and thereby preparing the solid dosage form;
Optionally combining the dried form with a pharmaceutically acceptable excipient prior to compressing.

In certain embodiments, the method comprises performing wet granulation on a powder prior to combining the powder and one or more (for example, one, two or three) excipients into a therapeutic composition, such as a solid dosage form. In some embodiments, the wet granulation comprises (i) mixing the powder with a granulating fluid (for example, water, ethanol, or isopropanol, alone or in combination). In some embodiments, the wet granulation comprises mixing the powder with water. In some embodiments, the wet granulation comprises (ii) drying mixed powder and granulating fluid (for example, drying on a fluid bed dryer). In some embodiments, the wet granulation comprises (iii) milling (for example, grinding) the dried powder and granulating fluid. The milled (for example, ground) powder and granulating fluid are then combined with the one or more (for example, one, two or three) excipients to prepare a therapeutic composition, such as a solid dosage form. In some embodiments, the powder is a lyophilized powder. In some embodiments, the powder is a spray-dried powder.

In some embodiments, a dried form described herein is reconstituted in a liquid (for example, a buffer, juice, or water) to prepare a therapeutic composition.

In some embodiments, a solution is resuspended (for example, diluted) in a liquid (for example, a buffer, juice, or water) to prepare a therapeutic composition.

In some embodiments, a therapeutic composition comprising a dried form described herein is reconstituted in a liquid (for example, a buffer, juice, or water) to prepare a suspension.

In some embodiments, a therapeutic composition comprising a solution is resuspended (for example, diluted) in a liquid (for example, a buffer, juice, or water) to prepare a suspension.

### Gamma-Irradiation

Powders and frozen biomass (for example, of EVs from bacteria) can be gamma-irradiated.

In some embodiments, powders (for example, of EVs from bacteria) are gamma-irradiated at 17.5 kGy radiation unit at ambient temperature.

In some embodiments, frozen biomasses (for example, of EVs from bacteria) are gamma-irradiated at 25 kGy radiation unit in the presence of dry ice.

### Additional Therapeutic Agents

In certain aspects, the methods provided herein include the administration to a subject of a therapeutic composition described herein either alone or in combination with an additional therapeutic agent. In some embodiments, the additional therapeutic agent is an immunosuppressant, an anti-inflammatory agent, a steroid, and/or a cancer therapeutic.

In some embodiments, the therapeutic composition comprising EVs from bacteria is administered to the subject before the additional therapeutic agent is administered (for example, at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, the therapeutic composition comprising EVs from bacteria is administered to the subject after the additional therapeutic agent is administered (for example, at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the therapeutic composition comprising EVs from bacteria and the additional therapeutic agent are administered to the subject simultaneously or nearly simultaneously (for example, administrations occur within an hour of each other).

In some embodiments, an antibiotic is administered to the subject before the therapeutic composition comprising EVs from bacteria is administered to the subject (for example, at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, an antibiotic is administered to the subject after therapeutic composition comprising EVs from bacteria is administered to the subject (for example, at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the therapeutic composition comprising EVs from bacteria and the antibiotic are administered to the subject simultaneously or nearly simultaneously (for example, administrations occur within an hour of each other).

In some embodiments, the additional therapeutic agent is a cancer therapeutic. In some embodiments, the cancer therapeutic is a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (for example, calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanibicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, for example, paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (for example, CPT-11); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the cancer therapeutic is a cancer immunotherapy agent. Immunotherapy refers to a treatment that uses a subject's immune system to treat cancer, for example, checkpoint inhibitors, cancer vaccines, cytokines, cell therapy, CAR-T cells, and dendritic cell therapy. Non-limiting examples of immunotherapies are checkpoint inhibitors include Nivolumab (BMS, anti-PD-1), Pembrolizumab (Merck, anti-PD-1), Ipilimumab (BMS, anti-CTLA-4), MEDI4736 (AstraZeneca, anti-PD-L1), and MPDL3280A (Roche, anti-PD-L1). Other immunotherapies may be tumor vaccines, such as Gardail, Cervarix, BCG, sipulencel-T, Gp100:209-217, AGS-003, DCVax-L, Algenpantucel-L, Tergenpantucel-L, TG4010, ProstAtak, Prostvac-V/R-TRICOM, Rindopepimul, E75 peptide acetate, IMA901, POL-103A, Belagenpumatucel-L, GSK1572932A, MDX-1279, GV1001, and Tecemotide. The immunotherapy agent may be administered via injection (for example, intravenously, intratumorally, subcutaneously, or into lymph nodes), but may also be administered orally, topically, or via aerosol. Immunotherapies may comprise adjuvants such as cytokines.

In some embodiments, the immunotherapy agent is an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0010718C (avelumab), AUR-012 and STI-A1010.

In some embodiments, the methods provided herein include the administration of a therapeutic composition described herein in combination with one or more additional therapeutic agents. In some embodiments, the methods disclosed herein include the administration of two immunotherapy agents (for example, immune checkpoint inhibitor). For example, the methods provided herein include the administration of a pharmaceutical composition described herein in combination with a PD-1 inhibitor (such as pemrolizumab or nivolumab or pidilizumab) or a CLTA-4 inhibitor (such as ipilimumab) or a PD-L1 inhibitor (such as avelumab).

In some embodiments, the immunotherapy agent is an antibody or antigen binding fragment thereof that, for example, binds to a cancer-associated antigen. Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MCIR, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secemin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-1b/GAGED2a. In some embodiments, the antigen is a neo-antigen.

In some embodiments, the immunotherapy agent is a cancer vaccine and/or a component of a cancer vaccine (for example, an antigenic peptide and/or protein). The cancer vaccine can be a protein vaccine, a nucleic acid vaccine or a combination thereof. For example, in some embodiments, the cancer vaccine comprises a polypeptide comprising an epitope of a cancer-associated antigen. In some embodiments, the cancer vaccine comprises a nucleic acid (for example, DNA or RNA, such as mRNA) that encodes an epitope of a cancer-associated antigen. Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MCIR, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secemin 1, SIRT2, SNRPD1, SOX10, Spi7, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-1b/GAGED2a. In some embodiments, the antigen is a neo-antigen. In some embodiments, the cancer vaccine is administered with an adjuvant. Examples of adjuvants include, but are not limited to, an immune modulatory protein, Adjuvant 65, α-GalCer, aluminum phosphate, aluminum hydroxide, calcium phosphate, J-Glucan Peptide, CpG ODN DNA, GPI-0100, lipid A, lipopolysaccharide, Lipovant, Montanide, N-acetyl-muramyl-L-alanyl-D-isoglutamine, Pam3CSK4, quil A, cholera toxin (CT) and heat-labile toxin from enterotoxigenic Escherichia coli (LT) including derivatives of these (CTB, mmCT, CTA1-DD, LTB, LTK63, LTR72, dmLT) and trehalose dimycolate.

In some embodiments, the immunotherapy agent is an immune modulating protein to the subject. In some embodiments, the immune modulatory protein is a cytokine or chemokine. Examples of immune modulating proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon alpha (“IFN-alpha”), Interferon beta (“IFN-beta”) Interferon gamma (“IFN-gamma”), Interlukin-1 alpha (“IL-1 alpha”), Interlukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17A-F (“IL-17A-F”), Interleukin-18 (“IL-18”), Interleukin-21 (“IL-21”), Interleukin-22 (“IL-22”), Interleukin-23 (“IL-23”), Interleukin-33 (“IL-33”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophage inflammatory protein-1-delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRG1-beta1, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”), Angiopoietin 1, Angiostatin, Catheprin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, Follistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgpl30, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor κB (“RANK”).

In some embodiments, the cancer therapeutic is an anti-cancer compound. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin dif itox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 131I-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®).

Exemplary anti-cancer compounds that modify the function of proteins that regulate gene expression and other cellular functions (for example, HDAC inhibitors, retinoid receptor ligants) are Vorinostat (Zolinza®), Bexarotene (Targretin®) and Romidepsin (Istodax®), Alitretinoin (Panretin®), and Tretinoin (Vesanoid®).

Exemplary anti-cancer compounds that induce apoptosis (for example, proteasome inhibitors, antifolates) are Bortezomib (Velcade®), Carfilzomib (Kyprolis™), and Pralatrexate (Folotyn®).

Exemplary anti-cancer compounds that increase anti-tumor immune response (for example, anti CD20, anti CD52; anti-cytotoxic T-lymphocyte-associated antigen-4) are Rituximab (Rituxan®), Alemtuzumab (Campath®), Ofatumumab (Arzerra®), and Ipilimumab (Yervoy™).

Exemplary anti-cancer compounds that deliver toxic agents to cancer cells (for example, anti-CD20-radionuclide fusions; IL-2-diphtheria toxin fusions; anti-CD30-monomethylauristatin E (MMAE)-fusions) are Tositumomab and 131I-tositumomab (Bexxar®) and Ibritumomab tiuxetan (Zevalin®), Denileukin diftitox (Ontak®), and Brentuximab vedotin (Adcetris®).

Other exemplary anti-cancer compounds are small molecule inhibitors and conjugates thereof of, for example. Janus kinase, ALK, Bcl-2, PARP, PI3K, VEGF receptor, Braf, MEK, CDK, and HSP90.

Exemplary platinum-based anti-cancer compounds include, for example, cisplatin, carboplatin, oxaliplatin, satraplatin, picoplatin, Nedaplatin, Triplatin, and Lipoplatin. Other metal-based drugs suitable for treatment include, but are not limited to ruthenium-based compounds, ferrocene derivatives, titanium-based compounds, and gallium-based compounds.

In some embodiments, the cancer therapeutic is a radioactive moiety that comprises a radionuclide. Exemplary radionuclides include, but are not limited to Cr-51, Cs-131, Ce-134, Se-75, Ru-97, I-125, Eu-149, Os-189m, Sb-119, I-123, Ho-161, Sb-117, Ce-139, In-111, Rh-103m, Ga-67, T1-201, Pd-103, Au-195, Hg-197, Sr-87m, Pt-191, P-33, Er-169, Ru-103, Yb-169, Au-199, Sn-121, Tm-167, Yb-175, In-113m, Sn-113, Lu-177, Rh-105, Sn-117m, Cu-67, Sc-47, Pt-195m, Ce-141, I-131, Tb-161, As-77, Pt-197, Sm-153, Gd-159, Tm-173, Pr-143, Au-198, Tm-170, Re-186, Ag-111, Pd-109, Ga-73, Dy-165, Pm-149, Sn-123, Sr-89, Ho-166, P-32, Re-188, Pr-142, Ir-194, In-114m/In-114, and Y-90.

In some embodiments, the cancer therapeutic is an antibiotic. For example, if the presence of a cancer-associated bacteria and/or a cancer-associated microbiome profile is detected according to the methods provided herein, antibiotics can be administered to eliminate the cancer-associated bacteria from the subject. “Antibiotics” broadly refers to compounds capable of inhibiting or preventing a bacterial infection. Antibiotics can be classified in a number of ways, including their use for specific infections, their mechanism of action, their bioavailability, or their spectrum of target microbe (for example, Gram-negative vs. Gram-positive bacteria, aerobic vs. anaerobic bacteria, etc.) and these may be used to kill specific bacteria in specific areas of the host (“niches”) (Leekha, et al 2011. General Principles of Antimicrobial Therapy. Mayo Clin Proc. 86(2): 156-167). In certain embodiments, antibiotics can be used to selectively target bacteria of a specific niche. In some embodiments, antibiotics known to treat a particular infection that includes a cancer niche may be used to target cancer-associated bacteria, including cancer-associated bacteria in that niche. In some embodiments, antibiotics are administered after the therapeutic composition comprising EVs from bacteria. In some embodiments, antibiotics are administered before therapeutic composition comprising EVs from bacteria.

In some aspects, antibiotics can be selected based on their bactericidal or bacteriostatic properties. Bactericidal antibiotics include mechanisms of action that disrupt the cell wall (for example, β-lactams), the cell membrane (for example, daptomycin), or bacterial DNA (for example, fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin. Aminoglycosides are effective, for example, against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin. Geldanamycin and Herbimycin are believed to inhibit or alter the function of Heat Shock Protein 90.

Carbacephems include, but are not limited to, Loracarbef. Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, and Ceftobiprole. Selected Cephalosporins are effective, for example, against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, for example, against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, for example, against anaerobic bacteria, as well as Staphylococcus and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, for example, against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, for example, against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, for example, against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin. Penicillins are effective, for example, against Gram-positive bacteria, facultative anaerobes, for example, Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to, Bacitracin, Colistin, and Polymyxin B and E. Polypeptide Antibiotics are effective, for example, against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, for example, against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, for example, against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin P1, clarithromycin, erythromycins, furazolidone, fusidic acid, Na fusidate, gramicidin, imipenem, indolicidin, josamycin, magainan II, metronidazole, nitroimidazoles, mikamycin, mutacin B-Ny266, mutacin B-JHl 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

In some embodiments, the additional therapeutic agent is an immunosuppressive agent, a DMARD, a pain-control drug, a steroid, a non-steroidal antiinflammatory drug (NSAID), or a cytokine antagonist, and combinations thereof. Representative agents include, but are not limited to, cyclosporin, retinoids, corticosteroids, propionic acid derivative, acetic acid derivative, enolic acid derivatives, fenamic acid derivatives, Cox-2 inhibitors, lumiracoxib, ibuprophen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetominophen, Celecoxib, Diclofenac, tramadol, piroxicam, meloxicam, tenoxicam, droxicam, lomoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs (for example, hydroxychloroquine and chloroquine), sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists (for example, TNF alpha antagonists or TNF alpha receptor antagonists), for example, ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MablIera®), ABATACEPT (Orencia®), TOCILIZUMAB (RoActemra/Actemra®), integrin antagonists (TYSABRI® (natalizumab)), IL-1 antagonists (ACZ885 (Ilaris)), Anakinra (Kineret®)), CD4 antagonists, IL-23 antagonists, IL-20 antagonists, IL-6 antagonists, BLyS antagonists (for example, Atacicept, Benlysta®/LymphoStat-B® (belimumab)), p38 Inhibitors, CD20 antagonists (Ocrelizumab, Ofatumumab (Arzerra®)), interferon gamma antagonists (Fontolizumab), prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin+Viusid, TwHF, Methoxsalen, Vitamin D—ergocalciferol, Milnacipran, Paclitaxel, rosig tazone, Tacrolimus (Prograf®), RADOOl, rapamune, rapamycin, fostamatinib, Fentanyl, XOMA 052, Fostamatinib disodium, rosightazone, Curcumin (Longvida™), Rosuvastatin, Maraviroc, ramipnl, Milnacipran, Cobiprostone, somatropin, tgAAC94 gene therapy vector, MK0359, GW856553, esomeprazole, everolimus, trastuzumab, JAKI and JAK2 inhibitors, pan JAK inhibitors, for example, tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists (Tysarbri® (natalizumab)), VGEF antagnosits, CXCL antagonists, MMP antagonists, defensin antagonists, IL-1 antagonists (including IL-1 beta antagonsits), and IL-23 antagonists (for example, receptor decoys, antagonistic antibodies, etc.).

In some embodiments, the additional therapeutic agent is an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines (for example, vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

### Administration

In certain aspects, provided herein is a method of delivering a therapeutic composition described herein (for example, a therapeutic composition comprising a solution or dried form described herein) to a subject. In some embodiments of the methods provided herein, the therapeutic composition is administered in conjunction with the administration of an additional therapeutic agent. In some embodiments, the therapeutic composition comprising a solution or dried form described herein is co-formulated with the additional therapeutic agent. In some embodiments, the therapeutic composition comprising a solution or dried form described herein is co-administered with the additional therapeutic agent. In some embodiments, the additional therapeutic agent is administered to the subject before administration of the therapeutic composition comprising a solution or dried form described herein (for example, about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic agent is administered to the subject after administration of the therapeutic composition comprising a solution or dried form described herein (for example, about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the therapeutic composition comprising a solution or dried form described herein and the additional therapeutic agent. In some embodiments, different modes of delivery are used to administer the therapeutic composition comprising a solution or dried form described herein and the additional therapeutic agent. For example, in some embodiments the therapeutic composition comprising a solution or dried form described herein is administered orally while the additional therapeutic agent is administered via injection (for example, an intravenous, intramuscular and/or intratumoral injection).

In some embodiments, the therapeutic composition described herein is administered once a day. In some embodiments, the therapeutic composition described herein is administered twice a day. In some embodiments, the therapeutic composition described herein is formulated for a daily dose. In some embodiments, the therapeutic composition described herein is formulated for twice a day dose, wherein each dose is half of the daily dose.

In certain embodiments, the therapeutic compositions described herein are administered in conjunction with any other conventional anti-cancer treatment, such as, for example, radiation therapy and surgical resection of the tumor. These treatments may be applied as necessary and/or as indicated and may occur before, concurrent with or after administration of the therapeutic composition comprising a solution or dried form described herein.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, for example, tumor size, and other compounds such as drugs being administered concurrently or near-concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of a therapeutic composition comprising a solution or dried form described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent disease (for example, autoimmune disease, inflammatory disease, metabolic disease, or cancer), delay its onset, or slow or stop its progression, or relieve one or more symptoms of the disease. One skilled in the art will recognize that dosage will depend upon a variety of factors including the strength of the particular agent (for example, therapeutic agent) employed, as well as the age, species, condition, and body weight of the subject. The size of the dose will also be determined by the route, timing, and frequency of administration as well as the existence, nature, and extent of any adverse side-effects that might accompany the administration of a particular therapeutic agent and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting for example, with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the therapeutic agents used in accordance with the invention vary depending on the active agent, the age, weight, and clinical condition of the recipient patient, and the experience and judgment of the clinician or practitioner administering the therapy, among other factors affecting the selected dosage. For example, for cancer treatment, the dose should be sufficient to result in slowing, and preferably regressing, the growth of a tumor and most preferably causing complete regression of the cancer, or reduction in the size or number of metastases As another example, the dose should be sufficient to result in slowing of progression of the disease for which the subject is being treated, and preferably amelioration of one or more symptoms of the disease for which the subject is being treated.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a pharmaceutical composition, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month.

In some embodiments, the delivery of an additional therapeutic agent in combination with the therapeutic composition described herein reduces the adverse effects and/or improves the efficacy of the additional therapeutic agent.

The effective dose of an additional therapeutic agent described herein is the amount of the additional therapeutic agent that is effective to achieve the desired therapeutic response for a particular subject, composition, and mode of administration, with the least toxicity to the subject. The effective dosage level can be identified using the methods described herein and will depend upon a variety of pharmacokinetic factors including the activity of the particular compositions or agents administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts. In general, an effective dose of an additional therapeutic agent will be the amount of the additional therapeutic agent which is the lowest dose effective to produce a therapeutic effect. Such an effective dose will generally depend upon the factors described above.

The toxicity of an additional therapeutic agent is the level of adverse effects experienced by the subject during and following treatment. Adverse events associated with additional therapy toxicity can include, but are not limited to, abdominal pain, acid indigestion, acid reflux, allergic reactions, alopecia, anaphylaxis, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypematremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heartbeat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

### Immune Disorders

In some embodiments, the methods and therapeutic compositions described herein relate to the treatment or prevention of a disease or disorder associated a pathological immune response, such as an autoimmune disease, an allergic reaction and/or an inflammatory disease. In some embodiments, the disease or disorder is an inflammatory bowel disease (for example, Crohn's disease or ulcerative colitis). In some embodiments, the disease or disorder is psoriasis. In some embodiments, the disease or disorder is atopic dermatitis.

The methods described herein can be used to treat any subject in need thereof. As used herein, a “subject in need thereof” includes any subject that has a disease or disorder associated with a pathological immune response (for example, an inflammatory bowel disease), as well as any subject with an increased likelihood of acquiring a such a disease or disorder.

The therapeutic compositions described herein can be used, for example, as a pharmaceutical composition for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma; an infectious disease, such as an infection with Clostridium difficile; an inflammatory disease such as a TNF-mediated inflammatory disease (for example, an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a supplement, food, or beverage for improving immune functions; or a reagent for suppressing the proliferation or function of immune cells.

In some embodiments, the methods provided herein are useful for the treatment of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to a immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and pharmaceutical compositions described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, for example, inactive and active forms) and ulcerative colitis (for example, inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and pharmaceutical compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and therapeutic compositions described herein may be used to treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, Goodpasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and therapeutic compositions described herein may be used to treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dust mite allergy) and gluten-sensitive enteropathy (Celiac disease).

Other immune disorders which may be treated with the methods and therapeutic compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonoitis, pharyngitis, pleuritis, pneumonitis, prostatistis, pyelonephritis, and stomatisi, transplant rejection (involving organs such as kidney, liver, heart, lung, pancreas (for example, islet cells), bone marrow, comea, small bowel, skin allografts, skin homografts, and heart valve xenografts, serum sickness, and graft vs host disease), acute pancreatitis, chronic pancreatitis, acute respiratory distress syndrome, Sexary's syndrome, congenital adrenal hyperplasia, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (for example, sepsis).

### Metabolic Disorders

In some embodiments, the methods and therapeutic compositions described herein relate to the treatment or prevention of a metabolic disease or disorder a, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), nonalcoholic steatohepatitis (NASH) or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema. In some embodiments, the methods and pharmaceutical compositions described herein relate to the treatment of nonalcoholic fatty liver disease (NAFLD) and nonalcoholic steatohepatitis (NASH).

The methods described herein can be used to treat any subject in need thereof. As used herein, a “subject in need thereof” includes any subject that has a metabolic disease or disorder, as well as any subject with an increased likelihood of acquiring a such a disease or disorder.

The therapeutic compositions described herein can be used, for example, for preventing or treating (reducing, partially or completely, the adverse effects of) a metabolic disease, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), nonalcoholic steatohepatitis (NASH), or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema.

### Cancer

In some embodiments, the methods and therapeutic compositions described herein relate to the treatment of cancer. In some embodiments, any cancer can be treated using the methods described herein. Examples of cancers that may treated by methods and pharmaceutical compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the methods and pharmaceutical compositions provided herein relate to the treatment of a leukemia. Non-limiting examples of leukemia diseases include, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

In some embodiments, the methods and therapeutic compositions provided herein relate to the treatment of a carcinoma. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, Schneiderian carcinoma, scirrhous carcinoma, and carcinoma scroti.

In some embodiments, the methods and therapeutic compositions provided herein relate to the treatment of a sarcoma. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and therapeutic compositions described herein include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, plasmacytoma, colorectal cancer, rectal cancer, and adrenal cortical cancer.

In some embodiments, the cancer treated is a melanoma. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

In some embodiments, the cancer comprises breast cancer (for example, triple negative breast cancer).

In some embodiments, the cancer comprises colorectal cancer (for example, microsatellite stable (MSS) colorectal cancer).

In some embodiments, the cancer comprises renal cell carcinoma.

In some embodiments, the cancer comprises lung cancer (for example, non-small cell lung cancer).

In some embodiments, the cancer comprises bladder cancer.

In some embodiments, the cancer comprises gastroesophageal cancer.

Particular categories of tumors that can be treated using methods and therapeutic compositions described herein include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypemephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, plasmacytoma, colorectal cancer, and rectal cancer.

Cancers treated in certain embodiments also include precancerous lesions, for example, actinic keratosis (solar keratosis), moles (dysplastic nevi), acitinic chelitis (farmer's lip), cutaneous homs, Barrett's esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, for example, of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

### Other Diseases and Disorders

In some embodiments, the methods and therapeutic compositions described herein relate to the treatment of liver diseases. Such diseases include, but are not limited to, Alagille Syndrome, Alcohol-Related Liver Disease, Alpha-1 Antitrypsin Deficiency, Autoimmune Hepatitis, Benign Liver Tumors, Biliary Atresia, Cirrhosis, Galactosemia, Gilbert Syndrome, Hemochromatosis, Hepatitis A, Hepatitis B, Hepatitis C, Hepatic Encephalopathy, Intrahepatic Cholestasis of Pregnancy (ICP), Lysosomal Acid Lipase Deficiency (LAL-D), Liver Cysts, Liver Cancer, Newborn Jaundice, Primary Biliary Cholangitis (PBC), Primary Sclerosing Cholangitis (PSC), Reye Syndrome, Type I Glycogen Storage Disease, and Wilson Disease.

The methods and therapeutic compositions described herein may be used to treat neurodegenerative and neurological diseases. In certain embodiments, the neurodegenerative and/or neurological disease is Parkinson's disease, Alzheimer's disease, prion disease, Huntington's disease, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy or post-operative cognitive dysfunction.

### Dysbiosis

The gut microbiome (also called the “gut microbiota”) can have a significant impact on an individual's health through microbial activity and influence (local and/or distal) on immune and other cells of the host (Walker, W. A., Dysbiosis. The Microbiota in Gastrointestinal Pathophysiology. Chapter 25. 2017; Weiss and Thierry, Mechanisms and consequences of intestinal dysbiosis. Cellular and Molecular Life Sciences. (2017) 74(16):2959-2977. Zurich Open Repository and Archive, doi: doi.org/10.1007/s00018-017-2509-x)).

A healthy host-gut microbiome homeostasis is sometimes referred to as a “eubiosis” or “normobiosis,” whereas a detrimental change in the host microbiome composition and/or its diversity can lead to an unhealthy imbalance in the microbiome, or a “dysbiosis” (Hooks and O'Malley. Dysbiosis and its discontents. American Society for Microbiology. October 2017. Vol. 8. Issue 5. mBio 8:e01492-17. https://doi.org/10.1128/mBio.01492-17). Dysbiosis, and associated local or distal host inflammatory or immune effects, may occur where microbiome homeostasis is lost or diminished, resulting in: increased susceptibility to pathogens; altered host bacterial metabolic activity; induction of host proinflammatory activity and/or reduction of host anti-inflammatory activity. Such effects are mediated in part by interactions between host immune cells (for example, T cells, dendritic cells, mast cells, NK cells, intestinal epithelial lymphocytes (IEC), macrophages and phagocytes) and cytokines, and other substances released by such cells and other host cells.

A dysbiosis may occur within the gastrointestinal tract (a “gastrointestinal dysbiosis” or “gut dysbiosis”) or may occur outside the lumen of the gastrointestinal tract (a “distal dysbiosis”). Gastrointestinal dysbiosis is often associated with a reduction in integrity of the intestinal epithelial barrier, reduced tight junction integrity and increased intestinal permeability. Citi, S. Intestinal Barriers protect against disease, Science 359:1098-99 (2018); Srinivasan et al., TEER measurement techniques for in vitro barrier model systems. J. Lab. Autom. 20:107-126 (2015). A gastrointestinal dysbiosis can have physiological and immune effects within and outside the gastrointestinal tract.

The presence of a dysbiosis can be associated with a wide variety of diseases and conditions including: infection, cancer, autoimmune disorders (for example, systemic lupus erythematosus (SLE)) or inflammatory disorders (for example, functional gastrointestinal disorders such as inflammatory bowel disease (IBD), ulcerative colitis, and Crohn's disease), neuroinflammatory diseases (for example, multiple sclerosis), transplant disorders (for example, graft-versus-host disease), fatty liver disease, type I diabetes, rheumatoid arthritis, Sjögren's syndrome, celiac disease, cystic fibrosis, chronic obstructive pulmonary disorder (COPD), and other diseases and conditions associated with immune dysfunction. Lynch et al., The Human Microbiome in Health and Disease, N. Engl. J. Med 0.375:2369-79 (2016), Carding et al., Dysbiosis of the gut microbiota in disease. Microb. Ecol. Health Dis. (2015); 26: 10: 3402/mehd.v26.2619; Levy et al, Dysbiosis and the Immune System, Nature Reviews Immunology 17:219 (April 2017)

In certain embodiments, exemplary therapeutic compositions disclosed herein can treat a dysbiosis and its effects by modifying the immune activity present at the site of dysbiosis. As described herein, such compositions can modify a dysbiosis via effects on host immune cells, resulting in, for example, an effect on secretion of cytokines, reducing inflammation in the subject recipient or via changes in metabolite production.

Exemplary therapeutic compositions disclosed herein that are useful for treatment of disorders associated with a dysbiosis contain one or more types of EVs derived from immunomodulatory bacteria. Such compositions are capable of affecting the recipient host's immune function, in the gastrointestinal tract, and/or a systemic effect at distal sites outside the subject's gastrointestinal tract.

Exemplary therapeutic compositions disclosed herein that are useful for treatment of disorders associated with a dysbiosis contain a population of immunomodulatory bacteria of a single bacterial species (for example, a single strain) and/or a population of EVs derived from immunomodulatory bacteria of a single bacterial species (for example, a single strain). Such compositions are capable of affecting the recipient host's immune function, in the gastrointestinal tract, and/or a systemic effect at distal sites outside the subject's gastrointestinal tract.

In one embodiment, therapeutic compositions containing an isolated population of EVs derived from immunomodulatory bacteria are administered (for example, orally) to a mammalian recipient in an amount effective to treat a dysbiosis and one or more of its effects in the recipient. The dysbiosis may be a gastrointestinal tract dysbiosis or a distal dysbiosis.

In some embodiments, therapeutic compositions of the instant invention can treat a gastrointestinal dysbiosis and one or more of its effects on host immune cells, resulting in an effect on cytokines secretion, reducing inflammation in the subject recipient.

In some embodiments, the therapeutic compositions can treat a gastrointestinal dysbiosis and one or more of its effects by modulating the recipient immune response via cellular and cytokine modulation to reduce gut permeability by increasing the integrity of the intestinal epithelial barrier.

In some embodiments, the therapeutic compositions can treat a distal dysbiosis and one or more of its effects by modulating the recipient immune response at the site of dysbiosis via modulation of host immune cells.

Other exemplary therapeutic compositions are useful for treatment of disorders associated with a dysbiosis, which compositions contain one or more types of bacteria and/or EVs capable of altering the relative proportions of host immune cell subpopulations, for example, subpopulations of T cells, immune lymphoid cells, dendritic cells, NK cells and other immune cells, or the function thereof, in the recipient.

Other exemplary therapeutic compositions are useful for treatment of disorders associated with a dysbiosis, which compositions contain a population of EVs of a single immunomodulatory bacterial species (for example, a single strain) capable of altering the relative proportions of immune cell subpopulations, for example, T cell subpopulations, immune lymphoid cells, NK cells and other immune cells, or the function thereof, in the recipient subject.

In one embodiment, the invention provides methods of treating a gastrointestinal dysbiosis and one or more of its effects by orally administering to a subject in need thereof a therapeutic composition which alters the microbiome population existing at the site of the dysbiosis. The therapeutic composition can contain one or more types of EVs from immunomodulatory bacteria or a population of EVs of a single immunomodulatory bacterial species (for example, a single strain).

In one embodiment, the invention provides methods of treating a distal dysbiosis and one or more of its effects by orally administering to a subject in need thereof a therapeutic composition which alters the subject's immune response outside the gastrointestinal tract. The therapeutic composition can contain one or more types of EVs from immunomodulatory bacteria or a population of EVs of a single immunomodulatory bacterial species (for example, a single strain).

In exemplary embodiments, therapeutic compositions useful for treatment of disorders associated with a dysbiosis stimulate secretion of one or more anti-inflammatory cytokines by host immune cells. Anti-inflammatory cytokines include, but are not limited to, IL-10, IL-13, IL-9, IL-4, IL-5, TGFβ, and combinations thereof. In other exemplary embodiments, pharmaceutical compositions useful for treatment of disorders associated with a dysbiosis that decrease (for example, inhibit) secretion of one or more pro-inflammatory cytokines by host immune cells. Pro-inflammatory cytokines include, but are not limited to, IFNγ, IL-12p70, IL-1α, IL-6, IL-8, MCP1, MIP1α, MIP1β, TNFα, and combinations thereof. Other exemplary cytokines are known in the art and are described herein.

In another aspect, the invention provides a method of treating or preventing a disorder associated with a dysbiosis in a subject in need thereof, comprising administering (for example, orally administering) to the subject a therapeutic composition in the form of a probiotic or medical food comprising bacteria or EVs in an amount sufficient to alter the microbiome at a site of the dysbiosis, such that the disorder associated with the dysbiosis is treated.

In some embodiments, a therapeutic composition of the instant invention in the form of a probiotic or medical food may be used to prevent or delay the onset of a dysbiosis in a subject at risk for developing a dysbiosis.

### Methods of Making Enhanced Bacteria

In certain aspects, provided herein are methods of making engineered bacteria for the production of the EVs described herein. In some embodiments, the engineered bacteria are modified to enhance certain desirable properties. For example, in some embodiments, the engineered bacteria are modified to enhance the immunomodulatory and/or therapeutic effect of the EVs (for example, either alone or in combination with another therapeutic agent), to reduce toxicity and/or to improve bacterial and/or EV manufacturing (for example, higher oxygen tolerance, improved freeze-thaw tolerance, shorter generation times). The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, CRISPR/Cas9, or any combination thereof.

In some embodiments of the methods provided herein, the bacterium is modified by directed evolution. In some embodiments, the directed evolution comprises exposure of the bacterium to an environmental condition and selection of bacterium with improved survival and/or growth under the environmental condition. In some embodiments, the method comprises a screen of mutagenized bacteria using an assay that identifies enhanced bacterium. In some embodiments, the method further comprises mutagenizing the bacteria (for example, by exposure to chemical mutagens and/or UV radiation) or exposing them to a therapeutic agent (for example, antibiotic) followed by an assay to detect bacteria having the desired phenotype (for example, an in vivo assay, an ex vivo assay, or an in vitro assay).

### EXAMPLES

### Example 1: Preparation of Lyophilates

The excipient stocks with the formulas provided in Table A to Table D were prepared (amounts shown are percentages of each component in the formula) as solutions. The formulas of the excipient stock fall in to 2 main categories: with and without polymers. The excipient stock solutions were mixed with a liquid preparation of extracellular vesicles. The resulting solutions were freeze-dried and analyzed.

In this Example, the extracellular vesicles (EVs) used in the studies were isolated from a strain of Prevotella histicola.

The data collected from the lyophilization of these mixtures is provided in Table E. All measured samples had a residual moisture content of less than 5%. Some samples were additionally tested in vivo using keyhole limpet hemocyanin (KLH)-specific inflammation in a delayed-type hypersensitivity (DTH) model. The samples tested in KLH-DTH showed efficacy.

TABLE A
Stocks comprising excipients for stabilizing extracellular
vesicles during lyophilization. The numerical values
given are on a weight percent basis in the solution.

| | | | | | | Malto- |
|---|---|---|---|---|---|---|
| Formula | Sucrose | Trehalose | Mannitol | Sorbitol | Dextran | dextrin |
| 1 | 40 | 15 | 20 | | 25 | |
| 2 | 20 | 20 | 50 | | 10 | |
| 3 | 50 | | 50 | | | |
| 4 | 40 | 10 | 50 | | | |
| 5 | | 10 | 70 | 0.5 | 19.5 | |
| 7 | | 19.5 | 80 | 0.5 | | |
| 7a | | 20 | 80 | | | |
| 7e | 27 | 20 | 53 | | | |
| 8 | | 10 | 75 | | 15 | |
| 15 | | 19.5 | 70 | 0.5 | 10 | |
| 16 | | 19.5 | 75 | 0.5 | 5 | |
| 17 | | 20 | 80 | | | |
| 18 | | 10 | 60 | | 30 | |
| 19 | | 10 | | | 30 | 60 |
| 20 | | | 100 | | | |

TABLE B
Stocks comprising excipients including polymers for stabilizing
extracellular vesicles during lyophilization.

| Formula | Sucrose | PVP-K30 | Ficoll | Citrate | Arginine |
|---|---|---|---|---|---|
| 6 | 20 | 78 | | 1 | 1 |
| 14 | 20 | | 78 | 1 | 1 |

The numerical values given are on a weight percent basis in the solution.

TABLE C
Stocks comprising excipients including polymers for stabilizing
extracellular vesicles during lyophilization. The numerical values
given are on a weight percent basis in the solution.

| | | | | PVP- | Hydroxypropyl- | |
|---|---|---|---|---|---|---|
| Formula | Sucrose | Trehalose | Mannitol | K30 | B-cyclodextrin | Ficoll |
| 9 | 20 | 10 | 50 | 20 | | |
| 10 | 10 | 10 | 50 | | 30 | |
| 13 | 20 | 10 | 50 | | | 20 |

TABLE D
Stocks comprising excipients including polymers for
stabilizing extracellular vesicles during lyophilization.
The numerical values given
are on a weight percent basis in the solution.

| | | Poloxamer |
|---|---|---|
| Formula | Mannitol | 188 |
| 11 | 95 | 5 |
| 12 | 90 | 10 |

TABLE E
Analytical data obtained for excipient stock solutions
used for the stabilization of extracellular vesicles.

| | % | % | Zave, | Particles per |
|---|---|---|---|---|
| Formula | Stabilizer | Moisture | nm | mass, p/mg |
| — | 0% | | 226.1 | 6.45E+11 |
| 4 | 34% | 2 | 206.2 | 6.28E+10 |
| 5 | 41% | | 209.1 | 6.76E+10 |
| 6 | 35% | 3.6 | 212.8 | 3.25E+10 |
| 7 | 47% | 2.7 | 204 | 7.02E+10 |
| 8 | 44% | 3 | 206.4 | 6.99E+10 |
| 9 | 34% | 2.5 | 187.3 | 7.15E+10 |
| 10 | 34% | 2.7 | 180.1 | 7.37E+10 |
| 11 | 56% | 1.8 | 205.2 | 7.08E+10 |
| 12 | 53% | 1.8 | 202 | 7.66E+10 |
| 13 | 30% | 3 | 172.3 | 7.77E+10 |
| 14 | 35% | 3.8 | 137.4 | 6.12E+10 |
| 15 | 41% | 2.9 | 205.8 | |
| 16 | 44% | 2.8 | 203.9 | |

“% Stabilizer” refers to the percentage of the stock solution formula that was added to a liquid preparation of EVs on a weight basis. “% Moisture” was determined by Karl Fischer titration. Zₐᵥₑ was determined by dynamic light scattering (DLS). For particles per mass, particle numbers were determined by Z-view or NTA instrument; mass (mg) were decided by analytical balance.

### Lyophilization Cycle for Extracellular Vesicles (EVs)

The lyophilization cycle is optimized for each excipient formulation. Differences in the critical temperature and collapse temperature of the mixtures mean the shelf temperature during lyophilization is adjusted accordingly. The optimization process involves 3 steps: initial screening, primary drying optimization, and secondary drying optimization. The final cycle is confirmed to be sufficient to dry the material below 5% residual moisture. In this example, the excipient formula chosen for optimization was excipient formula 7.

TABLE F
FORMULA 7 @ 300 MILLITORR

| SHELF TEMP (° C.) | SAMPLE TEMP (° C.) |
|---|---|
| −5 | −17.9 |
| −15 | −23.6 |
| −20 | −26.3 |
| −25 | −28.9 |

TABLE G

| Shelf Temp. | % | Primary Drying | Zₐᵥₑ |
|---|---|---|---|
| (° C.) | Moisture | (hrs) | (nm) |
| −25 | 2.9 | 31.5 | 189 |
| −20 | 2.8 | 28.2 | 215 |
| −15 | 3.3 | 20.8 | 224 |
| −5 | 1.5 | 18.2 | 202 |

TABLE H

| | % | Secondary Drying |
|---|---|---|
| Formula #7 | Moisture | Time (hrs) |
| Primary Drying | 2.8 | 2 |
| Secondary Drying | 2.6 | 29 |

TABLE I
Final lyophilization cycle optimized for extracellular vesicles
stabilized with 47% (by volume) of excipient formula 7.

| | Shelf Temp. | Ramp Time | Hold Time | Vacuum |
|---|---|---|---|---|
| Step | (° C.) | (min) | (min) | (mTorr) |
| Freezing (from RT) | −45 | 200 | 10 | 600K |
| Primary Drying | −20 | 75 | 2,151 | 300 |
| Secondary Drying | 25 | 126 | 300 | 300 |
| Hold | 25 | 0 | 0-300 | 300 |

### Example 2: Representative Strains as Sources for EVs

Extracellular vesicles (EVs) were isolated from the strains listed in Table J. Information on the Gram staining, cell wall structure, and taxonomic classification for each strain is also provided in Table J. EVs can be prepared or isolated from any of these strains to prepare a solution and/or dried form described herein.

TABLE J
Strains from which extracellular vesicles (EVs) were isolated.

| | | Cell | | | | |
|---|---|---|---|---|---|---|
| | | envelope | | | | |
| Strain | Gram-stain | structure | Phylum | Class | Order | Family |
| Parabacteroides distasonis | Gram-stain- | diderm | Bacteroidota | Bacteroidia | Bacteroidales | Porphyromonadaceae |
| DRLU022118 A ILEUM-6 | negative | | | | | |
| Parabacteroides goldsteinii S4 | Gram-stain- | diderm | Bacteroidota | Bacteroidia | Bacteroidales | Porphyromonadaceae |
| | negative | | | | | |
| Prevotella histicola | Gram-stain- | diderm | Bacteroidota | Bacteroidia | Bacteroidales | Prevotellaceae |
| | negative | | | | | |
| Prevotella histicola | Gram-stain- | diderm | Bacteroidota | Bacteroidia | Bacteroidales | Prevotellaceae |
| | negative | | | | | |
| Fournierella massiliensis S10 | Gram-stain- | monoderm | Firmicutes | Clostridia | Eubacteriales | Oscillospiraceae |
| GIMucosa-297 | negative | | | | | (formely |
| | | | | | | Ruminococcaceae) |
| Harryflintia acetispora S4-M5 | Gram-stain- | monoderm | Firmicutes | Clostridia | Eubacteriales | Oscillospiraceae |
| | negative | | | | | |
| Blautia massiliensis S1046-4A5 | Gram-stain- | monoderm | Firmicutes | Clostridia | Eubacteriales | Lachnospiraceae |
| | negative | | | | | |
| Mediterraneibacter/[Ruminococcus] | Gram-stain- | monoderm | Firmicutes | Clostridia | Eubacteriales | Lachnospiraceae |
| gnavus S10 GIMucosa-412 | negative | | | | | |
| Clostridioides difficile S10 | Gram-stain- | monoderm | Firmicutes | Clostridia | Eubacteriales | Peptostreptococcaceae |
| GImucosa-525 | positive | | | | | |
| Aminipila sp. S16-M4 | Gram-stain- | monoderm | Firmicutes | Clostridia | Eubacteriales | Clostridiales Family |
| | positive | | | | | XIII/Incertae sedis |
| | | | | | | 41/[Eubacteriales, no |
| | | | | | | family] |
| Megasphaera sp. S29-N3 | Gram-stain- | diderm | Firmicutes | Negativicutes | Veillonellales | Veillonellaceae |
| | negative | | | | | |
| Megasphaera sp. S1007 | Gram-stain- | diderm | Firmicutes | Negativicutes | Veillonellales | Veillonellaceae |
| | negative | | | | | |
| Selenomonas felix S34N-300R | Gram-stain- | diderm | Firmicutes | Negativicutes | Selenomonadales | Selenomonadaceae |
| | negative | | | | | |
| Veillonella parvula S14Ileum-201 | Gram-stain- | diderm | Firmicutes | Negativicutes | Veillonellales | Veillonellaceae |
| | negative | | | | | |
| Propionispora sp. DSM100705-1A | Gram-stain- | diderm | Firmicutes | Negativicutes | Selenomonadales | Sporomusaceae |
| | negative | | | | | |
| Rarimicrobium hominis | Gram-stain- | diderm | Synergistota | Synergistia | Synergistales | Synergistaceae |
| S24RS2-T2-5 | negative | | | | | |
| Cloacibacillus evryensis S29-M8 | Gram-stain- | diderm | Synergistota | Synergistia | Synergistales | Synergistaceae |
| | negative | | | | | |
| Veillonella parvula S14-205 | Gram-stain- | diderm | Firmicutes | Negativicutes | Veillonellales | Veillonellaceae |
| | negative | | | | | |
| Veillonella sp/dispar | Gram-stain- | diderm | Firmicutes | Negativicutes | Veillonellales | Veillonellaceae |
| ECD01-DP-201 | negative | | | | | |
| Veillonella parvula/dispar | Gram-stain- | diderm | Firmicutes | Negativicutes | Veillonellales | Veillonellaceae |
| ECD01-DP-223 | negative | | | | | |
| Veillonella parvula | Gram-stain- | diderm | Firmicutes | Negativicutes | Veillonellales | Veillonellaceae |
| S16 GIMucosa-95 | negative | | | | | |

### Example 3: Purification and Preparation of Extracellular Vesicles (EVs) from Bacteria Purification

Extracellular vesicles (such as smEVs) are purified and prepared from bacterial cultures using methods known to those skilled in the art (S. Bin Park, et al. PLoS ONE. 6(3):e17629 (2011)).

For example, bacterial cultures are centrifuged at 10,000-15,500×g for 10-40 min at 4° C., or room temperature to pellet bacteria. Culture supernatants are then filtered to include material ≤0.22 μm (for example, via a 0.22 μm or 0.45 μm filter) and to exclude intact bacterial cells. Filtered supernatants are concentrated using methods that may include, but are not limited to, ammonium sulfate precipitation, ultracentrifugation, or filtration. Briefly, for ammonium sulfate precipitation, 1.5-3 M ammonium sulfate is added to filtered supernatant slowly, while stirring at 4° C. Precipitations are incubated at 4° C. for 8-48 hours and then centrifuged at 11,000×g for 20-40 min at 4° C. The pellets contain EVs and other debris. Briefly, using ultracentrifugation, filtered supernatants are centrifuged at 100,000-200,000×g for 1-16 hours at 4° C. The pellet of this centrifugation contains EVs and other debris. Briefly, using a filtration technique, using an Amicon Ultra spin filter or by tangential flow filtration, supernatants are filtered so as to retain species of molecular weight >50, 100, 300, or 500 kDa.

Alternatively, EVs are obtained from bacterial cultures continuously during growth, or at selected time points during growth, by connecting a bioreactor to an alternating tangential flow (ATF) system (for example, XCell ATF from Repligen) according to manufacturer's instructions. The ATF system retains intact cells (>0.22 μm) in the bioreactor, and allows smaller components (for example, EVs, free proteins) to pass through a filter for collection. For example, the system may be configured so that the <0.22 μm filtrate is then passed through a second filter of 100 kDa, allowing species such as EVs between 0.22 μm and 100 kDa to be collected, and species smaller than 100 kDa to be pumped back into the bioreactor. Alternatively, the system may be configured to allow for medium in the bioreactor to be replenished and/or modified during growth of the culture. EVs collected by this method may be further purified and/or concentrated by ultracentrifugation or filtration as described above for filtered supernatants.

EVs obtained by methods described above may be further purified by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in 45% Optiprep in PBS. If filtration was used to concentrate the filtered supernatant, the concentrate is diluted using 60% Optiprep to a final concentration of 45% Optiprep. Samples are applied to a 0-45% discontinuous sucrose gradient and centrifuged at 200,000×g for 3-24 hours at 4° C. Alternatively, high resolution density gradient fractionation could be used to separate EVs based on density.

### Preparation

To confirm sterility and isolation of the EV preparations, EVs are serially diluted onto agar medium used for routine culture of the bacteria being tested and incubated using routine conditions. Non-sterile preparations are passed through a 0.22 μm filter to exclude intact cells. To further increase purity, isolated EVs may be DNase or proteinase K treated.

Alternatively, for preparation of EVs used for in vivo injections, purified EVs are processed as described previously (G. Norheim, et al. PLoS ONE. 10(9): e0134353 (2015)). Briefly, after sucrose gradient centrifugation, bands containing EVs are resuspended to a final concentration of 50 μg/mL in a solution containing 3% sucrose or other solution suitable for in vivo injection known to one skilled in the art. This solution may also contain adjuvant, for example aluminum hydroxide at a concentration of 0-0.5% (w/v).

To make samples compatible with further testing (for example, to remove sucrose prior to TEM imaging or in vitro assays), samples are buffer exchanged into PBS or 30 mM Tris, pH 8.0 using filtration (for example, Amicon Ultra columns), dialysis, or ultracentrifugation (following 15-fold or greater dilution in PBS, 200,000×g, 1-3 hours, 4° C.) and resuspension in PBS.

For all of these studies, EVs may be heated, irradiated, and/or lyophilized prior to administration (as described herein).

### Example 4: Manipulating Bacteria Through Stress to Produce Various Amounts of EVs and/or to Vary Content of EVs

Stress, and in particular envelope stress, has been shown to increase production of EVs (such as smEVs) by some bacterial strains (I. MacDonald, M. Kuehn. J Bacteriol 195(13): doi: 10/1128/JB.02267-12). In order to vary production of EVs by bacteria, bacteria are stressed using various methods.

Bacteria may be subjected to single stressors or stressors in combination. The effects of different stressors on different bacteria is determined empirically by varying the stress condition and determining the IC50 value (the conditions required to inhibit cell growth by 50%). EV purification, quantification, and characterization occurs. EV production is quantified (1) in complex samples of bacteria and EVs by nanoparticle tracking analysis (NTA) or transmission electron microscopy (TEM); or (2) following EV purification by NTA, lipid quantification, or protein quantification. EV content is assessed following purification by methods described above.

### Antibiotic Stress

Bacteria are cultivated under standard growth conditions with the addition of sublethal concentrations of antibiotics. This may include 0.1-1 μg/mL chloramphenicol, or 0.1-0.3 μg/mL gentamicin, or similar concentrations of other antibiotics (for example, ampicillin, polymyxin B). Host antimicrobial products such as lysozyme, defensins, and Reg proteins may be used in place of antibiotics. Bacterially-produced antimicrobial peptides, including bacteriocins and microcins may also be used.

### Temperature Stress

Bacteria are cultivated under standard growth conditions, but at higher or lower temperatures than are typical for their growth. Alternatively, bacteria are grown under standard conditions, and then subjected to cold shock or heat shock by incubation for a short period of time at low or high temperatures respectively. For example, bacteria grown at 37° C. are incubated for 1 hour at 4° C.-18° C. for cold shock or 42° C.-50° C. for heat shock.

### Starvation and Nutrient Limitation

To induce nutritional stress, bacteria are cultivated under conditions where one or more nutrients are limited. Bacteria may be subjected to nutritional stress throughout growth or shifted from a rich medium to a poor medium. Some examples of media components that are limited are carbon, nitrogen, iron, and sulfur. An example medium is M9 minimal medium (Sigma-Aldrich), which contains low glucose as the sole carbon source. Particularly for Prevotella spp., iron availability is varied by altering the concentration of hemin in media and/or by varying the type of porphyrin or other iron carrier present in the media, as cells grown in low hemin conditions were found to produce greater numbers of EVs (S. Stubbs et al. Letters in Applied Microbiology. 29:31-36 (1999). Media components are also manipulated by the addition of chelators such as EDTA and deferoxamine.

### Saturation

Bacteria are grown to saturation and incubated past the saturation point for various periods of time. Alternatively, conditioned media is used to mimic saturating environments during exponential growth. Conditioned media is prepared by removing intact cells from saturated cultures by centrifugation and filtration, and conditioned media may be further treated to concentrate or remove specific components.

### Salt Stress

Bacteria are cultivated in or exposed for brief periods to medium containing NaCl, bile salts, or other salts.

### UV Stress

UV stress is achieved by cultivating bacteria under a UV lamp or by exposing bacteria to UV using an instrument such as a Stratalinker (Agilent). UV may be administered throughout the entire cultivation period, in short bursts, or for a single defined period following growth.

### Reactive Oxygen Stress

Bacteria are cultivated in the presence of sublethal concentrations of hydrogen peroxide (250-1,000 μM) to induce stress in the form of reactive oxygen species. Anaerobic bacteria are cultivated in or exposed to concentrations of oxygen that are toxic to them.

### Detergent Stress

Bacteria are cultivated in or exposed to detergent, such as sodium dodecyl sulfate (SDS) or deoxycholate.

### pH Stress

Bacteria are cultivated in or exposed for limited times to media of different pH.

### Example 5: Profiling EV Composition and Content

EVs may be characterized by any one of various methods including, but not limited to, NanoSight characterization, SDS-PAGE gel electrophoresis, Western blot, ELISA, liquid chromatography-mass spectrometry and mass spectrometry, dynamic light scattering, lipid levels, total protein, lipid to protein ratios, nucleic acid analysis and/or zeta potential.

### NanoSight Characterization of EVs

Nanoparticle tracking analysis (NTA) is used to characterize the size distribution of purified EVs. Purified EV preparations are run on a NanoSight machine (Malvern Instruments) to assess EV size and concentration.

### SDS-PAGE Gel Electrophoresis

To identify the protein components of purified EVs, samples are run on a gel, for example a Bolt Bis-Tris Plus 4-12% gel (ThermoFisher Scientific), using standard techniques. Samples are boiled in 1×SDS sample buffer for 10 minutes, cooled to 4° C., and then centrifuged at 16,000×g for 1 min. Samples are then run on a SDS-PAGE gel and stained using one of several standard techniques (for example, Silver staining, Coomassie Blue, Gel Code Blue) for visualization of bands.

### Western Blot Analysis

To identify and quantify specific protein components of purified EVs, EV proteins are separated by SDS-PAGE as described above and subjected to Western blot analysis (Cvjetkovic et al., Sci. Rep. 6, 36338 (2016)) and are quantified via ELISA.

### EV Proteomics and Liquid Chromatography-Mass Spectrometry (LC-MSMS) and Mass Spectrometry (MS)

Proteins present in EVs are identified and quantified by Mass Spectrometry techniques. EV proteins may be prepared for LC-MS/MS using standard techniques including protein reduction using dithiotreitol solution (DTI) and protein digestion using enzymes such as LysC and trypsin as described in Erickson et al, 2017 (Molecular Cell, VOLUME 65, ISSUE 2, P361-370, Jan. 19, 2017). Alternatively, peptides are prepared as described by Liu et al. 2010 (JOURNAL OF BACTERIOLOGY, June 2010, p. 2852-2860 Vol. 192, No. 11), Kieselbach and Oscarsson 2017 (Data Brief. 2017 February; 10: 426-431), Vildhede et al, 2018 (Drug Metabolism and Disposition Feb. 8, 2018). Following digestion, peptide preparations are run directly on liquid chromatography and mass spectrometry devices for protein identification within a single sample. For relative quantitation of proteins between samples, peptide digests from different samples are labeled with isobaric tags using the iTRAQ Reagent-8plex Multiplex Kit (Applied Biosystems, Foster City, CA) or TMT 10plex and 11plex Label Reagents (Thermo Fischer Scientific, San Jose, CA, USA). Each peptide digest is labeled with a different isobaric tag and then the labeled digests are combined into one sample mixture. The combined peptide mixture is analyzed by LC-MS/MS for both identification and quantification. A database search is performed using the LC-MS/MS data to identify the labeled peptides and the corresponding proteins. In the case of isobaric labeling, the fragmentation of the attached tag generates a low molecular mass reporter ion that is used to obtain a relative quantitation of the peptides and proteins present in each EV.

Additionally, metabolic content is ascertained using liquid chromatography techniques combined with mass spectrometry. A variety of techniques exist to determine metabolomic content of various samples and are known to one skilled in the art involving solvent extraction, chromatographic separation and a variety of ionization techniques coupled to mass determination (Roberts et al 2012 Targeted Metabolomics. Curr Protoc Mol Biol. 30: 1-24; Dettmer et al 2007, Mass spectrometry-based metabolomics. Mass Spectrom Rev. 26(1):51-78). As a non-limiting example, a LC-MS system includes a 4000 QTRAP triple quadrupole mass spectrometer (AB SCIEX) combined with 1100 Series pump (Agilent) and an HTS PAL autosampler (Leap Technologies). Media samples or other complex metabolic mixtures (˜10 μL) are extracted using nine volumes of 74.9:24.9:0.2 (v/v/v) acetonitrile/methanol/formic acid containing stable isotope-labeled internal standards (valine-d8, Isotec; and phenylalanine-d8, Cambridge Isotope Laboratories). Standards may be adjusted or modified depending on the metabolites of interest. The samples are centrifuged (10 minutes, 9,000 g, 4° C.), and the supernatants (10 μL) are submitted to LCMS by injecting the solution onto the HILIC column (150×2.1 mm, 3 μm particle size). The column is eluted by flowing a 5% mobile phase [10 mM ammonium formate, 0.1% formic acid in water] for 1 minute at a rate of 250 μL/minute followed by a linear gradient over 10 minutes to a solution of 40% mobile phase [acetonitrile with 0.1% formic acid]. The ion spray voltage is set to 4.5 kV and the source temperature is 450° C.

The data are analyzed using commercially available software like Multiquant 1.2 from AB SCIEX for mass spectrum peak integration. Peaks of interest should be manually curated and compared to standards to confirm the identity of the peak. Quantitation with appropriate standards is performed to determine the number of metabolites present in the initial media, after bacterial conditioning and after tumor cell growth. A non-targeted metabolomics approach may also be used using metabolite databases, such as but not limited to the NIST database, for peak identification.

### Dynamic Light Scattering (DLS)

DLS measurements, including the distribution of particles of different sizes in different EV preparations are taken using instruments such as the DynaPro NanoStar (Wyatt Technology) and the Zetasizer Nano ZS (Malvern Instruments).

### Lipid Levels

Lipid levels are quantified using FM4-64 (Life Technologies), by methods similar to those described by A. J. McBroom et al. J Bacteriol 188:5385-5392. and A. Frias, et al. Microb Ecol. 59:476-486 (2010). Samples are incubated with FM4-64 (3.3 μg/mL in PBS for 10 minutes at 37° C. in the dark). After excitation at 515 nm, emission at 635 nm is measured using a Spectramax M5 plate reader (Molecular Devices). Absolute concentrations are determined by comparison of unknown samples to standards (such as palmitoyloleoylphosphatidylglycerol (POPG) vesicles) of known concentrations. Lipidomics can be used to identify the lipids present in the EVs.

### Total Protein

Protein levels are quantified by standard assays such as the Bradford and BCA assays. The Bradford assays are run using Quick Start Bradford 1× Dye Reagent (Bio-Rad), according to manufacturer's protocols. BCA assays are run using the Pierce BCA Protein Assay Kit (Thermo-Fisher Scientific). Absolute concentrations are determined by comparison to a standard curve generated from BSA of known concentrations. Alternatively, protein concentration can be calculated using the Beer-Lambert equation using the sample absorbance at 280 nm (A280) as measured on a Nanodrop spectrophotometer (Thermo-Fisher Scientific). In addition, proteomics may be used to identify proteins in the sample.

### Lipid:Protein Ratios

Lipid:protein ratios are generated by dividing lipid concentrations by protein concentrations. These provide a measure of the purity of vesicles as compared to free protein in each preparation.

### Nucleic Acid Analysis

Nucleic acids are extracted from EVs and quantified using a Qubit fluorometer. Size distribution is assessed using a BioAnalyzer and the material is sequenced.

### Zeta Potential

The zeta potential of different preparations are measured using instruments such as the Zetasizer ZS (Malvern Instruments).

### Example 6: Manufacturing Conditions

Enriched media is used to grow and prepare the bacteria for in vitro and in vivo use and, ultimately, for EV preparations. For example, media may contain sugar, yeast extracts, plant-based peptones, buffers, salts, trace elements, surfactants, anti-foaming agents, and vitamins. Composition of complex components such as yeast extracts and peptones may be undefined or partially defined (including approximate concentrations of amino acids, sugars etc.). Microbial metabolism may be dependent on the availability of resources such as carbon and nitrogen. Various sugars or other carbon sources may be tested. Alternatively, media may be prepared and the selected bacterium grown as shown by Saarela et al., J. Applied Microbiology. 2005. 99: 1330-1339, which is hereby incorporated by reference. Influence of fermentation time, cryoprotectant and neutralization of cell concentrate on freeze-drying survival, storage stability, and acid and bile exposure of the selected bacterium produced without milk-based ingredients.

At large scale, the media is sterilized. Sterilization may be accomplished by Ultra High Temperature (UHT) processing. The UHT processing is performed at very high temperature for short periods of time. The UHT range may be from 135-180° C. For example, the medium may be sterilized from between 10 to 30 seconds at 135° C.

Inoculum can be prepared in flasks or in smaller bioreactors and growth is monitored. For example, the inoculum size may be between approximately 0.5 and 3% of the total bioreactor volume. Depending on the application and need for material, bioreactor volume can be at least 2 L, 10 L, 80 L, 100 L, 250 L, 1000 L, 2500 L, 5000 L, 10,000 L.

Before the inoculation, the bioreactor is prepared with medium at desired pH, temperature, and oxygen concentration. The initial pH of the culture medium may be different that the process set-point. pH stress may be detrimental at low cell centration; the initial pH could be between pH 7.5 and the process set-point. For example, pH may be set between 4.5 and 8.0. During the fermentation, the pH can be controlled through the use of sodium hydroxide, potassium hydroxide, or ammonium hydroxide. The temperature may be controlled from 25° C. to 45° C., for example at 37° C. Anaerobic conditions are created by reducing the level of oxygen in the culture broth from around 8 mg/L to 0 mg/L. For example, nitrogen or gas mixtures (N₂, CO₂, and H₂) may be used in order to establish anaerobic conditions. Alternatively, no gases are used and anaerobic conditions are established by cells consuming remaining oxygen from the medium. Depending on strain and inoculum size, the bioreactor fermentation time can vary. For example, fermentation time can vary from approximately 5 hours to 48 hours.

Reviving bacteria from a frozen state may require special considerations. Production medium may stress cells after a thaw; a specific thaw medium may be required to consistently start a seed train from thawed material. The kinetics of transfer or passage of seed material to fresh medium, for the purposes of increasing the seed volume or maintaining the microbial growth state, may be influenced by the current state of the bacteria (ex. exponential growth, stationary growth, unstressed, stressed).

Inoculation of the production fermenter(s) can impact growth kinetics and cellular activity. The initial state of the bioreactor system must be optimized to facilitate successful and consistent production. The fraction of seed culture to total medium (for example, a percentage) has a dramatic impact on growth kinetics. The range may be 1-5% of the fermenter's working volume. The initial pH of the culture medium may be different from the process set-point. pH stress may be detrimental at low cell concentration; the initial pH may be between pH 7.5 and the process set-point. Agitation and gas flow into the system during inoculation may be different from the process set-points. Physical and chemical stresses due to both conditions may be detrimental at low cell concentration.

Process conditions and control settings may influence the kinetics of microbial growth and cellular activity. Shifts in process conditions may change membrane composition, production of metabolites, growth rate, cellular stress, etc. Optimal temperature range for growth may vary with strain. The range may be 20-40° C. Optimal pH for cell growth and performance of downstream activity may vary with strain. The range may be pH 5-8. Gasses dissolved in the medium may be used by cells for metabolism. Adjusting concentrations of O₂, CO₂, and N₂ throughout the process may be required. Availability of nutrients may shift cellular growth. Bacteria may have alternate kinetics when excess nutrients are available.

The state of bacteria at the end of a fermentation and during harvesting may impact cell survival and activity. Bacteria may be preconditioned shortly before harvest to better prepare them for the physical and chemical stresses involved in separation and downstream processing. A change in temperature (often reducing to 20-5° C.) may reduce cellular metabolism, slowing growth (and/or death) and physiological change when removed from the fermenter. Effectiveness of centrifugal concentration may be influenced by culture pH. Raising pH by 1-2 points can improve effectiveness of concentration but can also be detrimental to cells. Bacteria may be stressed shortly before harvest by increasing the concentration of salts and/or sugars in the medium. Cells stressed in this way may better survive freezing and lyophilization during downstream.

Separation methods and technology may impact how efficiently bacteria are separated from the culture medium. Solids may be removed using centrifugation techniques. Effectiveness of centrifugal concentration can be influenced by culture pH or by the use of flocculating agents. Raising pH by 1-2 points may improve effectiveness of concentration but can also be detrimental to cells. Bacteria may be stressed shortly before harvest by increasing the concentration of salts and/or sugars in the medium. Cells stressed in this way may better survive freezing and lyophilization during downstream. Additionally, Bacteria may also be separated via filtration. Filtration is superior to centrifugation techniques for purification if the cells require excessive g-minutes to successfully centrifuge. Excipients can be added before after separation. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets mixed with excipients are submerged in liquid nitrogen.

Harvesting can be performed by continuous centrifugation. Product may be resuspended with various excipients to a desired final concentration. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets mixed with excipients are submerged in liquid nitrogen.

Lyophilization of material, including live bacteria, vesicles, or other bacterial derivative includes a freezing, primary drying, and secondary drying phase. Lyophilization begins with freezing. The product material may or may not be mixed with a lyoprotectant or stabilizer prior to the freezing stage. A product may be frozen prior to the loading of the lyophilizer, or under controlled conditions on the shelf of the lyophilizer. During the next phase, the primary drying phase, ice is removed via sublimation. Here, a vacuum is generated and an appropriate amount of heat is supplied to the material. The ice will sublime while keeping the product temperature below freezing, and below the material's critical temperature (T_{c}). The temperature of the shelf on which the material is loaded and the chamber vacuum can be manipulated to achieve the desired product temperature. During the secondary drying phase, product-bound water molecules are removed. Here, the temperature is generally raised higher than in the primary drying phase to break any physico-chemical interactions that have formed between the water molecules and the product material. After the freeze-drying process is complete, the chamber may be filled with an inert gas, such as nitrogen. The product may be sealed within the freeze dryer under dry conditions, in a glass vial or other similar container, preventing exposure to atmospheric water and contaminates.

### Example 7: EV Preparation

Prevotella histicola and Veillonella parvula smEVs were prepared as follows.

EVs: Downstream processing of EVs began immediately following harvest of the bioreactor. Centrifugation at 20,000 g was used to remove the cells from the broth. The resulting supernatant was clarified using 0.22 μm filter. The EVs were concentrated and washed using tangential flow filtration (TFF) with flat sheet cassettes ultrafiltration (UF) membranes with 100 kDa molecular weight cutoff (MWCO). Diafiltration (DF) was used to washout small molecules and small proteins using 5 volumes of phosphate buffer solution (PBS). The retentate from TFF was spun down in an ultracentrifuge at 200,000 g for 1 hour to form a pellet rich in EVs called a high-speed pellet (HSP). The pellet was resuspended with minimal PBS and a gradient was prepared with Optiprep™ density gradient medium and ultracentrifuged at 200,000 g for 16 hours. Of the resulting fractions, 2 middle bands contained EVs. The fractions were washed with 15-fold PBS and the EVs spun down at 200,000 g for 1 hour to create the fractionated HSP or fHSP. It was subsequently resuspended with minimal PBS, pooled, and analyzed for particles per mL and protein content. Dosing was prepared from the particle/mL count to achieve desired concentration. The EVs were characterized using a NanoSight NS300 by Malvern Panalytical in scatter mode using the 532 nm laser.

### Example 8: EV Isolation and Enumeration

The equipment used in EV isolation includes a Sorvall RC-5C centrifuge with SLA-3000 rotor; an Optima XE-90 Ultracentrifuge by Beckman-Coulter 45Ti rotor; a Sorvall wX+ Ultra Series Centrifuge by Thermo Scientific; and a Fiberlite F37L-8x100 rotor.

### Bacterial Supernatant Collection and Filtration

Bacteria must be pelleted and filtered away from supernatant in order to recover EVs and not bacteria.

Pellet bacterial culture is generated by using a Sorvall RC-5C centrifuge with the SLA-3000 rotor and centrifuge culture for a minimum of 15 min at a minimum of 7,000 rpm. And then decanting the supernatant into new and sterile container.

The supernatant is filtered through a 0.2 μm filter. For supernatants with poor filterability (less than 300 ml of supernatant pass through filter) a 0.45 μm capsule filter is attached ahead of the 0.2 μm vacuum filter. The filtered supernatant is stored at 4° C. The filtered supernatant can then be concentrated using TFF.

### Isolation of EVs Using Ultracentrifugation

Concentrated supernatant is centrifuged in the ultracentrifuge to pellet EVs and isolate the EVs from smaller biomolecules. The speed is for 200,000×g, time for 1 hour, and temperature at 4° C. When rotor has stopped, tubes are removed from the ultracentrifuge and the supernatant is gently poured off. More supernatant is added the tubes are centrifuged again. After all concentrated supernatant has been centrifuged, the pellets generated are referred to as ‘crude’ EV pellets. Sterile 1×PBS is added to pellets, which are placed in a container. The container is placed on a shaker set at speed 70, in a 4° C. fridge overnight or longer. The EV pellets are resuspended with additional sterile 1×PBS. The resuspended crude EV samples are stored at 4° C., or at −80° C.

### EV Purification Using Density Gradients

Density gradients are used for EV purification. During ultracentrifugation, particles in the sample will move, and separate, within the graded density medium based on their ‘buoyant’ densities. In this way EVs are separated from other particles, such as sugars, lipids, or other proteins, in the sample.

For EV purification, four different percentages of the density medium (60% Optiprep) are used, a 45% layer, a 35% layer, a 25%, and a 15% layer. This will create the graded layers. A 0% layer is added at the top consisting of sterile 1×PBS. The 45% gradient layer should contain the crude EV sample. 5 ml of sample is added to 15 ml of Optiprep. If crude EV sample is less than 5 ml, bring up to volume using sterile 1×PBS.

Using a serological pipette, the 45% gradient mixture is pipetted up and down to mix. The sample is then pipetted into a labeled clean and sterile ultracentrifuge tube. Next, a 10 ml serological pipette is used to slowly add 13 ml of 35% gradient mixture. Next 13 ml of the 25% gradient mixture is added, followed by 13 ml of the 15% mixture and finally 6 ml of sterile 1×PBS. The ultracentrifuge tubes are balanced with sterile 1×PBS. The gradients are carefully placed in a rotor and the ultracentrifuge is set for 200,000×g and 4° C. The gradients are centrifuged for a minimum of 16 hours.

A clean pipette is used to remove fraction(s) of interest, which are added to 15 ml conical tube. These ‘purified’ EV samples are kept at 4° C.

In order to clean and remove residual optiprep from EVs, 10× volume of PBS are added to purified EVs. The ultracentrifuge is set for 200,000×g and 4° C. Centrifuge and spun for 1 hour. The tubes are carefully removed from ultracentrifuge and the supernatant decanted. The purified EVs are washed until all sample has been pelleted. 1×PBS is added to the purified pellets, which are placed in a container. The container is placed on a shaker set at speed 70 in a 4° C. fridge overnight or longer. The ‘purified’ EV pellets are resuspended with additional sterile 1×PBS. The resuspended purified EV samples are stored at 4° C., or at −80° C.

### Example 9: Prevotella EVs Lyophilate: DTH Efficacy

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with Prevotella histicola EVs or dosed intraperitoneally with dexamethasone (positive control) at 1 mg/kg from days 6-8. After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours.

In this Example, the Prevotella smEVs used in the studies were isolated from Prevotella Strain B (NRRL accession number B 50329). The EVs were lyophilized in excipient formula 7a.

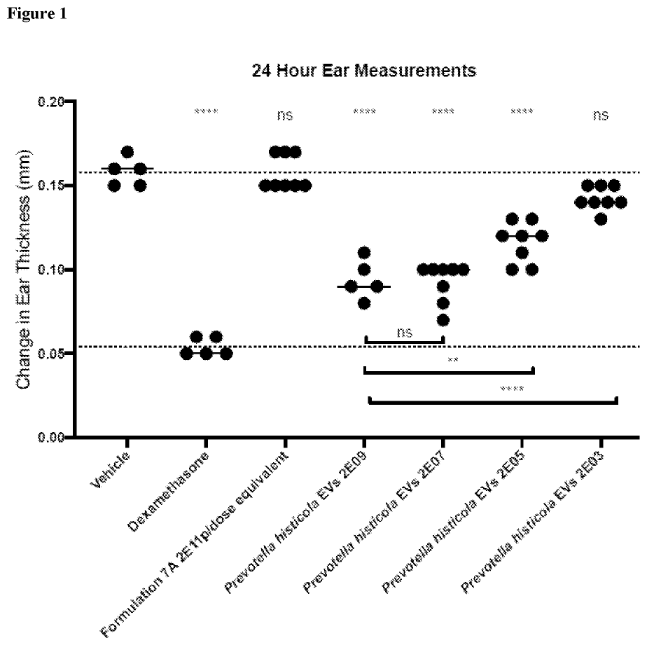

The 24 hour ear measurement results are shown in FIG. 1. EVs made from Prevotella histicola and lyophilized in the excipient of formula 7a were tested in a dose range study with four doses (2E09, 2E07, 2E05, 2E03) for three days of dosing. All doses of the Prevotella histicola EVs were efficacious compared to vehicle except for the lowest dose (2E03) and there was a dose response trend seen. As a negative control, formulation 7a alone was used (with excipient components at a dose equivalent to the amounts present as if 2e11 EVs had been formulated).

### Example 10: Isolation and Characterization of EVs (smEVs)

Strains Isolated from the Families Listed Below:
All EVs were lyophilized in Formulation 7a (20% Trehalose, 80% Mannitol).
Taxonomy is from lpsn.dsmz.de/

Prevotellaceae:
Domain: Bacteria, Phylum: Bacteroidetes, Class Bacteroidia, Order: Bacteroidales
Gram negative cell wall structure (diderm)
Prevotella nigrescens
Prevotella copri
Prevotella oralis
Prevotella buccae

Oscillospiraceae
Domain: Bacteria, Phylum: Firmicutes, Class: Clostridia, Order: Eubacteriales
Gram positive cell wall structure (monoderm)
Anaerotruncus colihominus (batch 1, grown in glucose)
Anaerotruncus colihominus (batch 2, grown in NAG)
Subdoligranulum variable
Harryflintia acetispora (batch 1, grown in tween)
Harryflintia acetispora (batch 2, grown without tween)
Acutalibacter sp.

Veillonellaceae
Domain: Bacteria, Phylum: Firmicutes, Class: Negativicutes, Order: Veillonellales
Gram negative cell wall structure (diderm)
Veillonella parvula (batch 1, strain A)
Veillonella parvula (batch 2, strain B)
Megasphaera vaginalis
Megasphaera sp.
Veillonella atypica

Tannerellaceae
Domain: Bacteria, Phylum: Bacteroidetes, Class: Bacteroidia, Order: Bacteroidales
gram negative cell wall structure (diderm)
   Parabacteroides distastonis
Parabacteroides gordonii
Parabacteroides merdae
Parabacteroides goldeinii

Clostridiaceae
Domain: Bacteria, Phylum: Firmicutes, Class: Clostridia, Order: Eubacteriales
Gram positive cell wall structure (monoderm)
Anaeromassilibacillus sp.
Clostridium cadaveris
Clostridium butyricum

Lahnospiraeeae
Domain: Bacteria, Phylum: Firmicutes, Class: Clostridia, Order: Eubacteriales
Gram positive cell wall structure (monoderm)
Dorea longicatena
Mediterraneibacter/[Ruminococcus] gnavus
Blautia massiliensis

Rikenellaceae
Domain: Bacteria, Phylum: Bacteroidetes, Class Bacteroidia, Order: Bacteroidales
Gram negative cell wall structure (diderm)
Alistipes indistinctus
Alistipes timonensis

Selenomonadaceae
Domain: Bacteria, Phylum: Firmicutes, Class: Negativicutes Order: Selenomonadales
Gram negative cell wall structure (diderm)
Selenomonas felix
Selenomonas sputigena

Sporomusaceae
Domain: Bacteria, Phylum: Firmicutes, Class: Negativicutes Order: Selenomonadales
Gram negative cell wall structure (diderm)
Propionispora sp.

Christensenellaceae
Domain: Bacteria, Phylum: Firmicutes, Class: Eubacteriales, Order: Christensenellales
Gram negative cell wall structure (diderm)
Christensenella minuta

Synergistaceae
Domain: Bacteria, Phylum: Synergistetes, Class: Synergistia, Order: Synergistales
Gram negative cell wall structure (diderm)
Cloacibacillus evryensis

Akkermansiaceae
Domain: Bacteria, Phylum: Verrucomicrobia, Class: Verrucomicrobiae, Order: Verrucomicrobiales
Gram negative cell wall structure (diderm)
Akkermansia muciniphila

EV Isolation Process

Preparation of EVs for lyophilization was as follows:

After growth of microbe in bioreactor, the culture was pelleted with Sorvall RC-5C centrifuge with the SLA-3000 rotor for a minimum of 15 minutes at a minimum of 7,000 rotations per minute. Supernatants were collected and passed through a 0.2 μm Pall Vacu-Cap filter (VWR, 28139-706). For supernatants with poor filterability (less than 300 ml of supernatant passed through filter) a 0.45 μm capsule filter (VWR, 28145-870) was attached ahead of the 0.2 μm vacuum filter.

Bacterial supernatants were concentrated using flat-sheet TFF (tangential flow filtration). Supernatants derived from monoderm bacteria were concentrated through two 300 kDa re-usable cassettes (Repligen). Supernatants derived from diderm bacteria were concentrated through two 300 kDa (Millipore) re-usable cassettes to limit the possible transfer of LPS (endotoxin) to supernatants from non-LPS containing bacteria. Prior to supernatant concentration, flat sheet TFF system, feed, retentate, and permeate lines were flushed with 0.5 M NaOH followed by 0.22 μm sterile filtered deionized (DI) water until the outflow DI water showed neutral pH (approximately 4 L). Supernatants were then concentrated by placing permeate line in a waste reservoir and feed and retentate lines in the bottle containing the supernatant. Supernatant was concentrated until volume reached 100 ml. Pressure sensors were monitored and kept below 10 psi. After concentrated supernatant reached 100 ml, material was diafiltrated to remove small metabolites and media components. Sterile filtered DI water was slowly added to a volume of 500 ml or 1 L depending on the sample. To remove and collect remaining material in tubing, the permeate line was clamped and the feed and retentate lines were placed in a smaller, secondary bottle containing 50 ml of 0.22 μm sterile filtered DI water. The lines were flushed with sterile DI water. The resulting material was collected and added to the concentrated supernatant. Concentrated retentate was lyophilized in formulation 7a (20% Trehalose, 80% Mannitol).

For Gradient Purification Only:

Filtered and concentrated supernatants were then split into clean ultracentrifuge tubes, material was spun Filtered and concentrated supernatants were then split into clean ultracentrifuge tubes, material was spun down in ultracentrifuge at 4° C. 200,000×g for 1 hour samples were resuspended in sterile PBS. Crude pellets were then gradient purified on Optiprep (Sigma Aldrich, D1556-250ML) gradients at concentrations of 15%, 35/6, and 45%. Material was ultracentrifuged (Optima XE-90 Ultracentrifuge by Beckman-Coulter with 45Ti rotor or Sorvall wX+ Ultra Series Centrifuge by Thermo Scientific with Fiberlite F37L-8x100 rotor) at 4° C. at 200,000×g 10-16 hours. To remove Optiprep, sample was washed in 10× volume of PBS. Washed sample was spun down at 4° C. at 200,000×g for 1 hour (2 washes were needed to collect all material from sample layer). Sample was resuspended in sterile PBS.

### Lyophilization

Lyophilization conditions are provided in Table 6.

TABLE 6
Composite cycle for microbiology EV samples

| | Ramp time | Hold time | Shelf temp | Vacuum |
|---|---|---|---|---|
| Step | (min) | (min) | (C.) | (m Torr) |
| Freezing | 200 | 360 | −45 | 100-300 |
| Primary drying | 75 | 5000-6200 | −20 | 100-300 |
| Secondary drying | 180 | 300-1000 | 25 | 100-300 |
| Hold | 0 | N/A | 25 | 100-300 |

### EV Characterization Assays

1. Particle Enumeration from Lyophilized Powder

Objective: To determine the number of particles present in the powders using nanoparticle tracking analysis on the ZetaView.

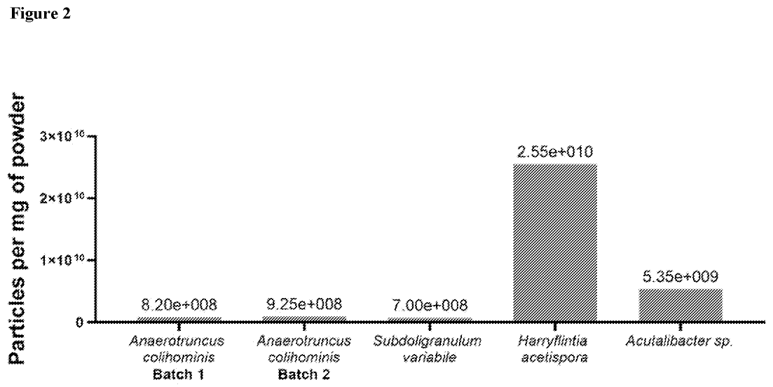

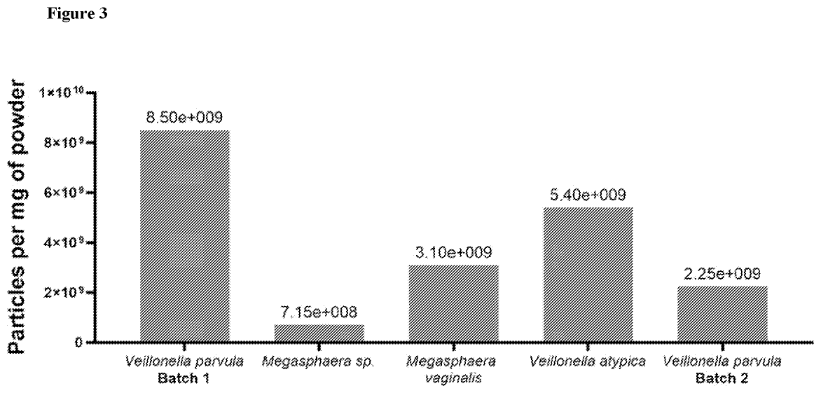

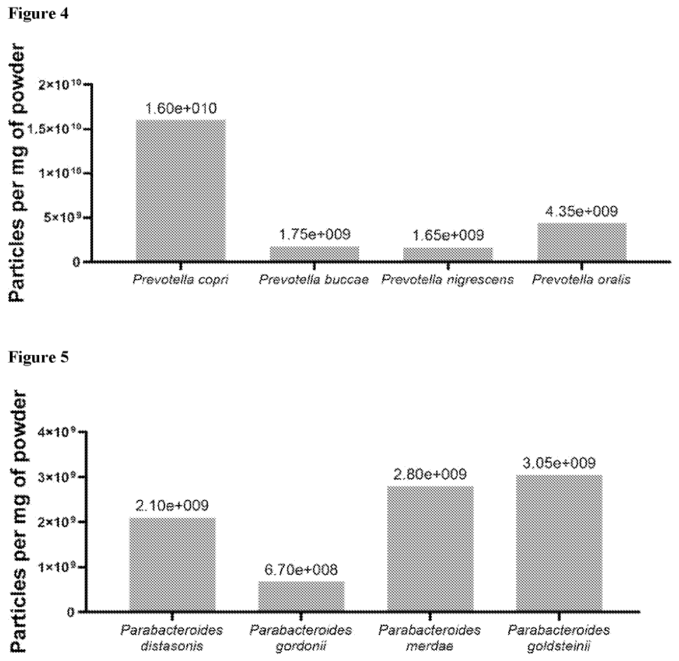

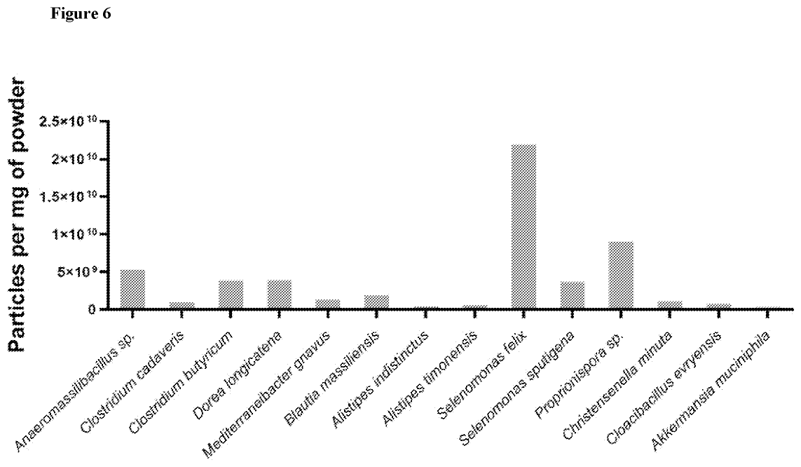

Protocol: Briefly, 50 mg of each powder weighed on an analytical balance was resuspended in 5 mL of MilliQ water (Millipore) and a series of dilutions (10⁻³, 10⁻⁴, and 10⁻⁵ weight per volume) was prepared to test on the Zetaview (Particle Metrix) to have the optimal reading range of 50-400 particles per field of view as specified in the manufacturer's protocol. Zetaview camera was aligned using 100 nm PS alignment beads (Particle Metrix, cat. 110-0020) and a 1E8 particles per milliliter concentration of 100 nm colloidal silica beads (Kanomax) were used as a reference standard. The laser was set to 488 scatter and camera sensitivity was set to 80 and shutter set to 300. Samples were measured in duplicate and average values were reported. Results are shown in FIGS. 2-6. The y-axis provides the particles per mg of powder extrapolated from a 5 mgs per mL resuspension in milliQ water.

2. Dynamic Light Scattering Measuring Particle Size and Charge

Objective: Size and charge of particles are physical properties that may impact efficacy and potency of EVs in vitro and in vivo. There is evidence that size and charge of particles impact interaction with immune cells including phagocytosis (Paul et al. (2013) Biophys J. 105(5):1143-50). Charge may also impact filterability of the supernatants containing the EVs and stability of EVs in solution (Getnet Midekessa et al. (2020) ACS Omega. 5(27): 16701-10). Dynamic light scattering (DLS) is a bulk method of particle detection that measures the intensity of scattered light as a function of time to determine features of particles including diameter (size) and zeta potential (charge) (Szatanek et al. (2017) Int J Mol Sci. 18(6):1153. The advantage of using DLS as a method to measure size is that is can detect a wide range of particle sizes. In heterogeneous or polydisperse samples such as vesicle preparations, DLS can detect up to three sub-populations.

Protocols: All samples were run on the Malvern Zeta Sizer Nano ZS using DTS1070 cuvettes. Sample was diluted either 100× or 1000× in 0.1×PBS (Gibco). For the settings, the refractive index was set to 1.39 based off of literature reports for refractive index of vesicles (Welsh et al. (2020) J Extracell Vesicles, 9(1):1816641) and material absorption was set to 0.01. The dispersant used to dilute samples was 0.1×PBS (Gibco). Trace salts were added to limit long distance interactions between particles. Measurement angle was set to 173°. A total of 5 replicate measurements was taken to determine size and 3 measurements were taken to determine zeta potential. All runs were averaged together to determine the average size and charge values. The final report gave the “Z-average” or the intensity weighted mean of the entire population and a distribution of sizes or charges. Both the Z-average values and the mean of the most dominant peak were recorded.

### A. Size Determination

### Results:

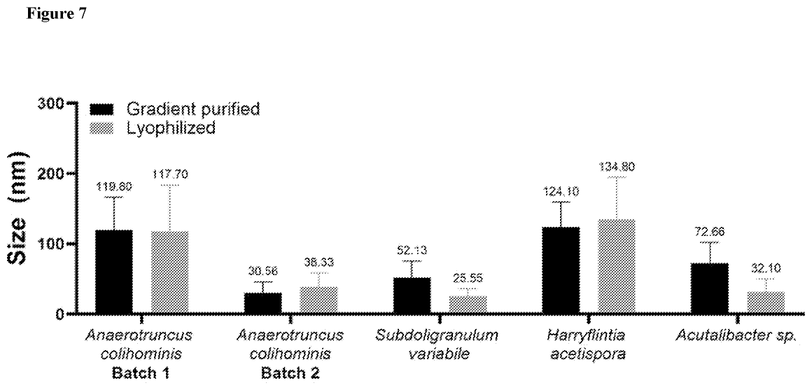

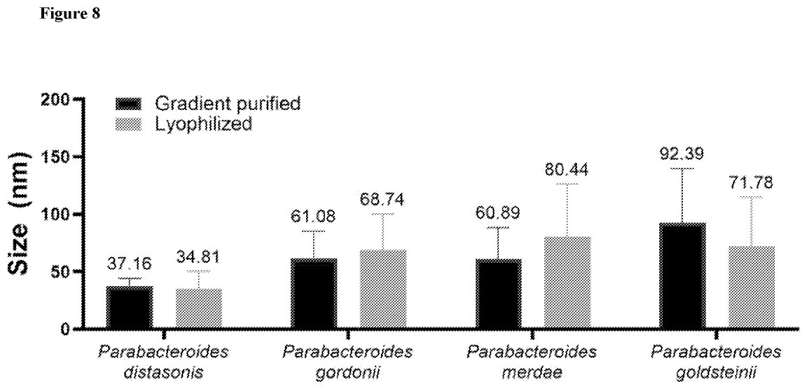

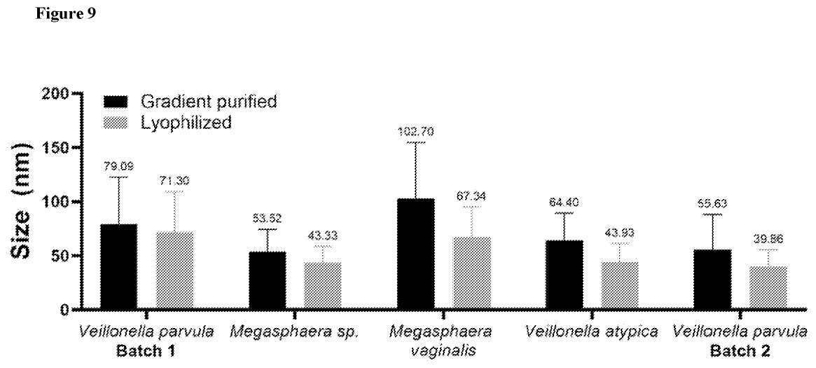

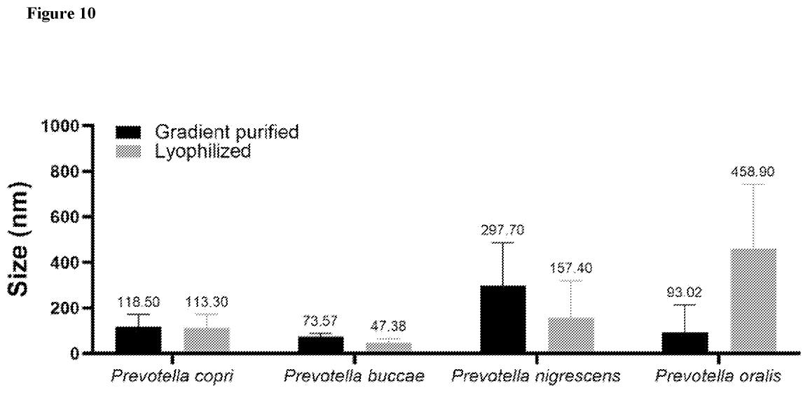

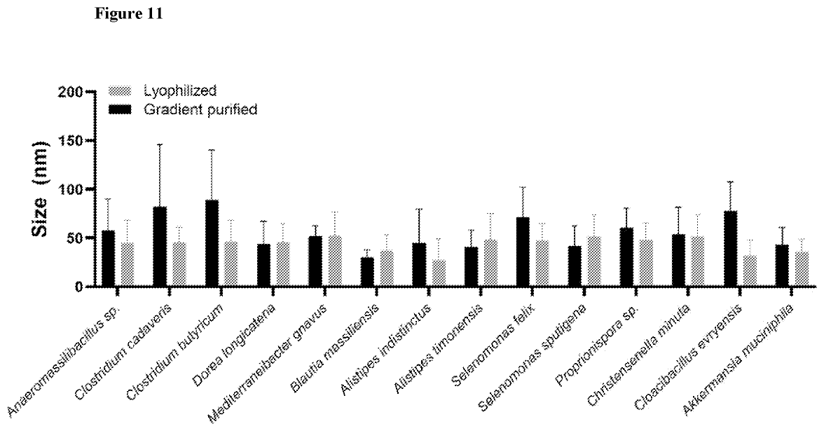

The results for size determination for both gradient purified and lyophilized EVs are shown in FIGS. 7-11. The y-axes on the plots represent the size reported in nanometers. Bars represent the mean of the most dominant DLS integrated peak and error bars show standard deviations from the mean.

### B. Charge (Zeta Potential) Determination

### Results:

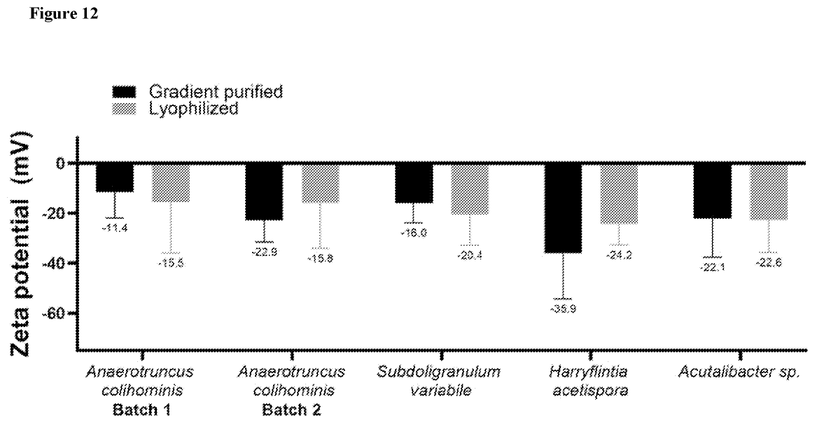

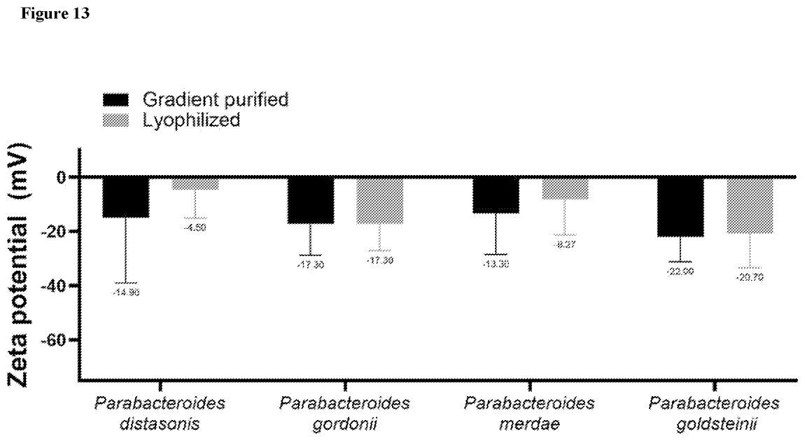

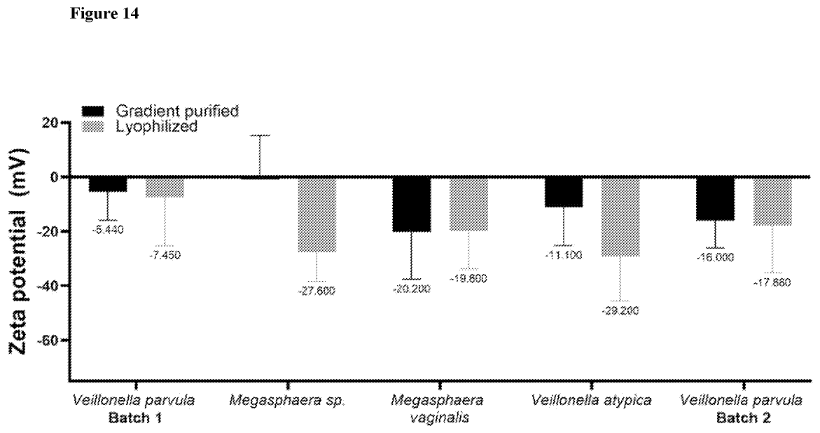

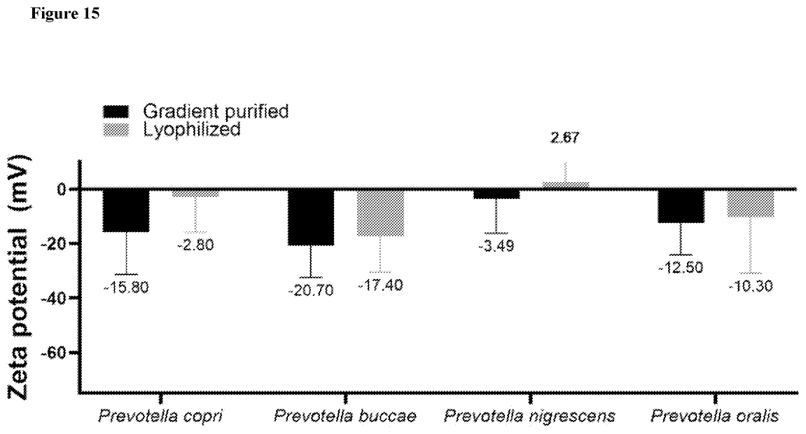

The results for charge for both gradient purified and lyophilized EVs are shown in FIGS. 12-16. The y-axes on the plots provide the zeta potential (mV). The charge has been calculated for the most dominant DLS integrated peak. Bars represent the mean and error bars show standard deviation from the mean.

### Conclusions:

The size of EVs range from 25 nm to ˜500 nm.

The gradient purified material and lyophilized material often had similar sizes and charges, but not in all cases.

Subdoligranulum variabile, Acutalibacter sp., Prevotella nigresens, Clostridium cadaveris, Clostridium butyricum, and Cloacibacilus evryensis were larger after gradient purification. This could be due to aggregation of gradient purified material or elimination of proteins or other small particle contaminants by gradient purification.

Prevotella oralis had much greater size after lyophilization, which is likely due to aggregation.

Almost all the EVs were negatively charged. The only exceptions were Prevotella nigrescens and the gradient purified material from Alistipes timonensis. Cloacibacilus evryensis, and Megasphaera sp.

3. Z Average Measurements of Particle Size and Charge

Here the Z average size and charge measurements are reported. The Z-average value represents the mean size or charge calculated from the entire sample population rather than the most dominant peak.

### A. Size Determination

Results:

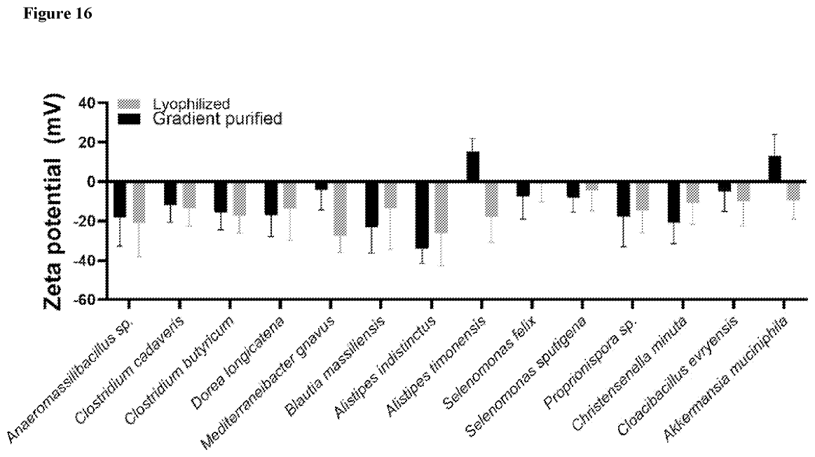

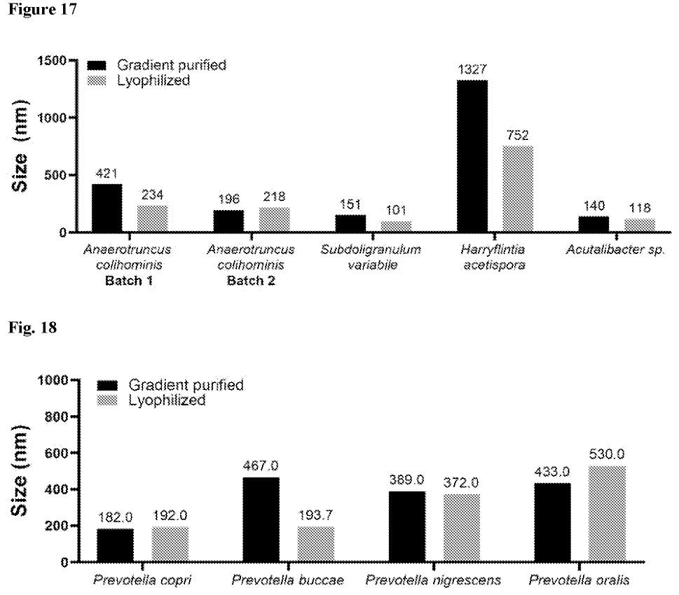

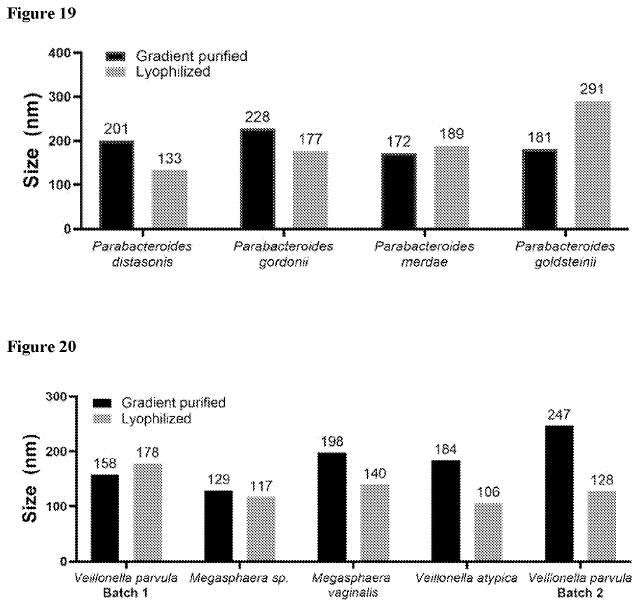

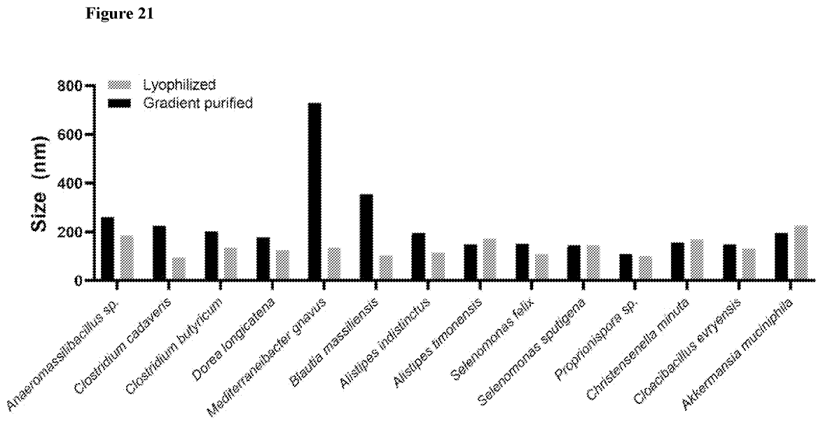

The results for Zₐᵥₑ size determination for both gradient purified and lyophilized EVs are shown in FIGS. 17-21. The y-axis on the plot represents the size (in nm). Bars represent the intensity weighted mean or z-average.

### B. Charge (Zeta Potential) Determination

### Results:

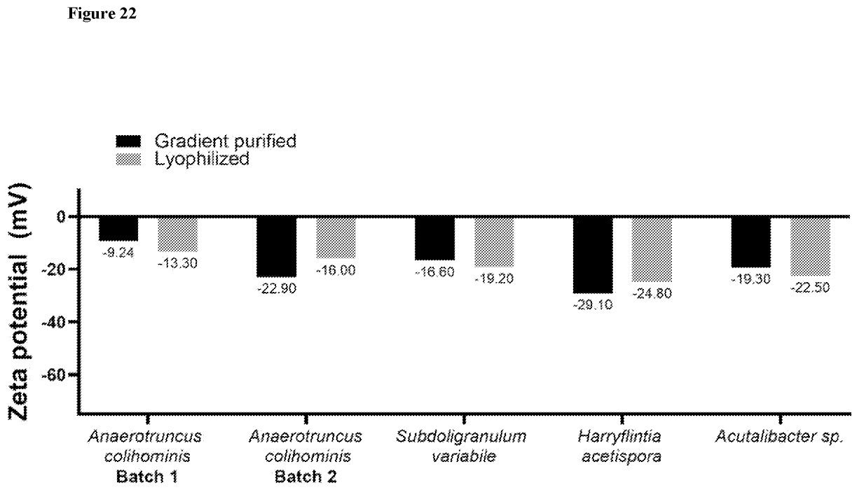

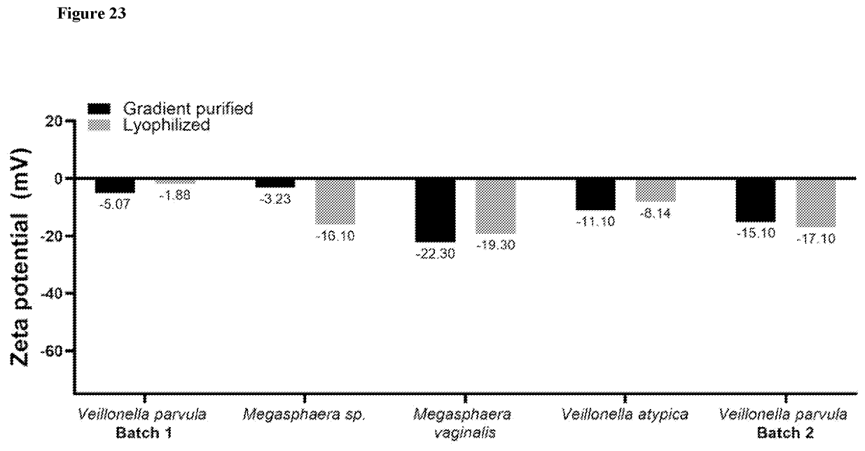

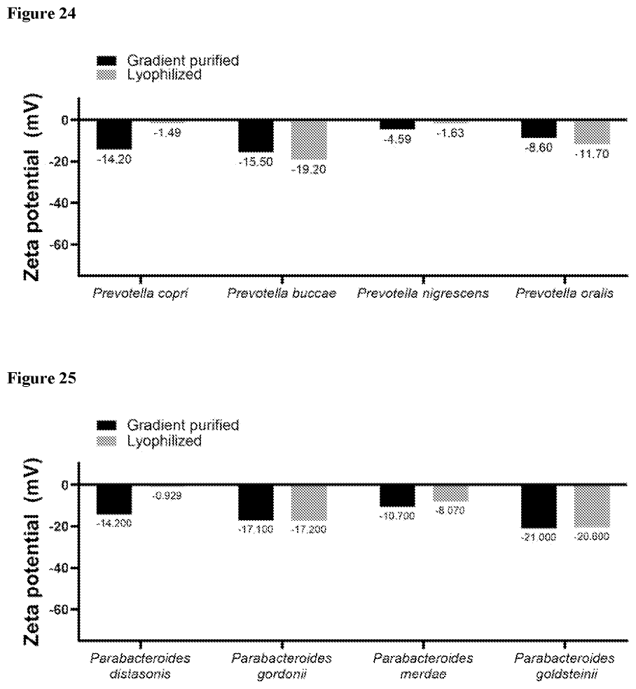

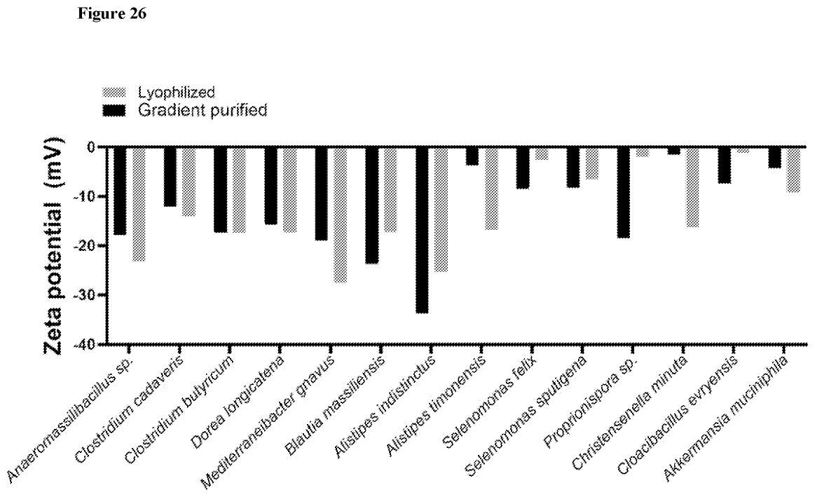

The results for the average charge for both gradient purified and lyophilized EVs are shown in FIGS. 22-26. The y-axes on the plots provide the zeta potential (mV). Bars represent the intensity weighted mean or z-average.

### Conclusions:

the Z-average size overall is greater than the average size of the most dominant peak likely due to a low number of very large particles present in the EV preparations that skew the average.

Gradient purified material and lyophilized material often had similar sizes and charges, but not in all cases.

Gradient-purified material derived from Mediterraneibacter gnavus, Blautia massiliensis, Parabacteroides distasonis, Parabacteroides gordonin, Anaerotruncus colinohominus batch 1. Harryflintia acetispora were larger relative to lyophilized material. This could be due to aggregation or environmental contamination of gradient purified material or elimination of proteins or other small particle contaminants by gradient purification.

All of the Z-average charge values were negative. This indicates that most of the material in the EV preparations is negatively charged.

Gradient purified and lyophilized material derived from Parabacteroides distasonis, Prevotella copri, Alistipes timonensis, Selenomonas felix, Proprionispora sp., Christensenella minuta, Mediterraneibacter gnavus, and Cloacibacilus evryensis had different charge values. This could be due to stabilization of charge from the excipients or the presence of contaminants skewing the average.

4. Karl Fischer Moisture Content

Objective: The purpose of this experiment is to measure water content present in powders of lyophilized extracellular vesicles (EVs).

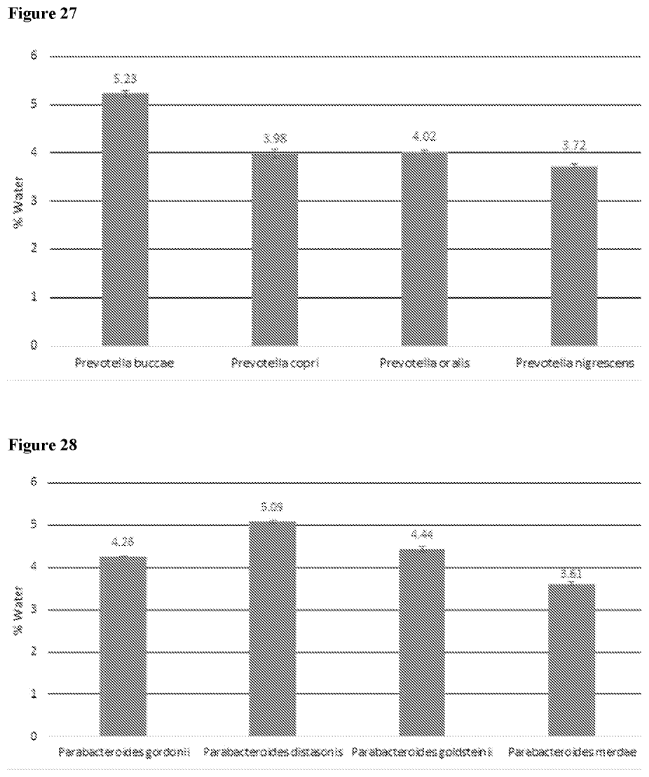

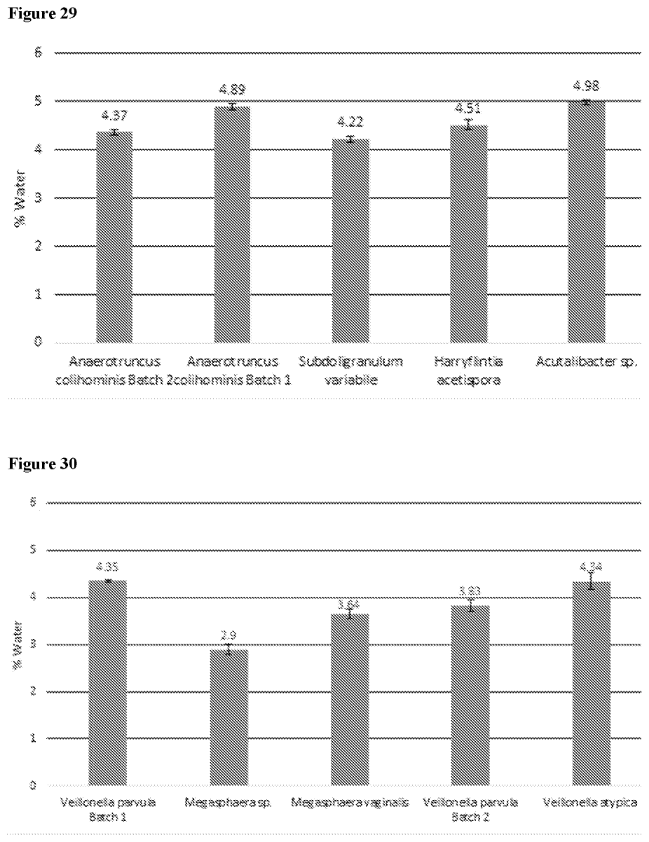

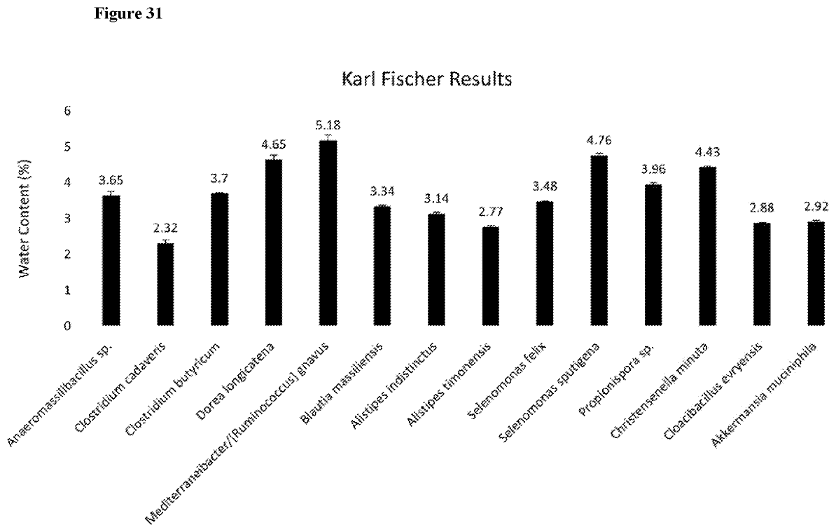

Protocol: System suitability was assessed by weighing 4 mL of Hydranal Water Standard 1.0 (Fluka, CAT #34828-40ML) and injecting into the reaction vessel with a syringe and needle. To calculate percent water, reported water content was divided by the mass of the standard added. The percent water measured of the standard was verified to be within 5% of the value provided by the manufacturer before proceeding to sample analysis. To analyze sample material, ˜30 mg of sample was weighed in a weigh boat on an analytical balance. The exact final weight of sample was recorded, and the sample was transferred into reaction vessel. Percent water was calculated as described above for triplicate samples. Mean water content and relative standard deviations are reported. Results are shown in FIGS. 27-31.

### Conclusions:

Water content values for all samples range from 2.3% to 5.2% with the average value across all samples being 3.99% (Standard deviation=0.76).

Repeatability of individual measurements was precise with some exceptions. RSD of Anaeromassilibacillus sp., Dorea longicatena, and Mediterraneibacter/[Ruminococcus] gnavus were ≥10% which is likely due to the physical state of each powder. Each of these powders had a flakey appearance as opposed to small granules seen in many of the other batches analyzed which led to difficulties in dissolving sample in Karl Fischer reaction vessel.

5. U937 In Vitro Characterization

Objective: Macrophages are likely some of the first immune cells that EVs interact with in the small intestine after oral administration. Microbial associated molecular patterns (MAMPs) such as membrane proteins, nucleic acids, lipids and glycans can be detected by immune pattern recognition receptors (PRRs) such as TLRs (Toll-like receptors), CLRs (C-type lectin receptors) and NLRs (Nod like receptors) and initiate an immune response (Kuipers et al. (2018) Front Microbiol., 9:2182, doi:10.3389/fmicb.2018.02182). Based on the complex composition of macromolecules contained in the EVs, interaction with host macrophages may initiate a more pro-inflammatory, M1-like response characterized by the secretion of cytokines like IL-6, TNFα, and IL-1β or a more tolerogenic, M2-like cytokine response characterized by the secretion of IL-10. In this assay we have developed an in-vitro method to observe U937 macrophages response to EVs isolated from different microbial sources and formulated as powders in formulation 7a.

Protocol: U-937 cells were plated in a 96-well plate at a density of 100,000 cells per well in complete RPMI 1640 media. To differentiate cells, PMA was added at a final concentration of 20 nM for 72 hours at 37° C. in a 5% CO₂ incubator. Cells were washed and incubated in fresh media 24 hours prior to the start the experiment. Extracellular vesicles were diluted in RPMI media to concentrations of 106, 107, 108, and 109 particles per well. The LPS and FSL control were diluted to a final concentration of 10 ng/ml in RPMI 1640 media. A total volume of 200 μL of the diluted EVs and LPS and FSL controls were added in triplicate wells. The plate was placed in a container with a moist paper towel and incubated in a 37° C. incubator for 24 hours under aerobic condition with 5% CO₂. After 24 hours, cell death was assayed with a lactate dehydrogenase detection kit (Promega CytoTox 96 Non-Radioactive Cytotoxicity Assay, cat. G1780). Supernatants were collected and an MSD U-Plex assay was performed to measure IL-10, IL-1β, IL-6, IP-10, and TNFα according to the attached manufacturer's protocol.

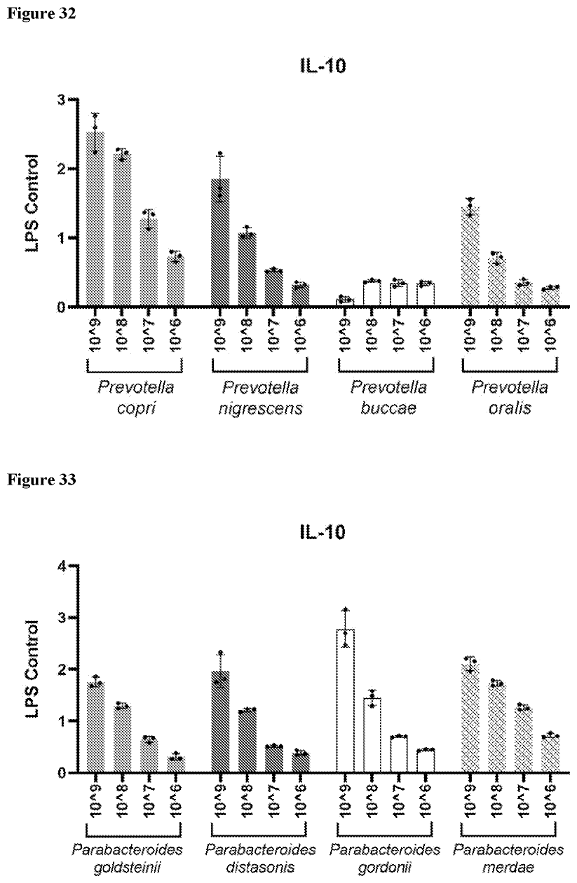

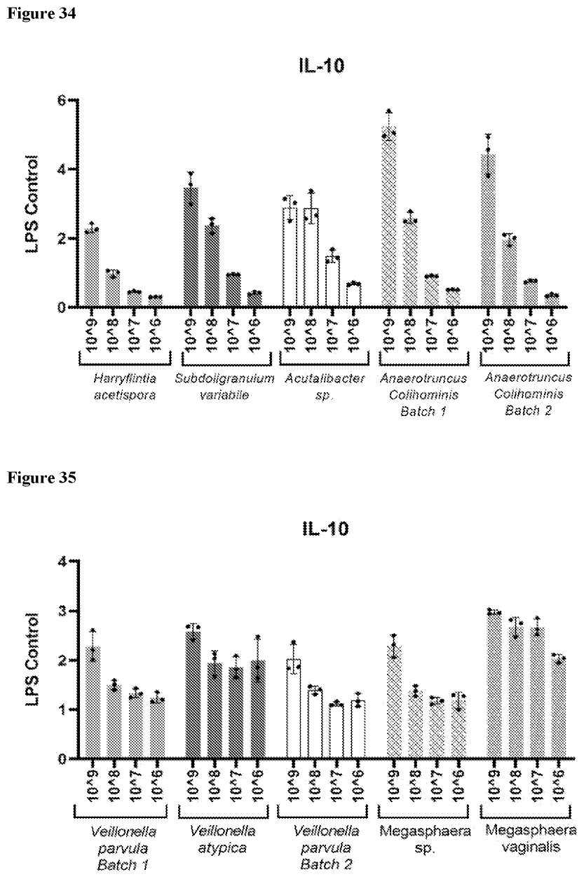

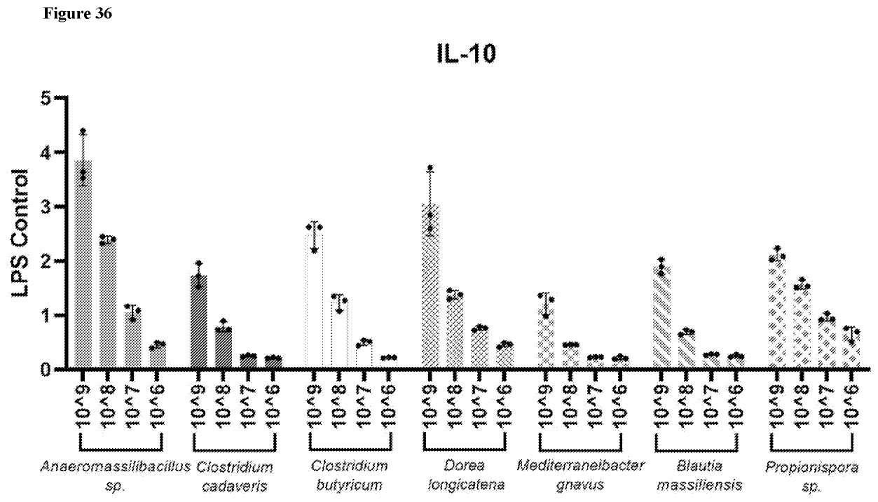

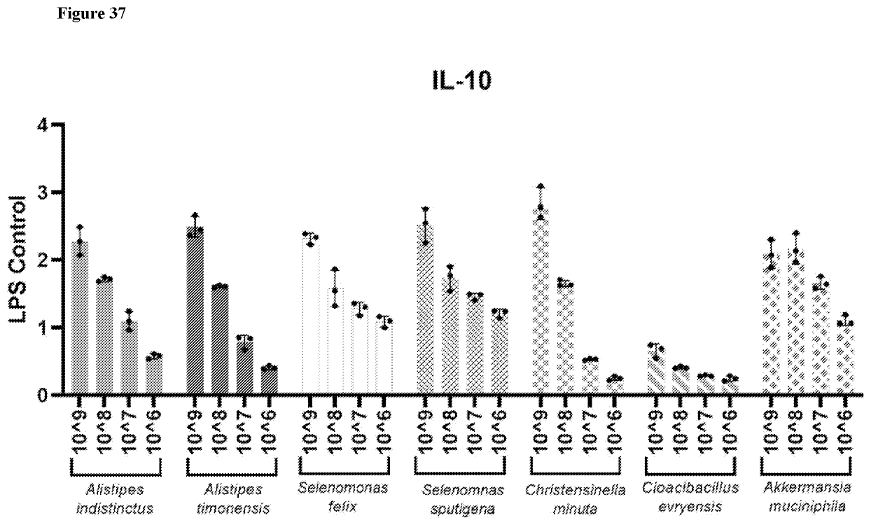

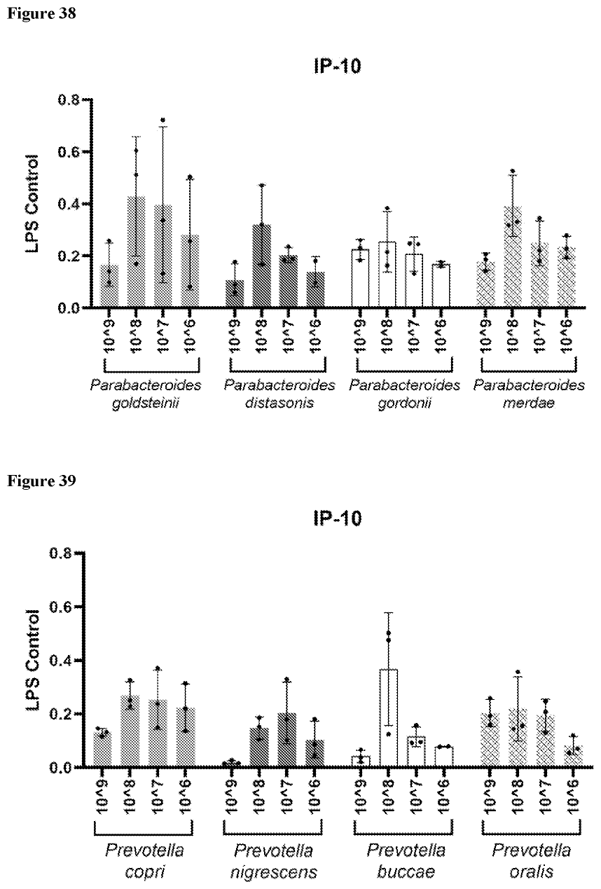

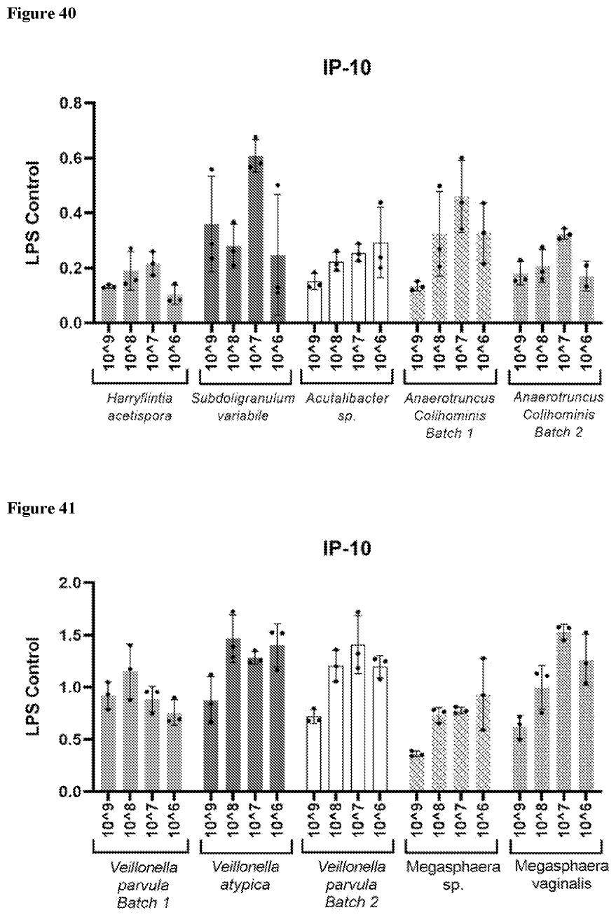

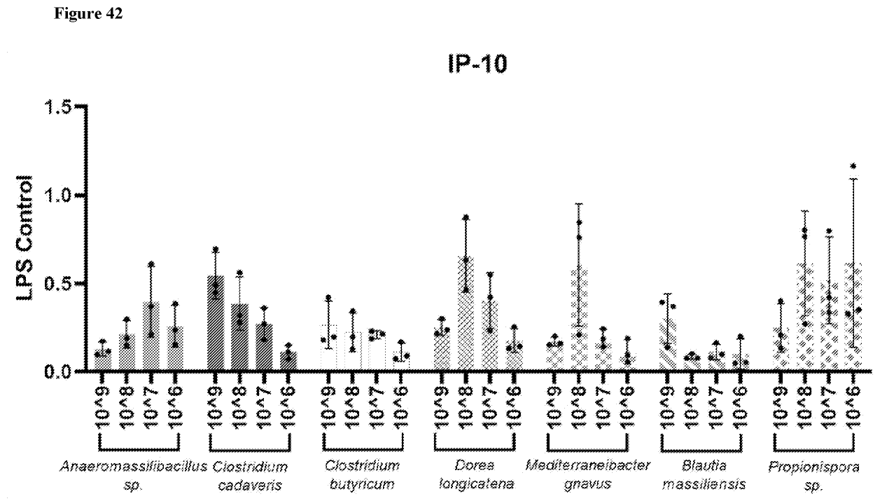

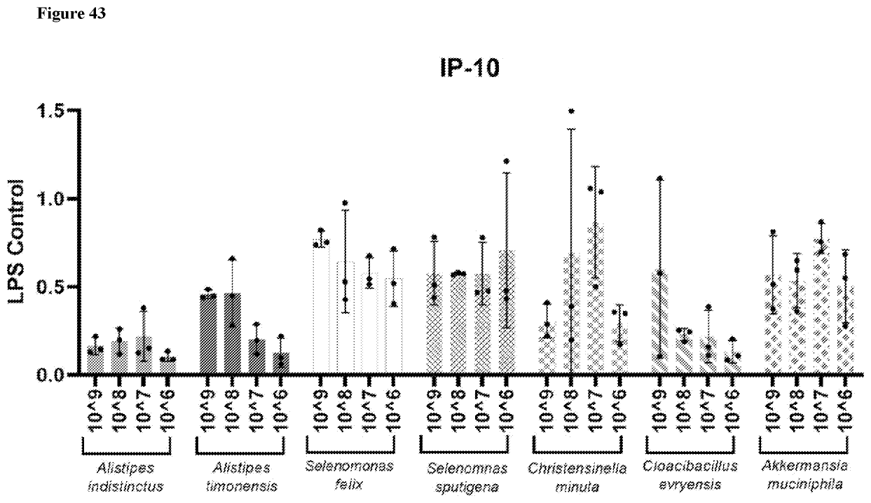

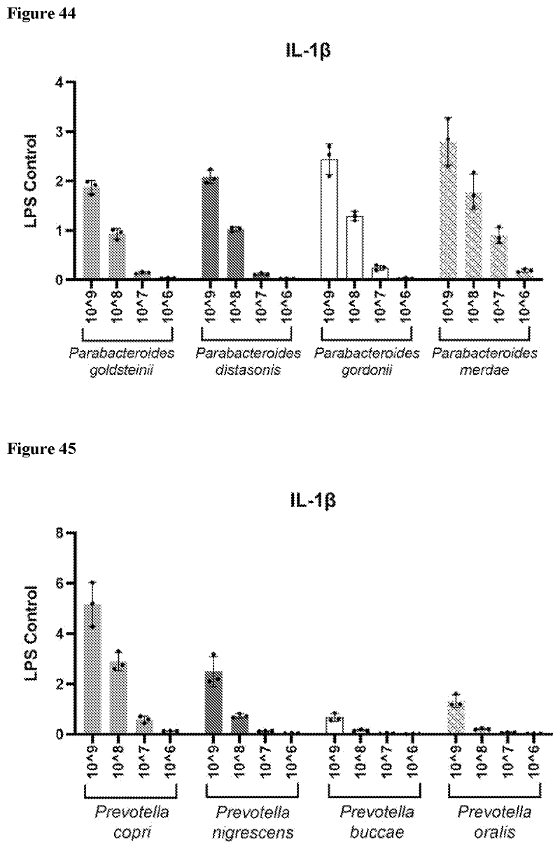

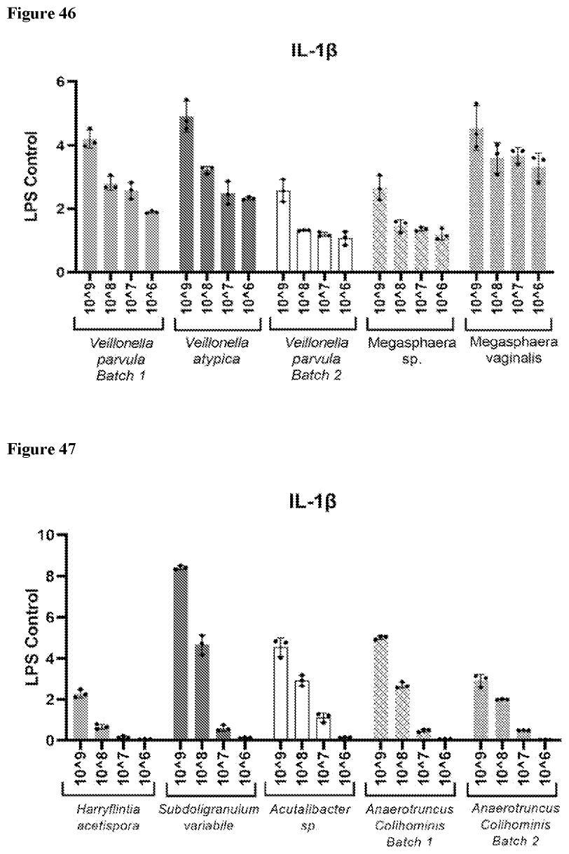

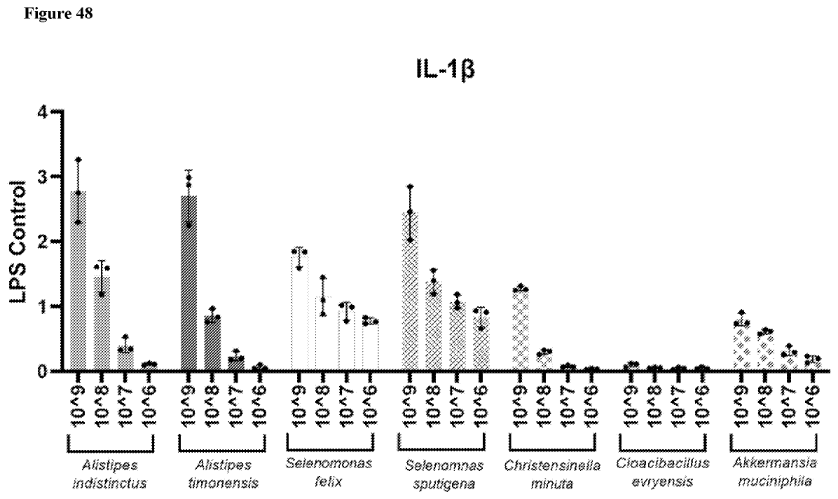

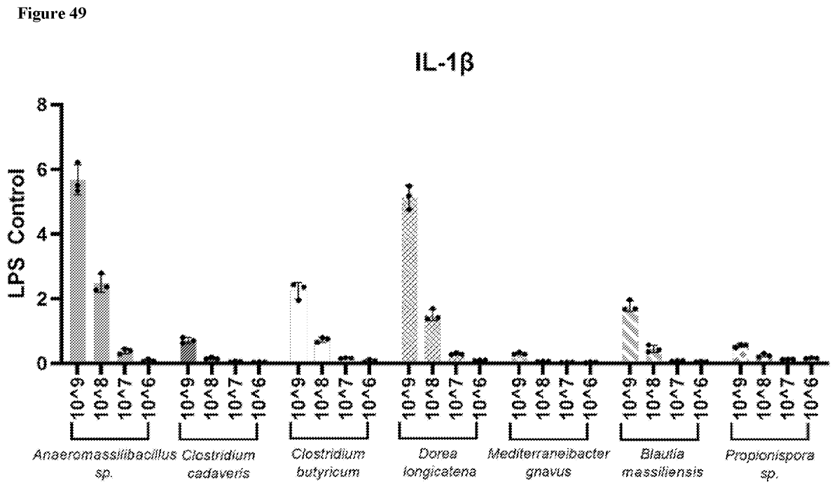

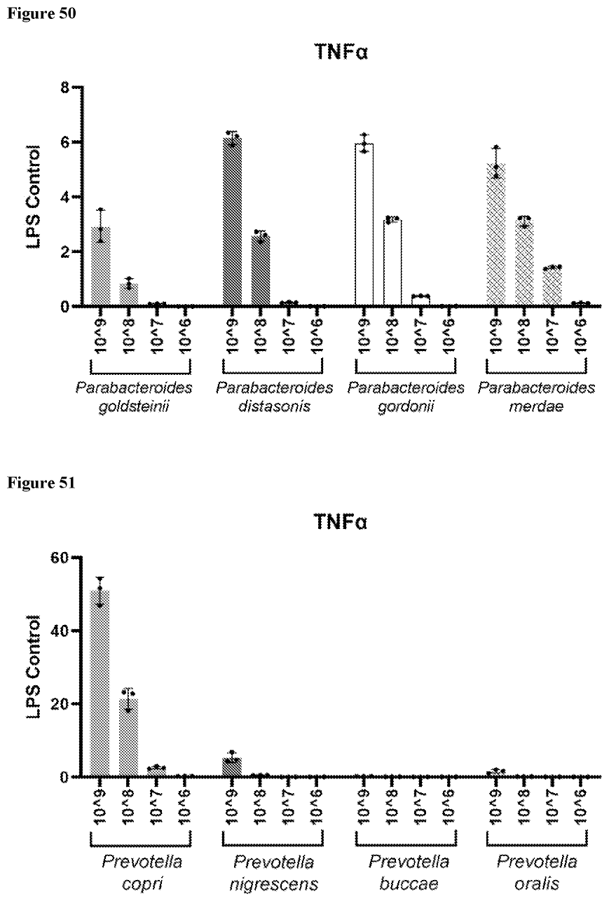

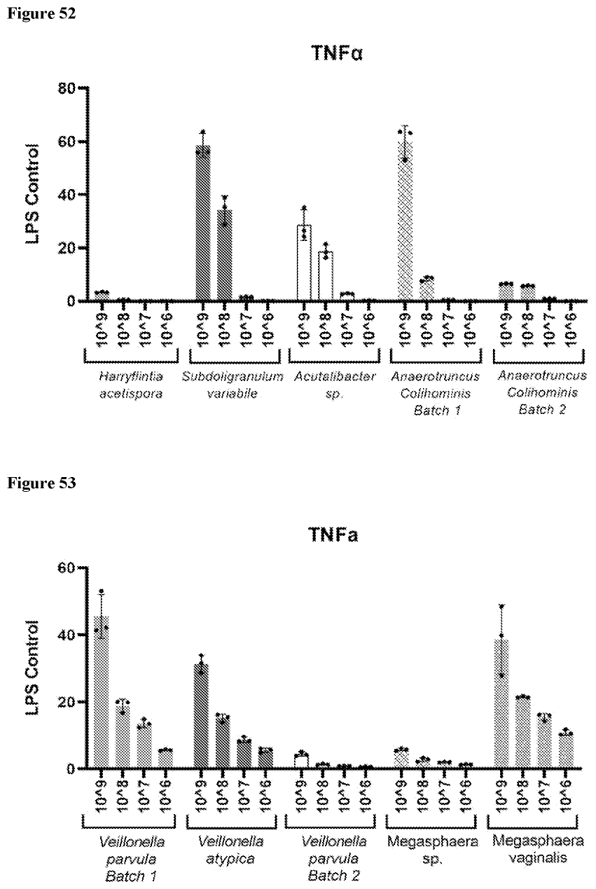

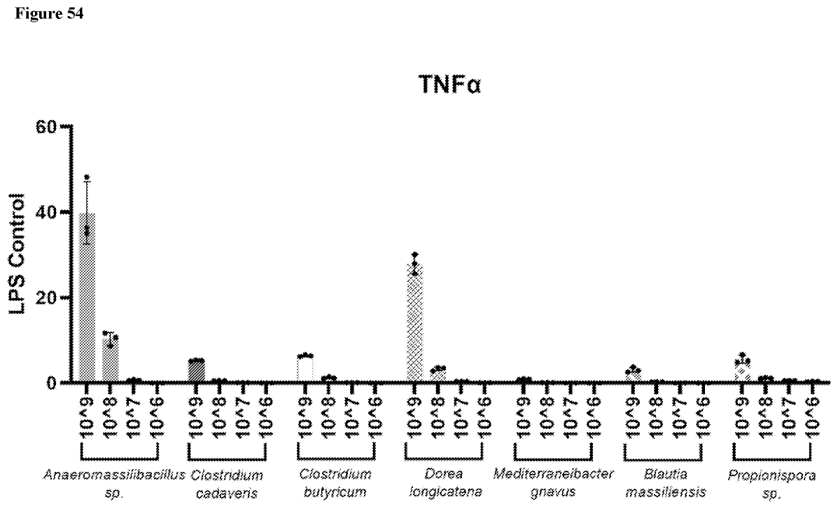

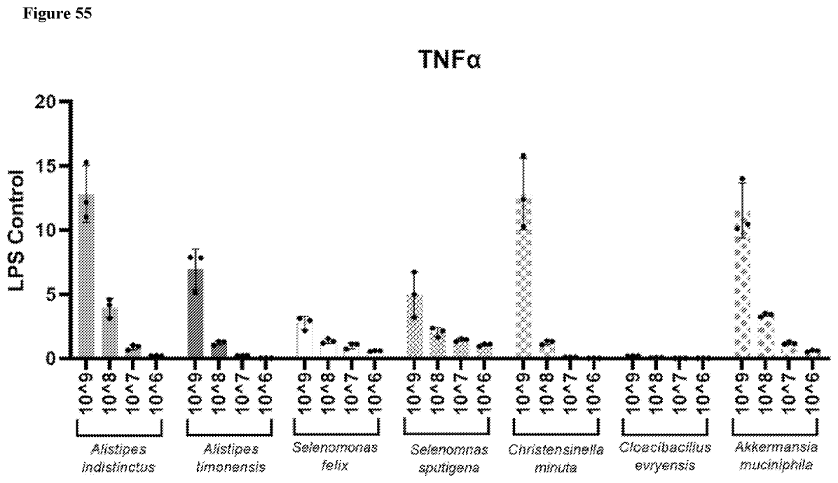

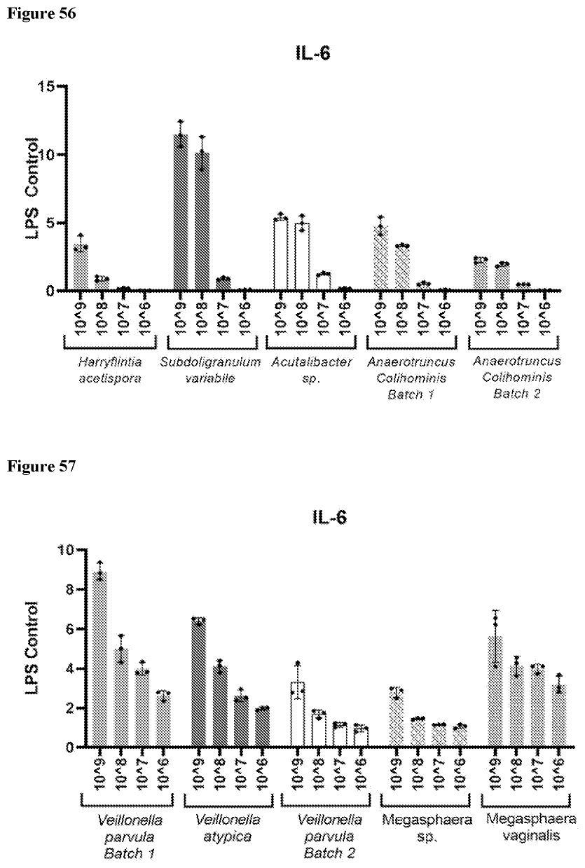

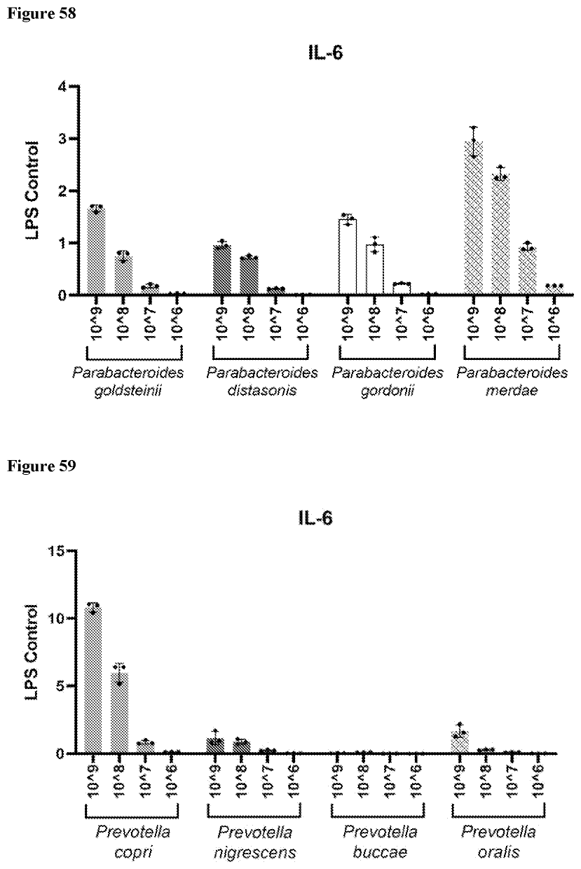

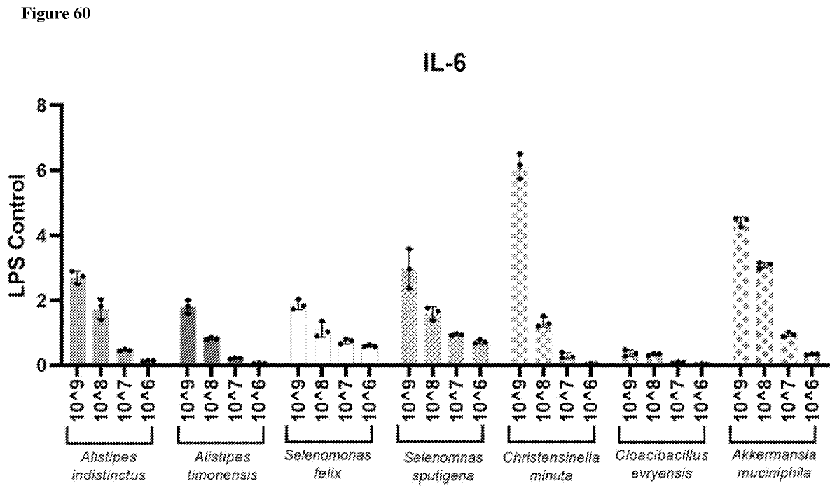

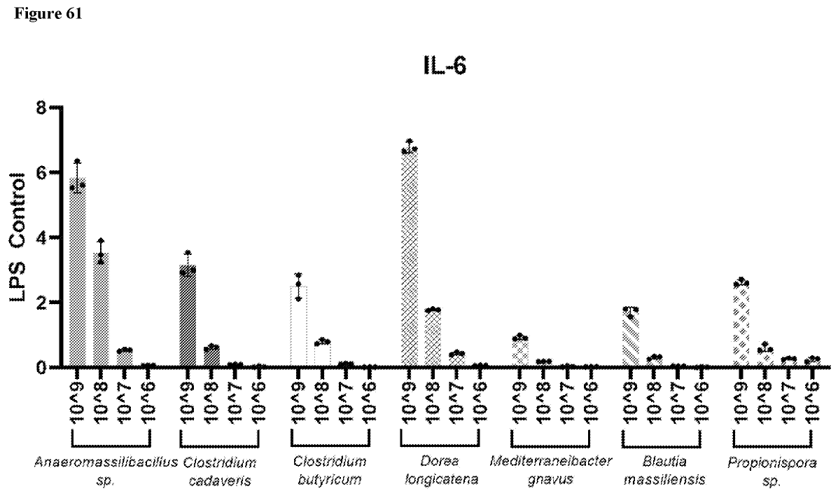

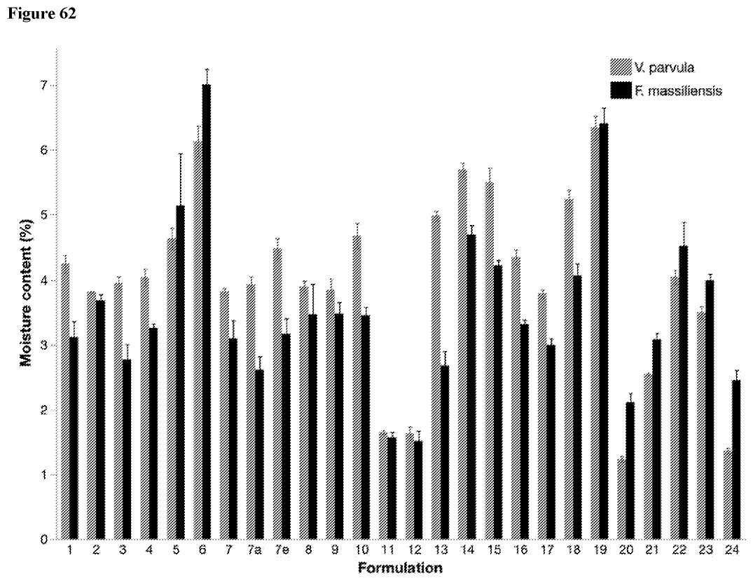

Results are shown in FIGS. 32-37 (IL-10), FIGS. 38-43 (IP-10), FIGS. 44-49 (IL-1β), FIGS. 50-55 (TNFα) and FIGS. 56-61 (IL-6). Specifically, FIGS. 32-37 show IL-10 secretion by U937 macrophages in response to EVs at specified doses; FIGS. 38-43 show IP-10 secretion by U937 macrophages in response to EVs at specified doses; FIGS. 44-49 show IL-1B secretion by U937 macrophages in response to EVs at specified doses; FIGS. 50-55 show TNFα secretion by U937 macrophages in response to EVs at specified doses; and FIGS. 56-61 show IL-6 secretion by U937 macrophages in response to EVs at specified doses. The results show that EVs formulated as lyophilized powders have effects in this in vitro assay.

### Conclusions:

Prevotella copri induced the highest IL-10 expression of the Prevotellaceae family.

IL-10 levels were similar between all the Tannerellaceae but were highest in Parabacteroides gordonii and Parabacteroides merdae.

Anaerotruncus colihominus batch 1 (glucose) induced the highest IL-10 expression of the Oscillospiraceae.

Megasphaera vaginalis induced the highest IL-10 expression out of the Veillonellaceae family.

Anaeromassilibacillus sp. from the Clostridiaceae family also strongly induced IL-10.

Many of the EVs tested activated low secretion of IP-10 compared to LPS. All of the Veillonellaceae family members induced highest IP-10.

Parabacteroides merdae induced the highest IL-1β expression out of the Tannerellaceae family.

Prevotella copri induced the highest IL-1β expression of the Prevotellaceae family.

Megasphaera vaginalis and Veillonella atypica induced the highest IL-1β expression from the Veillonellaceae family.

Subdoligranulum variabile induced the highest IL-1β expression of all the bacterially-derived EVs and was highest in the Oscillospiraceae family.

Veillonella parvula batch 1 was the most pro-inflammatory bacteria of the Veillonellaceae, triggering the most IL-6 and TNFα expression.

Prevotella copri was the most pro-inflammatory bacteria of the Prevotellaceae with highest IL-6 and TNFα expression.

Subdoligranulum variabile was the most pro-inflammatory bacteria with the highest IL-6 and TNFα of all the EVs.

### Overall Conclusions:

Size of bacterial EVs ranged from 25 nm to 457 nm. Larger size of EVs may have been due to aggregation.

Prevotellaceae and Oscillospiraceae had greater size distributions than Tannerellaceae and Veillonellaceae.

On average, the Oscillospiraceae family had the highest protein concentration per E9 particles.

Almost all EVs are negatively charged. The only positively charged EV was Prevotella nigrescens.

Veillonellaceae elicited the greatest cytokine response.

### Example 11: Veillonella Parvula and Fournierella Massiliensis smEVs

The purpose of these studies was to collect data on the physical characteristics of EVs. EVs were isolated from the media of a Veillonella parvula (V. parvula) strain culture and a Fournierella massiliensis (F. massiliensis) strain culture, blended with an excipient formulation, lyophilized, and ground to a lyophilate powder. The properties of the resulting powders were evaluated.

The Veillonella parvula strain used as a source of EVs was the Veillonella bacteria deposited as ATCC designation number PTA-125691. See also WO 2019/157003. The Fournierella massiliensis strain used as a source of EVs was the Fournierella massiliensis bacteria deposited as ATCC designation number PTA-126696. See also PCT/US21/36927.

EVs purified from these strains were lyophilized using a selection of 24 stabilizer mixtures (excipient stocks).

### Preparation of Lyophilized EV Powders:

50 L cultures of each of the Veillonella parvula strain and the Fournierella massiliensis strain were grown under batch fermentation conditions to generate EV material. Each culture was first clarified by centrifugation followed by either sterile filtration or a combination of depth filtration and sterile filtration.

Clarified supernatant was then concentrated approximately 25-fold by Tangential Flow Filtration (TFF) using a 300 kd mPES hollow fiber filter. Concentrates were further purified via a 5-volume continuous diafiltration to remove residual medium components and waste products.

Each stock comprising excipients (see Table K) was prepared as a 15% (w/w) stock solution for the purpose of mixing with purified EV concentrate. Stocks were filter sterilized with a 0.2 μm bottle top filter and stored under ambient conditions until use. Each stabilizer solution was mixed by mass with purified concentrate in a ratio of 0.5875:1 to create an EV-stabilizer “slurry.”

Slurry was transferred to plastic lyophilization trays such that each tray was filled to a depth of approximately 1 cm. Trays were transferred to a shelf lyophilizer where they were frozen and lyophilized using a conservative lyophilization cycle to accommodate the variety of stabilizers being tested (see Table L). Each condition yielded a powder that was 95-99% stabilizer by mass.

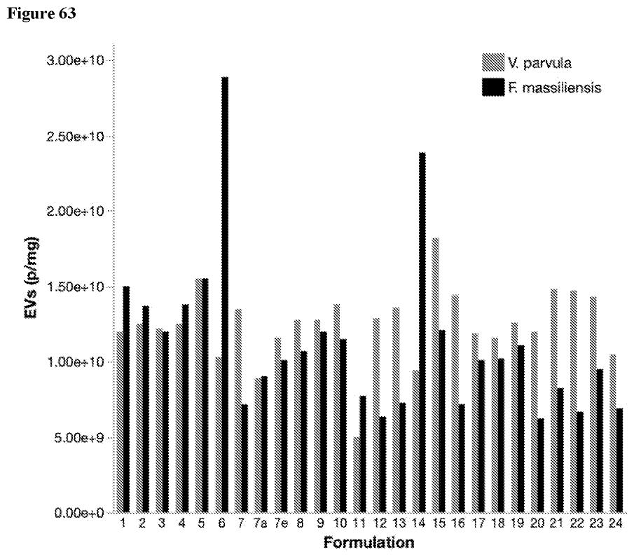

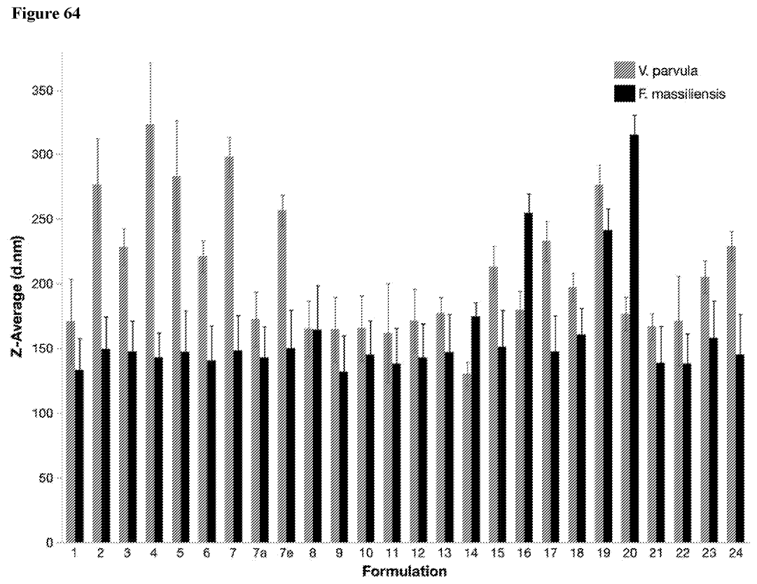

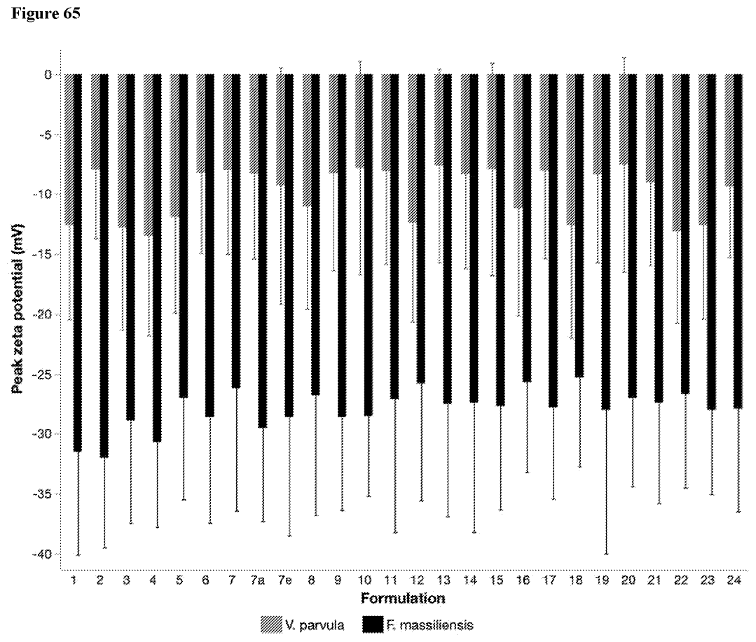

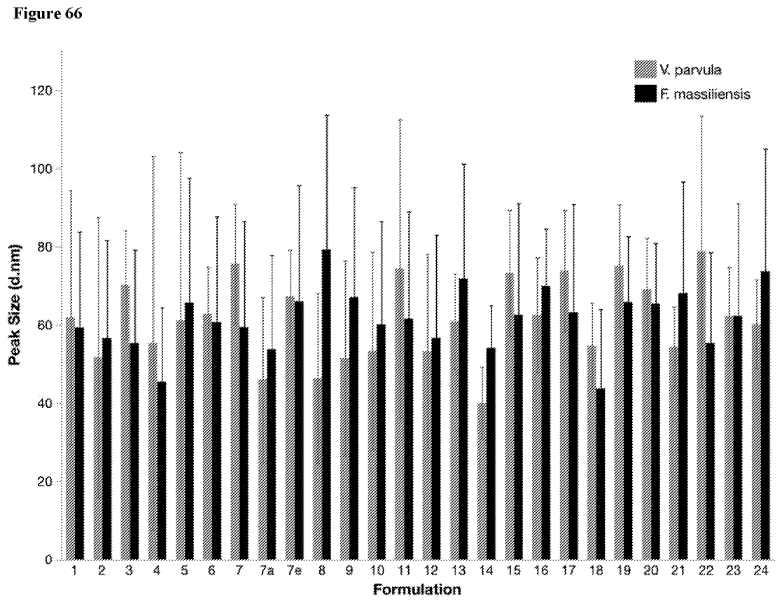

Post lyophilization, each powder was analyzed for moisture content (by Karl Fischer titration (KF)), particle size distribution, and zeta-potential (by dynamic light scattering (DLS)), and particles (quantification by nanoparticle tracking analysis (NTA) with Zetaview). Results are reported in Tables M, N, and O and shown in FIGS. 62-66. The DLS method measures particle size and electrokinetic potential (charge) of nano particles based on the diffraction of monochromatic light from a laser source. DLS measurements (either size or charge) may be analyzed as an average of the entire sample distribution or broken down into up to three distinct subpopulations per sample. Results are reported as either the average of the entire distribution (e.g., z-average), or as the average of the most dominant subpopulation (e.g., peak size, peak zeta-potential).

TABLE K
Stocks comprising excipients by relative concentration (% w:w)

| For- | |
|---|---|
| mula | Full composition |
| 1 | 40% Sucrose, 15% Trehalose, 20% Mannitol, 25% Dextran 40k |
| 2 | 20% Sucrose, 20% Trehalose, 50% Mannitol, 10% Dextran 40k |
| 3 | 50% Sucrose, 50% Mannitol |
| 4 | 40% Sucrose, 10% Trehalose, 50% Mannitol |
| 5 | 10% Trehalose, 70% Mannitol, 0.5% Sorbitol, 19.5% Dextran 40k |
| 6 | 20% Sucrose, 78% PVP-K30, 1% Citrate, 1% Arginine |
| 7 | 19.5% Trehalose, 80% Mannitol, 0.5% Sorbitol |
| 7a | 20% Trehalose, 80% Mannitol |
| 7e | 27% Sucrose, 20% Trehalose, 53% Mannitol |
| 8 | 10% Trehalose, 75% Mannitol, 15% Dextran 40k |
| 9 | 20% Sucrose, 10% Trehalose, 50% Mannitol, 20% PVP-K30 |
| 10 | 10% Sucrose, 10% Trehalose, 50% Mannitol, 30% B-Cyclodextrin |
| 11 | 95% Mannitol, 5% Poloxamer 188 |
| 12 | 90% Mannitol, 10% Poloxamer 188 |
| 13 | 20% Sucrose, 10% Trehalose, 50% Mannitol, 20% Ficoll |
| 14 | 20% Sucrose, 78% Ficoll, 1% Citrate, 1% Arginine |
| 15 | 19.5% Trehalose, 70% Mannitol, 0.5% Sorbitol, 10% Dextran 40k |
| 16 | 19.5% Trehalose, 75% Mannitol, 0.5% Sorbitol, 5% Dextran 40k |
| 17 | 20% Trehalose, 80% Mannitol |
| 18 | 10% Trehalose, 60% Mannitol, 30% Dextran 40k |
| 19 | 10% Trehalose, 30% Dextran 40k, 60% Maltodextrin |
| 20 | 100% Mannitol |
| 21 | 20% Mannitol, 60% PEG 6000, 20% Trehalose |
| 22 | 10% Mannitol, 60% PEG 6000, 30% Trehalose |
| 23 | 70% PEG 6000, 30% Trehalose |
| 24 | 70% PEG 6000, 30% Mannitol |

TABLE L
General conservative lyophilization cycle for EVs.

| | Ramp time | Hold time | Shelf temp | Vacuum |
|---|---|---|---|---|
| Step | (min) | (min) | (C.) | (mTorr) |
| Freezing | 200 | 360 | −45 | 100-300 |
| Primary drying | 75 | 5000 | −20 | 100-300 |
| Secondary drying | 180 | 1000 | 25 | 100-300 |
| Hold | 0 | N/A | 25 | 100-300 |

TABLE M
Particle count by NTA and moisture content by KF.

| | | EVs | Moisture | Moisture |
|---|---|---|---|---|
| Product | Formulation | (p/mg) | content (%) | STDEV (%) |
| F. | 1 | 1.50E+10 | 3.11 | 0.25 |
| massiliensis | 2 | 1.37E+10 | 3.68 | 0.10 |
| | 3 | 1.20E+10 | 2.77 | 0.23 |
| | 4 | 1.38E+10 | 3.26 | 0.06 |
| | 5 | 1.55E+10 | 5.14 | 0.80 |
| | 6 | 2.89E+10 | 7.01 | 0.24 |
| | 7 | 7.14E+09 | 3.09 | 0.28 |
| | 7a | 9.03E+09 | 2.62 | 0.21 |
| | 7e | 1.01E+10 | 3.17 | 0.23 |
| | 8 | 1.07E+10 | 3.46 | 0.47 |
| | 9 | 1.20E+10 | 3.48 | 0.18 |
| | 10 | 1.15E+10 | 3.45 | 0.13 |
| | 11 | 7.72E+09 | 1.57 | 0.09 |
| | 12 | 6.36E+09 | 1.51 | 0.15 |
| | 13 | 7.26E+09 | 2.68 | 0.22 |
| | 14 | 2.39E+10 | 4.70 | 0.14 |
| | 15 | 1.21E+10 | 4.22 | 0.09 |
| | 16 | 7.17E+09 | 3.31 | 0.07 |
| | 17 | 1.01E+10 | 3.00 | 0.10 |
| | 18 | 1.02E+10 | 4.06 | 0.18 |
| | 19 | 1.11E+10 | 6.41 | 0.24 |
| | 20 | 6.24E+09 | 2.11 | 0.14 |
| | 21 | 8.23E+09 | 3.08 | 0.10 |
| | 22 | 6.68E+09 | 4.52 | 0.37 |
| | 23 | 9.49E+09 | 3.99 | 0.09 |
| | 24 | 6.88E+09 | 2.45 | 0.16 |
| V. | 1 | 1.20E+10 | 4.25 | 0.14 |
| parvula | 2 | 1.25E+10 | 3.81 | 0.01 |
| | 3 | 1.22E+10 | 3.96 | 0.09 |
| | 4 | 1.25E+10 | 4.04 | 0.13 |
| | 5 | 1.55E+10 | 4.63 | 0.17 |
| | 6 | 1.03E+10 | 6.14 | 0.24 |
| | 7 | 1.35E+10 | 3.82 | 0.05 |
| | 7a | 8.90E+09 | 3.93 | 0.11 |
| | 7e | 1.16E+10 | 4.48 | 0.15 |
| | 8 | 1.28E+10 | 3.90 | 0.09 |
| | 9 | 1.28E+10 | 3.85 | 0.17 |
| | 10 | 1.38E+10 | 4.68 | 0.19 |
| | 11 | 5.00E+09 | 1.65 | 0.03 |
| | 12 | 1.29E+10 | 1.63 | 0.10 |
| | 13 | 1.36E+10 | 4.99 | 0.07 |
| | 14 | 9.41E+09 | 5.70 | 0.11 |
| | 15 | 1.82E+10 | 5.51 | 0.22 |
| | 16 | 1.44E+10 | 4.35 | 0.11 |
| | 17 | 1.19E+10 | 3.79 | 0.06 |
| | 18 | 1.16E+10 | 5.24 | 0.14 |
| | 19 | 1.26E+10 | 6.35 | 0.18 |
| | 20 | 1.20E+10 | 1.24 | 0.05 |
| | 21 | 1.48E+10 | 2.54 | 0.02 |
| | 22 | 1.47E+10 | 4.04 | 0.11 |
| | 23 | 1.43E+10 | 3.50 | 0.10 |
| | 24 | 1.05E+10 | 1.36 | 0.04 |

TABLE N
Particle size distribution determined by DLS, including
average size of the distribution and the size of
the dominant subpopulation (peak size).

| | | | | Z-Average |
|---|---|---|---|---|
| | | Z-Average | Peak Size | STDEV |
| Product | Formulation | (d · nm) | (d · nm) | (d · nm) |
| F. | 1 | 133.4 | 59.33 | 24.39 |
| massiliensis | 2 | 149.6 | 56.59 | 24.97 |
| | 3 | 147.6 | 55.28 | 23.82 |
| | 4 | 143.2 | 45.4 | 18.99 |
| | 5 | 147.4 | 65.59 | 31.95 |
| | 6 | 140.7 | 60.67 | 26.97 |
| | 7 | 148.5 | 59.39 | 27.02 |
| | 7a | 143.1 | 53.71 | 23.96 |
| | 7e | 150.3 | 66 | 29.59 |
| | 8 | 164.4 | 79.18 | 34.44 |
| | 9 | 132 | 67.02 | 28.08 |
| | 10 | 145.3 | 60.07 | 26.34 |
| | 11 | 138.3 | 61.48 | 27.43 |
| | 12 | 143 | 56.63 | 26.35 |
| | 13 | 147 | 71.69 | 29.38 |
| | 14 | 174.8 | 54.05 | 10.88 |
| | 15 | 151.3 | 62.56 | 28.4 |
| | 16 | 255 | 69.87 | 14.65 |
| | 17 | 147.6 | 63.17 | 27.65 |
| | 18 | 160.9 | 43.72 | 20.27 |
| | 19 | 241.4 | 65.81 | 16.77 |
| | 20 | 315.2 | 65.31 | 15.49 |
| | 21 | 139 | 68.01 | 28.52 |
| | 22 | 138.4 | 55.27 | 23.2 |
| | 23 | 158.3 | 62.19 | 28.73 |
| | 24 | 145.3 | 73.61 | 31.35 |
| V. | 1 | 171.1 | 61.82 | 32.54 |
| parvula | 2 | 276.9 | 51.61 | 35.79 |
| | 3 | 228.5 | 70.14 | 13.89 |
| | 4 | 323.5 | 55.31 | 47.68 |
| | 5 | 283.4 | 61.09 | 43.02 |
| | 6 | 221.5 | 62.76 | 11.98 |
| | 7 | 298.2 | 75.6 | 15.35 |
| | 7a | 172.7 | 45.96 | 21.03 |
| | 7e | 256.9 | 67.23 | 11.87 |
| | 8 | 165.3 | 46.22 | 21.75 |
| | 9 | 164.8 | 51.42 | 24.91 |
| | 10 | 165.7 | 53.35 | 25.27 |
| | 11 | 162 | 74.26 | 38.15 |
| | 12 | 171.4 | 53.28 | 24.73 |
| | 13 | 177.5 | 60.84 | 12.15 |
| | 14 | 130.4 | 40 | 9.135 |
| | 15 | 213.2 | 73.21 | 16.04 |
| | 16 | 179.8 | 62.46 | 14.69 |
| | 17 | 233 | 73.76 | 15.5 |
| | 18 | 197.4 | 54.58 | 10.98 |
| | 19 | 276.5 | 75.07 | 15.59 |
| | 20 | 176.9 | 69.06 | 13.05 |
| | 21 | 167.1 | 54.38 | 10.14 |
| | 22 | 171.3 | 78.8 | 34.64 |
| | 23 | 205.5 | 62.22 | 12.52 |
| | 24 | 229.2 | 60.13 | 11.38 |

TABLE O
Electrokinetic potential of the dominant
subpopulation determined by DLS.

| | | Peak Zeta | Zeta |
|---|---|---|---|
| | | Potential | Deviation |
| Product | Formulation | (mV) | (mV) |
| F. | 1 | −31.5 | 8.57 |
| massiliensis | 2 | −32 | 7.47 |
| | 3 | −28.9 | 8.53 |
| | 4 | −30.7 | 7.07 |
| | 5 | −27 | 8.46 |
| | 6 | −28.6 | 8.81 |
| | 7 | −26.2 | 10.2 |
| | 7a | −29.5 | 7.78 |
| | 7e | −28.6 | 9.88 |
| | 8 | −26.8 | 9.98 |
| | 9 | −28.6 | 7.76 |
| | 10 | −28.5 | 6.67 |
| | 11 | −27.1 | 11.1 |
| | 12 | −25.8 | 9.74 |
| | 13 | −27.5 | 9.4 |
| | 14 | −27.4 | 10.8 |
| | 15 | −27.7 | 8.6 |
| | 16 | −25.7 | 7.48 |
| | 17 | −27.8 | 7.59 |
| | 18 | −25.3 | 7.42 |
| | 19 | −28 | 12 |
| | 20 | −27 | 7.37 |
| | 21 | −27.4 | 8.36 |
| | 22 | −26.7 | 7.76 |
| | 23 | −28 | 7.03 |
| | 24 | −27.9 | 8.57 |
| V. | 1 | −12.6 | 7.85 |
| parvula | 2 | −7.95 | 5.74 |
| | 3 | −12.8 | 8.5 |
| | 4 | −13.5 | 8.29 |
| | 5 | −11.9 | 8 |
| | 6 | −8.24 | 6.69 |
| | 7 | −8.02 | 6.96 |
| | 7a | −8.31 | 7.09 |
| | 7e | −9.3 | 9.84 |
| | 8 | −11 | 8.6 |
| | 9 | −8.28 | 8.08 |
| | 10 | −7.81 | 8.89 |
| | 11 | −8.09 | 7.75 |
| | 12 | −12.4 | 8.25 |
| | 13 | −7.64 | 8.07 |
| | 14 | −8.34 | 7.85 |
| | 15 | −7.91 | 8.85 |
| | 16 | −11.2 | 8.89 |
| | 17 | −8.07 | 7.27 |
| | 18 | −12.6 | 9.36 |
| | 19 | −8.37 | 7.32 |
| | 20 | −7.54 | 8.95 |
| | 21 | −9.04 | 6.84 |
| | 22 | 13.1 | 7.64 |
| | 23 | −12.6 | 7.79 |
| | 24 | −9.37 | 5.92 |

### Example 12: Purification and Preparation of Membranes from Bacteria to Obtain Processed Microbial Extracellular Vesicles (pmEVs)

### Purification

Processed microbial extracellular vesicles (pmEVs) are purified and prepared from bacterial cultures (e.g., bacteria listed in Table 1, Table 2, and/or Table 3) using methods known to those skilled in the art (Thein et al. (2010) J. Proteome Res., 9(12): 6135-47; Sandrini et al. (2014) Bio-Protocol. 4(21) doi: 10.21769/BioProtoc.1287).

Alternatively, pmEVs are purified by methods adapted from Thein et al. For example, bacterial cultures are centrifuged at 10,000-15,500×g for 10-30 minutes at room temperature or at 4° C. Supernatants are discarded and cell pellets are frozen at −80° C. Cell pellets are thawed on ice and resuspended in 100 mM Tris-HCl, pH 7.5, and may be supplemented with 1 mg/mL DNase I and/or 100 mM NaCl. Thawed cells are incubated in 500 μg/ml lysozyme, 40 μg/ml lyostaphin, and/or 1 mg/ml DNaseI for 40 minutes to facilitate cell lysis. Additional enzymes may be used to facilitate the lysing process (e.g., EDTA (5 mM), PMSF (Sigma Aldrich), and/or benzamidine (Sigma Aldrich). Cells are then lysed using an Emulsiflex C-3 (Avestin, Inc.) under conditions recommended by the manufacturer. Alternatively, pellets may be frozen at −80° C. and thawed again prior to lysis. Debris and unlysed cells are pelleted by centrifugation at 10,000-12,500×g for 15 minutes at 4° C. Supernatants are then centrifuged at 120,000×g for 1 hour at 4° C. Pellets are resuspended in ice-cold 100 mM sodium carbonate, pH 11, incubated with agitation for 1 hour at 4° C. Alternatively, pellets are centrifuged at 120,000×g for 1 hour at 4° C. in sodium carbonate immediately following resuspension. Pellets are resuspended in 100 mM Tris-HCl, pH 7.5 supplemented with 100 mM NaCl re-centrifuged at 120,000×g for 20 minutes at 4° C., and then resuspended in 100 mM Tris-HCl, pH 7.5 supplemented with up to or around 100 mM NaCl or in PBS. Samples are stored at −20° C. To protect the pmEV preparation during the freeze/thaw steps, 250 mM sucrose and up to 500 mM NaCl may be added to the final preparation to stabilize the vesicles in the pmEV preparation.

Alternatively, pmEVs are obtained by methods adapted from Sandrini et al, 2014. After, bacterial cultures are centrifuged at 10,000-15,500×g for 10-15 minutes at room temperature or at 4° C., cell pellets are frozen at −80° C. and supernatants are discarded. Then, cell pellets are thawed on ice and resuspended in 10 mM Tris-HCl, pH 8.0, 1 mM EDTA supplemented with 0.1 mg/mL lysozyme. Samples are then incubated with mixing at room temperature or at 37° C. for 30 min. In an optional step, samples are re-frozen at −80° C. and thawed again on ice. DNase I is added to a final concentration of 1.6 mg/mL and MgCl₂ to a final concentration of 100 mM. Samples are sonicated using a QSonica Q500 sonicator with 7 cycles of 30 sec on and 30 sec off. Debris and unlysed cells are pelleted by centrifugation at 10,000×g for 15 min. at 4° C. Supernatants are then centrifuged at 110,000×g for 15 minutes at 4° C. Pellets are resuspended in 10 mM Tris-HCl, pH 8.0 and incubated 30-60 minutes with mixing at room temperature. Samples are centrifuged at 110,000×g for 15 minutes at 4° C. Pellets are resuspended in PBS and stored at −20° C.

Optionally, pmEVs can be separated from other bacterial components and debris using methods known in the art. Size-exclusion chromatography or fast protein liquid chromatography (FPLC) may be used for pmEV purification. Additional separation methods that could be used include field flow fractionation, microfluidic filtering, contact-free sorting, and/or immunoaffinity enrichment chromatography. Alternatively, high resolution density gradient fractionation could be used to separate pmEV particles based on density.

### Preparation

Bacterial cultures are centrifuged at 10,000-15,500×g for 10-30 minutes at room temperature or at 4° C. Supernatants are discarded and cell pellets are frozen at −80° C. Cell pellets are thawed on ice and resuspended in 100 mM Tris-HCl, pH 7.5, 100 mM NaCl, 500 μg/ml lysozyme and/or 40 μg/ml Lysostaphin to facilitate cell lysis; up to 0.5 mg/ml DNaseI to reduce genomic DNA size, and EDTA (5 mM), PMSF (1 mM, Sigma Aldrich), and Benzamidine (1 mM, Sigma Aldrich) to inhibit proteases. Cells are then lysed using an Emulsiflex C-3 (Avestin, Inc.) under conditions recommended by the manufacturer. Alternatively, pellets may be frozen at −80° C. and thawed again prior to lysis. Debris and unlysed are pelleted by centrifugation at 10,000-12,500×g at for 15 minutes at 4° C. Supernatants are subjected to size exclusion chromatography (Sepharose 4 FF, GE Healthcare) using an FPLC instrument (AKTA Pure 150, GE Healthcare) with PBS and running buffer supplemented with up to 0.3 M NaCl. Pure pmEVs are collected in the column void volume, concentrated and stored at −20° C. Concentration may be performed by a number of methods. For example, ultra-centrifugation may be used (140,000×g, 1 hour, 4° C., followed by resuspension in small volume of PBS). To protect the pmEV preparation during the freeze-thaw steps, 250 mM sucrose and up to 500 mM NaCl may be added to the final preparation to stabilize the vesicles in the pmEV preparation. Additional separation methods that could be used include field flow fractionation, microfluidic filtering, contact-free sorting, and/or immunoaffinity enrichment chromatography. Other techniques that may be employed using methods known in the arts include Whipped Film Evaporation, Molecular Distillation, Short Pass Distillation, and/or Tangential Flow Filtration.

In some instances, pmEVs are weighed and are administered at varying doses (in μg/ml). Optionally, pmEVs are assessed for particle count and size distribution using Nanoparticle Tracking Analysis (NTA), using methods known in the art. For example, a Malvern NS300 instrument may be used according to manufacturer's instructions or as described by Bachurski et al. 2019. Journal of Extracellular Vesicles. Vol. 8(1). Alternatively, for the pmEVs, total protein may be measured using Bio-rad assays (Cat #5000205) performed per manufacturer's instructions and administered at varying doses based on protein content/dose.

For the studies described herein, the pmEVs may be irradiated, heated, and/or lyophilized prior to administration.

pmEVs can be lyophilized using the excipients and drying conditions described herein to produce a lyophilate or powder.

### Example 13: Spray-Dried Powders of Prevotella histicola smEVs

In this Example, the extracellular vesicles (smEVs) used in the studies were isolated from Prevotella histicola Strain B.

The smEVs were spray dried as follows:

EV retentate was mixed with one of the excipients provided in Table P.

TABLE P
Stocks comprising excipients by relative concentration (% w:w)

| Formula | Full composition |
|---|---|
| 7a | 80% Mannitol, 20% Trehalose |
| 25 | 100% Trehalose |
| 26 | Maltodextrin-Trehalose (20:60:20) |
| 27 | Maltodextrin-Trehalose (70:30) |
| 28 | PEG6000-Trehalose (70:30) |
| 29 | Mannitol- Maltodextrin -Trehalose (20:60:20) |

The spray drying was performed at 100° C., or 130° C. Temperatures are also included in Table Q.

Post spray drying, each powder was analyzed for moisture content (MC) (by Karl Fischer titration (KF)) and particles (particles/mg spray-dried powder (p/mg)) (quantification by nanoparticle tracking analysis (NTA) using Zetaview). Results are shown in Table Q. EXP7A is stock of formula 7a.

TABLE Q
Moisture content and particle content of spray dried samples

| | Inlet | Moisture | |
|---|---|---|---|
| | temperature | Content | Particles/ |
| Sample ID | (° C.) | (%) | mg |
| EXP7A-130° C. inlet | 130 | 3.64 | 1.30E+10 |
| EXP7A-100° C. inlet | 100 | 2.54 | 1.25E+10 |
| Man-Malt-Tre (20:60:20) | 130 | 5.35 | 1.40E+10 |
| Malt-Tre (70:30) | 130 | 8.38 | 2.00E+10 |
| 100% Trehalose | 130 | 6.37 | 1.60E+10 |

Spray drying was also performed using a stock that consisted of PEG6000-Mannitol-Trehalose (60:20:20). However, reduced recovery of dried product relative to other methods described herein was obtained.

Prevotella histicola smEVs were spray dried or lyophilized in stock of formulation 7a (F7A) at two concentrations: 25× and 500×, with an inlet temperature of 130° C.

The comparison of particles/mg spray-dried powder and size are shown in Table R. SD=spray dried; L0.47=lyophilized; 0.47 refers to the stock ratio used: 47 g of excipient with every 100 g of retentate.

The particle packing and size of the spray dried and lyophilized EVs were similar with both methods of drying.

TABLE R

| Sample | Particles/mg | CV | Size (nm) |
|---|---|---|---|
| 25X L0.47 F7A | 3.35e+9 | 2.10% | 172.7 |
| 25X SD F7A | 3.95e+9 | 0.00% | 164.8 |
| 500X L0.47 F7A | 5.50e+10 | 0.00% | 165.1 |
| 500X SD F7A | 4.88e+10 | 0.70% | 163.8 |

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A dried form comprising extracellular vesicles (EVs) from bacteria, wherein the dried form has a moisture content of below about 6%.

2. The dried form of claim 1, wherein the dried form has a moisture content of below about 5%.

3. The dried form of claim 1, wherein the dried form has a moisture content of below about 4%.

4. The dried form of claim 1, wherein the dried form has a moisture content of between about 1% to about 4%.

5. The dried form of claim 1, wherein the dried form has a moisture content of between about 2% to about 3%.

6. The dried form of claim 1, wherein the dried form comprises a powder.

7. The dried form of claim 1, wherein the dried form comprises a lyophilate.

8. The dried form of claim 1, wherein the dried form comprises EVs from a bacterial strain that is associated with mucus.

9. The dried form of claim 1, wherein the dried form comprises EVs from anaerobic bacteria.

10. The dried form of claim 1, wherein the anaerobic bacteria are obligate anaerobes.

11. The dried form of claim 1, wherein the anaerobic bacteria are facultative anaerobes.

12. The dried form of claim 1, wherein the anaerobic bacteria are aerotolerant anaerobes.

13. The dried form of claim 1, wherein the dried form comprises EVs from monoderm bacteria.

14. The dried form of claim 1, wherein the dried form comprises EVs from diderm bacteria.

15. The dried form of claim 1, wherein the dried form comprises EVs from Gram negative bacteria.

16. The dried form of claim 1, wherein the dried form comprises EVs from bacteria of the Prevotellaceae; Veillonellaceae; Tannerellaceae; Rikenellaceae; Selenomonadaceae; Sporomusaceae; Synergistaceae; Christensenellaceae; or Akkermaniaceae family.

17. The dried form of claim 1, wherein the dried form comprises EVs from Gram positive bacteria.

18. The dried form of claim 1, wherein the dried form comprises EVs from bacteria of the Oscillospiraceae; Clostridiaceae; or Lachnospiraceae family.

19. The dried form of claim 1, wherein the dried form comprises EVs from bacteria of the genus Prevotella.

20. The dried form of claim 1, wherein the dried form comprises EVs from bacteria of the genus Veillonella.

21. The dried form of claim 1, wherein the dried form comprises EVs from bacteria of the genus Parabacteroides.

22. The dried form of claim 1, wherein the dried form comprises EVs from bacteria of the Oscillospiraceae family.

23. The dried form of claim 1, wherein the dried form comprises EVs from bacteria of the Tannerellaceae family.

24. The dried form of claim 1, wherein the dried form comprises EVs from bacteria of the Prevotellaceae family.

25. The dried form of claim 1, wherein the dried form comprises EVs from bacteria of the Veillonellaceae family.

26. A therapeutic composition comprising the dried form of any one of claims 1 to 25.
